(19) United States
(12) Patent Application Publication
(10) Pub. No.: US 2024/0324452 A1
(43) Pub. Date: Sep. 26, 2024

(54) PLURALITY OF HOST MATERIALS, ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Yea-Mi SONG, Gyeonggi-do (KR); Hyo-Soon PARK, Gyeonggi-do (KR); Jeong-Eun YANG, Gyeonggi-do (KR); Tae-Jun HAN, Gyeonggi-do (KR); Mi-Ja LEE, Gyeonggi-do (KR); Kyoung-Jin PARK, Gyeonggi-do (KR); Doo-Hyeon MOON, Gyeonggi-do (KR)

(21) Appl. No.: 18/436,429

(22) Filed: Feb. 8, 2024

(30) Foreign Application Priority Data
Mar. 13, 2023 (KR) 10-2023-0032551

(51) Int. Cl. H10K 85/60; C07B 59/00; C09K 11/02; H10K 50/12; H10K 85/30

(52) U.S. Cl. CPC H10K 85/654; C07B 59/004; C09K 11/02; H10K 50/12; H10K 85/342; H10K 85/615; H10K 85/622; H10K 85/6572; H10K 85/6574; H10K 85/6576; C07B 2200/05

(57) ABSTRACT

The present disclosure relates to a plurality of host materials comprising at least one first host compound and at least one second host compound, an organic electroluminescent compound, and an organic electroluminescent device comprising the same. It is possible to produce an organic electroluminescent device having low driving voltage, high luminous efficiency, and/or excellent lifetime properties, either by utilizing the plurality of host materials comprising a specific combination of host compounds, or by utilizing the organic electroluminescent compound represented by a specific Formula.

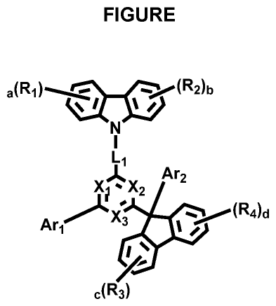

### TECHNICAL FIELD

The present disclosure relates to a plurality of host materials, an organic electroluminescent compound, and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, using small aromatic diamine molecules and aluminum complexes as materials for forming a light-emitting layer (see Appl. Phys. Lett. 51, 913, 1987).

An organic electroluminescent device has a multi-layer structure including a hole injection layer, a hole transport layer, a light-emitting layer, an electron transport layer, and an electron injection layer to increase efficiency and stability. The selection of compounds comprised in the light-emitting layer has been recognized as one of the means by which to improve the properties of the device, such as driving voltage, luminous efficiency, and lifetime.

Korean Patent Application Laid-Open No. 10-2022-0094124 discloses a plurality of host materials for the phosphorescent light-emitting layer, and an organic electroluminescent compound used in the light-emitting layer, etc. However, the aforementioned reference does not specifically disclose the plurality of host materials comprising a specific combination of compounds according to the present disclosure, nor does it disclose the organic electroluminescent compound represented by a specific formula according to the present disclosure. Therefore, the development of host materials and an organic electroluminescent compound to improve OLED performance is still required.

### DISCLOSURE OF INVENTION

### Technical Problem

The objective of the present disclosure is to provide a plurality of host materials and an organic electroluminescent compound that can provide an organic electroluminescent device improved driving voltage, luminous efficiency and/or lifetime properties.

### Solution to Problem

As a result of intensive studies conducted to solve the above technical problems, the present inventors have found that the above objective can be achieved by an organic electroluminescent compound represented by the following Formula 1, and a plurality of host materials comprising a first host compound comprising the compound represented by the following Formula 1 and a second host compound comprising the compound represented by the following Formula 2.

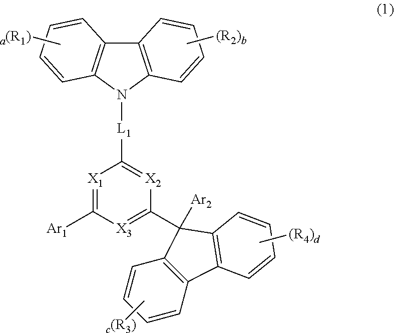

In Formula 1,
X₁ to X₃, each independently, represent N or CRₐ, with a proviso that at least two of X₁ to X₃ represent N;
L₁ represents a single bond, or a substituted or unsubstituted (C6-C30)arylene;
Rₐ, and R₁ to R₄, each independently, represent hydrogen, deuterium, a halogen, a cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di- (C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
Ar₁ and Ar₂, each independently, represent hydrogen, deuterium, a substituted or unsubstituted (C6-C30)aryl, or a substituted or unsubstituted (3- to 30-membered)heteroaryl;
a to d, each independently, represent an integer of 1 to 4; and
if a to d represent an integer of 2 or more, each of R₁ to each of R₄ may be the same as or different from each other.

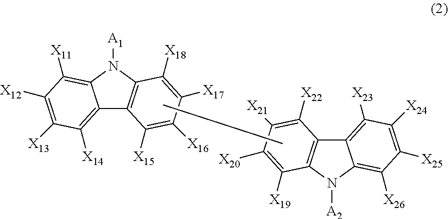

In Formula 2,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;
one of X₁₅ to X₁₈ links to one of X₁₉ to X₂₂ to form a single bond; and
X₁₁ to X₁₄, X₂₃ to X₂₆, and X₁₅ to X₂₂ not forming a single bond, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s).

### Advantageous Effects of Invention

An organic electroluminescent device having low driving voltage, high luminous efficiency, and/or excellent lifetime properties can be achieved by comprising the plurality of host materials or the organic electroluminescent compound of the present disclosure.

### BRIEF DESCRIPTION OF THE FIGURE

The Figure illustrates a representative formula for the organic electroluminescent compound according to the present disclosure.

### MODE FOR THE INVENTION

Hereinafter, the present disclosure will be described in detail. However, the following description is intended to explain the present disclosure, and is not meant in any way to restrict the scope of the present disclosure.

The present disclosure relates to a plurality of host materials comprising at least one first host compound and at least one second host compound, an organic electroluminescent compound, and an organic electroluminescent device comprising the same.

The term “organic electroluminescent compound” in the present disclosure means a compound that may be used in an organic electroluminescent device, and may be comprised in any layer constituting an organic electroluminescent device, as necessary. For example, the organic electroluminescent compound may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc. The hole transfer zone material may be at least one selected from the group consisting of a hole transport material, a hole injection material, an electron blocking material, a hole auxiliary material and a light-emitting auxiliary material.

The term “a plurality of organic electroluminescent materials” in the present disclosure means an organic electroluminescent material that is a combination of at least two compounds, which may be comprised in any layer constituting an organic electroluminescent device. It may mean both a material before being comprised in an organic electroluminescent device (e.g. before vapor deposition) and a material after being comprised in an organic electroluminescent device (e.g. after vapor deposition).

For example, the plurality of organic electroluminescent materials of the present disclosure may be a combination of at least two compounds, which may be comprised in at least one layer(s) of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The organic electroluminescent material that is a combination of at least two of these compounds may be comprised in the same layer or different layers, and may be mixture-deposited, co-deposited, or separately deposited.

The term “a plurality of host materials” in the present disclosure means organic electroluminescent materials which are a combination of at least two host materials. It may mean both materials before being comprised in an organic electroluminescent device (e.g. before vapor deposition) and materials after being comprised in an organic electroluminescent device (e.g. after vapor deposition). The plurality of host materials of the present disclosure may be comprised in any light-emitting layer constituting an organic electroluminescent device. In addition, at least two compounds comprised in the plurality of host materials may be comprised together in one light-emitting layer or may respectively be comprised in different light-emitting layers. When at least two host materials are comprised in one layer, for example, they may be mixture-deposited to form a layer, or they may be co-deposited separately and simultaneously to form a layer.

Herein, the term “(C1-C30)alkyl” is meant to be a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc.

Herein, the term “(C3-C30)cycloalkyl” is meant to be a mono- or polycyclic hydrocarbon having 3 to 30 ring backbone carbon atoms. According to one embodiment of the present disclosure, the number of ring backbone carbon atoms may be 3 to 20, and according to another embodiment of the present disclosure, the number of ring backbone carbon atoms may be 3 to 7. The above cycloalkyl may include cyclopropyl, cyclobutyl, cyclopentyl, cyclohexyl, cyclopentylmethyl, cyclohexylmethyl, etc.

Herein, the term “(3- to 7-membered)heterocycloalkyl” is meant to be a cycloalkyl having 3 to 7 ring backbone atoms and containing at least one heteroatom. For example, the number of ring backbone atoms may be 5 to 7. According to one embodiment of the present disclosure, the heteroatom may be at least one selected from the group consisting of B, N, O, S, Si, and P, and according to another embodiment of the present disclosure, the heteroatom may be at least one selected from the group consisting of O, S, and N. The above heterocycloalkyl may include tetrahydrofuran, pyrrolidine, thiolan, tetrahydropyran, etc.

Herein, the term “(C6-C30)aryl(ene)” is meant to be a monocyclic or fused ring-type radical derived from an aromatic hydrocarbon having 6 to 30 ring backbone carbon atoms that can be partially saturated. According to one embodiment of the present disclosure, the number of ring backbone carbon atoms may be 6 to 20, and according to another embodiment of the present disclosure, the number of ring backbone carbon atoms may be 6 to 15. The above aryl may comprise a spiro structure. The above aryl(ene) may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzoanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluoren]yl, spiro[fluorene-benzofluoren]yl, azulenyl, tetramethyl-dihydrophenanthrenyl, etc. Specifically, the above aryl may include o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the term “(3- to 30-membered)heteroaryl(ene)” is meant to be an aryl group having 3 to 30 ring backbone atoms, and comprising at least one heteroatom selected from the group consisting of B, N, O, S, Si, P, Se and Ge. According to one embodiment of the present disclosure, the number of ring backbone atoms may be 3 to 30 and according to another embodiment of the present disclosure, the number of ring backbone atoms may be 5 to 20. According to one embodiment of the present disclosure, the number of the heteroatoms may be 1 to 4. The above heteroaryl or heteroarylene may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc.

Herein, “a fused ring group of a (C3-C30)aliphatic ring(s) and a (C6-C30)aromatic ring(s)” is meant to be a functional group of a ring in which at least one aliphatic ring having 3 to 30 ring backbone carbon atoms is fused with at least one aromatic ring having 6 to 30 ring backbone carbon atoms. According to one embodiment of the present disclosure, the (C3-C30) aliphatic ring may have 3 to 25 ring backbone carbon atoms, and according to another embodiment of the present disclosure, it may have 3 to 18 ring backbone carbon atoms. According to one embodiment of the present disclosure, the (C6-C30) aromatic ring may have 6 to 25 ring backbone carbon atoms, and according to another embodiment of the present disclosure, it may have 6 to 18 ring backbone carbon atoms. Specific examples of the fused ring group include a fused ring group of one or more benzene and one or more cyclohexane, or a fused ring group of one or more naphthalene and one or more cyclopentane, etc. Herein, the carbon atom of the fused ring group of a (C3-C30)aliphatic ring(s) and a (C6-C30)aromatic ring(s) may be replaced with one or more heteroatoms selected from B, N, O, S, Si, and P, and for example, one or more heteroatoms selected from N, O, and S.

Herein, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other; for example, when two substituents in a benzene derivative occupy positions 1 and 2, it is called an ortho position. Meta indicates that two substituents are at positions 1 and 3; for example, when two substituents in a benzene derivative occupy positions 1 and 3, it is called a meta position. Para indicates that two substituents are at positions 1 and 4; for example, when two substituents in a benzene derivative occupy positions 1 and 4, it is called a para position.

Herein, “a ring formed by being linked to an adjacent substituent(s)” means that at least two adjacent substituents are linked to or fused with each other to form a substituted or unsubstituted, mono- or polycyclic, (3- to 30-membered) alicyclic or aromatic ring, or a combination thereof. For example, the ring may be a substituted or unsubstituted, mono- or polycyclic, (5- to 25-membered) alicyclic or aromatic ring, or a combination thereof. According to one embodiment of the present disclosure, the number of the ring backbone atoms may be 5 to 20-membered, and according to another embodiment of the present disclosure, the number of the ring backbone atoms may be 5 to 15-membered. In addition, the ring may contain at least one heteroatom selected from B, N, O, S, Si, and P, and for example at least one heteroatom selected from N, O, and S. For example, the ring may be, a substituted or unsubstituted, dibenzothiophene ring, dibenzofuran ring, naphthalene ring, phenanthrene ring, fluorene ring, benzofluorene ring, benzothiophene ring, benzofuran ring, indole ring, indene ring, benzene ring, or carbazole ring, etc.

Herein, “substituted” in the expression “substituted or unsubstituted” means that a hydrogen atom in a certain functional group is replaced with another atom or another functional group, i.e., a substituent. The substituent also includes those in which two or more substituents are linked. For example, the substituent formed by linking two or more substituents may be pyridine-triazine. That is, pyridine-triazine may be interpreted as a heteroaryl, or as a substituent(s) with two heteroaryls linked.

Herein, the substituent(s) of the substituted (C1-C30)alkyl, the substituted (C2-C30)alkenyl, the substituted (C6-C30)aryl, the substituted (3- to 30-membered)heteroaryl, the substituted (C3-C30)cycloalkyl, the substituted unsubstituted (C3-C30)cycloalkenyl, the substituted (3- to 7-membered)heterocycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted fused ring group of a (C3-C30)aliphatic ring(s) and a (C6-C30)aromatic ring(s), the substituted mono- or di- (C1-C30)alkylamino, the substituted mono- or di- (C2-C30)alkenylamino, the substituted mono- or di- (C6-C30)arylamino, the substituted mono- or di- (3- to 30-membered)heteroarylamino, the substituted (C1-C30)alkyl(C2-C30)alkenylamino, the substituted (C1-C30)alkyl(C6-C30)arylamino, the substituted (C1-C30)alkyl(3- to 30-membered)heteroarylamino, the substituted (C2-C30)alkenyl(C6-C30)arylamino, the substituted (C2-C30)alkenyl(3- to 30-membered)heteroarylamino, or the substituted (C6-C30)aryl(3- to 30-membered)heteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of deuterium, a halogen, a cyano, a (C1-C30)alkyl(s), a (C6-C30)aryl(s), a (3- to 30-membered)heteroaryl(s) and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di- (C1-C30)alkylamino; a mono- or di- (C2-C30)alkenylamino; a mono- or di- (C6-C30)arylamino; a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a (C6-C30)arylphosphine; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. For example, the substituent(s), each independently, may be at least one selected from the group consisting of deuterium, a phenyl, a biphenyl, a dibenzofurany, a dibenzothiophenyl, a carbazolyl, and a benzocarbazolyl.

Herein, “deuteration” means that the hydrogen at the replaceable position of the compound is replaced with deuterium, and “x % as substitution rate of deuterium” means that x % of the hydrogen at the replaceable position of the compound is replaced with deuterium. For example, the substitution rate of deuterium in naphthalene is 25%, which means that two out of eight hydrogens at replaceable positions in naphthalene are replaced with deuterium.

In the formula according to the present disclosure, when there are a plurality of substituents represented by the same symbol, each substituent represented by the same symbol may be identical to or different from each other.

Hereinafter, a plurality of host materials, an organic electroluminescent compound, and an organic electroluminescent device according to the present disclosure, will be described.

A plurality of host materials according to the present disclosure comprises at least one first host compound and at least one second host compound.

The first host compound is represented by the following Formula 1.

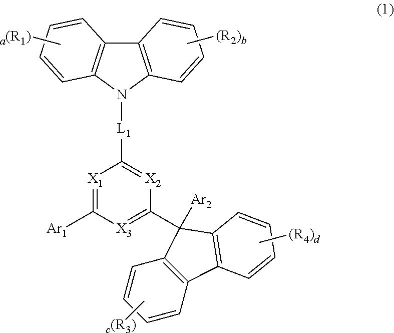

In Formula 1, X₁ to X₃, each independently, represent N or CRₐ, with a proviso that at least two of X₁ to X₃ represent N.

In Formula 1, L₁ represents a single bond, or a substituted or unsubstituted (C6-C30)arylene. For example, L₁ may be a single bond, or a phenylene.

In Formula 1, Rₐ, and R₁ to R₄, each independently, represent hydrogen, deuterium, a halogen, a cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di- (C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino. According to one embodiment of the present disclosure, Rₐ, and R₁ to R₄, each independently, may represent hydrogen, deuterium, a substituted or unsubstituted (C6-C24)aryl, a substituted or unsubstituted (5- to 18-membered)heteroaryl, or a combination thereof. According to another embodiment of the present disclosure, Rₐ, and R₁ to R₄, each independently, represent hydrogen, deuterium, a phenyl unsubstituted or substituted with deuterium, a biphenyl unsubstituted or substituted with deuterium, a phenanthrenyl unsubstituted or substituted with deuterium, a terphenyl unsubstituted or substituted with deuterium, a quaterphenyl unsubstituted or substituted with deuterium, a naphthyl unsubstituted or substituted with deuterium, a phenylnaphthyl unsubstituted or substituted with deuterium, a naphthylphenyl unsubstituted or substituted with deuterium, a dibenzofuranyl unsubstituted or substituted with deuterium, a carbazolyl unsubstituted or substituted with deuterium, a dibenzothiophenyl unsubstituted or substituted with deuterium, or a combination thereof. For example, Rₐ, and R₁ to R₄, each independently, may be hydrogen, deuterium, a phenyl unsubstituted or substituted with deuterium, a p-biphenyl unsubstituted or substituted with deuterium, a m-biphenyl unsubstituted or substituted with deuterium, a o-biphenyl unsubstituted or substituted with deuterium, or a combination thereof.

In Formula 1, a to d, each independently, represent an integer of 1 to 4. If a to d represent an integer of 2 or more, each of R₁ to each of R₄ may be the same as or different from each other.

In Formula 1, Ar₁ and Ar₂, each independently, represent hydrogen, deuterium, a substituted or unsubstituted (C6-C30)aryl, or a substituted or unsubstituted (3- to 30-membered)heteroaryl. According to one embodiment of the present disclosure, Ar and Ar₂, each independently, may represent hydrogen, deuterium, a substituted or unsubstituted (C6-C20)aryl, or a substituted or unsubstituted (5- to 18-membered)heteroaryl. According to another embodiment of the present disclosure, Ar₁ and Ar₂, each independently, represent a phenyl unsubstituted or substituted with deuterium, a biphenyl unsubstituted or substituted with deuterium, a terphenyl unsubstituted or substituted with deuterium, a naphthyl unsubstituted or substituted with deuterium, a dibenzofuranyl unsubstituted or substituted with deuterium, a dibenzothiophenyl unsubstituted or substituted with deuterium, a carbazolyl unsubstituted or substituted with deuterium, or a combination thereof. For example, Ar₁ and Ar₂, each independently, may be a phenyl unsubstituted or substituted with at least one of deuterium, a dibenzofuranyl(s) and a dibenzothiophenyl(s); a p-biphenyl; a m-biphenyl; an o-biphenyl; a p-terphenyl; a m-terphenyl; an o-terphenyl; a naphthyl; a dibenzofuranyl unsubstituted or substituted with deuterium; a dibenzothiophenyl; and a phenylcarbazolyl.

According to one embodiment of the present disclosure, the compound represented by Formula 1 may have a substitution rate of deuterium of 15% to 100%, according to another embodiment, 25% to 100%, according to another embodiment, 45% to 100%, and according to another embodiment, 75% to 100%. The upper limit of the substitution rate of deuterium may be 100%, or may be less than 100%, for example, about 99%. That is, the compound represented by Formula 1 may be a compound in which all of the hydrogen at replaceable positions is replaced with deuterium, or it may be a compound in which the hydrogen at replaceable positions is partially replaced with deuterium.

When the compound represented by Formula 1 contains deuterium substituted according to the above-mentioned rate, the stability of the compound represented by Formula 1 may be improved as the bond dissociation energy due to deuteration increases. An organic electroluminescent device comprising the compound represented by Formula 1 may exhibit improved lifetime properties.

The compound represented by Formula 1 may be selected from the group consisting of the following compounds, but is not limited thereto.

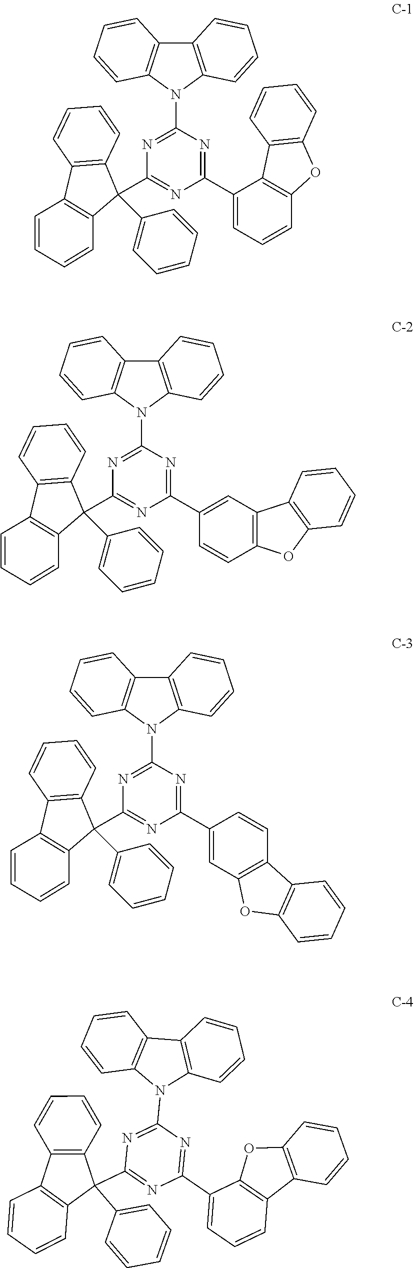 [SMILES: c1ccc(C2(c3nc(-c4cccc5oc6ccccc6c45)nc(-n4c5ccccc5c5ccccc54)n3)c3ccccc3-c3ccccc32)cc1 ; c1ccc(C2(c3nc(-c4ccc5oc6ccccc6c5c4)nc(-n4c5ccccc5c5ccccc54)n3)c3ccccc3-c3ccccc32)cc1 ; c1ccc(C2(c3nc(-c4ccc5c(c4)oc4ccccc45)nc(-n4c5ccccc5c5ccccc54)n3)c3ccccc3-c3ccccc32)cc1 ; c1ccc(C2(c3nc(-c4cccc5c4oc4ccccc45)nc(-n4c5ccccc5c5ccccc54)n3)c3ccccc3-c3ccccc32)cc1]

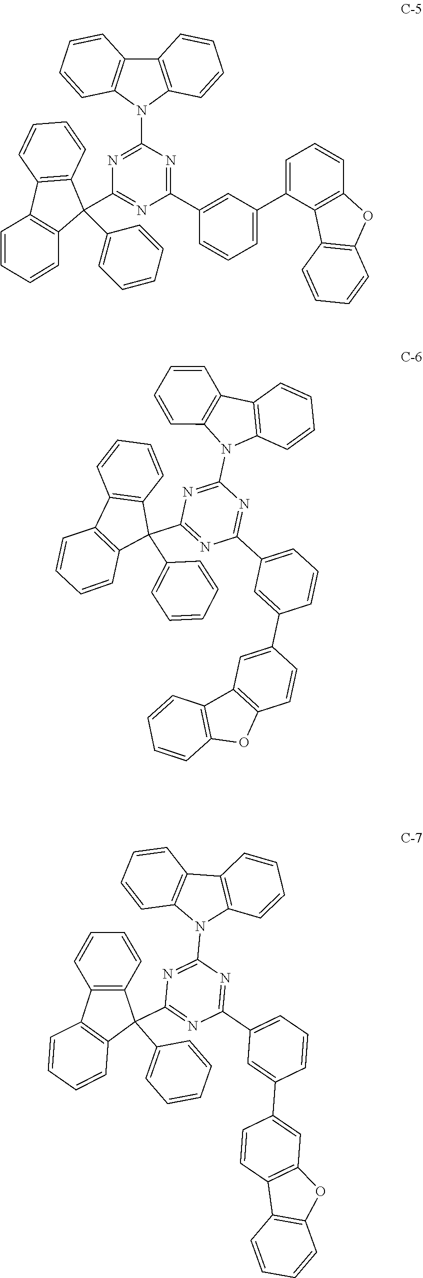 [SMILES: c1ccc(C2(c3nc(-c4cccc(-c5cccc6oc7ccccc7c56)c4)nc(-n4c5ccccc5c5ccccc54)n3)c3ccccc3-c3ccccc32)cc1 ; c1ccc(C2(c3nc(-c4cccc(-c5ccc6oc7ccccc7c6c5)c4)nc(-n4c5ccccc5c5ccccc54)n3)c3ccccc3-c3ccccc32)cc1 ; c1ccc(C2(c3nc(-c4cccc(-c5ccc6c(c5)oc5ccccc56)c4)nc(-n4c5ccccc5c5ccccc54)n3)c3ccccc3-c3ccccc32)cc1]

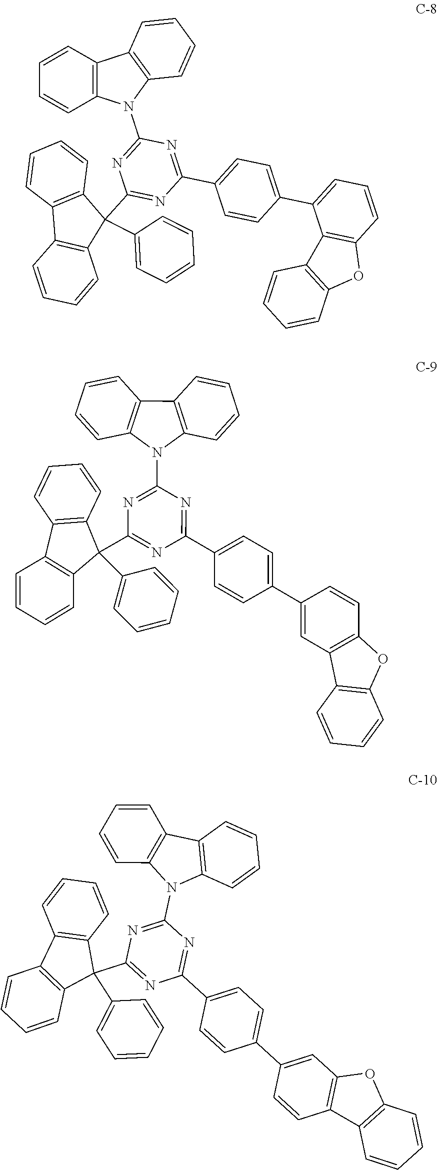 [SMILES: c1ccc(C2(c3nc(-c4ccc(-c5cccc6oc7ccccc7c56)cc4)nc(-n4c5ccccc5c5ccccc54)n3)c3ccccc3-c3ccccc32)cc1 ; c1ccc(C2(c3nc(-c4ccc(-c5ccc6oc7ccccc7c6c5)cc4)nc(-n4c5ccccc5c5ccccc54)n3)c3ccccc3-c3ccccc32)cc1 ; c1ccc(C2(c3nc(-c4ccc(-c5ccc6c(c5)oc5ccccc56)cc4)nc(-n4c5ccccc5c5ccccc54)n3)c3ccccc3-c3ccccc32)cc1]

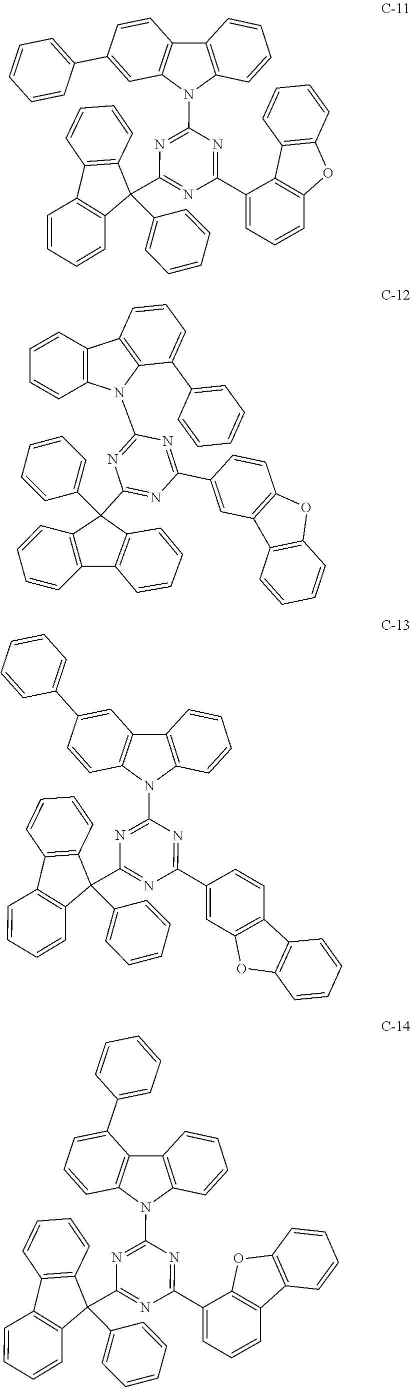

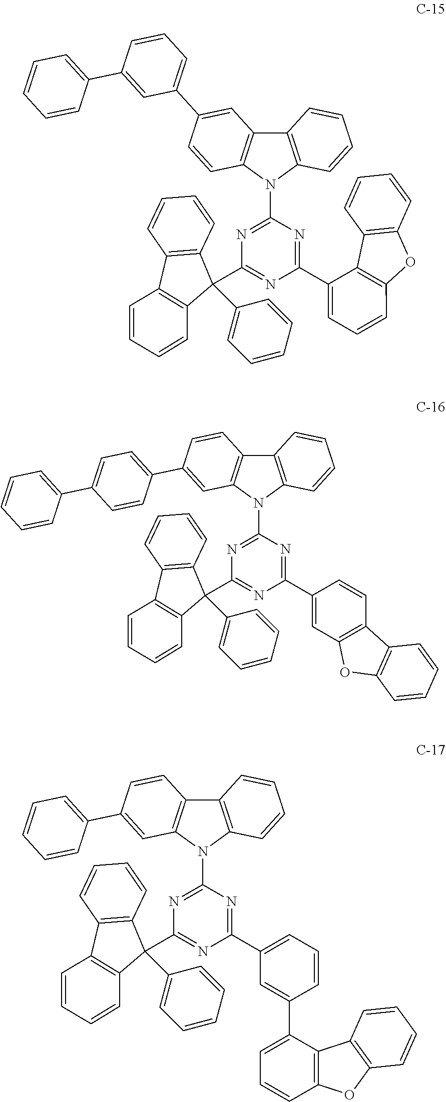 [SMILES: c1ccc(-c2cccc(-c3ccc4c(c3)c3ccccc3n4-c3nc(-c4cccc5oc6ccccc6c45)nc(C4(c5ccccc5)c5ccccc5-c5ccccc54)n3)c2)cc1 ; c1ccc(-c2ccc(-c3ccc4c5ccccc5n(-c5nc(-c6ccc7c(c6)oc6ccccc67)nc(C6(c7ccccc7)c7ccccc7-c7ccccc76)n5)c4c3)cc2)cc1 ; c1ccc(-c2ccc3c4ccccc4n(-c4nc(-c5cccc(-c6cccc7oc8ccccc8c67)c5)nc(C5(c6ccccc6)c6ccccc6-c6ccccc65)n4)c3c2)cc1]

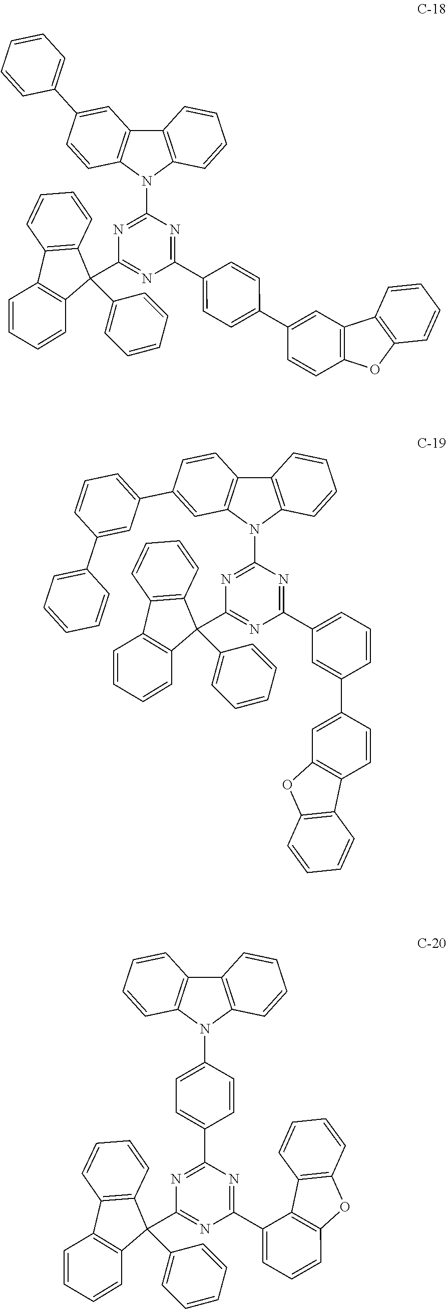 [SMILES: c1ccc(-c2ccc3c(c2)c2ccccc2n3-c2nc(-c3ccc(-c4ccc5oc6ccccc6c5c4)cc3)nc(C3(c4ccccc4)c4ccccc4-c4ccccc43)n2)cc1 ; c1ccc(-c2cccc(-c3ccc4c5ccccc5n(-c5nc(-c6cccc(-c7ccc8c(c7)oc7ccccc78)c6)nc(C6(c7ccccc7)c7ccccc7-c7ccccc76)n5)c4c3)c2)cc1 ; c1ccc(C2(c3nc(-c4ccc(-n5c6ccccc6c6ccccc65)cc4)nc(-c4cccc5oc6ccccc6c45)n3)c3ccccc3-c3ccccc32)cc1]

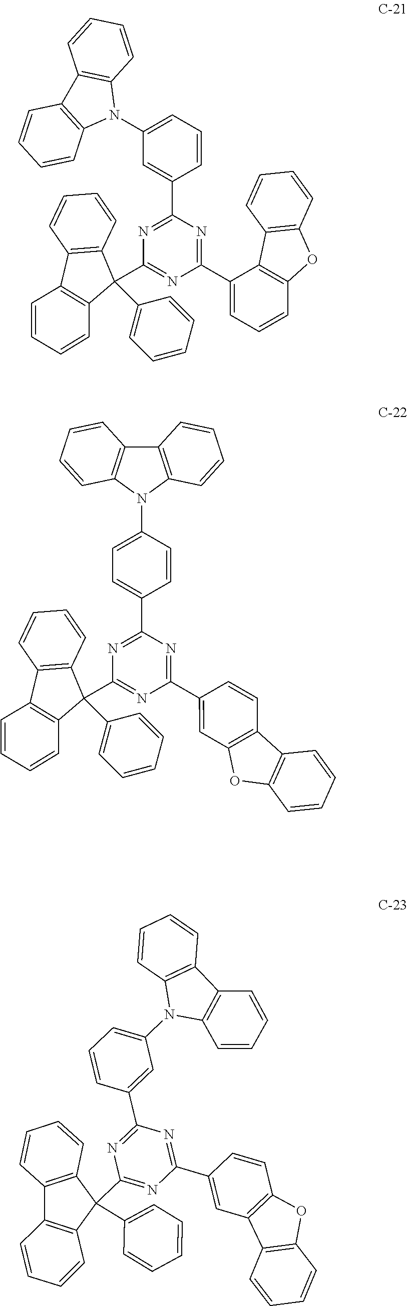 [SMILES: c1ccc(C2(c3nc(-c4cccc(-n5c6ccccc6c6ccccc65)c4)nc(-c4cccc5oc6ccccc6c45)n3)c3ccccc3-c3ccccc32)cc1 ; c1ccc(C2(c3nc(-c4ccc(-n5c6ccccc6c6ccccc65)cc4)nc(-c4ccc5c(c4)oc4ccccc45)n3)c3ccccc3-c3ccccc32)cc1 ; c1ccc(C2(c3nc(-c4cccc(-n5c6ccccc6c6ccccc65)c4)nc(-c4ccc5oc6ccccc6c5c4)n3)c3ccccc3-c3ccccc32)cc1]

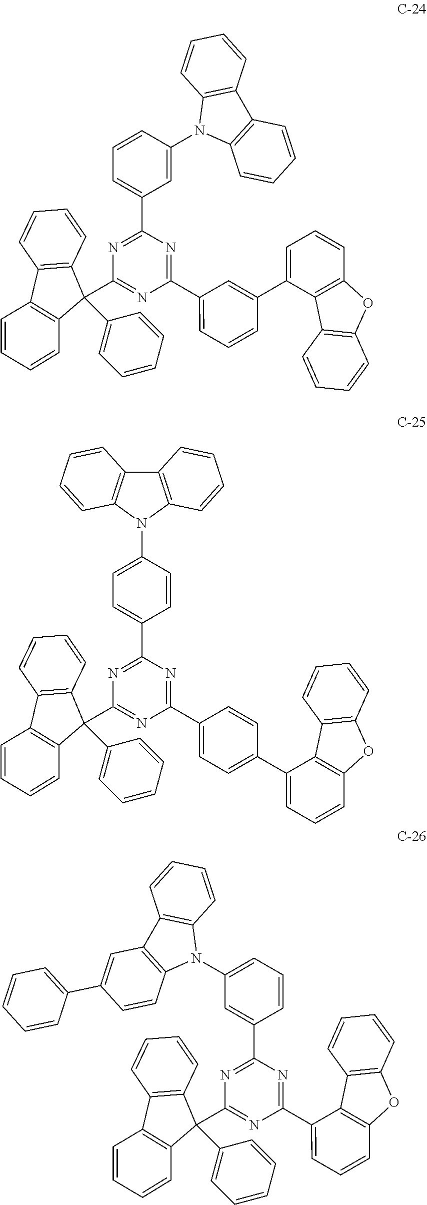 [SMILES: c1ccc(C2(c3nc(-c4cccc(-c5cccc6oc7ccccc7c56)c4)nc(-c4cccc(-n5c6ccccc6c6ccccc65)c4)n3)c3ccccc3-c3ccccc32)cc1 ; c1ccc(C2(c3nc(-c4ccc(-c5cccc6oc7ccccc7c56)cc4)nc(-c4ccc(-n5c6ccccc6c6ccccc65)cc4)n3)c3ccccc3-c3ccccc32)cc1 ; c1ccc(-c2ccc3c(c2)c2ccccc2n3-c2cccc(-c3nc(-c4cccc5oc6ccccc6c45)nc(C4(c5ccccc5)c5ccccc5-c5ccccc54)n3)c2)cc1]

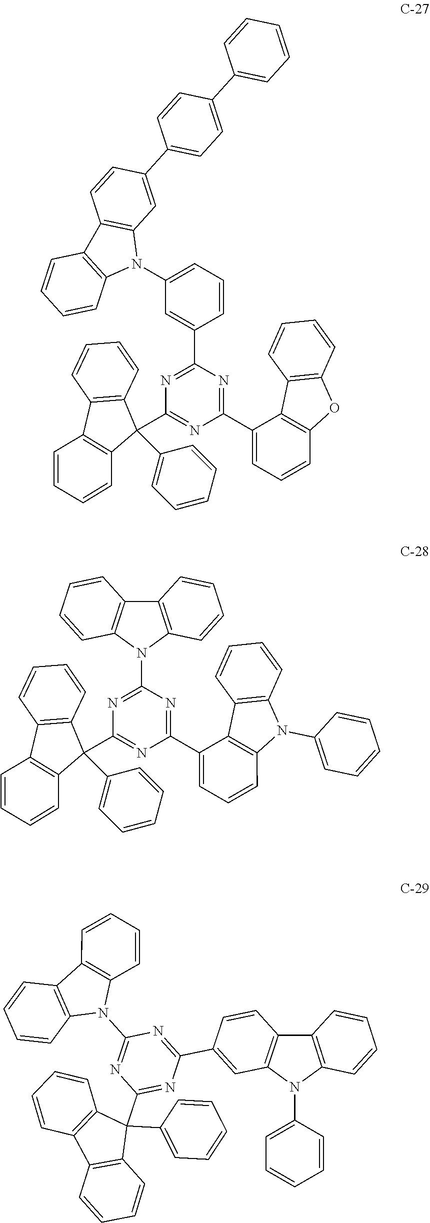 [SMILES: c1ccc(-c2ccc(-c3ccc4c5ccccc5n(-c5cccc(-c6nc(-c7cccc8oc9ccccc9c78)nc(C7(c8ccccc8)c8ccccc8-c8ccccc87)n6)c5)c4c3)cc2)cc1 ; c1ccc(-n2c3ccccc3c3c(-c4nc(-n5c6ccccc6c6ccccc65)nc(C5(c6ccccc6)c6ccccc6-c6ccccc65)n4)cccc32)cc1 ; c1ccc(-n2c3ccccc3c3ccc(-c4nc(-n5c6ccccc6c6ccccc65)nc(C5(c6ccccc6)c6ccccc6-c6ccccc65)n4)cc32)cc1]

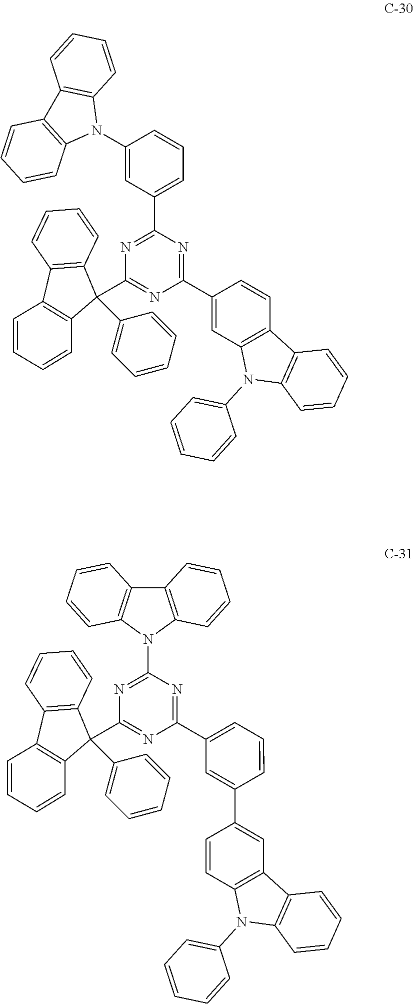 [SMILES: c1ccc(-n2c3ccccc3c3ccc(-c4nc(-c5cccc(-n6c7ccccc7c7ccccc76)c5)nc(C5(c6ccccc6)c6ccccc6-c6ccccc65)n4)cc32)cc1 ; c1ccc(-n2c3ccccc3c3cc(-c4cccc(-c5nc(-n6c7ccccc7c7ccccc76)nc(C6(c7ccccc7)c7ccccc7-c7ccccc76)n5)c4)ccc32)cc1]

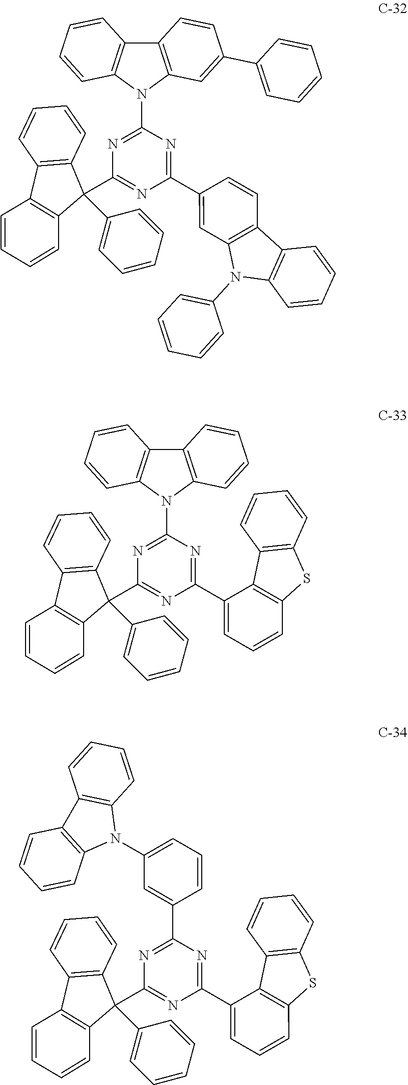 [SMILES: c1ccc(-c2ccc3c4ccccc4n(-c4nc(-c5ccc6c7ccccc7n(-c7ccccc7)c6c5)nc(C5(c6ccccc6)c6ccccc6-c6ccccc65)n4)c3c2)cc1 ; c1ccc(C2(c3nc(-c4cccc5sc6ccccc6c45)nc(-n4c5ccccc5c5ccccc54)n3)c3ccccc3-c3ccccc32)cc1 ; c1ccc(C2(c3nc(-c4cccc(-n5c6ccccc6c6ccccc65)c4)nc(-c4cccc5sc6ccccc6c45)n3)c3ccccc3-c3ccccc32)cc1]

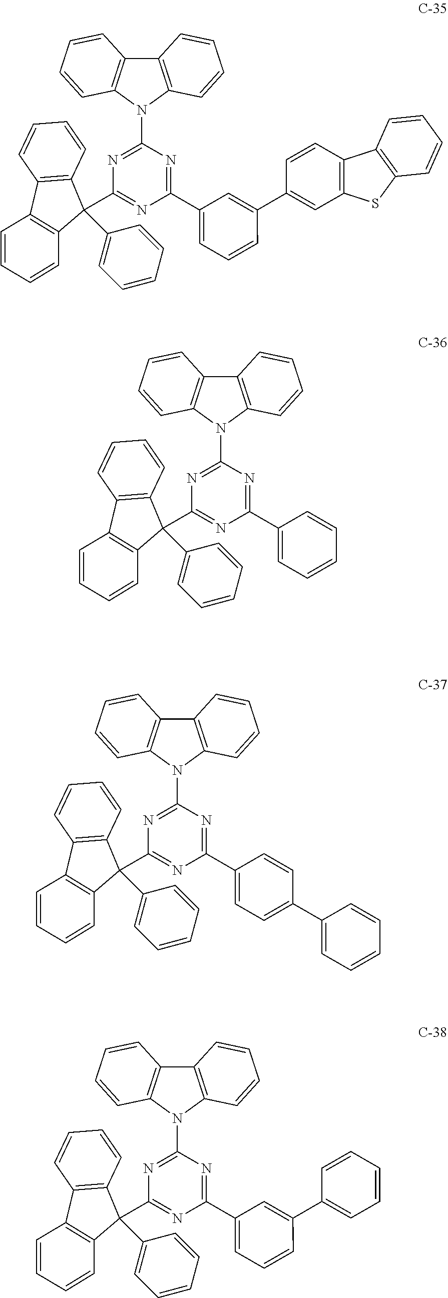 [SMILES: c1ccc(C2(c3nc(-c4cccc(-c5ccc6c(c5)sc5ccccc56)c4)nc(-n4c5ccccc5c5ccccc54)n3)c3ccccc3-c3ccccc32)cc1 ; c1ccc(-c2nc(-n3c4ccccc4c4ccccc43)nc(C3(c4ccccc4)c4ccccc4-c4ccccc43)n2)cc1 ; c1ccc(-c2ccc(-c3nc(-n4c5ccccc5c5ccccc54)nc(C4(c5ccccc5)c5ccccc5-c5ccccc54)n3)cc2)cc1 ; c1ccc(-c2cccc(-c3nc(-n4c5ccccc5c5ccccc54)nc(C4(c5ccccc5)c5ccccc5-c5ccccc54)n3)c2)cc1]

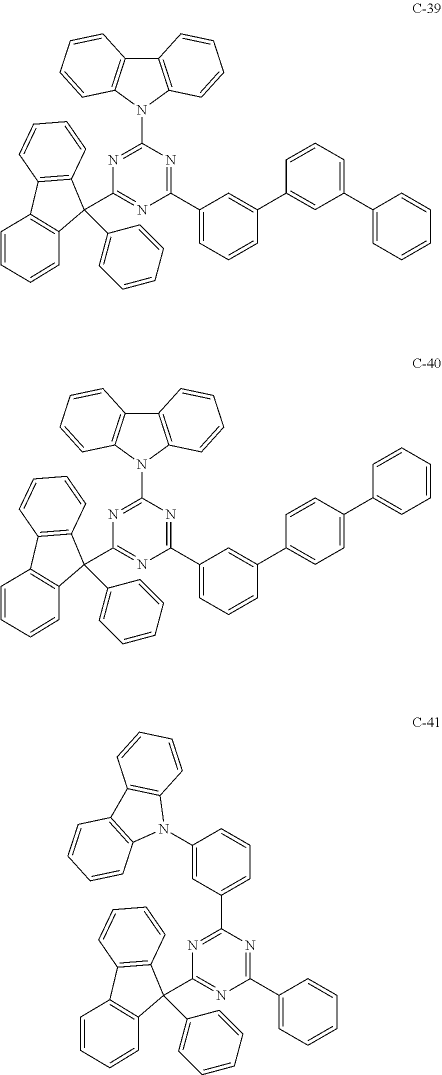 [SMILES: c1ccc(-c2cccc(-c3cccc(-c4nc(-n5c6ccccc6c6ccccc65)nc(C5(c6ccccc6)c6ccccc6-c6ccccc65)n4)c3)c2)cc1 ; c1ccc(-c2ccc(-c3cccc(-c4nc(-n5c6ccccc6c6ccccc65)nc(C5(c6ccccc6)c6ccccc6-c6ccccc65)n4)c3)cc2)cc1 ; c1ccc(-c2nc(-c3cccc(-n4c5ccccc5c5ccccc54)c3)nc(C3(c4ccccc4)c4ccccc4-c4ccccc43)n2)cc1]

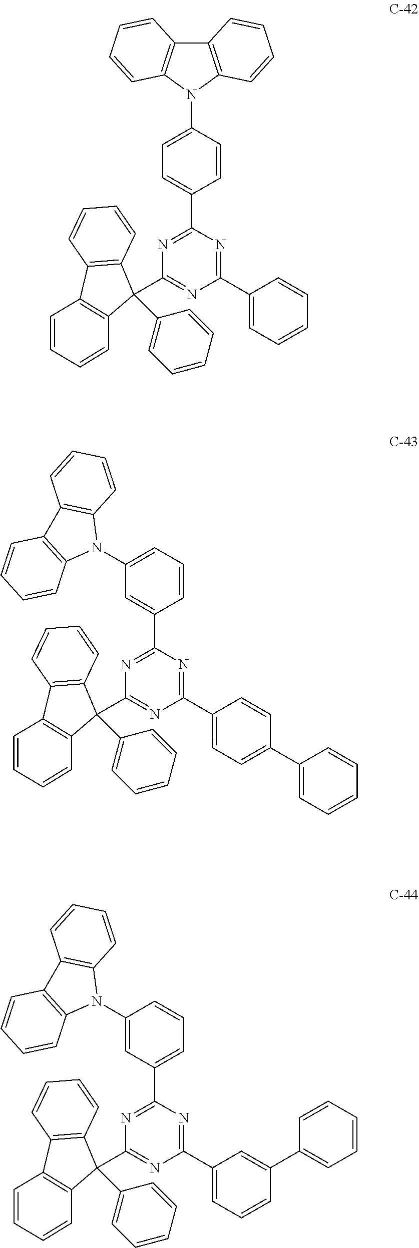 [SMILES: c1ccc(-c2nc(-c3ccc(-n4c5ccccc5c5ccccc54)cc3)nc(C3(c4ccccc4)c4ccccc4-c4ccccc43)n2)cc1 ; c1ccc(-c2ccc(-c3nc(-c4cccc(-n5c6ccccc6c6ccccc65)c4)nc(C4(c5ccccc5)c5ccccc5-c5ccccc54)n3)cc2)cc1 ; c1ccc(-c2cccc(-c3nc(-c4cccc(-n5c6ccccc6c6ccccc65)c4)nc(C4(c5ccccc5)c5ccccc5-c5ccccc54)n3)c2)cc1]

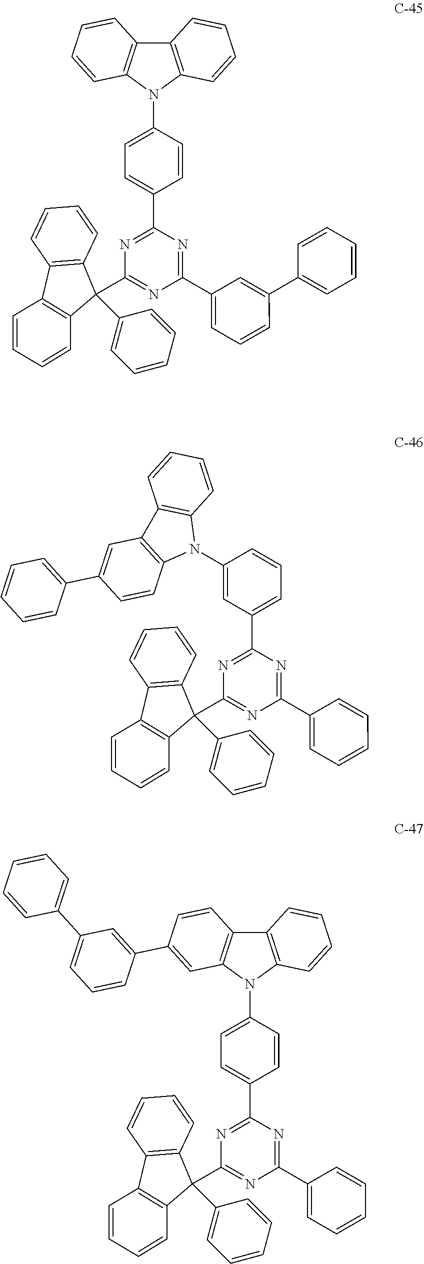 [SMILES: c1ccc(-c2cccc(-c3nc(-c4ccc(-n5c6ccccc6c6ccccc65)cc4)nc(C4(c5ccccc5)c5ccccc5-c5ccccc54)n3)c2)cc1 ; c1ccc(-c2ccc3c(c2)c2ccccc2n3-c2cccc(-c3nc(-c4ccccc4)nc(C4(c5ccccc5)c5ccccc5-c5ccccc54)n3)c2)cc1 ; c1ccc(-c2cccc(-c3ccc4c5ccccc5n(-c5ccc(-c6nc(-c7ccccc7)nc(C7(c8ccccc8)c8ccccc8-c8ccccc87)n6)cc5)c4c3)c2)cc1]

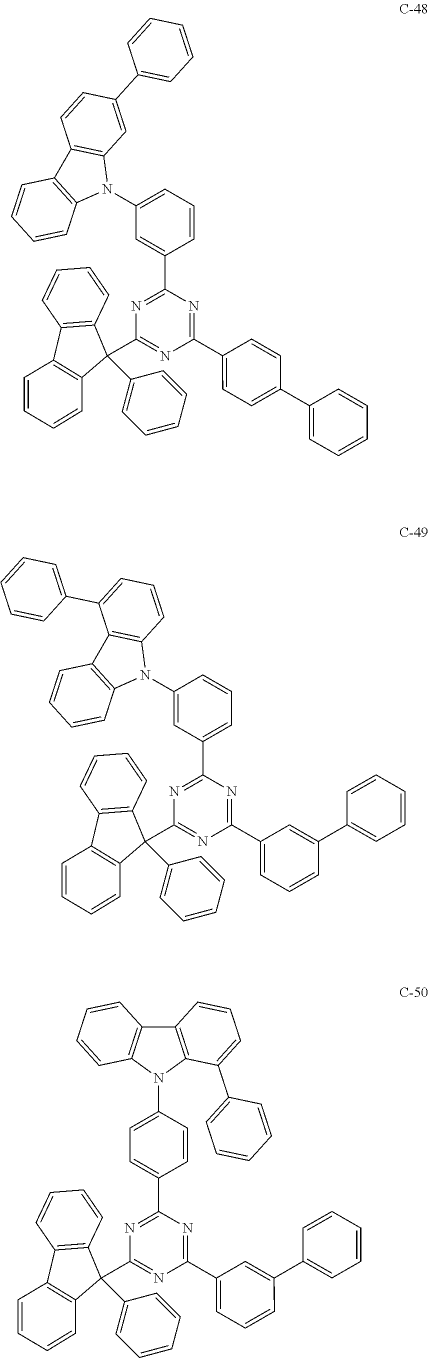 [SMILES: c1ccc(-c2ccc(-c3nc(-c4cccc(-n5c6ccccc6c6ccc(-c7ccccc7)cc65)c4)nc(C4(c5ccccc5)c5ccccc5-c5ccccc54)n3)cc2)cc1 ; c1ccc(-c2cccc(-c3nc(-c4cccc(-n5c6ccccc6c6c(-c7ccccc7)cccc65)c4)nc(C4(c5ccccc5)c5ccccc5-c5ccccc54)n3)c2)cc1 ; c1ccc(-c2cccc(-c3nc(-c4ccc(-n5c6ccccc6c6cccc(-c7ccccc7)c65)cc4)nc(C4(c5ccccc5)c5ccccc5-c5ccccc54)n3)c2)cc1]

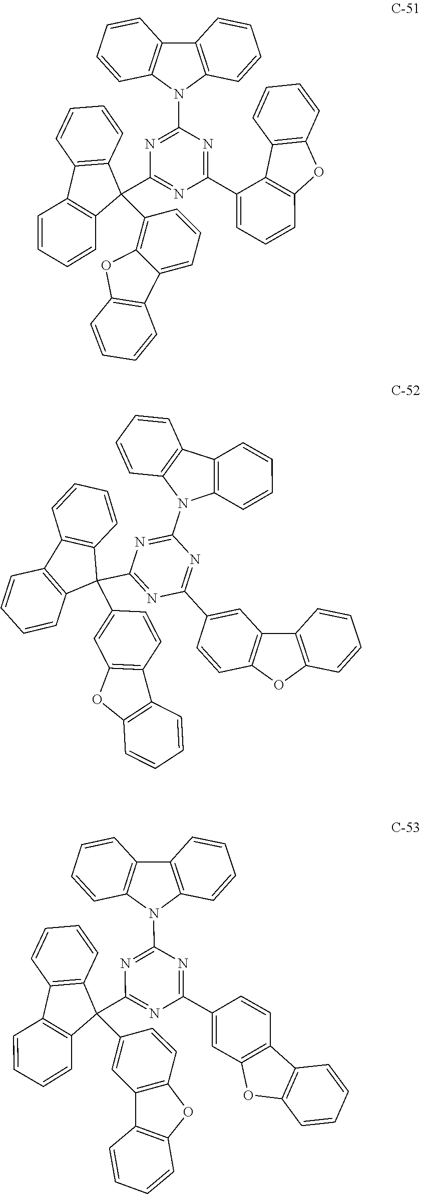 [SMILES: c1ccc2c(c1)-c1ccccc1C2(c1nc(-c2cccc3oc4ccccc4c23)nc(-n2c3ccccc3c3ccccc32)n1)c1cccc2c1oc1ccccc12 ; c1ccc2c(c1)-c1ccccc1C2(c1ccc2c(c1)oc1ccccc12)c1nc(-c2ccc3oc4ccccc4c3c2)nc(-n2c3ccccc3c3ccccc32)n1 ; c1ccc2c(c1)-c1ccccc1C2(c1ccc2oc3ccccc3c2c1)c1nc(-c2ccc3c(c2)oc2ccccc23)nc(-n2c3ccccc3c3ccccc32)n1]

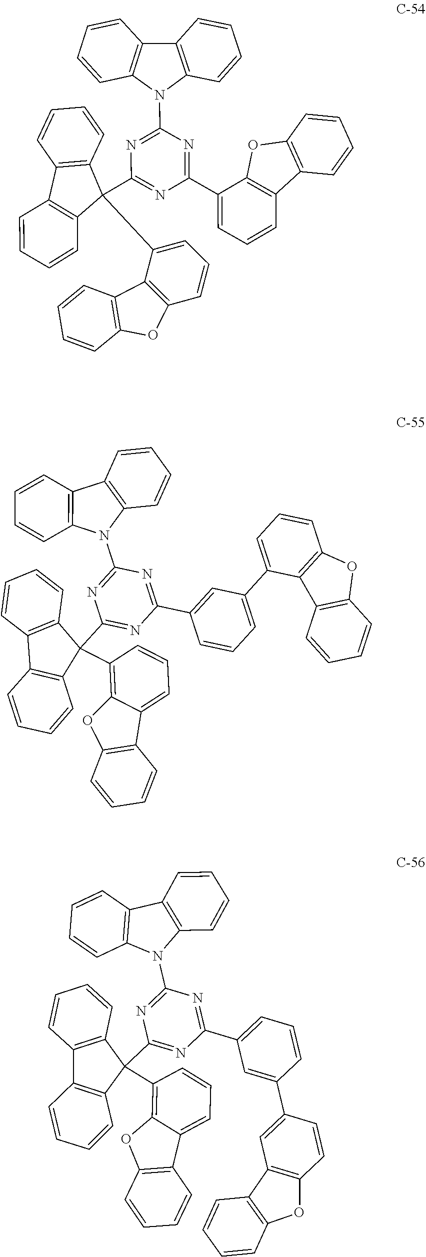 [SMILES: c1ccc2c(c1)-c1ccccc1C2(c1nc(-c2cccc3c2oc2ccccc23)nc(-n2c3ccccc3c3ccccc32)n1)c1cccc2oc3ccccc3c12 ; c1cc(-c2nc(-n3c4ccccc4c4ccccc43)nc(C3(c4cccc5c4oc4ccccc45)c4ccccc4-c4ccccc43)n2)cc(-c2cccc3oc4ccccc4c23)c1 ; c1cc(-c2ccc3oc4ccccc4c3c2)cc(-c2nc(-n3c4ccccc4c4ccccc43)nc(C3(c4cccc5c4oc4ccccc45)c4ccccc4-c4ccccc43)n2)c1]

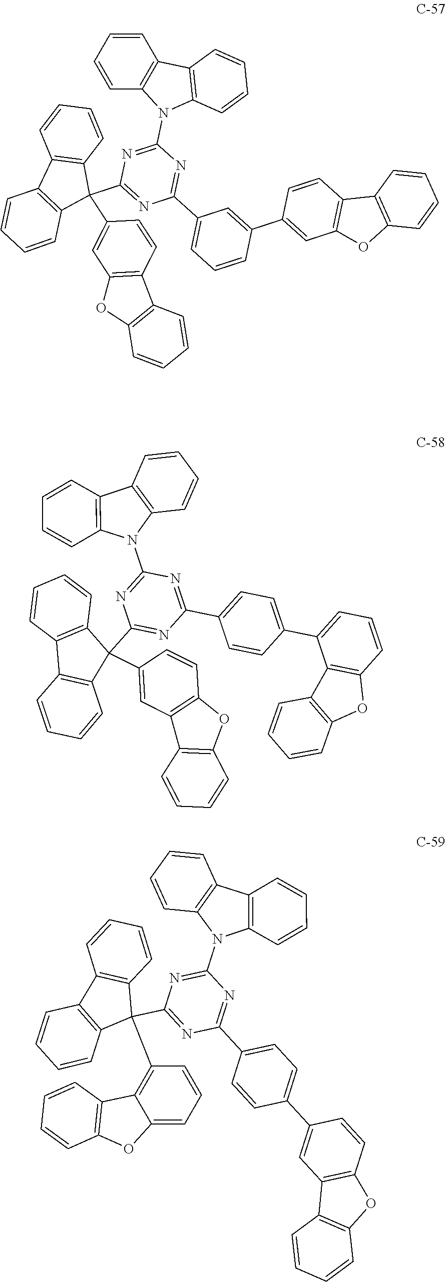

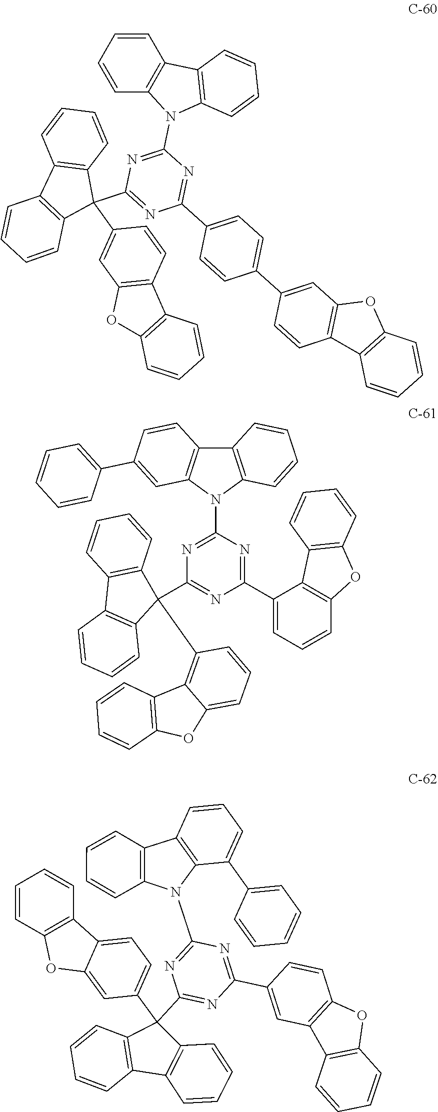

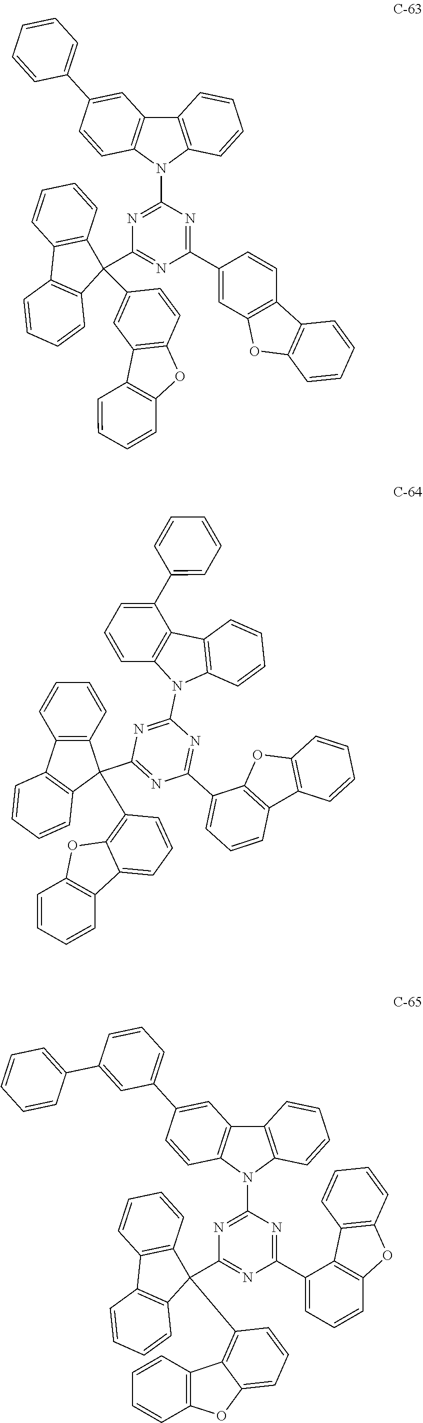 [SMILES: c1ccc(-c2ccc3c(c2)c2ccccc2n3-c2nc(-c3ccc4c(c3)oc3ccccc34)nc(C3(c4ccc5oc6ccccc6c5c4)c4ccccc4-c4ccccc43)n2)cc1 ; c1ccc(-c2cccc3c2c2ccccc2n3-c2nc(-c3cccc4c3oc3ccccc34)nc(C3(c4cccc5c4oc4ccccc45)c4ccccc4-c4ccccc43)n2)cc1 ; c1ccc(-c2cccc(-c3ccc4c(c3)c3ccccc3n4-c3nc(-c4cccc5oc6ccccc6c45)nc(C4(c5cccc6oc7ccccc7c56)c5ccccc5-c5ccccc54)n3)c2)cc1]

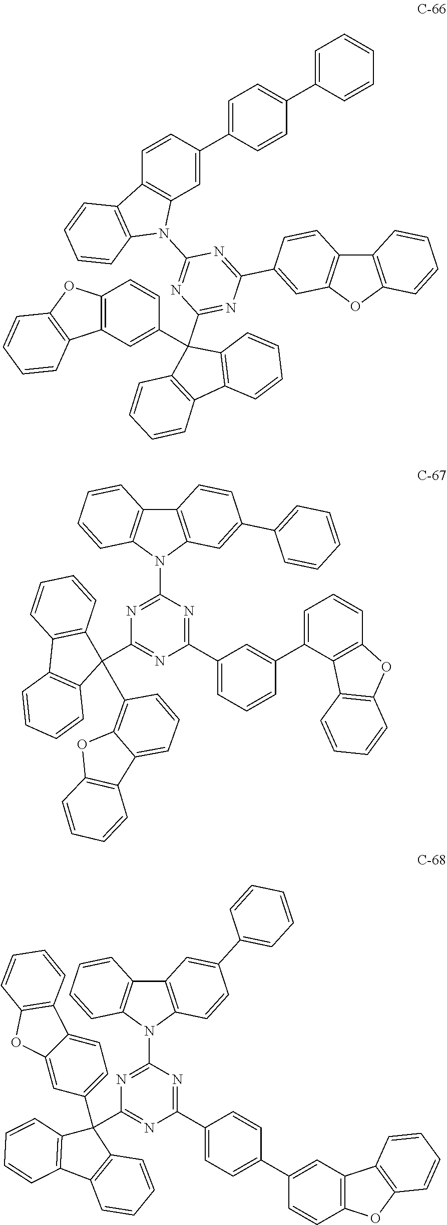 [SMILES: c1ccc(-c2ccc(-c3ccc4c5ccccc5n(-c5nc(-c6ccc7c(c6)oc6ccccc67)nc(C6(c7ccc8oc9ccccc9c8c7)c7ccccc7-c7ccccc76)n5)c4c3)cc2)cc1 ; c1ccc(-c2ccc3c4ccccc4n(-c4nc(-c5cccc(-c6cccc7oc8ccccc8c67)c5)nc(C5(c6cccc7c6oc6ccccc67)c6ccccc6-c6ccccc65)n4)c3c2)cc1 ; c1ccc(-c2ccc3c(c2)c2ccccc2n3-c2nc(-c3ccc(-c4ccc5oc6ccccc6c5c4)cc3)nc(C3(c4ccc5c(c4)oc4ccccc45)c4ccccc4-c4ccccc43)n2)cc1]

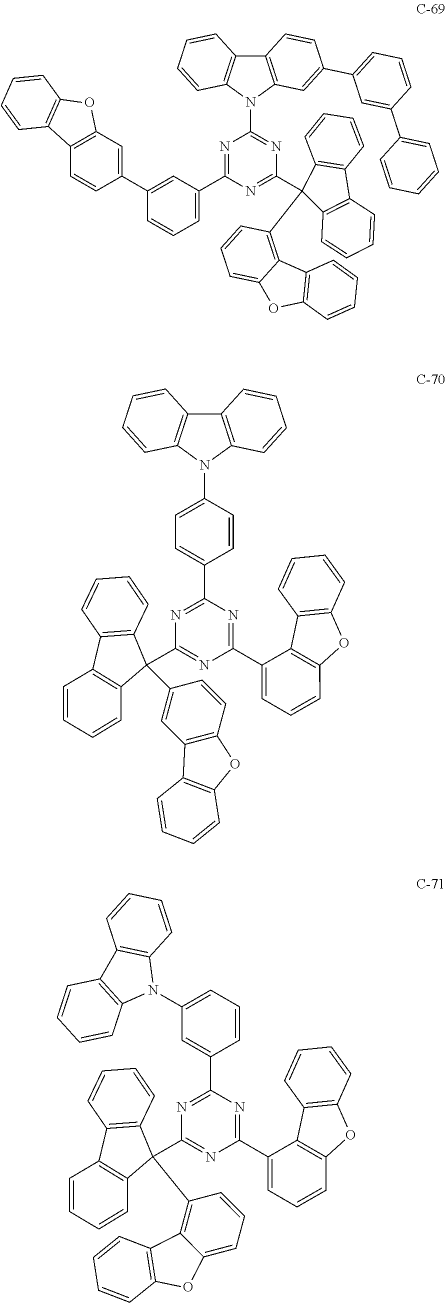 [SMILES: c1ccc(-c2cccc(-c3ccc4c5ccccc5n(-c5nc(-c6cccc(-c7ccc8c(c7)oc7ccccc78)c6)nc(C6(c7cccc8oc9ccccc9c78)c7ccccc7-c7ccccc76)n5)c4c3)c2)cc1 ; c1ccc2c(c1)-c1ccccc1C2(c1ccc2oc3ccccc3c2c1)c1nc(-c2ccc(-n3c4ccccc4c4ccccc43)cc2)nc(-c2cccc3oc4ccccc4c23)n1 ; c1cc(-c2nc(-c3cccc4oc5ccccc5c34)nc(C3(c4cccc5oc6ccccc6c45)c4ccccc4-c4ccccc43)n2)cc(-n2c3ccccc3c3ccccc32)c1]

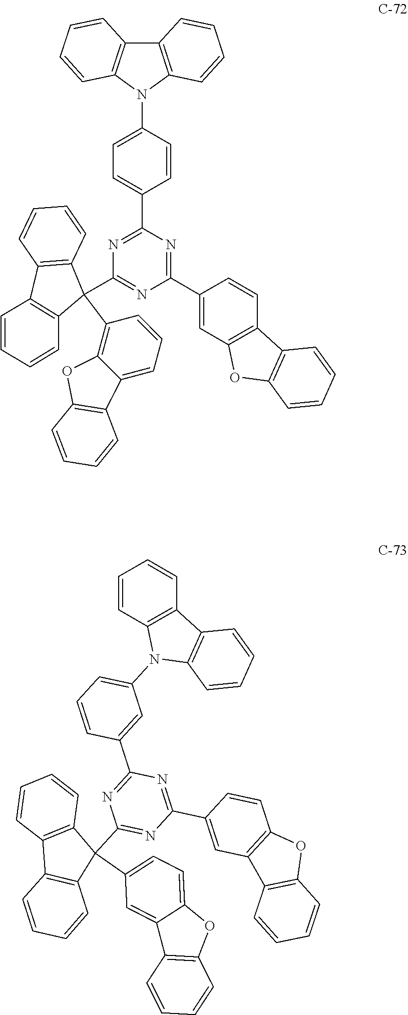 [SMILES: c1ccc2c(c1)-c1ccccc1C2(c1nc(-c2ccc(-n3c4ccccc4c4ccccc43)cc2)nc(-c2ccc3c(c2)oc2ccccc23)n1)c1cccc2c1oc1ccccc12 ; c1cc(-c2nc(-c3ccc4oc5ccccc5c4c3)nc(C3(c4ccc5oc6ccccc6c5c4)c4ccccc4-c4ccccc43)n2)cc(-n2c3ccccc3c3ccccc32)c1]

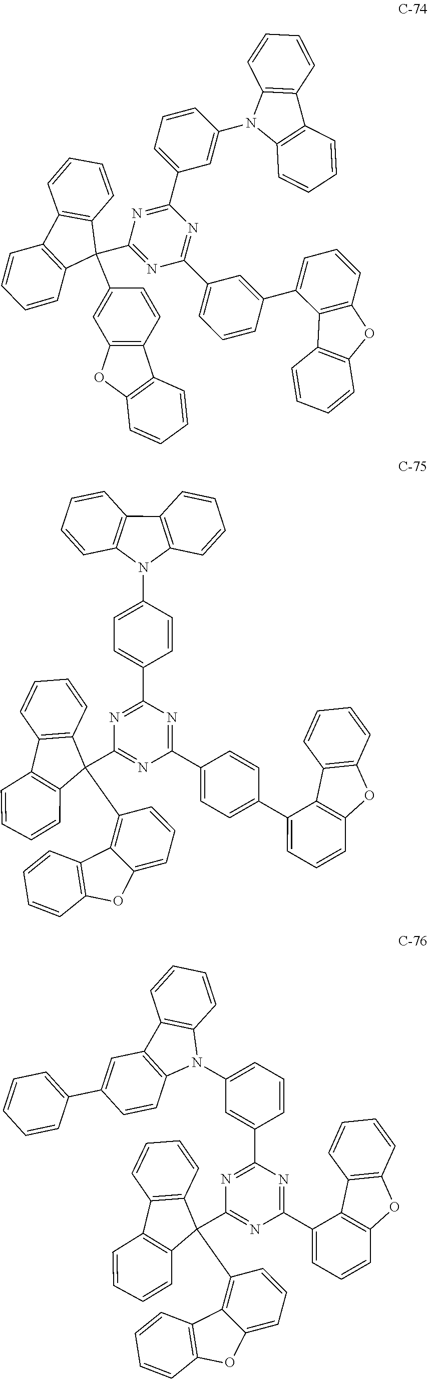 [SMILES: c1cc(-c2nc(-c3cccc(-n4c5ccccc5c5ccccc54)c3)nc(C3(c4ccc5c(c4)oc4ccccc45)c4ccccc4-c4ccccc43)n2)cc(-c2cccc3oc4ccccc4c23)c1 ; c1ccc2c(c1)-c1ccccc1C2(c1nc(-c2ccc(-c3cccc4oc5ccccc5c34)cc2)nc(-c2ccc(-n3c4ccccc4c4ccccc43)cc2)n1)c1cccc2oc3ccccc3c12 ; c1ccc(-c2ccc3c(c2)c2ccccc2n3-c2cccc(-c3nc(-c4cccc5oc6ccccc6c45)nc(C4(c5cccc6oc7ccccc7c56)c5ccccc5-c5ccccc54)n3)c2)cc1]

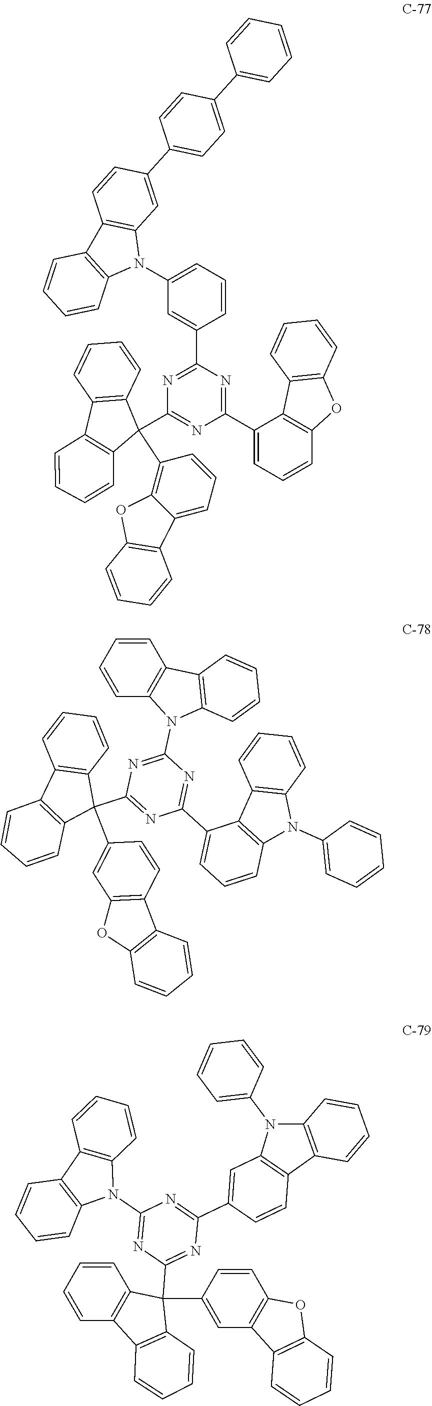

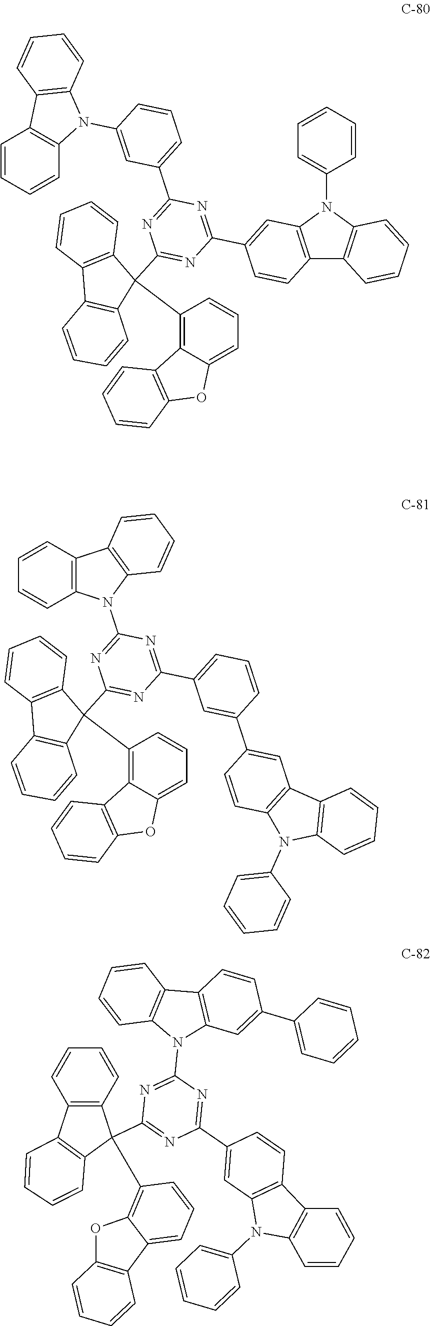

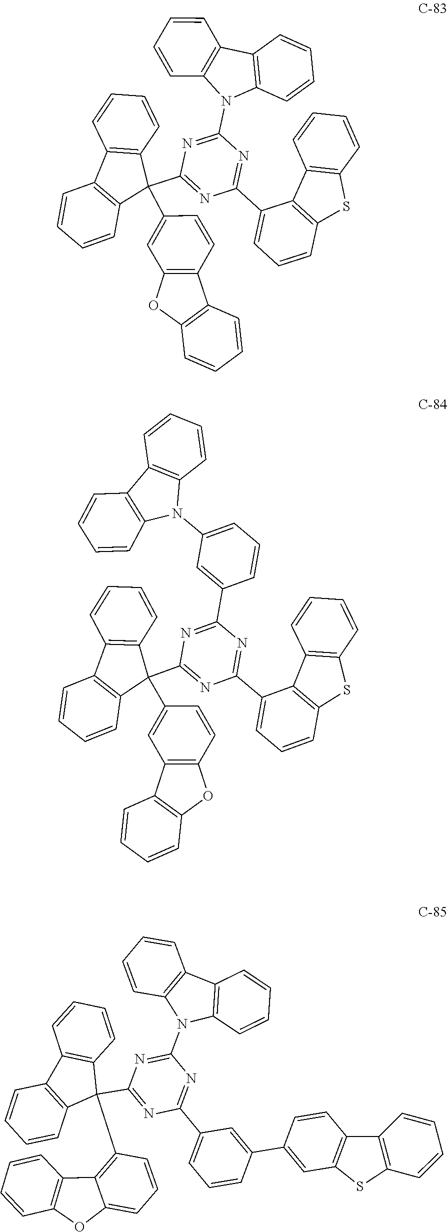 [SMILES: c1ccc2c(c1)-c1ccccc1C2(c1ccc2c(c1)oc1ccccc12)c1nc(-c2cccc3sc4ccccc4c23)nc(-n2c3ccccc3c3ccccc32)n1 ; c1cc(-c2nc(-c3cccc4sc5ccccc5c34)nc(C3(c4ccc5oc6ccccc6c5c4)c4ccccc4-c4ccccc43)n2)cc(-n2c3ccccc3c3ccccc32)c1 ; c1cc(-c2ccc3c(c2)sc2ccccc23)cc(-c2nc(-n3c4ccccc4c4ccccc43)nc(C3(c4cccc5oc6ccccc6c45)c4ccccc4-c4ccccc43)n2)c1]

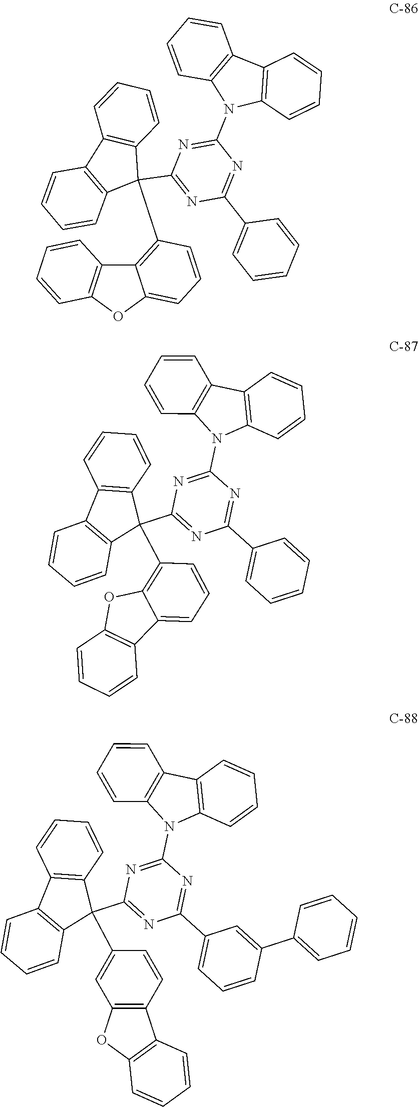

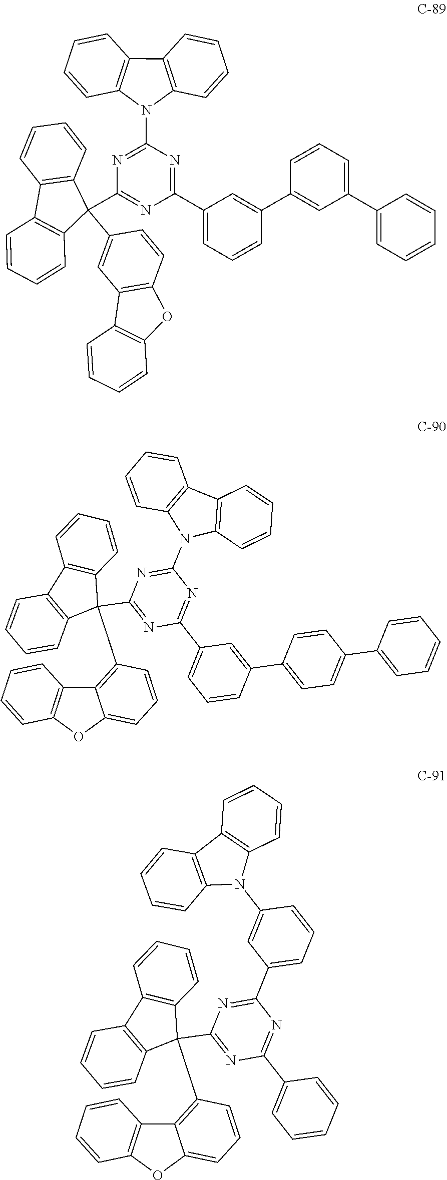 [SMILES: c1ccc(-c2cccc(-c3cccc(-c4nc(-n5c6ccccc6c6ccccc65)nc(C5(c6ccc7oc8ccccc8c7c6)c6ccccc6-c6ccccc65)n4)c3)c2)cc1 ; c1ccc(-c2ccc(-c3cccc(-c4nc(-n5c6ccccc6c6ccccc65)nc(C5(c6cccc7oc8ccccc8c67)c6ccccc6-c6ccccc65)n4)c3)cc2)cc1 ; c1ccc(-c2nc(-c3cccc(-n4c5ccccc5c5ccccc54)c3)nc(C3(c4cccc5oc6ccccc6c45)c4ccccc4-c4ccccc43)n2)cc1]

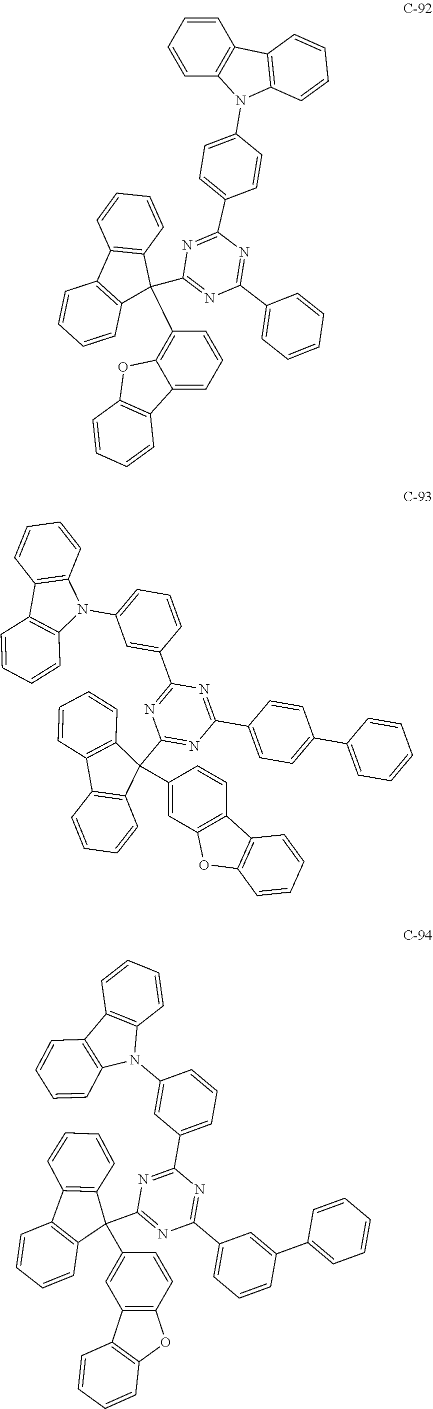 [SMILES: c1ccc(-c2nc(-c3ccc(-n4c5ccccc5c5ccccc54)cc3)nc(C3(c4cccc5c4oc4ccccc45)c4ccccc4-c4ccccc43)n2)cc1 ; c1ccc(-c2ccc(-c3nc(-c4cccc(-n5c6ccccc6c6ccccc65)c4)nc(C4(c5ccc6c(c5)oc5ccccc56)c5ccccc5-c5ccccc54)n3)cc2)cc1 ; c1ccc(-c2cccc(-c3nc(-c4cccc(-n5c6ccccc6c6ccccc65)c4)nc(C4(c5ccc6oc7ccccc7c6c5)c5ccccc5-c5ccccc54)n3)c2)cc1]

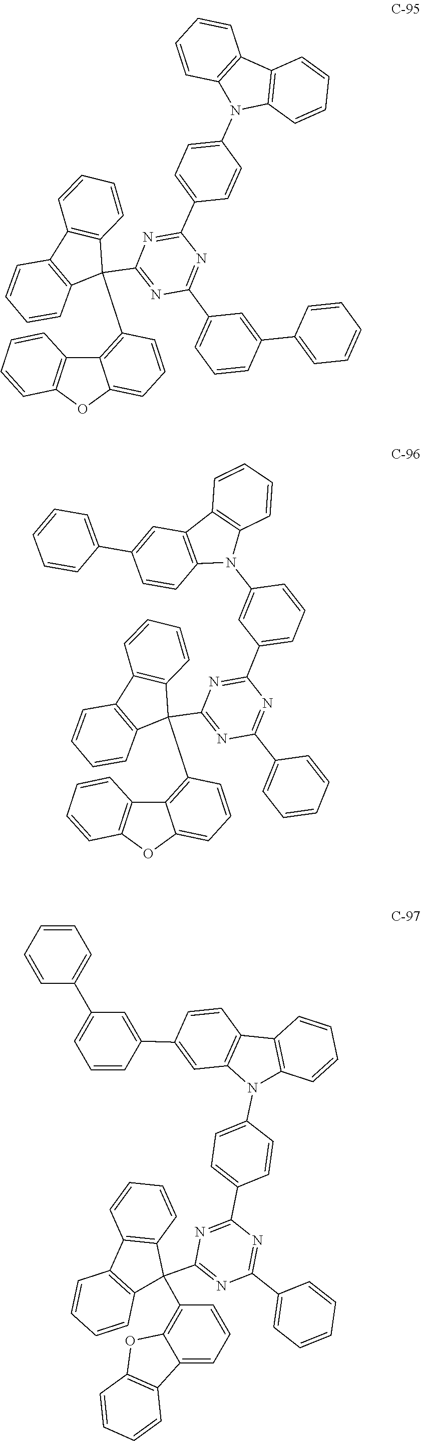 [SMILES: c1ccc(-c2cccc(-c3nc(-c4ccc(-n5c6ccccc6c6ccccc65)cc4)nc(C4(c5cccc6oc7ccccc7c56)c5ccccc5-c5ccccc54)n3)c2)cc1 ; c1ccc(-c2ccc3c(c2)c2ccccc2n3-c2cccc(-c3nc(-c4ccccc4)nc(C4(c5cccc6oc7ccccc7c56)c5ccccc5-c5ccccc54)n3)c2)cc1 ; c1ccc(-c2cccc(-c3ccc4c5ccccc5n(-c5ccc(-c6nc(-c7ccccc7)nc(C7(c8cccc9c8oc8ccccc89)c8ccccc8-c8ccccc87)n6)cc5)c4c3)c2)cc1]

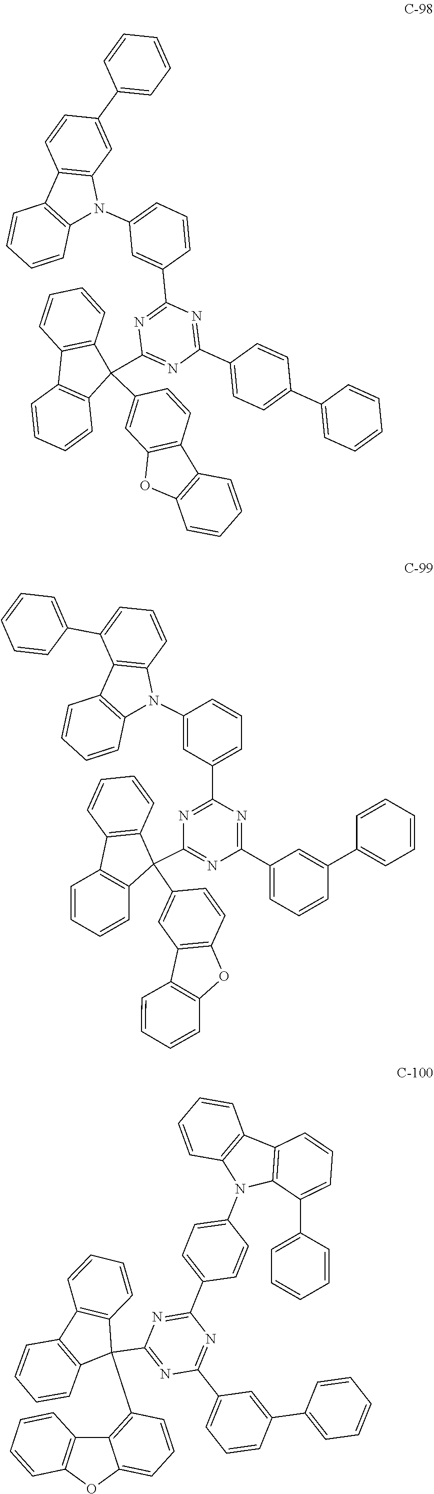 [SMILES: c1ccc(-c2ccc(-c3nc(-c4cccc(-n5c6ccccc6c6ccc(-c7ccccc7)cc65)c4)nc(C4(c5ccc6c(c5)oc5ccccc56)c5ccccc5-c5ccccc54)n3)cc2)cc1 ; c1ccc(-c2cccc(-c3nc(-c4cccc(-n5c6ccccc6c6c(-c7ccccc7)cccc65)c4)nc(C4(c5ccc6oc7ccccc7c6c5)c5ccccc5-c5ccccc54)n3)c2)cc1 ; c1ccc(-c2cccc(-c3nc(-c4ccc(-n5c6ccccc6c6cccc(-c7ccccc7)c65)cc4)nc(C4(c5cccc6oc7ccccc7c56)c5ccccc5-c5ccccc54)n3)c2)cc1]

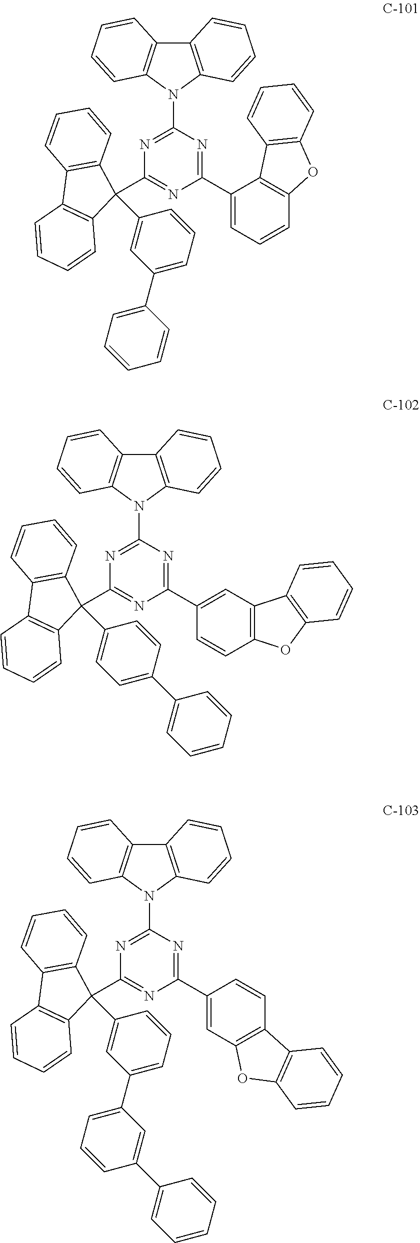 [SMILES: c1ccc(-c2cccc(C3(c4nc(-c5cccc6oc7ccccc7c56)nc(-n5c6ccccc6c6ccccc65)n4)c4ccccc4-c4ccccc43)c2)cc1 ; c1ccc(-c2ccc(C3(c4nc(-c5ccc6oc7ccccc7c6c5)nc(-n5c6ccccc6c6ccccc65)n4)c4ccccc4-c4ccccc43)cc2)cc1 ; c1ccc(-c2cccc(-c3cccc(C4(c5nc(-c6ccc7c(c6)oc6ccccc67)nc(-n6c7ccccc7c7ccccc76)n5)c5ccccc5-c5ccccc54)c3)c2)cc1]

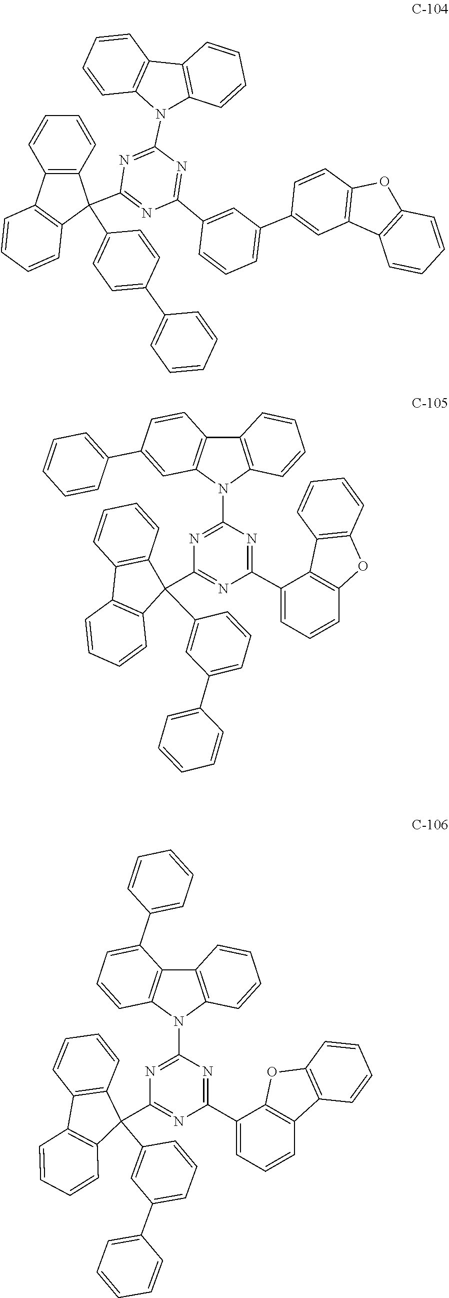

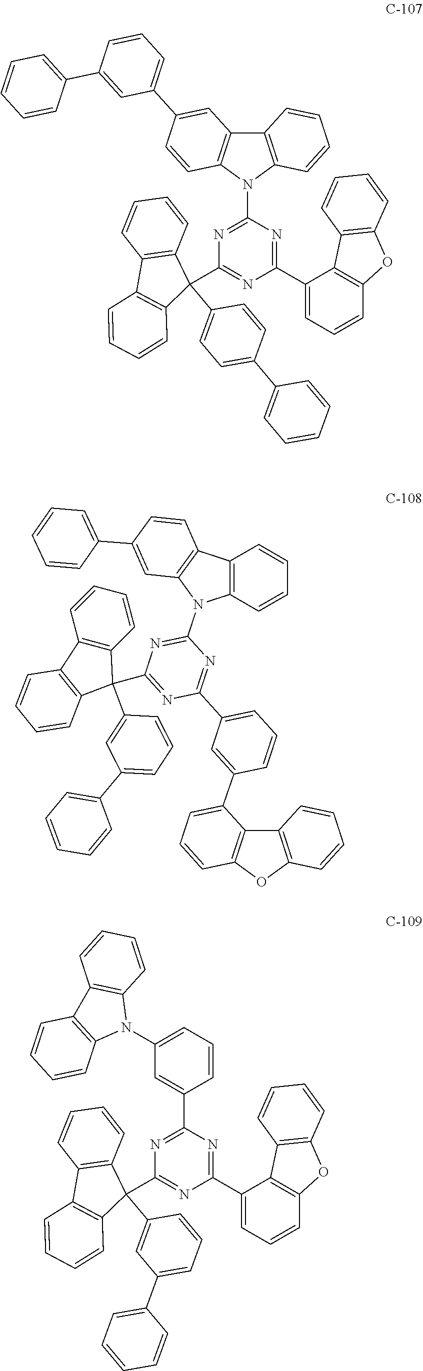

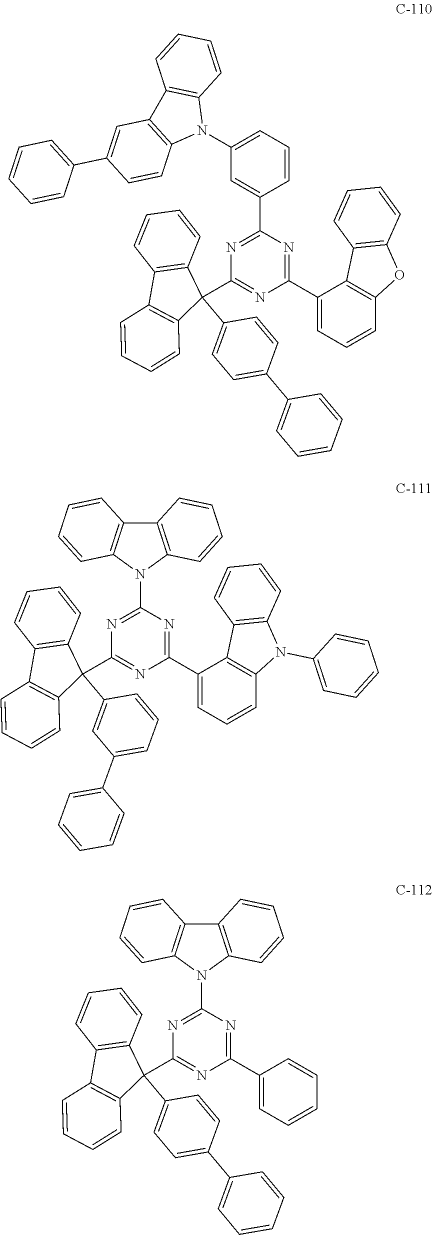 [SMILES: c1ccc(-c2ccc(C3(c4nc(-c5cccc(-n6c7ccccc7c7cc(-c8ccccc8)ccc76)c5)nc(-c5cccc6oc7ccccc7c56)n4)c4ccccc4-c4ccccc43)cc2)cc1 ; c1ccc(-c2cccc(C3(c4nc(-c5cccc6c5c5ccccc5n6-c5ccccc5)nc(-n5c6ccccc6c6ccccc65)n4)c4ccccc4-c4ccccc43)c2)cc1 ; c1ccc(-c2ccc(C3(c4nc(-c5ccccc5)nc(-n5c6ccccc6c6ccccc65)n4)c4ccccc4-c4ccccc43)cc2)cc1]

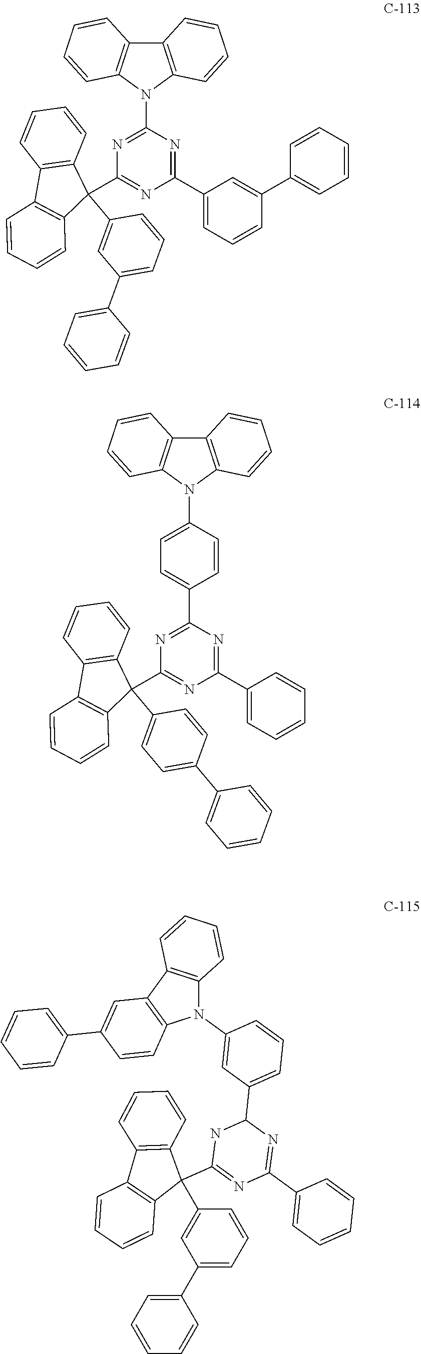

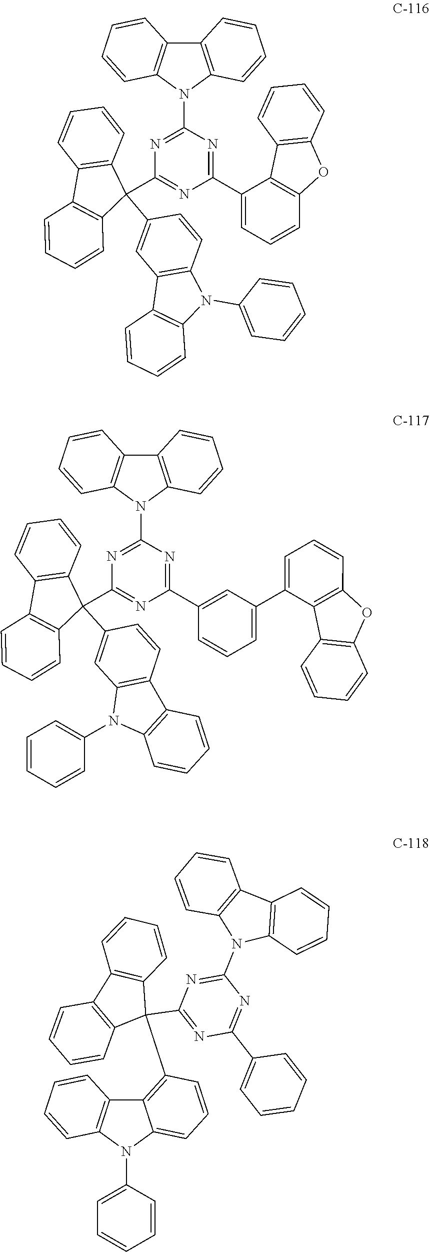 [SMILES: c1ccc(-n2c3ccccc3c3cc(C4(c5nc(-c6cccc7oc8ccccc8c67)nc(-n6c7ccccc7c7ccccc76)n5)c5ccccc5-c5ccccc54)ccc32)cc1 ; c1ccc(-n2c3ccccc3c3ccc(C4(c5nc(-c6cccc(-c7cccc8oc9ccccc9c78)c6)nc(-n6c7ccccc7c7ccccc76)n5)c5ccccc5-c5ccccc54)cc32)cc1 ; c1ccc(-c2nc(-n3c4ccccc4c4ccccc43)nc(C3(c4cccc5c4c4ccccc4n5-c4ccccc4)c4ccccc4-c4ccccc43)n2)cc1]

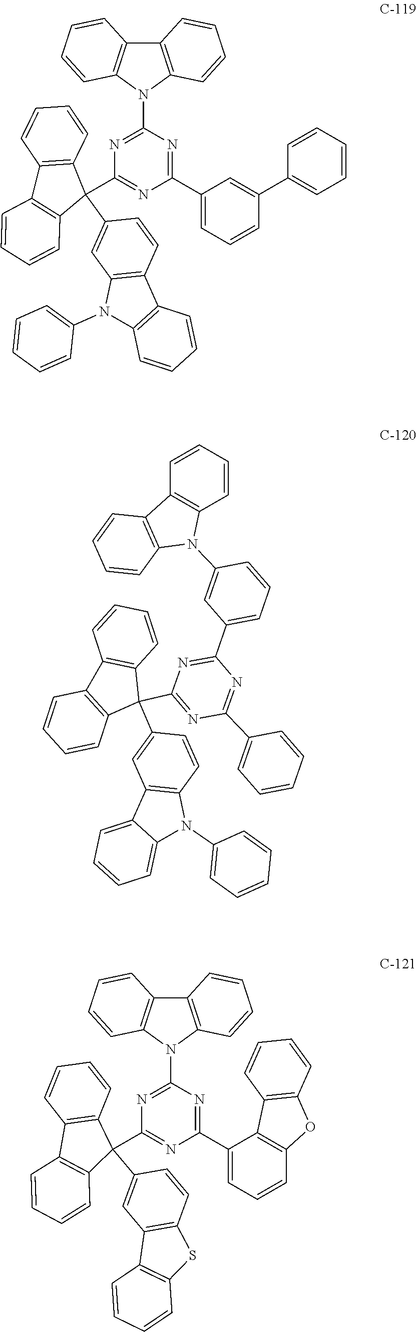 [SMILES: c1ccc(-c2cccc(-c3nc(-n4c5ccccc5c5ccccc54)nc(C4(c5ccc6c7ccccc7n(-c7ccccc7)c6c5)c5ccccc5-c5ccccc54)n3)c2)cc1 ; c1ccc(-c2nc(-c3cccc(-n4c5ccccc5c5ccccc54)c3)nc(C3(c4ccc5c(c4)c4ccccc4n5-c4ccccc4)c4ccccc4-c4ccccc43)n2)cc1 ; c1ccc2c(c1)-c1ccccc1C2(c1ccc2sc3ccccc3c2c1)c1nc(-c2cccc3oc4ccccc4c23)nc(-n2c3ccccc3c3ccccc32)n1]

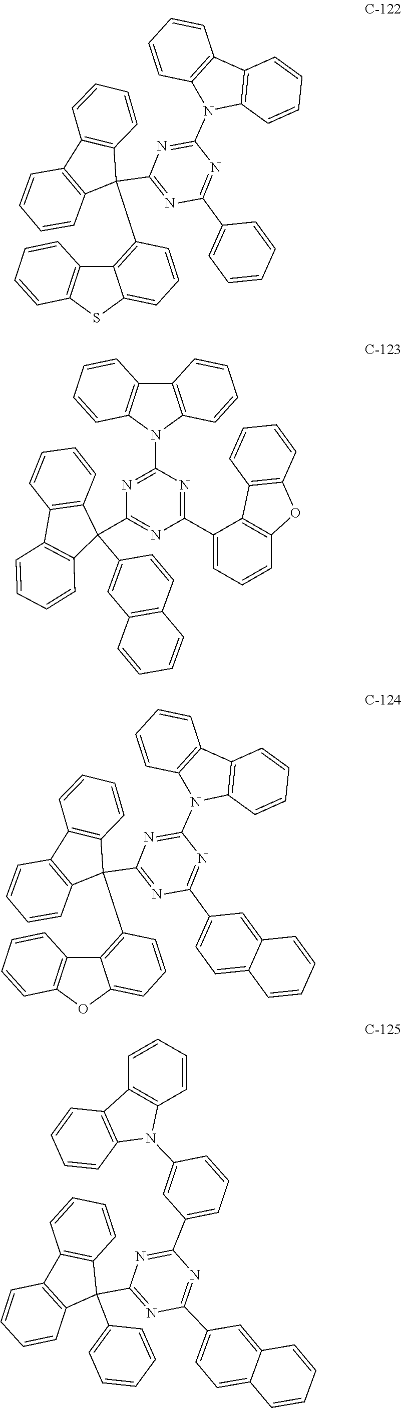

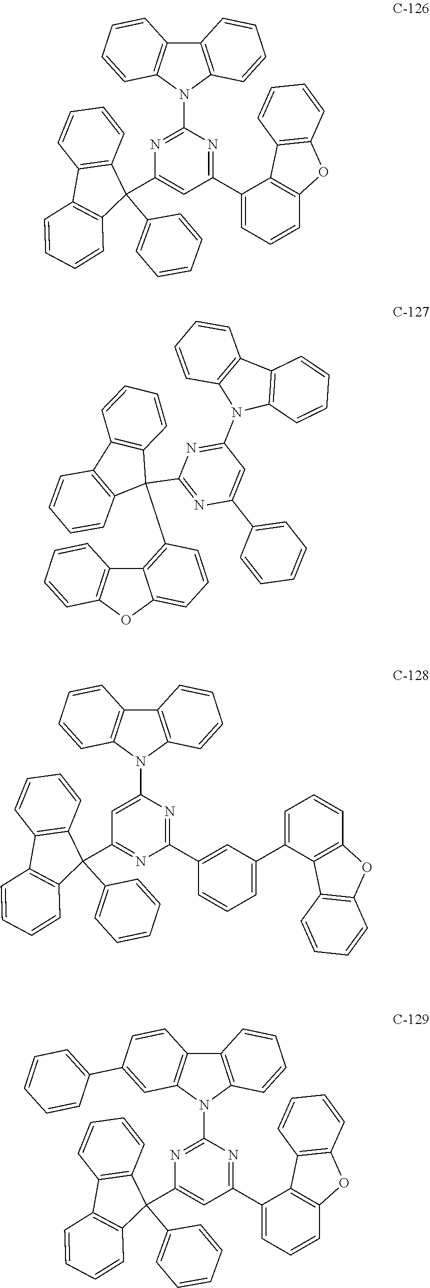 [SMILES: c1ccc(C2(c3cc(-c4cccc5oc6ccccc6c45)nc(-n4c5ccccc5c5ccccc54)n3)c3ccccc3-c3ccccc32)cc1 ; c1ccc(-c2cc(-n3c4ccccc4c4ccccc43)nc(C3(c4cccc5oc6ccccc6c45)c4ccccc4-c4ccccc43)n2)cc1 ; c1ccc(C2(c3cc(-n4c5ccccc5c5ccccc54)nc(-c4cccc(-c5cccc6oc7ccccc7c56)c4)n3)c3ccccc3-c3ccccc32)cc1 ; c1ccc(-c2ccc3c4ccccc4n(-c4nc(-c5cccc6oc7ccccc7c56)cc(C5(c6ccccc6)c6ccccc6-c6ccccc65)n4)c3c2)cc1]

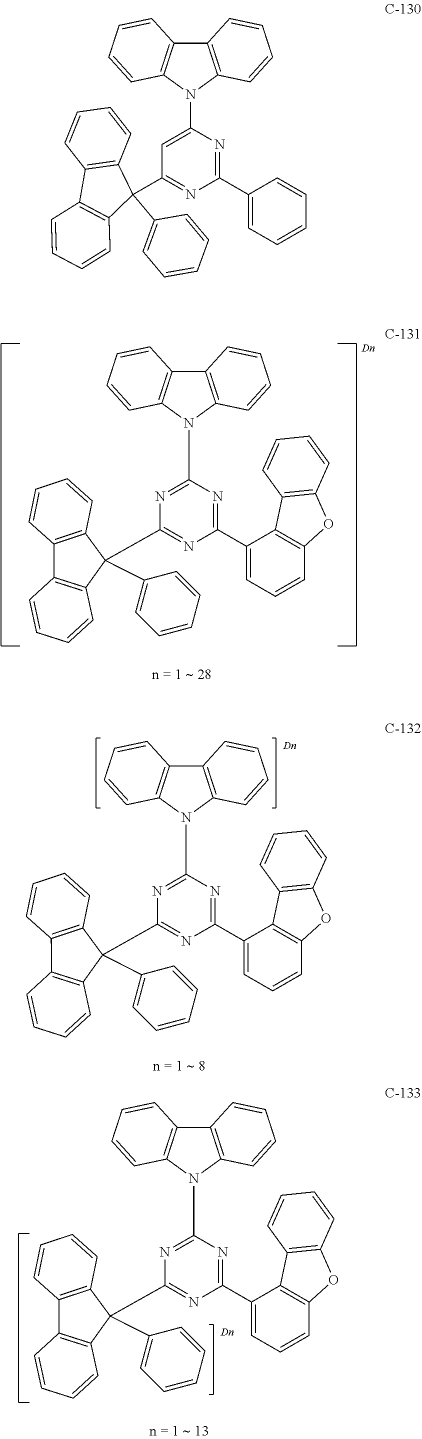

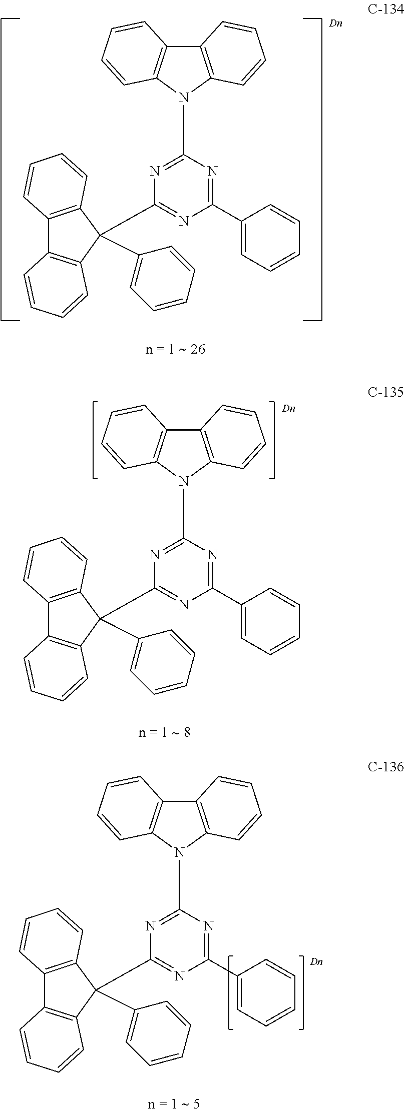

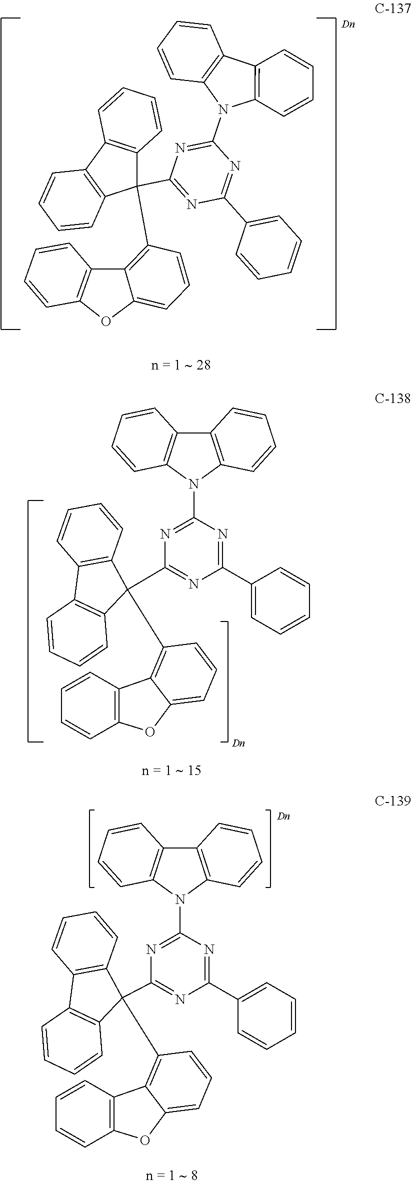

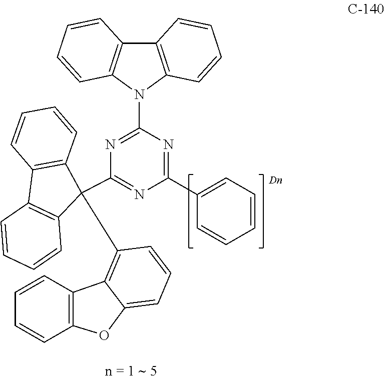

Among the above compounds, Dn signifies that n hydrogens are replaced with deuterium.

The compound represented by Formula 1 according to the present disclosure may be produced by referring to the following Reaction Scheme 1, but is not limited thereto.

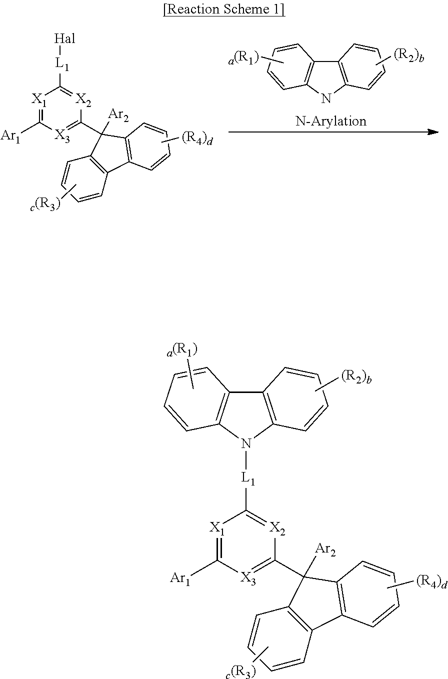

In reaction scheme 1, X₁ to X₃, L₁, R₁ to R₄, Ar₁, Ar₂, and a to d are as defined in Formula 1.

Although illustrative synthesis example of the compound represented by Formula 1 is described above, one skilled in the art will be able to readily understand that all of them are based on a Buchwald-Hartwig cross-coupling reaction, an N-arylation reaction, an H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a cyclic dehydration reaction, an SN₁ substitution reaction, an SN₂ substitution reaction, a phosphine-mediated reductive cyclization reaction, etc., and the above reaction proceeds even when substituents which are defined in Formula 1, but are not specified in the specific synthesis example, are bonded.

The second host compound is represented by the following Formula 2.

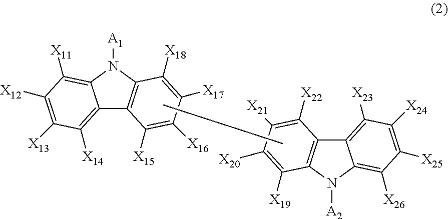

In Formula 2, A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment of the present disclosure, A₁ and A₂, each independently, may represent a substituted or unsubstituted (C6-C25)aryl, a substituted or unsubstituted dibenzofuranyl, a substituted or unsubstituted dibenzothiophenyl, or a substituted or unsubstituted carbazolyl. According to another embodiment of the present disclosure, A₁ and A₂, each independently, may represent a phenyl unsubstituted or substituted with deuterium; a biphenyl unsubstituted or substituted with deuterium; a terphenyl unsubstituted or substituted with deuterium; a naphthyl unsubstituted or substituted with deuterium; a fluorenyl unsubstituted or substituted with at least one of deuterium, a (C1-C30)alkyl(s), and a (C6-C30)aryl(s); a benzofluorenyl unsubstituted or substituted with at least one of deuterium, a (C1-C30)alkyl(s), and a (C6-C30)aryl(s); a triphenylenyl unsubstituted or substituted with deuterium; a fluoranthenyl unsubstituted or substituted with deuterium; a phenanthrenyl unsubstituted or substituted with deuterium; a dibenzofuranyl unsubstituted or substituted with deuterium; a dibenzothiophenyl unsubstituted or substituted with deuterium; a carbazolyl unsubstituted or substituted with deuterium; or a combination thereof. For example, A₁ and A₂, each independently, may be a substituted or unsubstituted, phenyl, p-biphenyl, m-biphenyl, o-biphenyl, p-terphenyl, m-terphenyl, o-terphenyl, naphthyl, fluorenyl, benzofluorenyl, triphenylenyl, fluoranthenyl, phenanthrenyl, dibenzofuranyl, dibenzothiophenyl, or carbazolyl.

Wherein, the substituents of A₁ and A₂, each independently, may be at least one of deuterium, a (C6-C30)aryl(s), and a (3- to 30-membered)heteroaryl(s), and according to one embodiment of the present disclosure, at least one of deuterium, a (C6-C18)aryl(s), and a (5- to 20-membered)heteroaryl(s). For example, the substituents of A₁ and A₂, each independently, may be at least one of a phenyl(s), a naphthyl(s), a triphenylenyl(s), a dibenzofuranyl(s) and a dibenzothiophenyl(s), and may be further substituted with deuterium.

In Formula 2, one of X₁₅ to X₁₈ links to one of X₁₉ to X₂₂ to form a single bond. Specifically, Formula 2 may be represented by any one of the following Formulas 2-1 to 2-8.

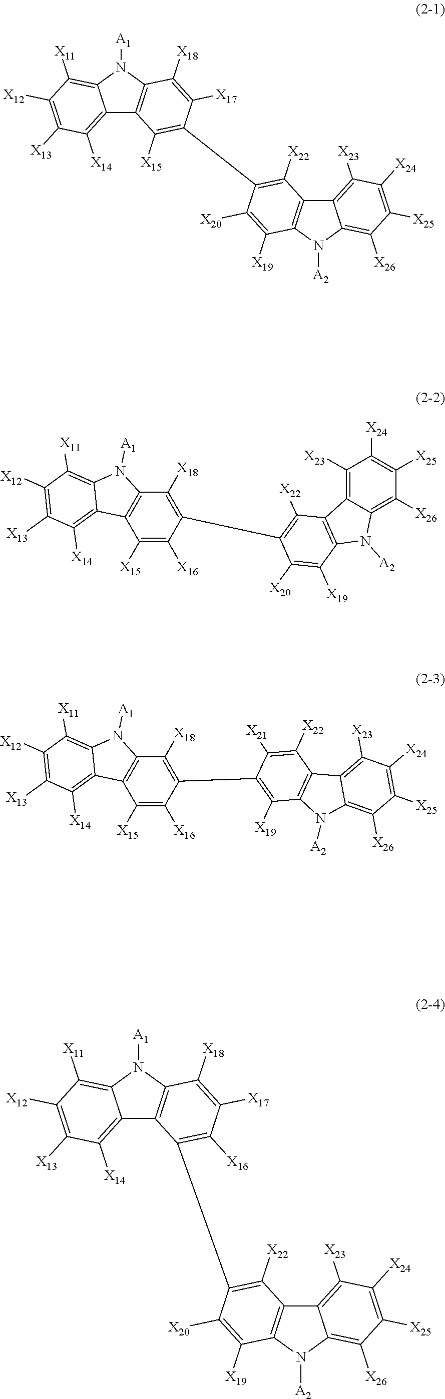

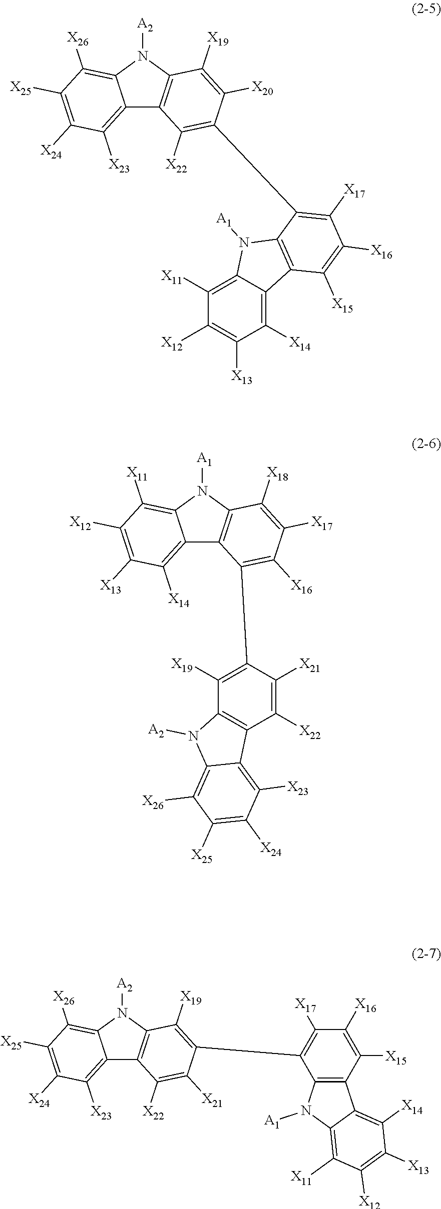

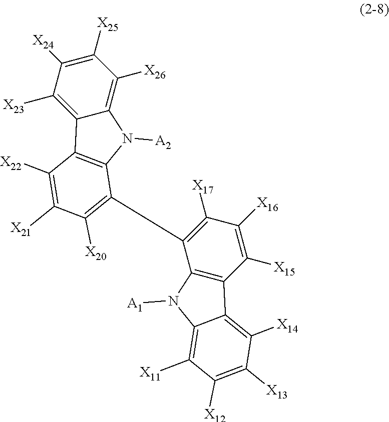

In Formulas 2-1 to 2-8, A₁, A₂, and X₁₁ to X₂₆ are as defined in Formula 2.

In Formula 2, X₁₁ to X₁₄, X₂₃ to X₂₆, and X₁₅ to X₂₂ not forming a single bond, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s). According to one embodiment of the present disclosure, X₁₁ to X₁₄, X₂₃ to X₂₆, and X₁₅ to X₂₂ not forming a single bond, each independently, represent hydrogen, deuterium, a substituted or unsubstituted (C6-C30)aryl, or a substituted or unsubstituted (5- to 30-membered)heteroaryl. According to another embodiment of the present disclosure, X₁₁ to X₁₄, X₂₃ to X₂₆, and X₁₅ to X₂₂ not forming a single bond, each independently, represent hydrogen or deuterium. For example, at least four of X₁₁ to X₁₄, X₂₃ to X₂₆, and X₁₅ to X₂₂ not forming a single bond may be deuterium.

According to one embodiment of the present disclosure, at least one, according to another embodiment, at least two, according to another embodiment, at least three, according to another embodiment, all of X₁₁, X₁₈, X₁₉, and X₂₆ in Formula 2 may be deuterium. For example, when X₁₆ and X₂₁ are linked to each other to form a single bond, all of X₁₁, X₁₈, X₁₉, and X₂₆ may be deuterium; and when X₁₈ and X₁₉ are linked to each other to form a single bond, both X₁₁ and X₂₆ may be deuterium.

According to one embodiment of the present disclosure, the substitution rate of deuterium of X₁₁ to X₂₆ in Formula 2 may be 25% to 100%, according to another embodiment of the present disclosure, 35% to 100%, according to another embodiment of the present disclosure, 45% to 100%, and according to another embodiment of the present disclosure, 55% to 100%. The upper limit of the substitution rate of deuterium may be 100%, or may be less than 100%, for example, about 99%.

According to one embodiment of the present disclosure, the substitution rate of deuterium of the compound represented by Formula 2 is 40% to 100%, according to another embodiment of the present disclosure, 50% to 100%, according to another embodiment of the present disclosure, 60% to 100%, and according to another embodiment of the present disclosure, 70% to 100%. The upper limit of the substitution rate of deuterium may be 100%, or may be less than 100%, for example, about 99%. That is, the compound represented by Formula 2 may be a compound in which all of the hydrogen at replaceable positions is replaced with deuterium, or it may be a compound in which the hydrogen at replaceable positions is partially replaced with deuterium.

When the compound represented by Formula 2 contains deuterium substituted according to the above-mentioned number or rate, the stability of the compound represented by Formula 2 may be improved as the bond dissociation energy due to deuteration increases, and an organic electroluminescent device comprising the compound represented by Formula 2 may exhibit improved lifetime properties.

The compound represented by Formula 2 may be selected from the group consisting of the following compounds, but is not limited thereto.

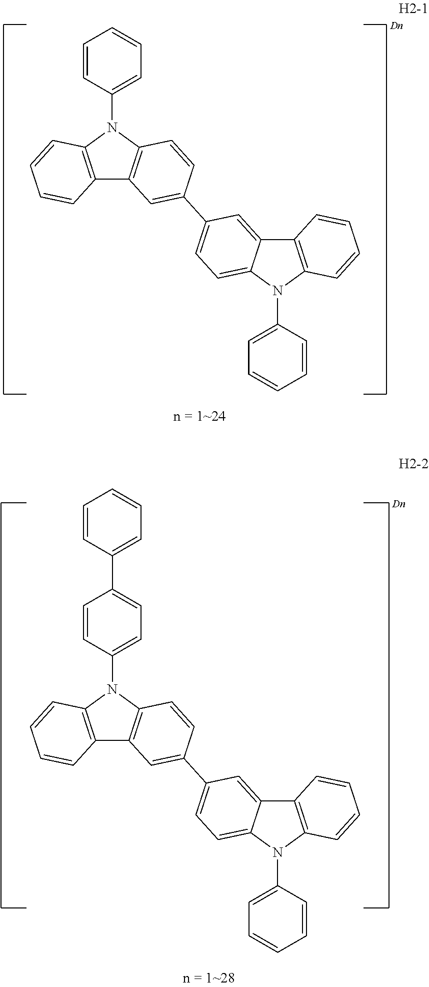

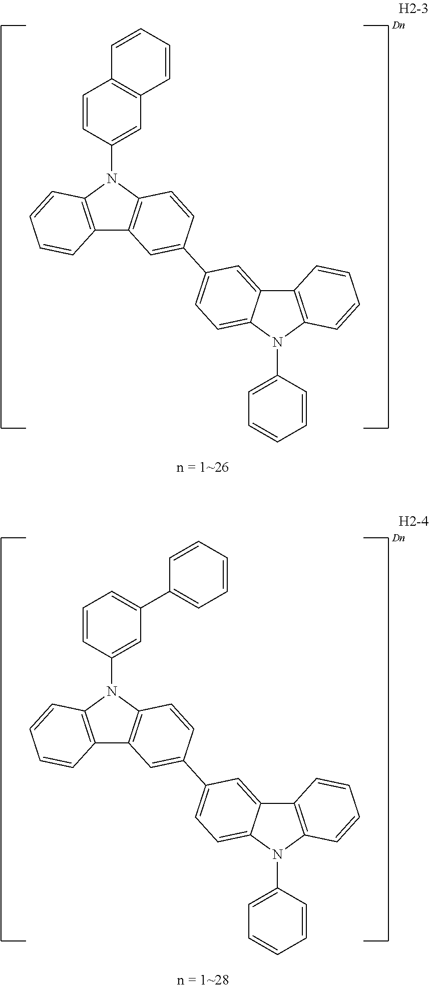

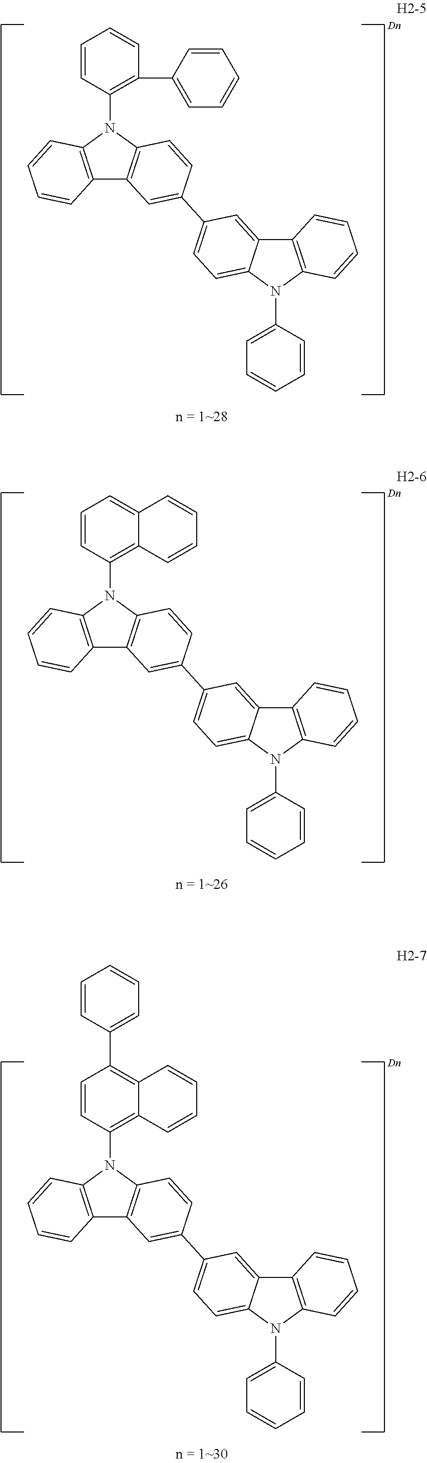

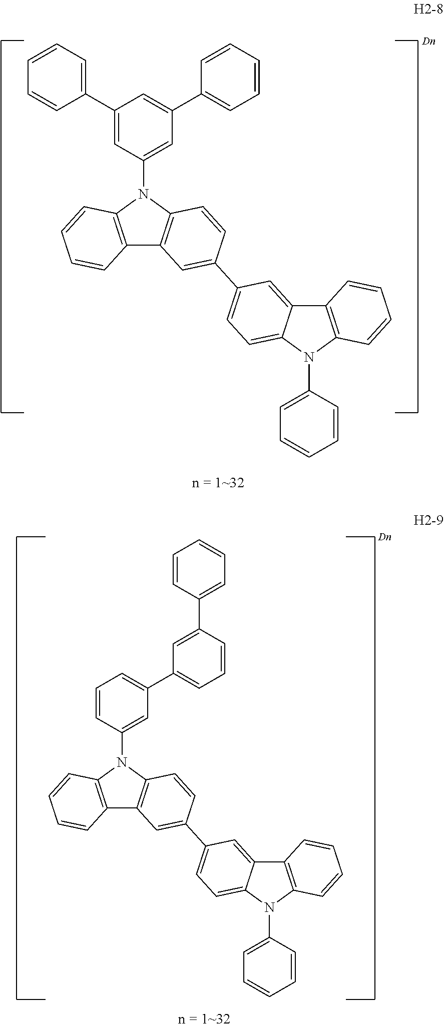

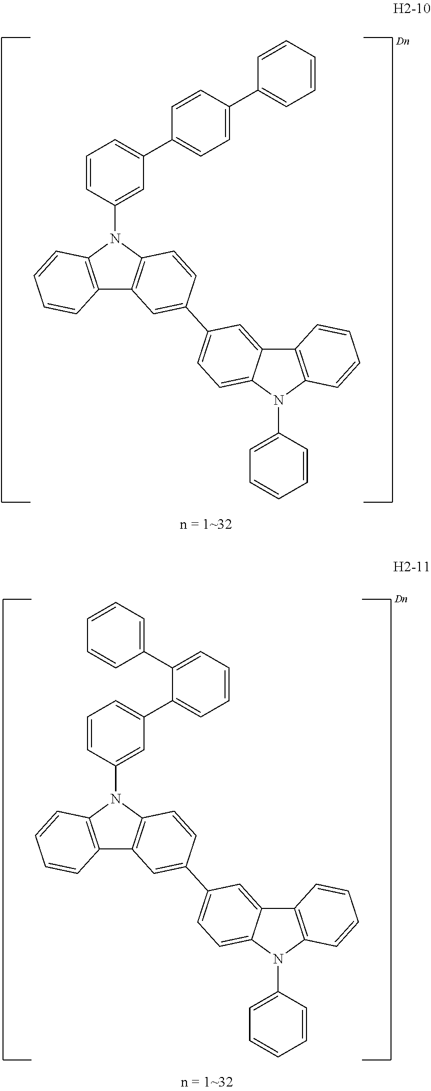

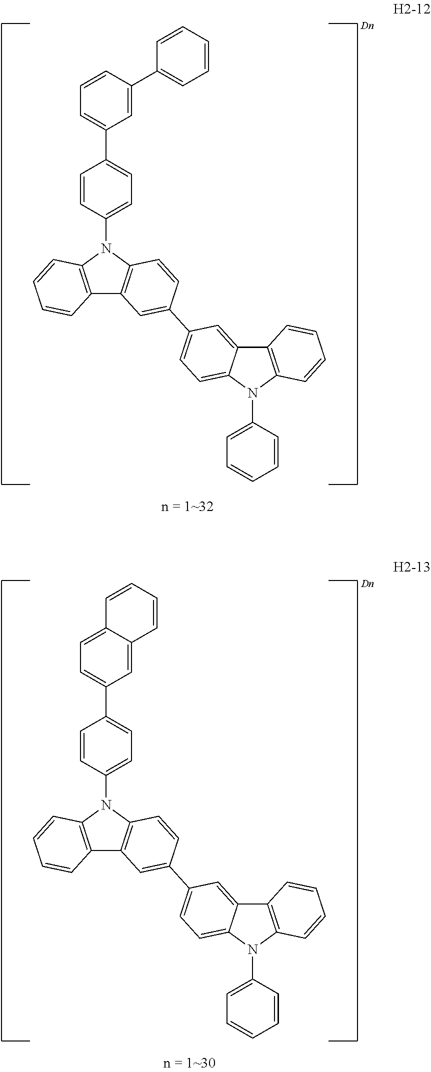

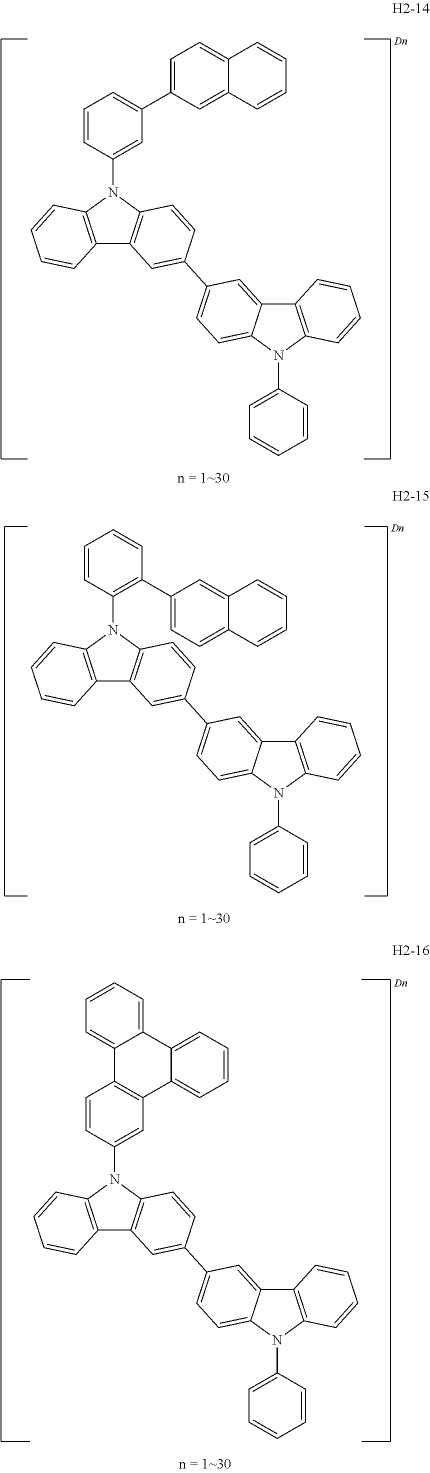

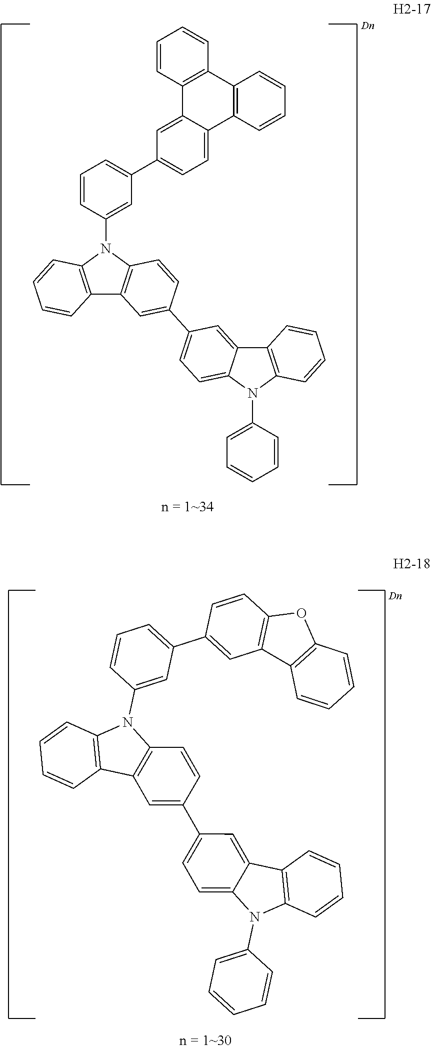

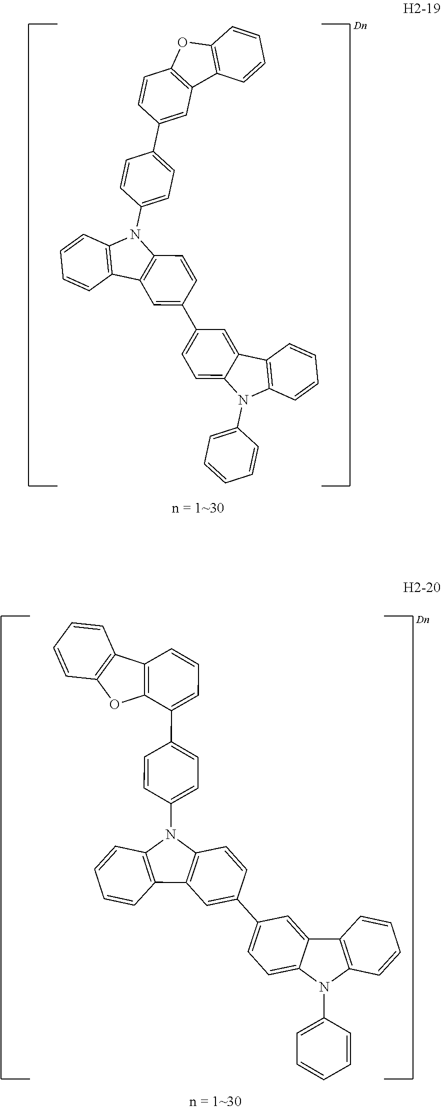

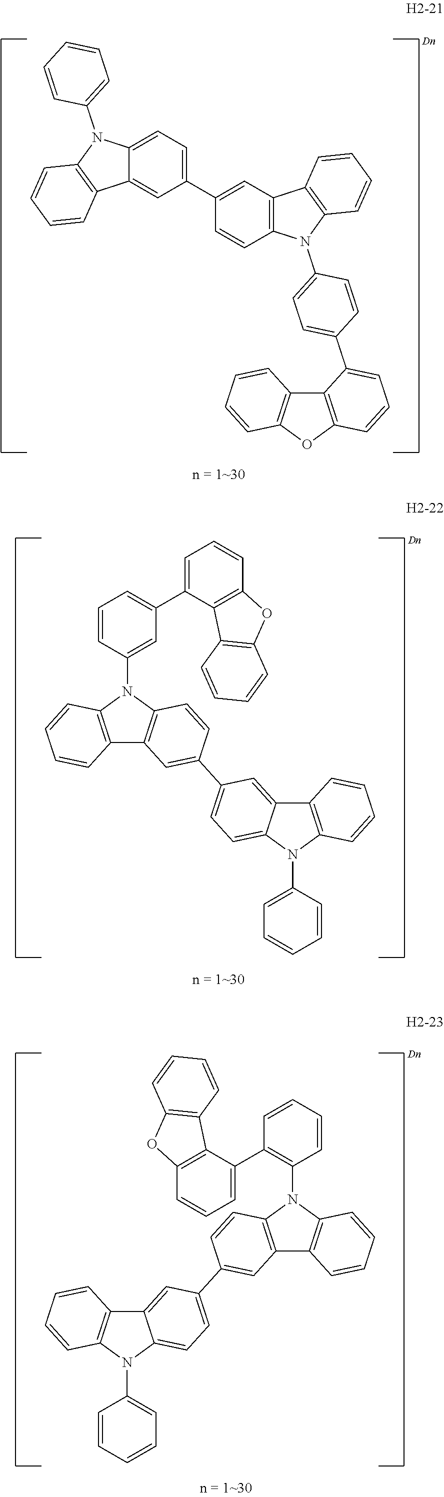

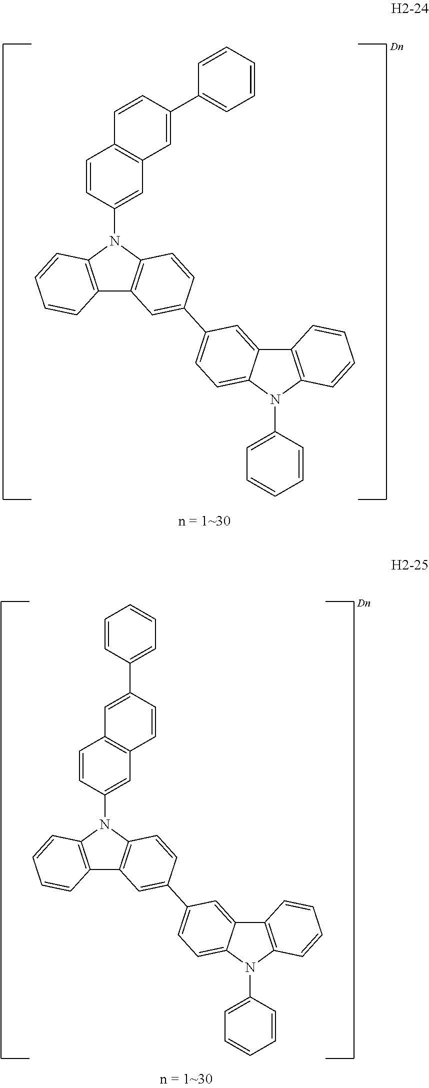

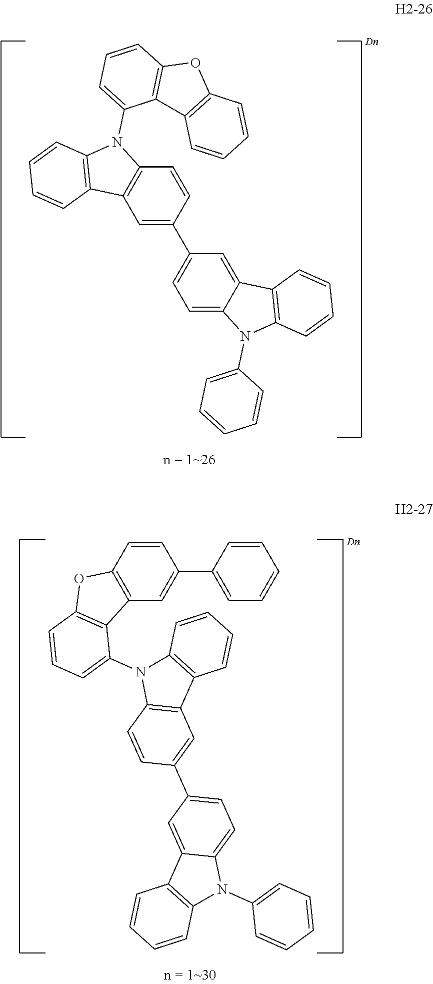

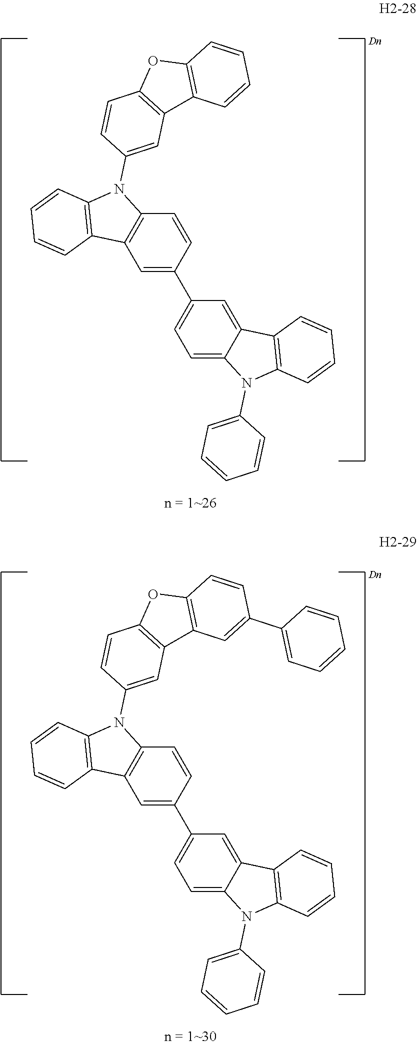

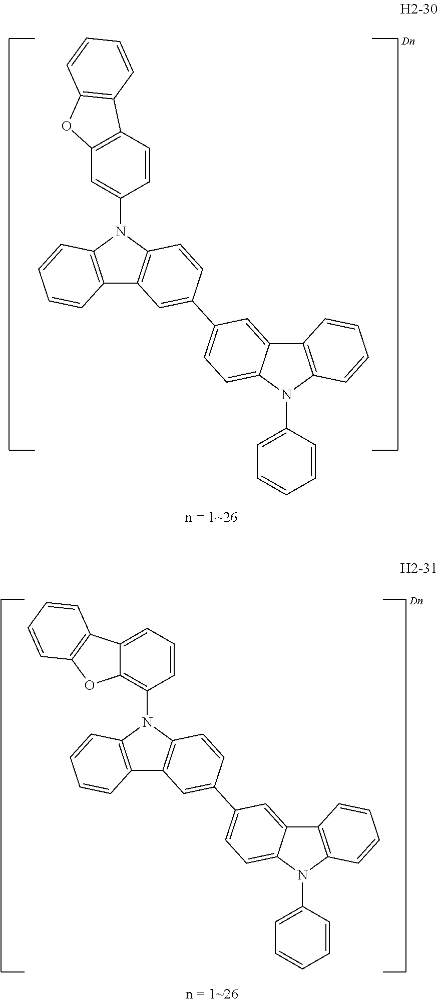

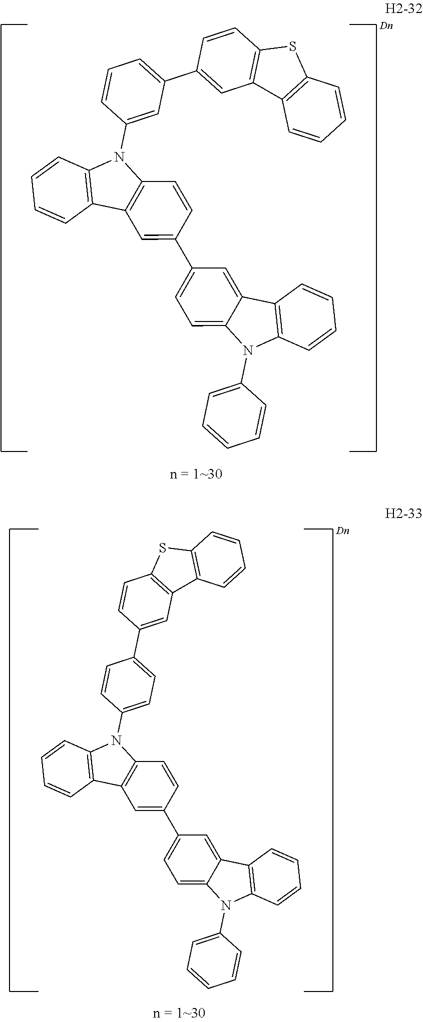

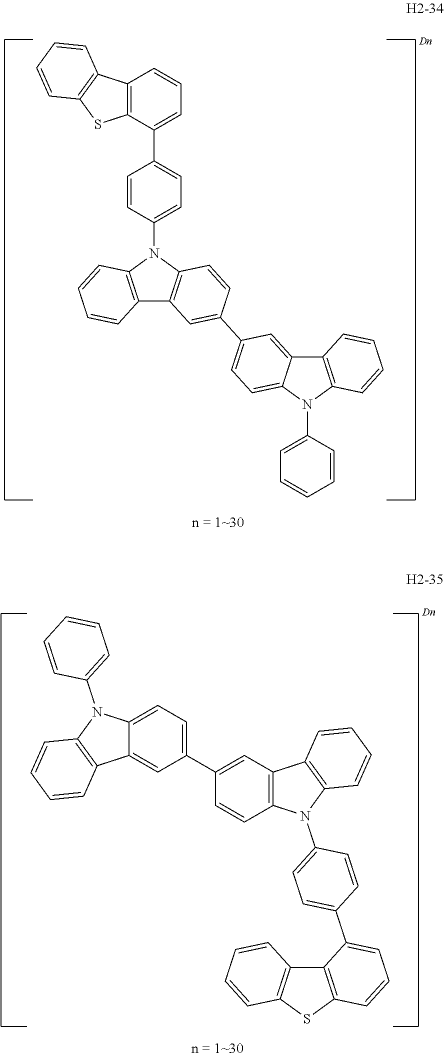

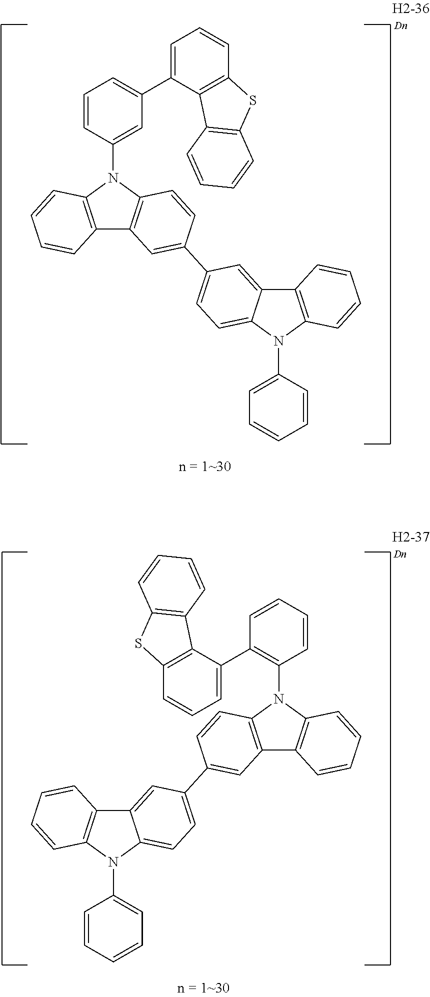

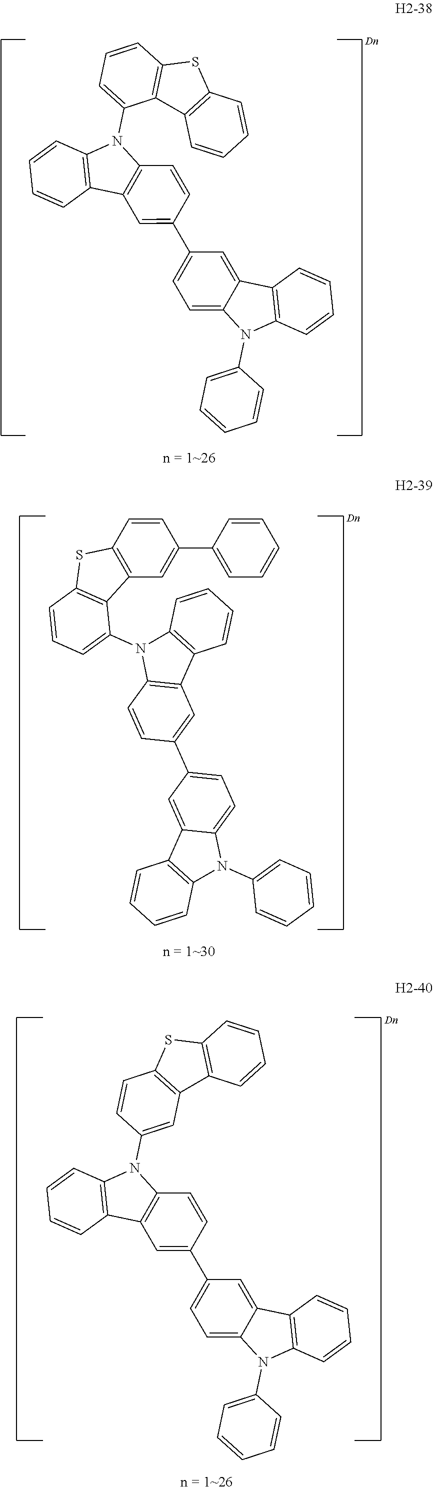

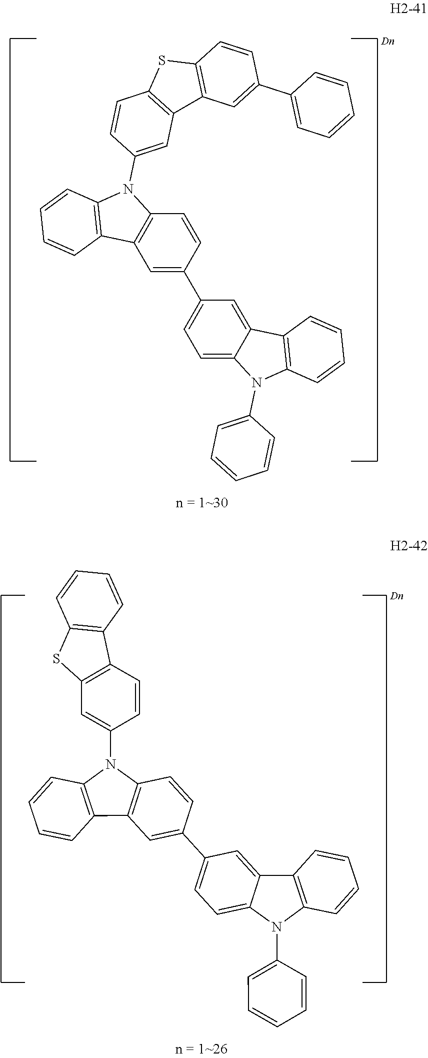

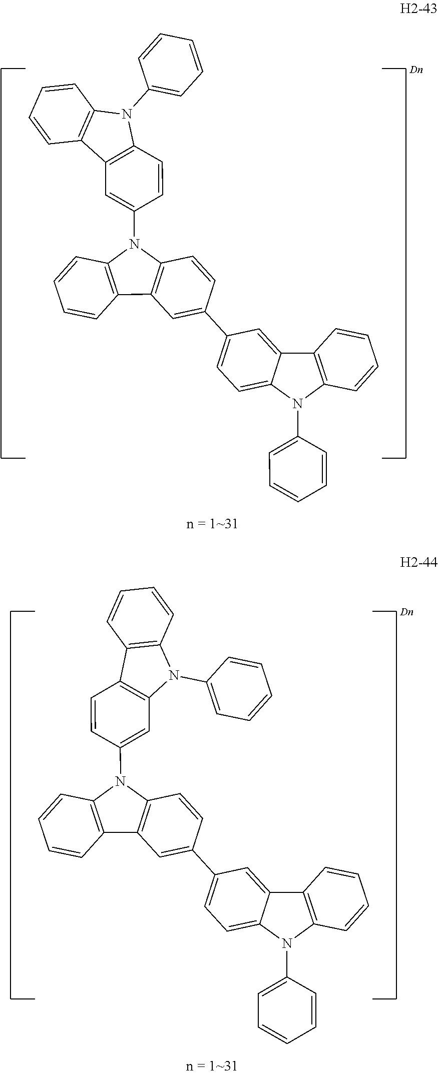

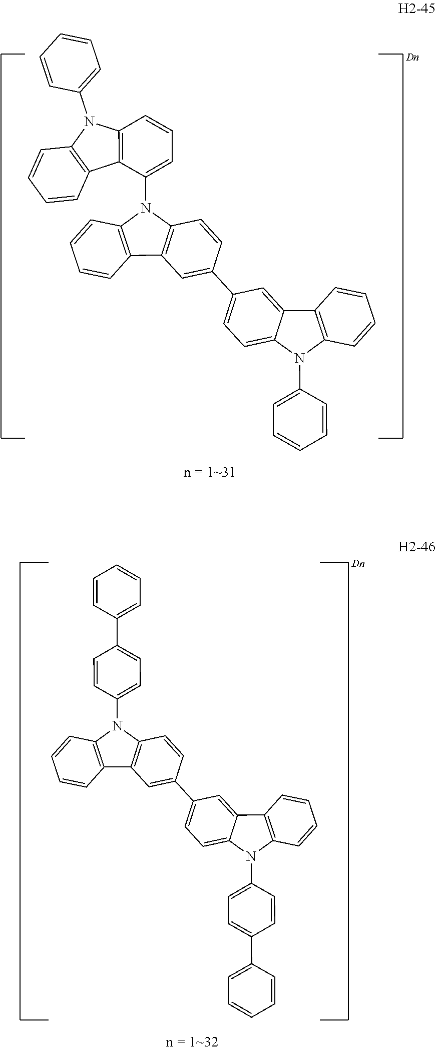

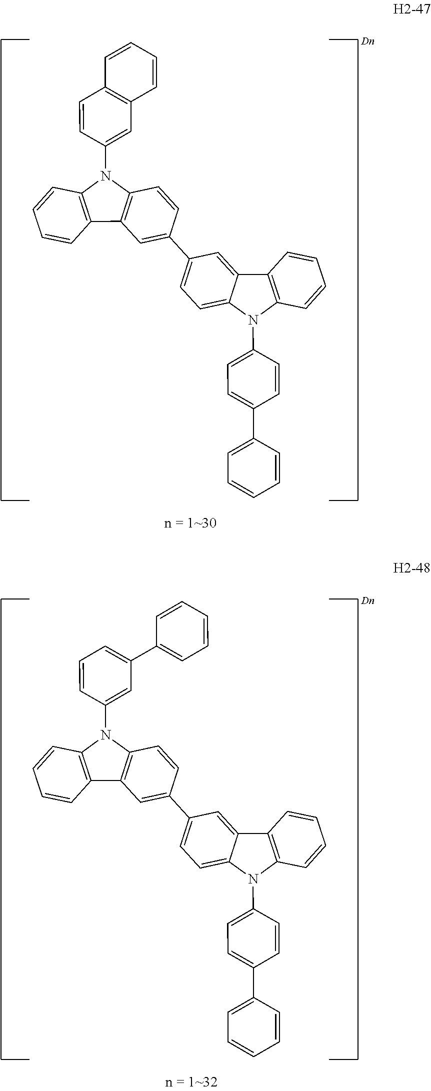

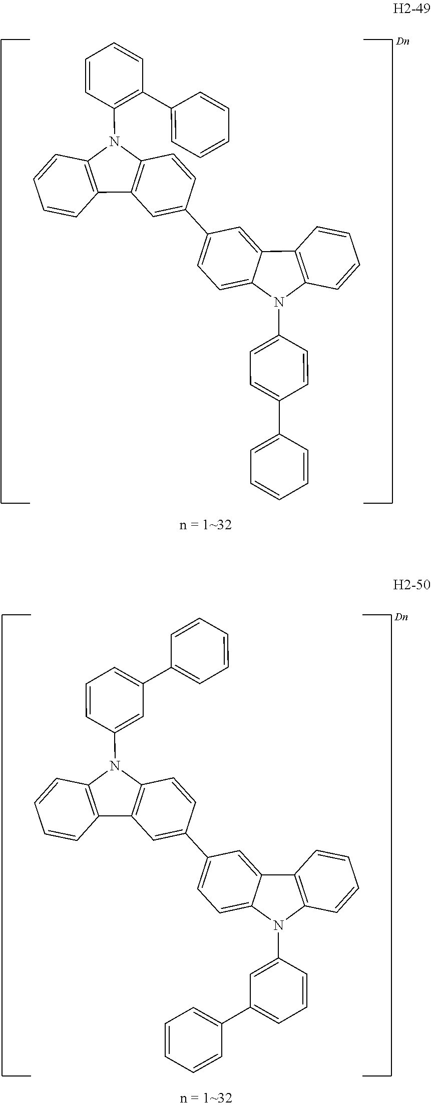

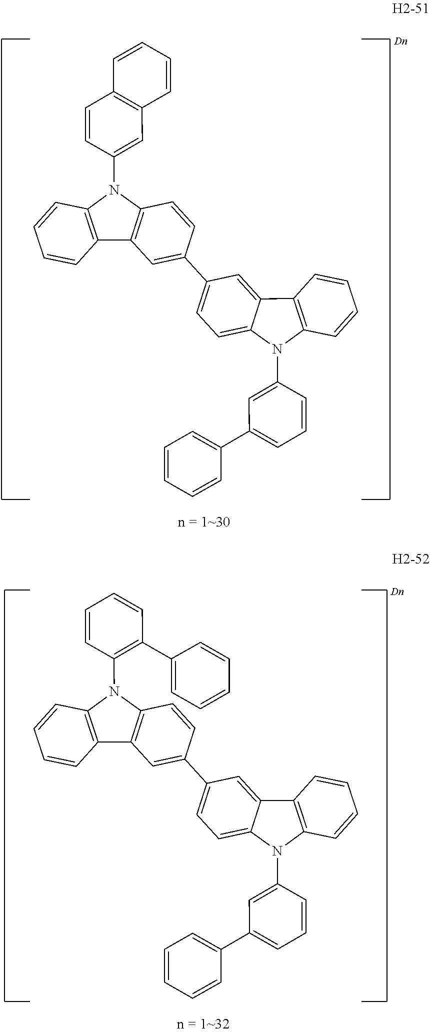

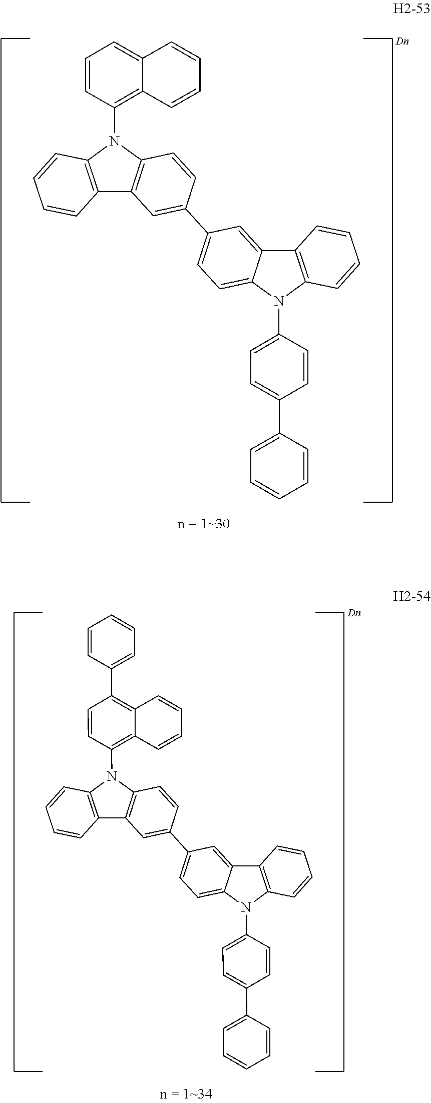

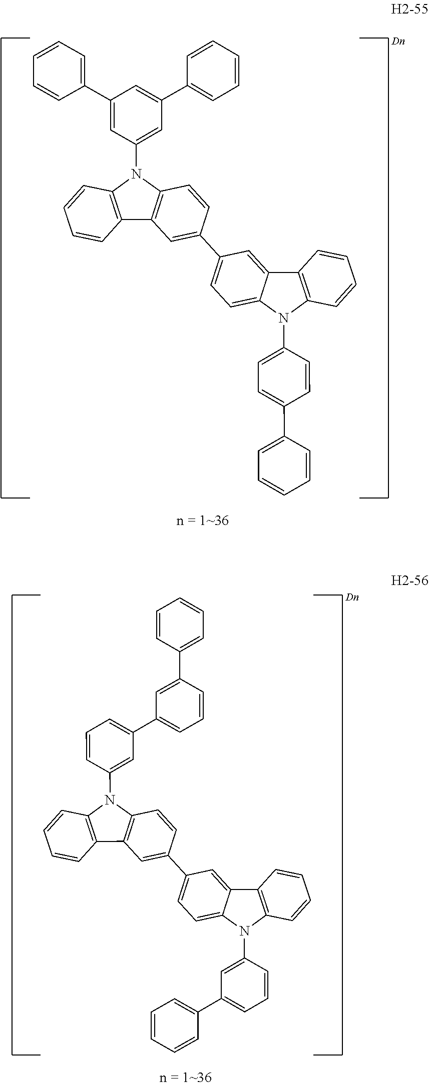

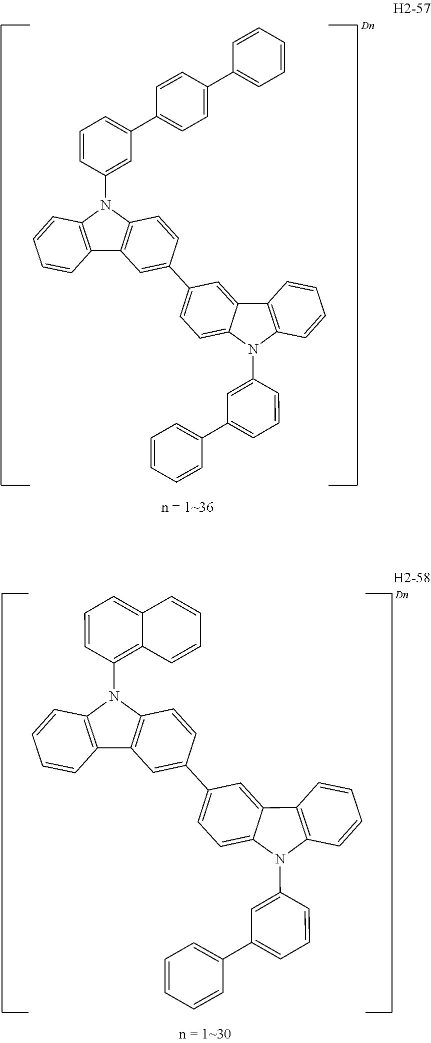

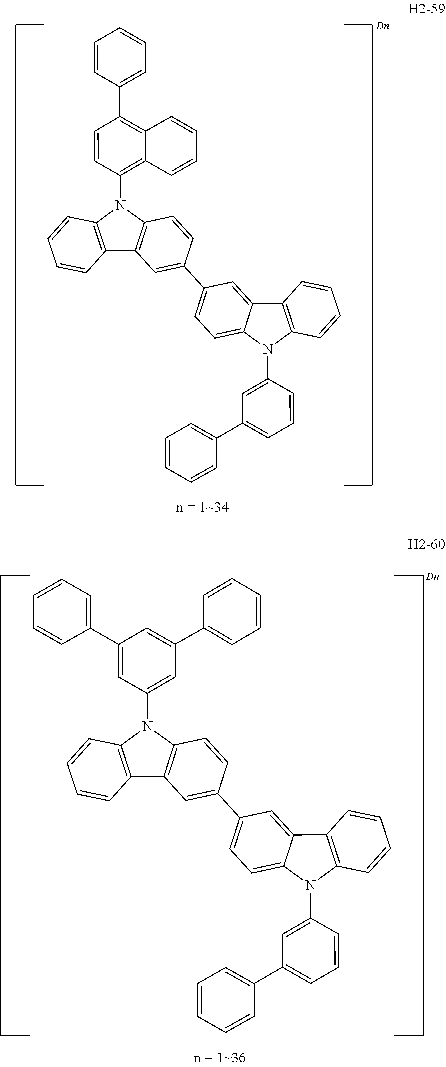

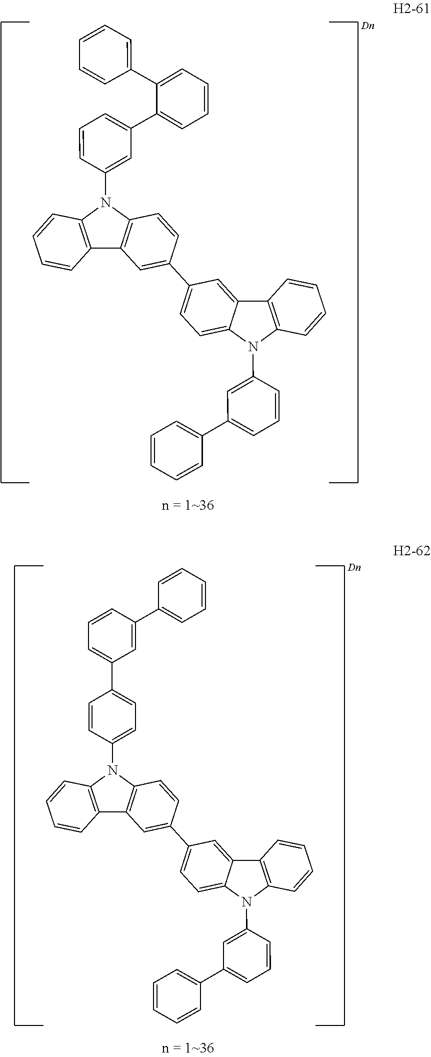

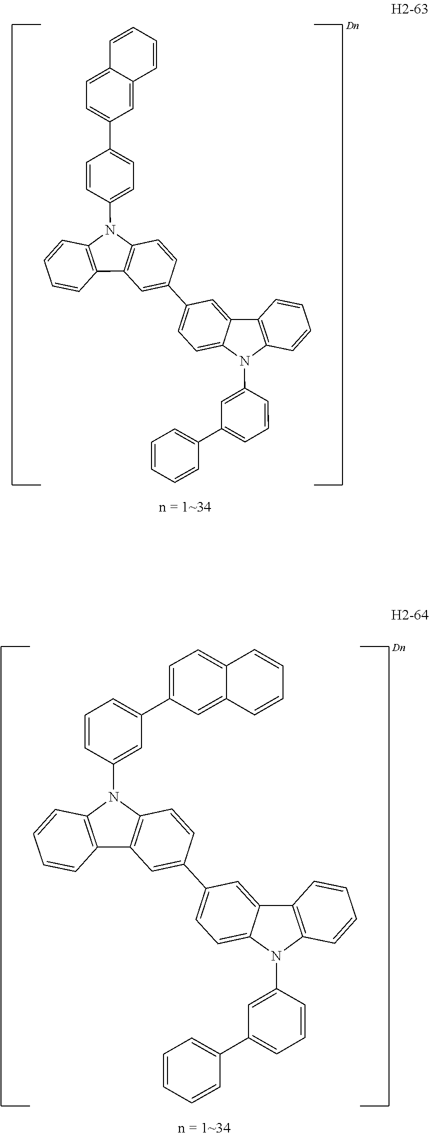

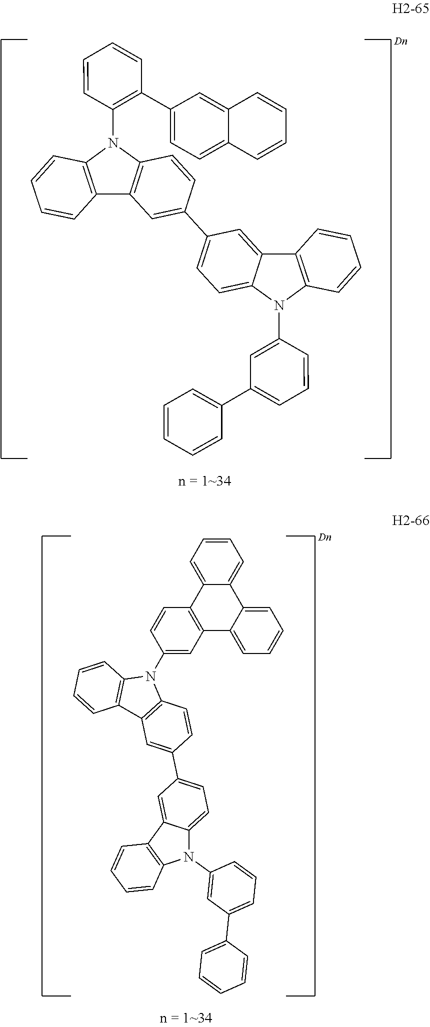

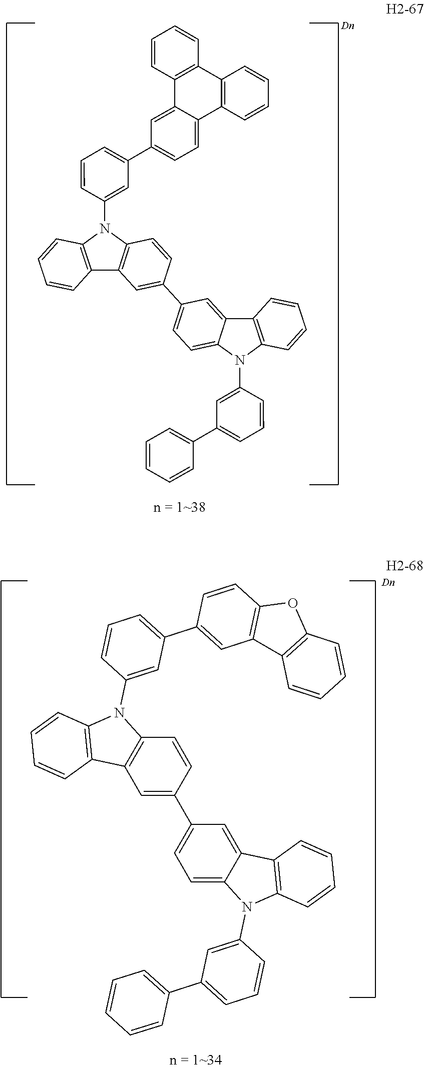

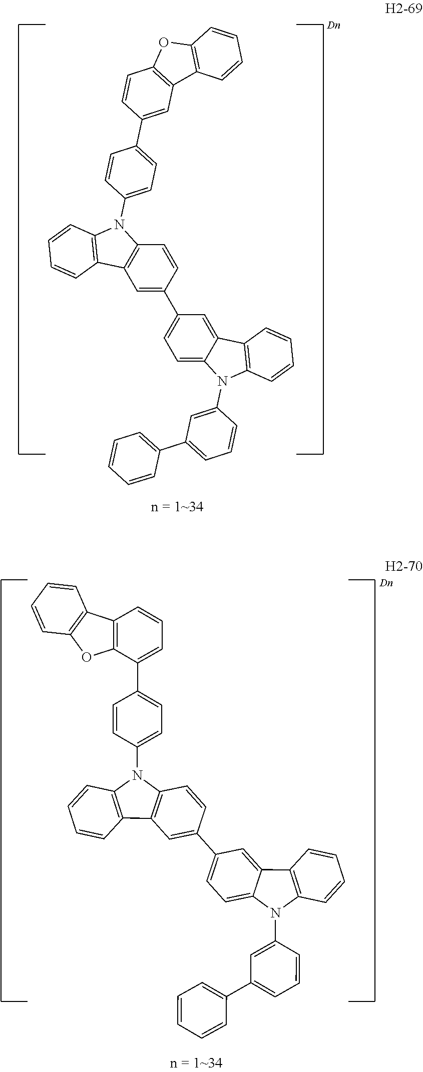

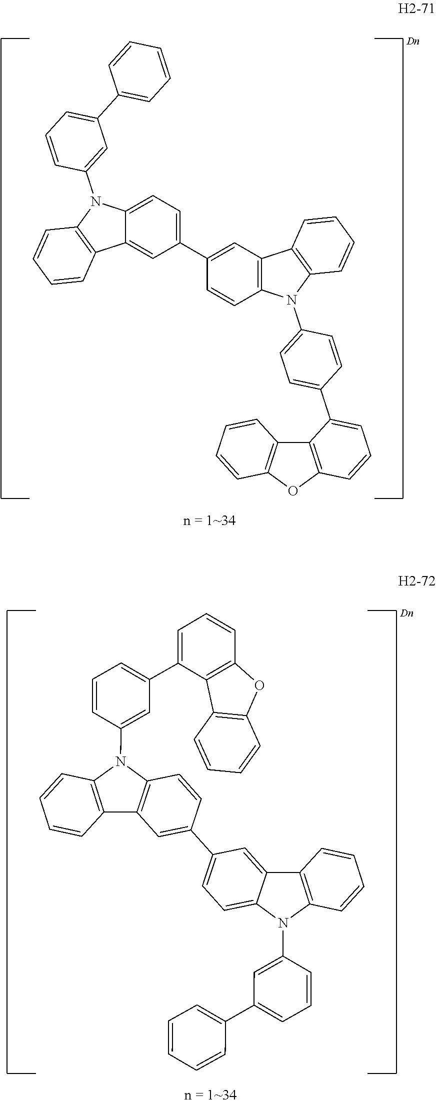

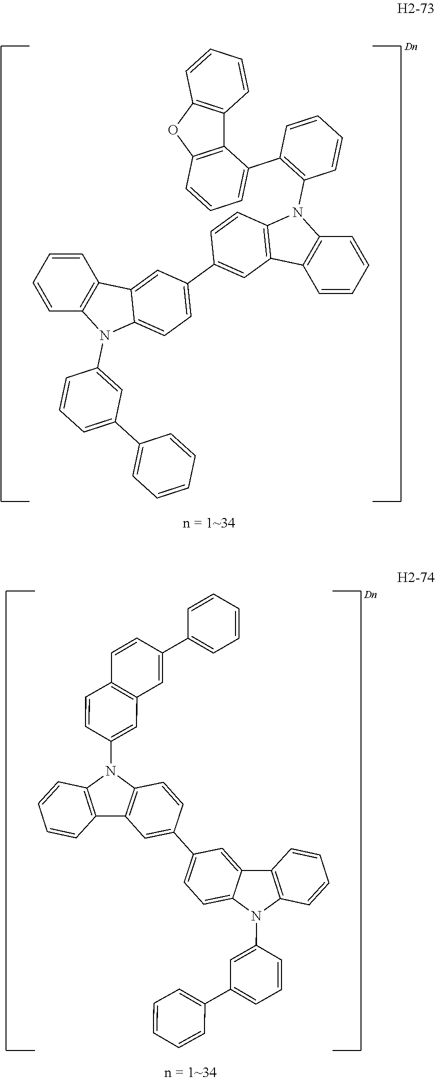

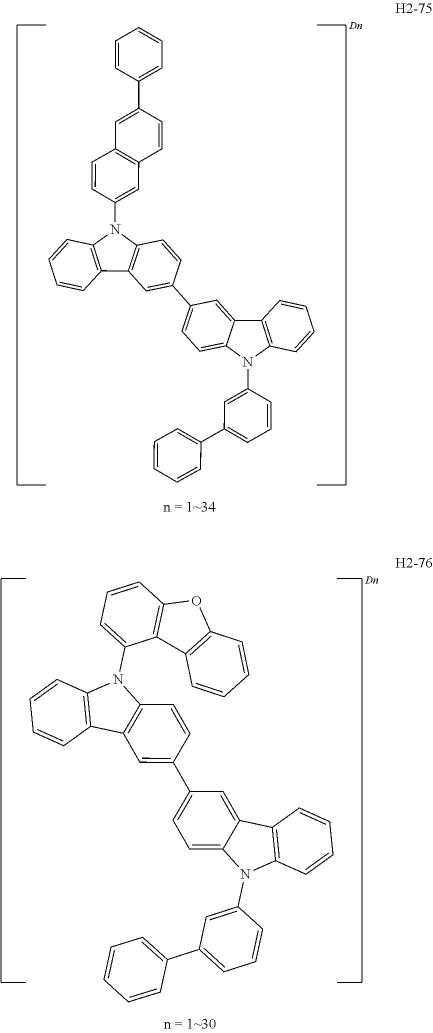

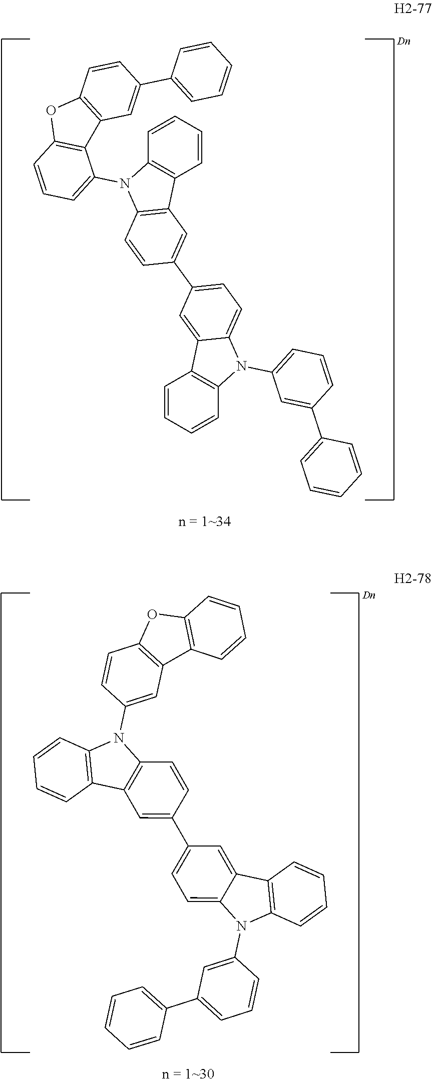

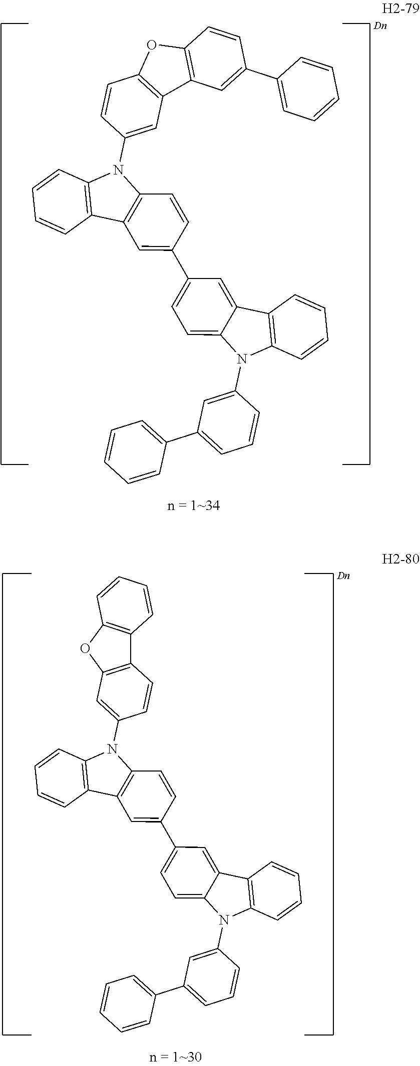

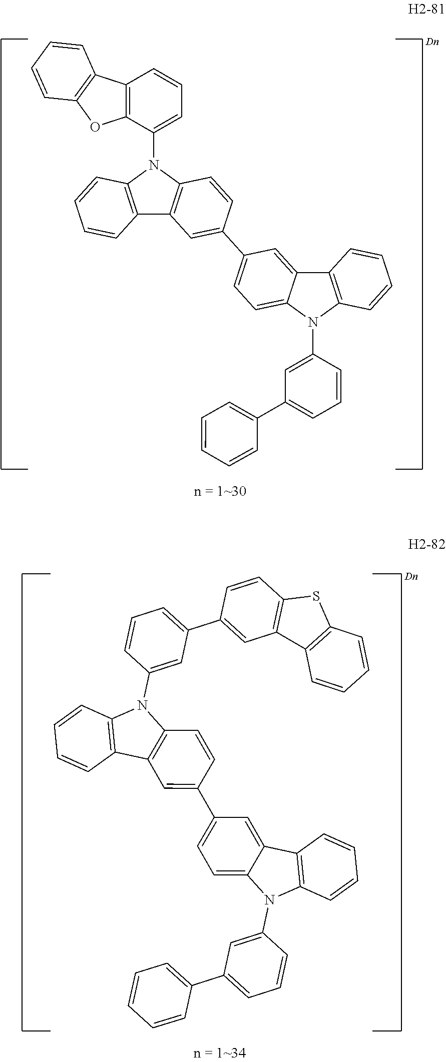

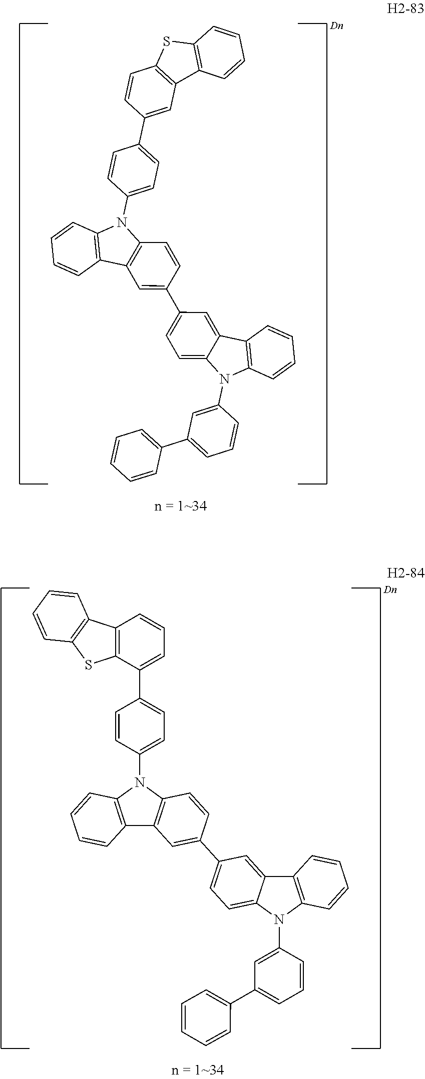

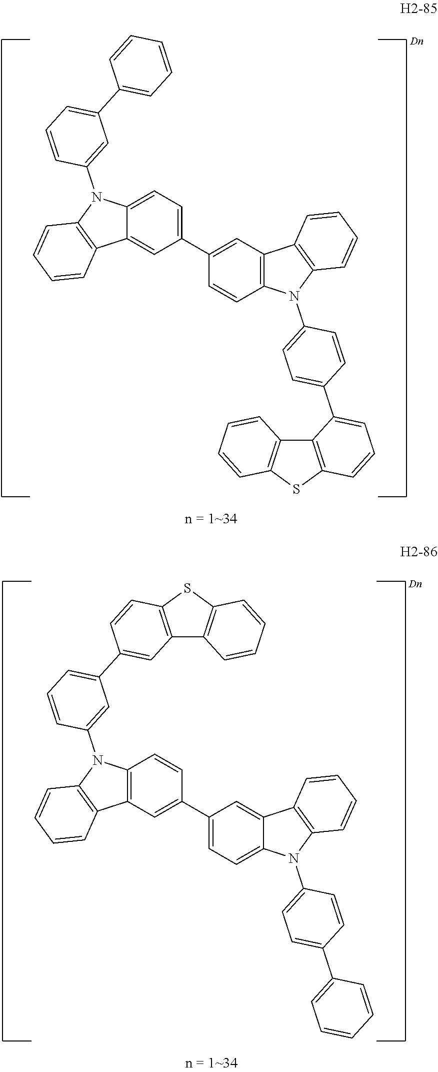

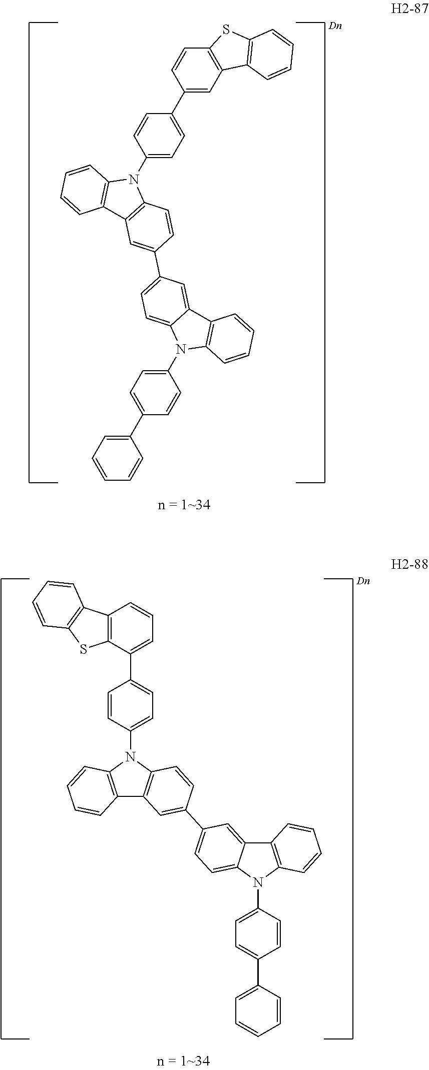

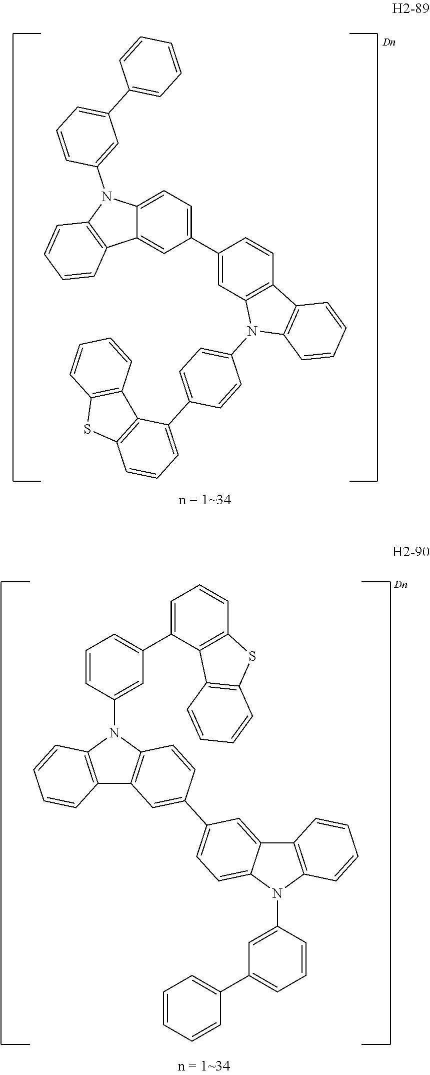

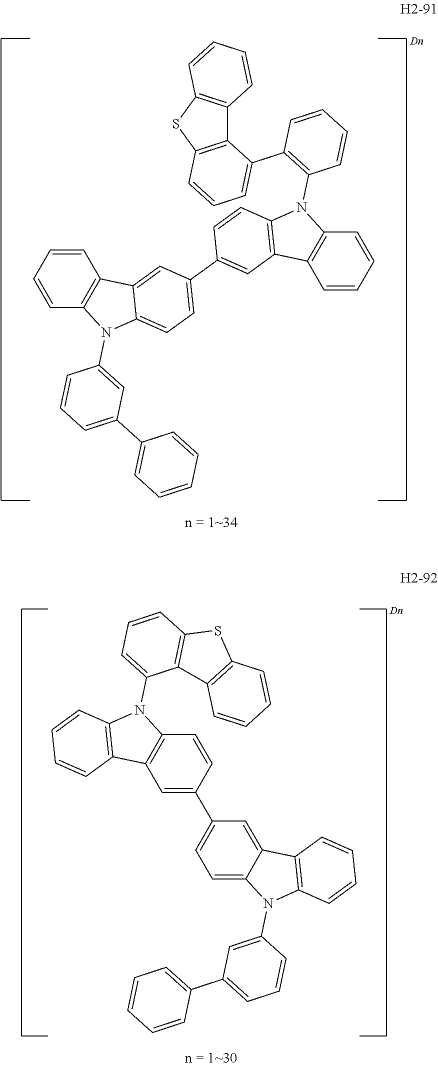

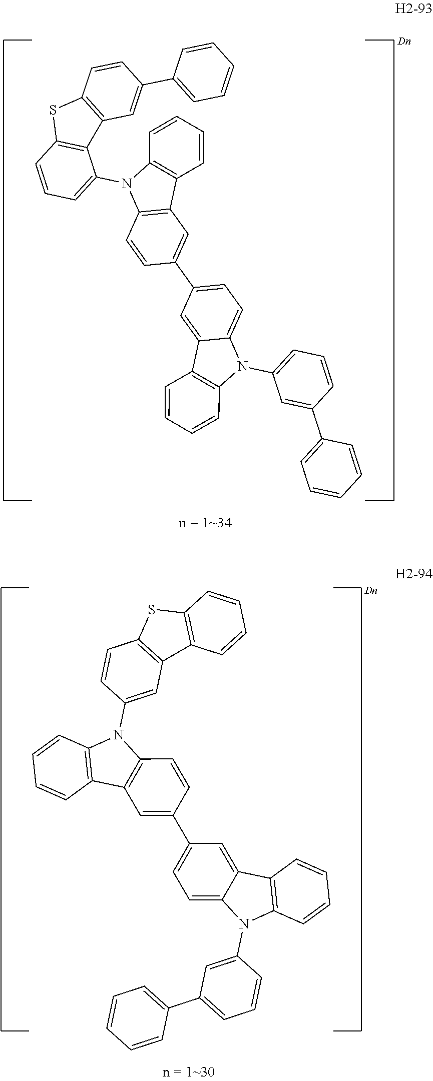

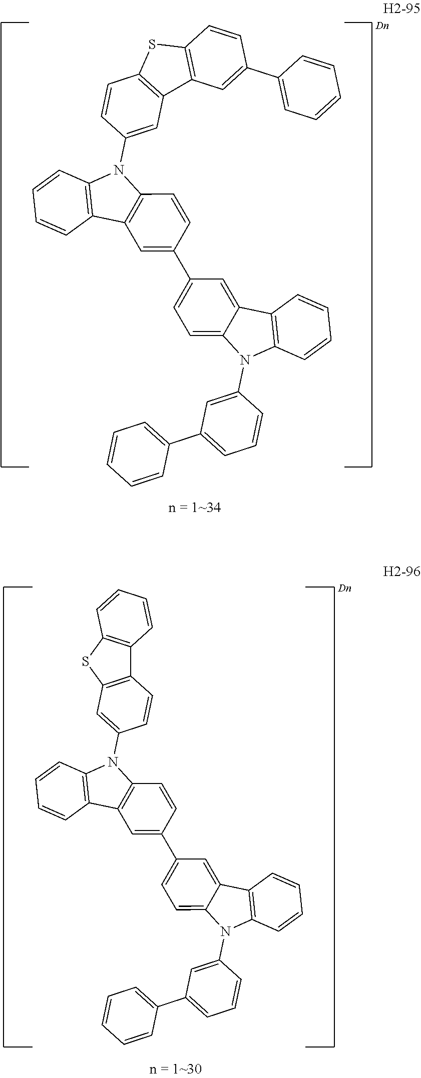

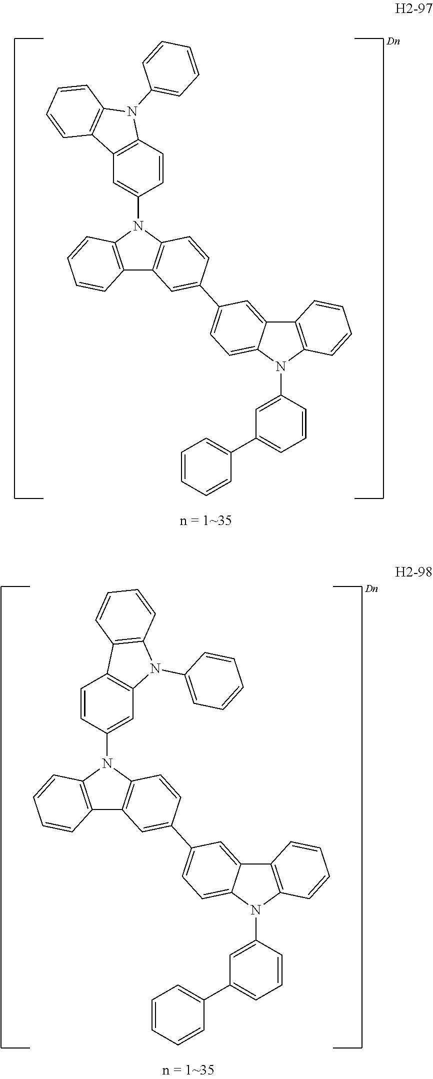

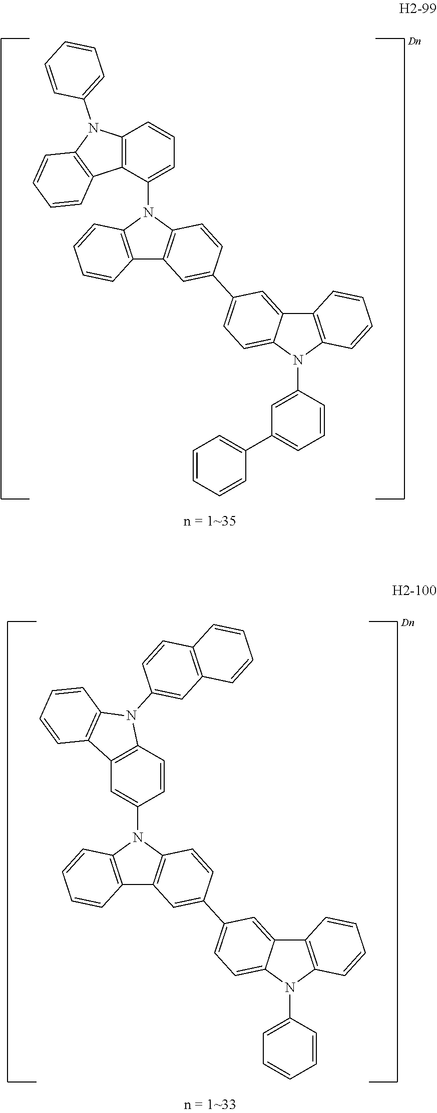

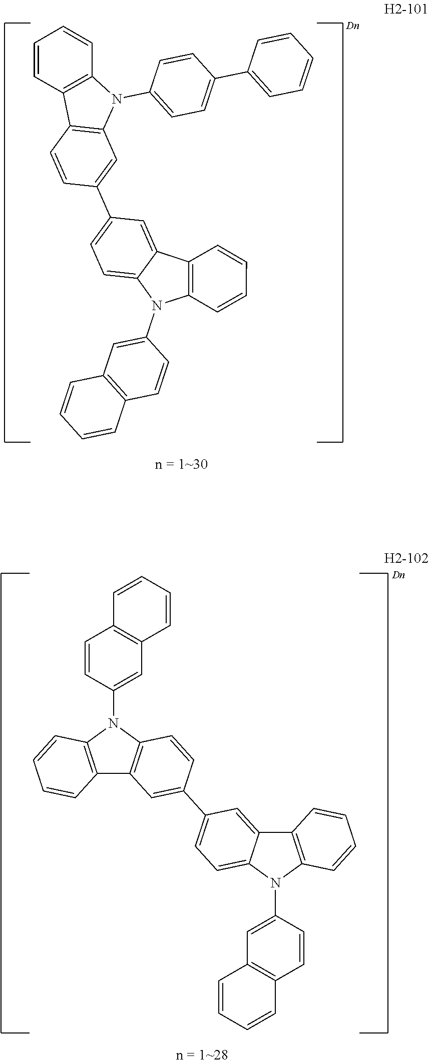

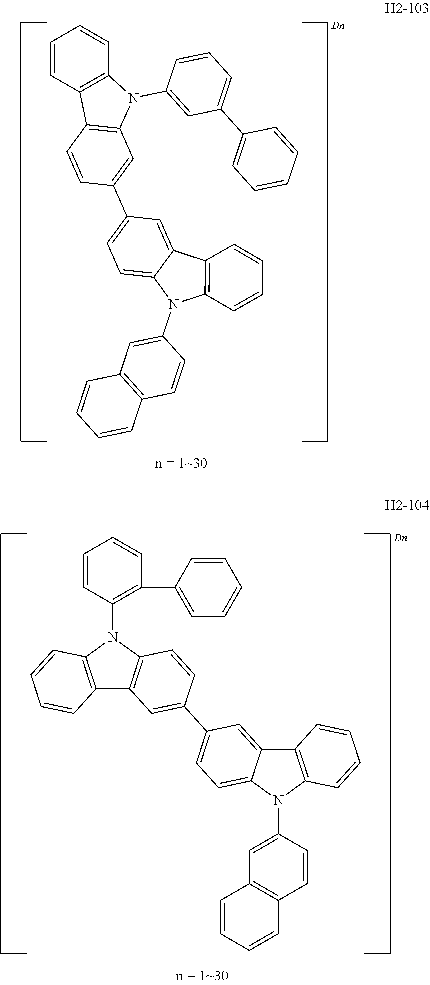

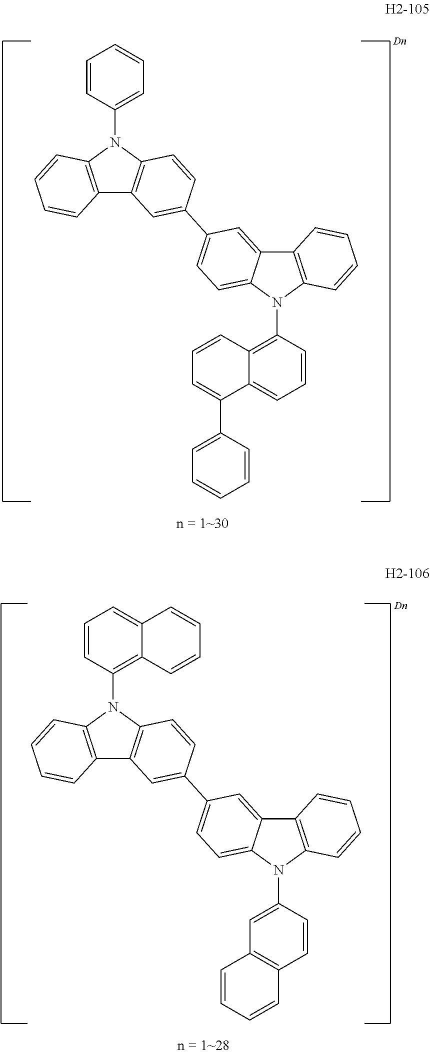

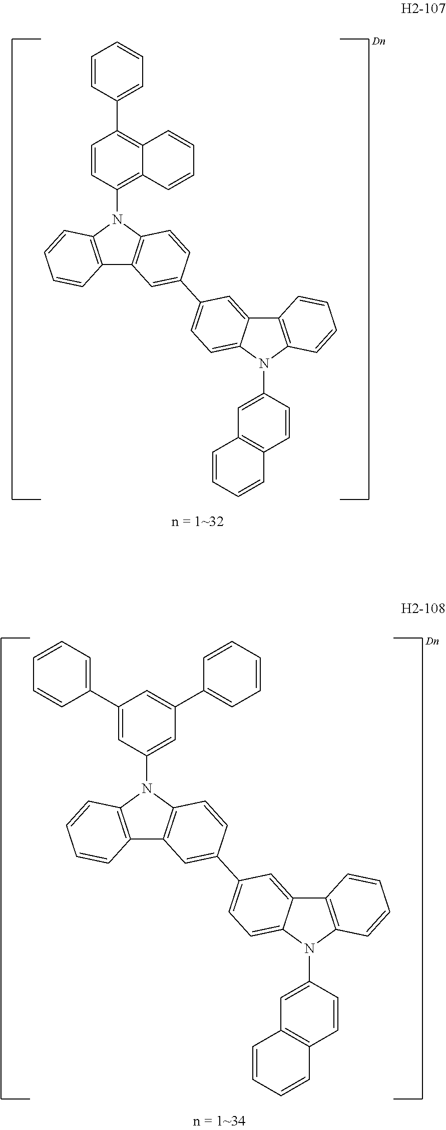

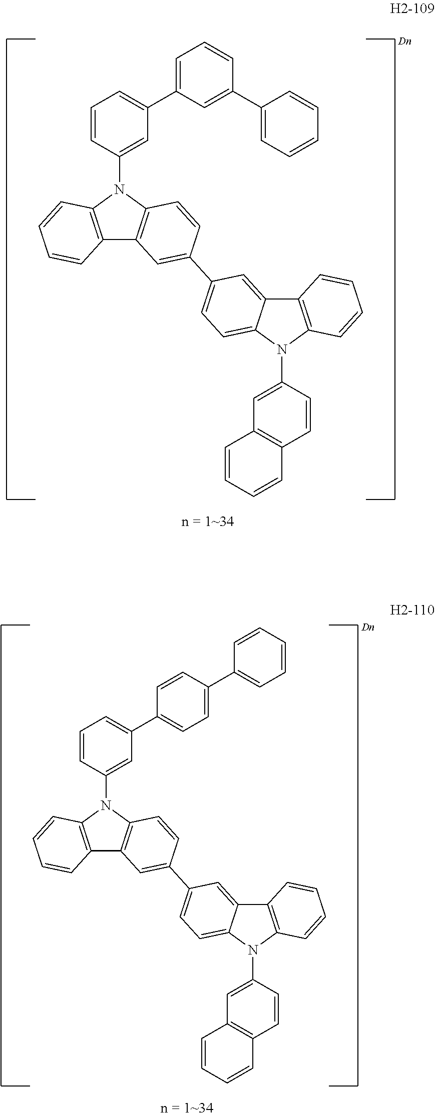

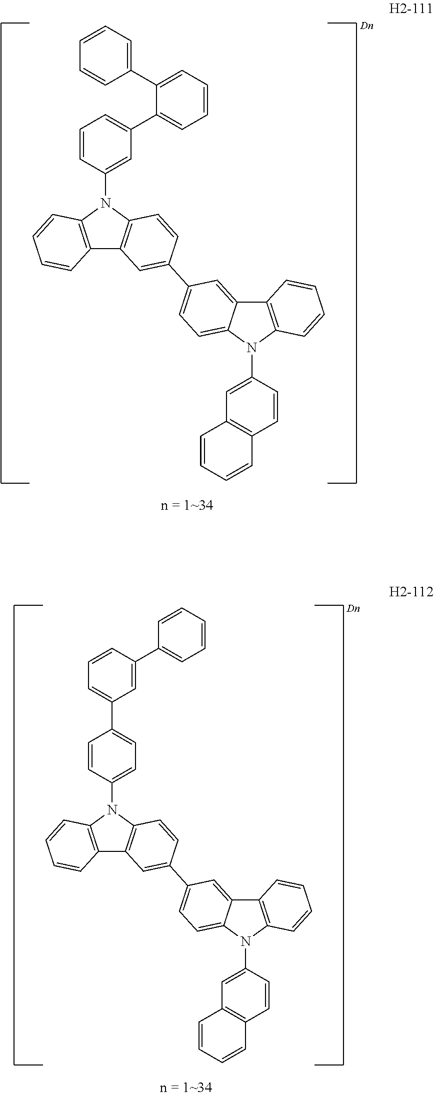

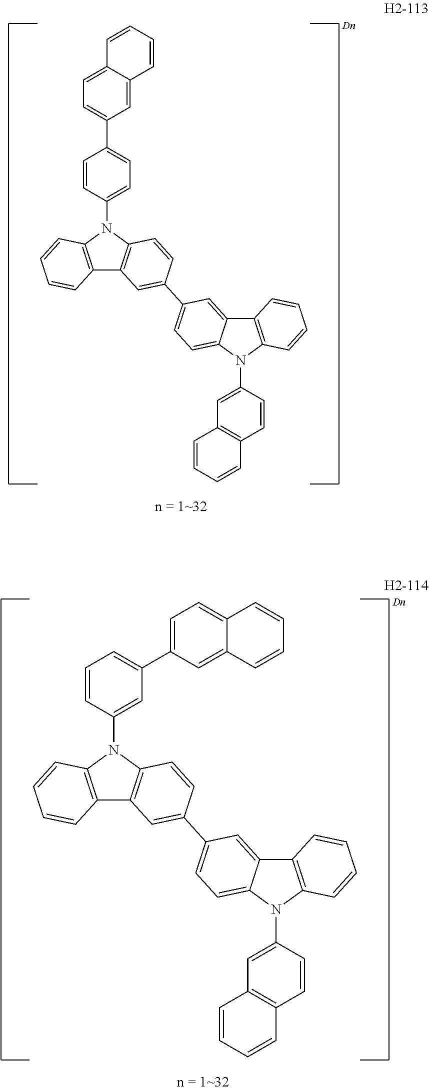

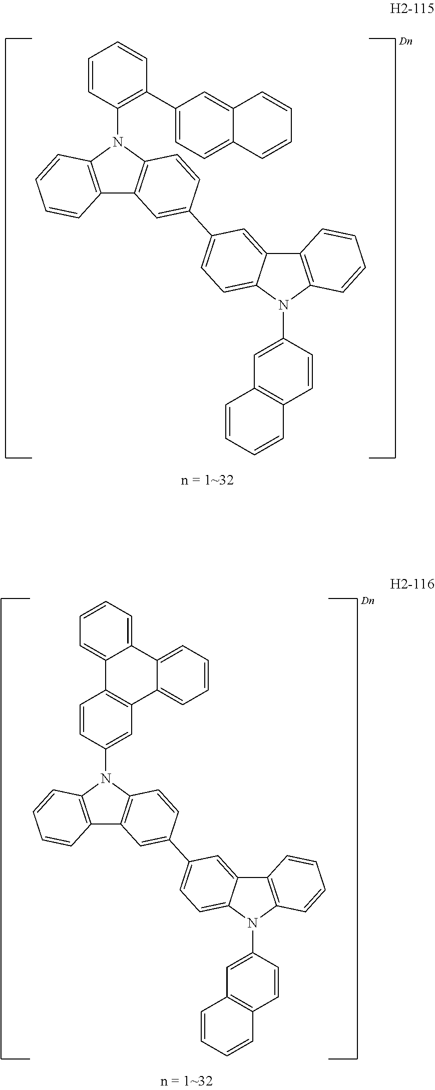

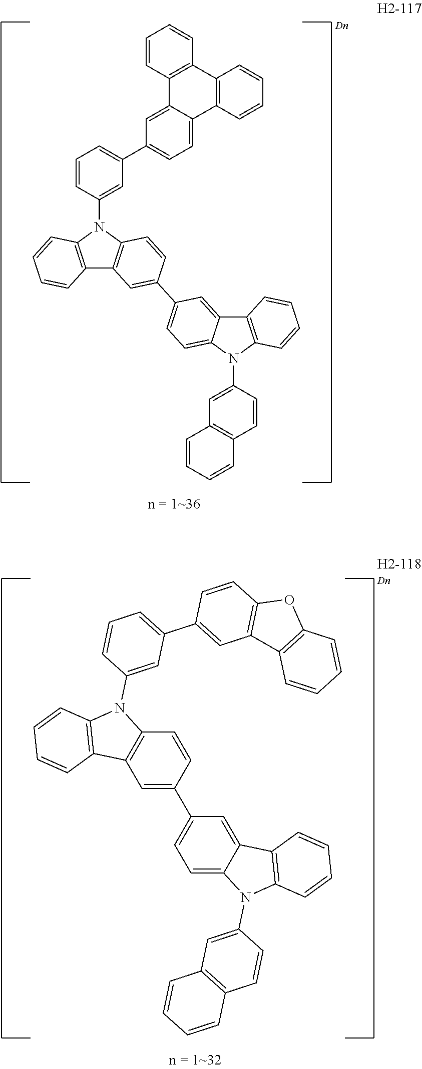

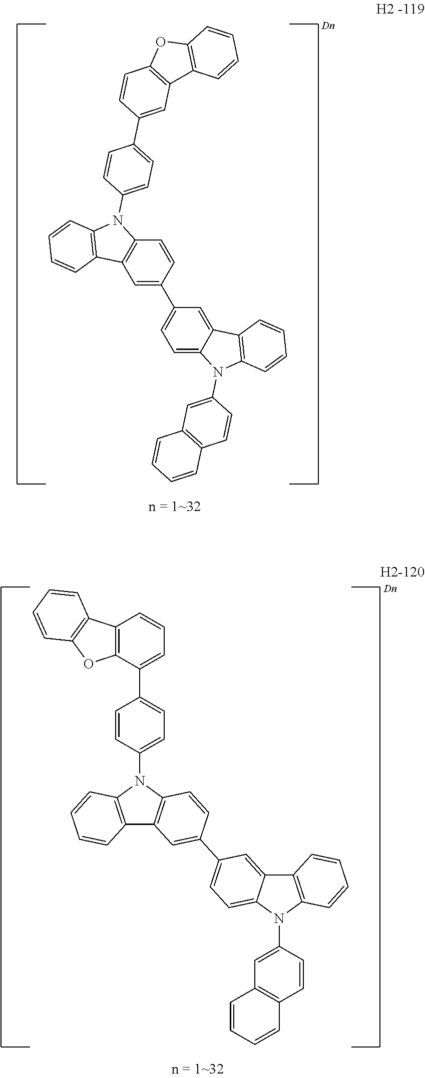

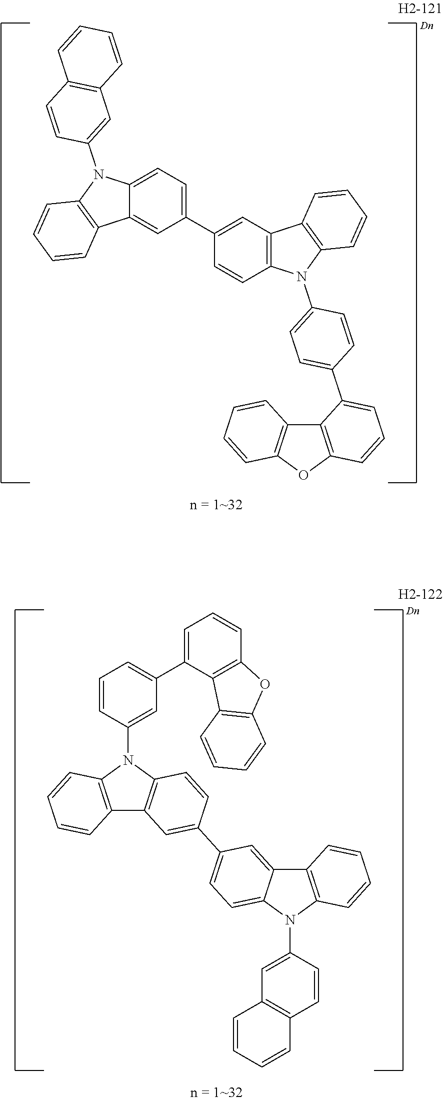

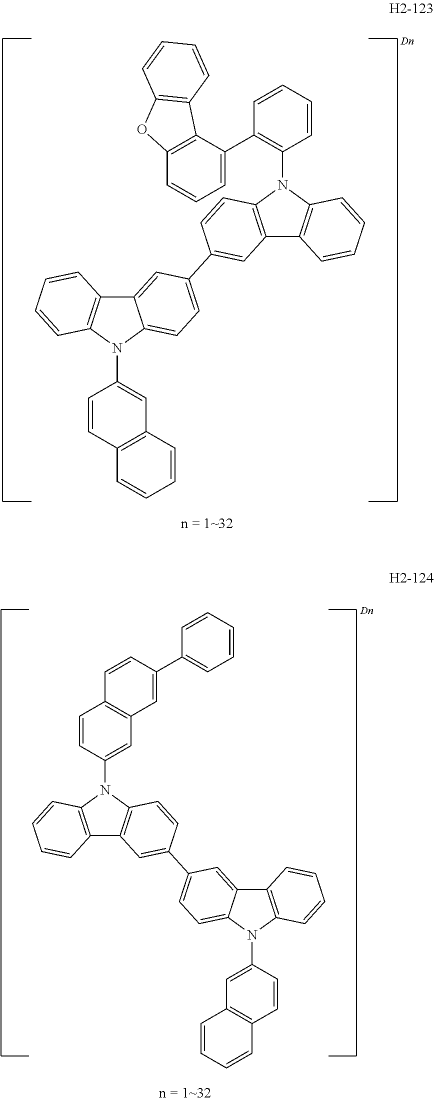

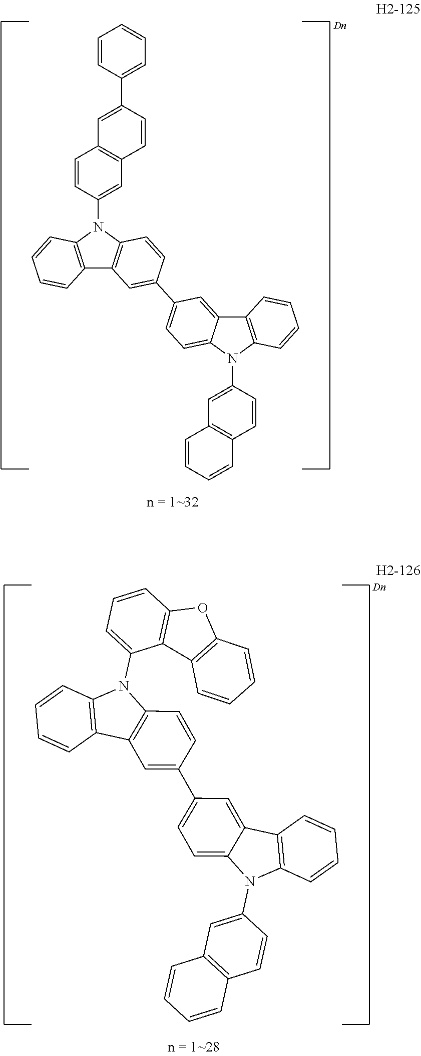

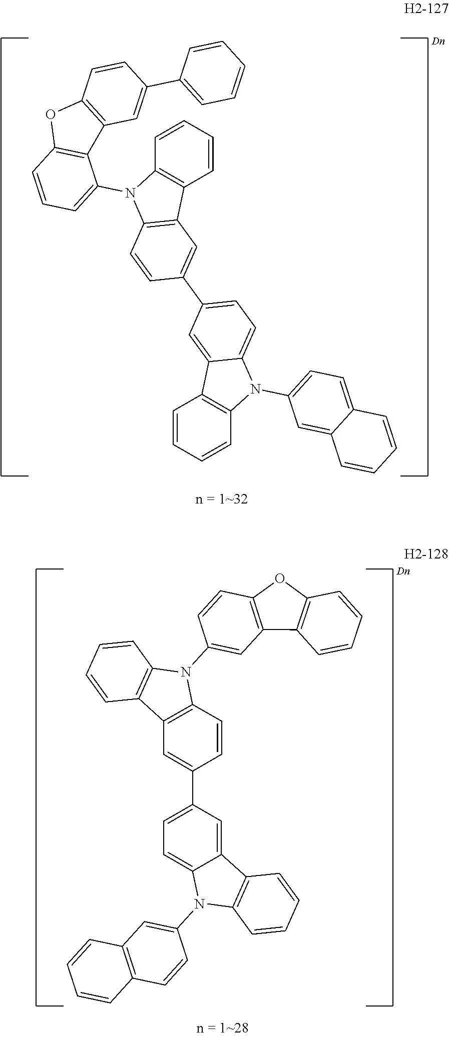

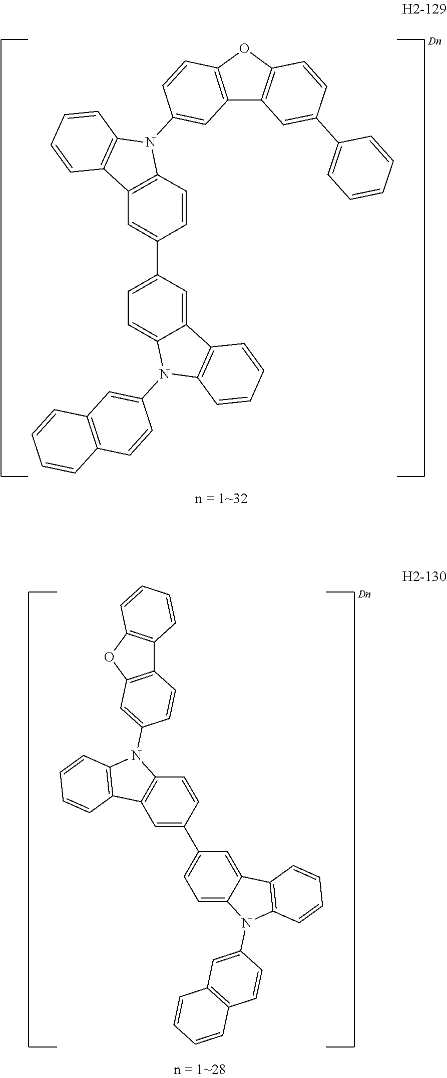

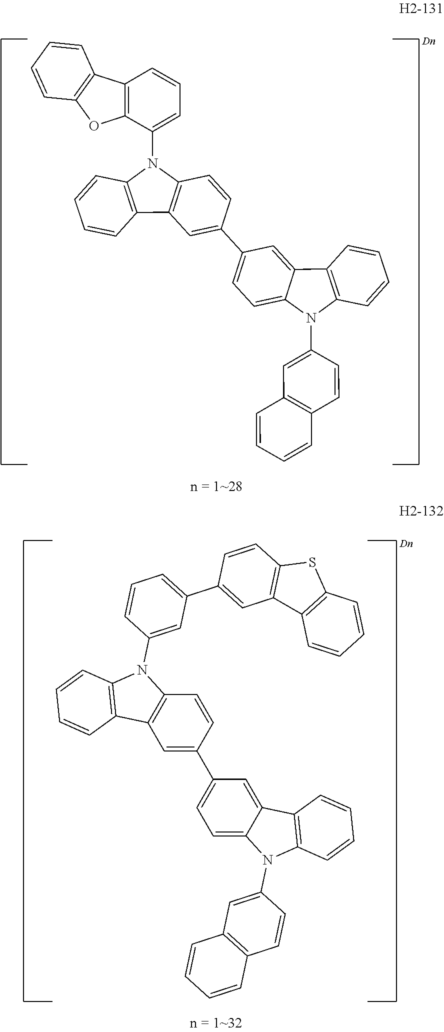

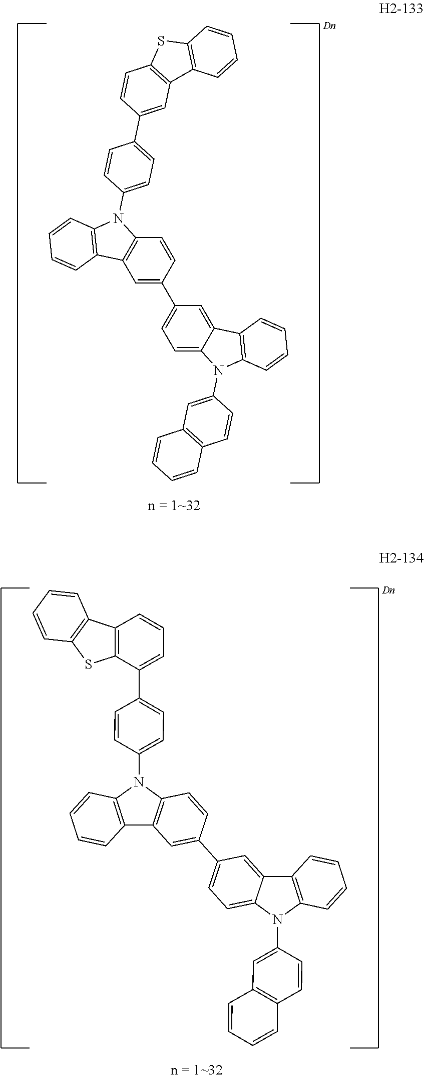

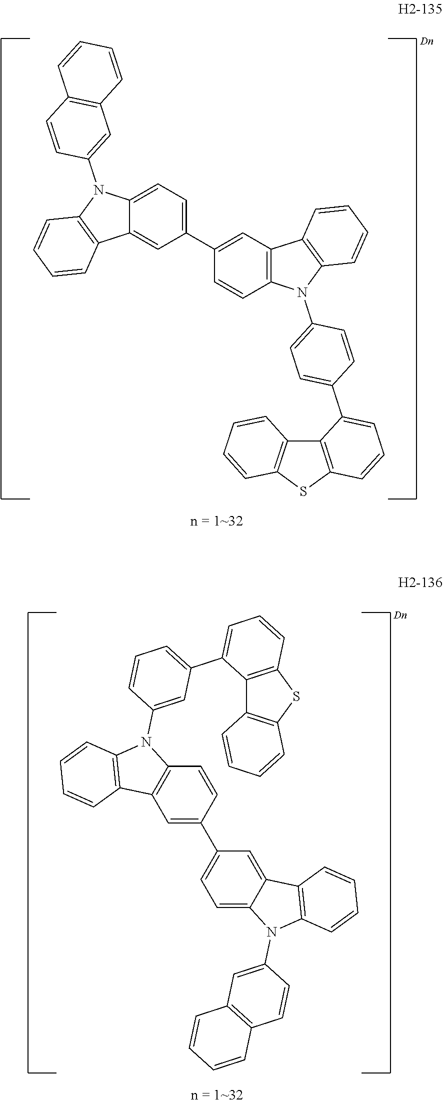

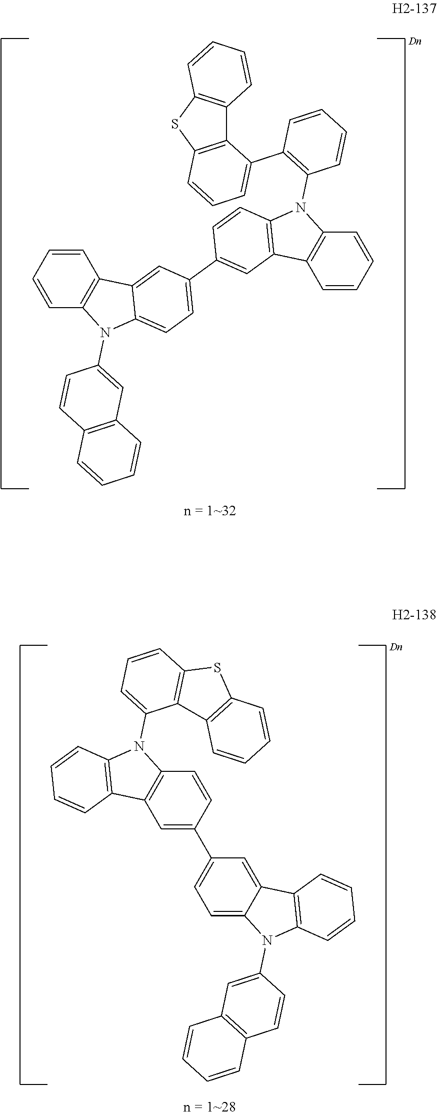

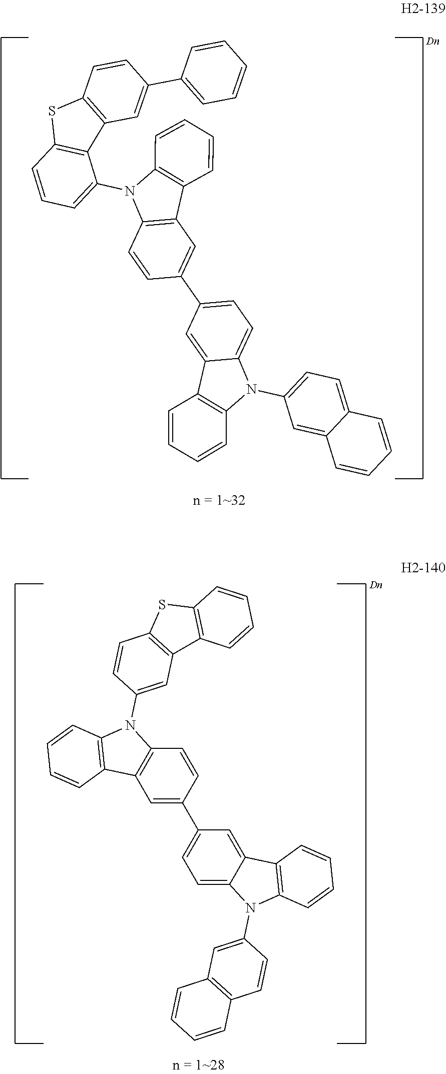

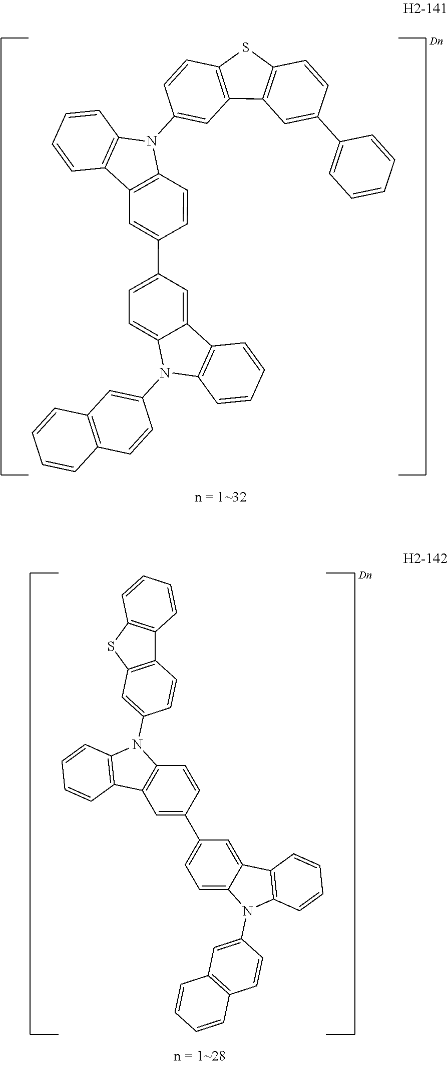

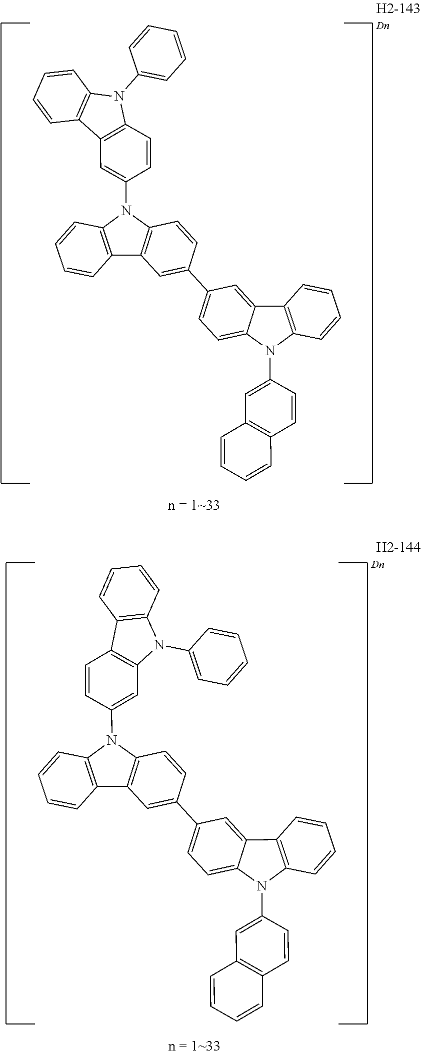

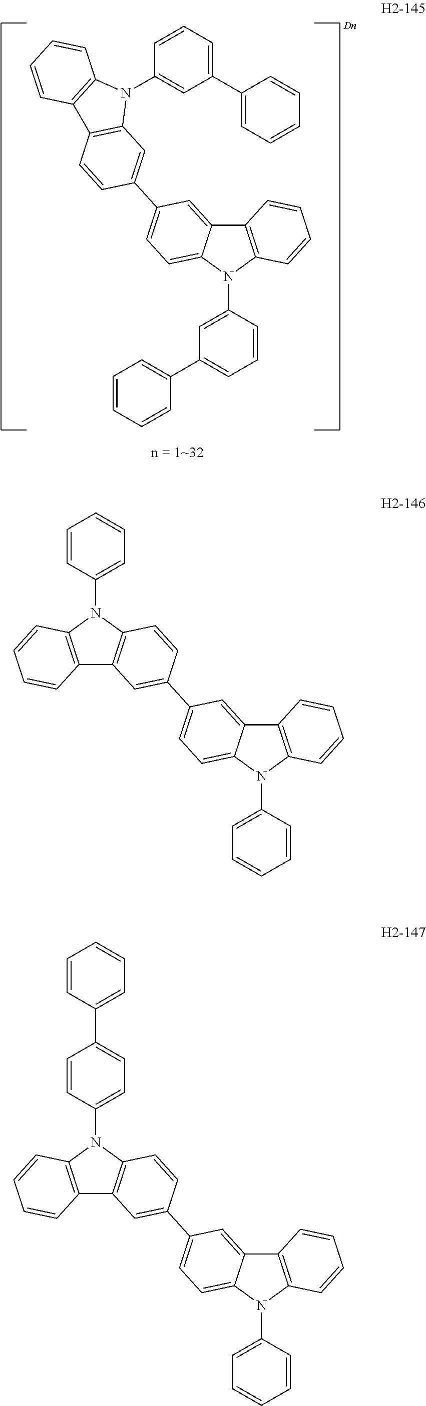

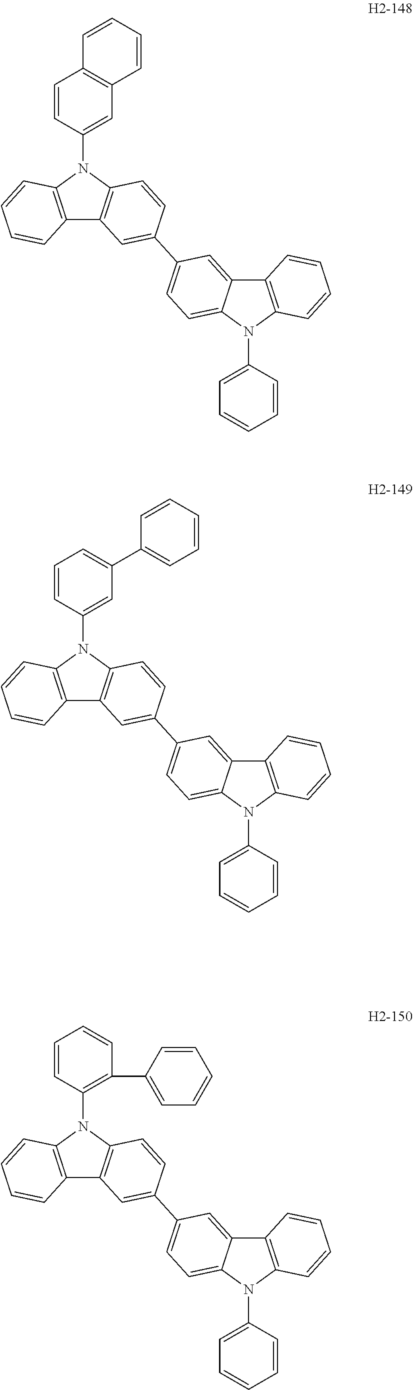 [SMILES: c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1]

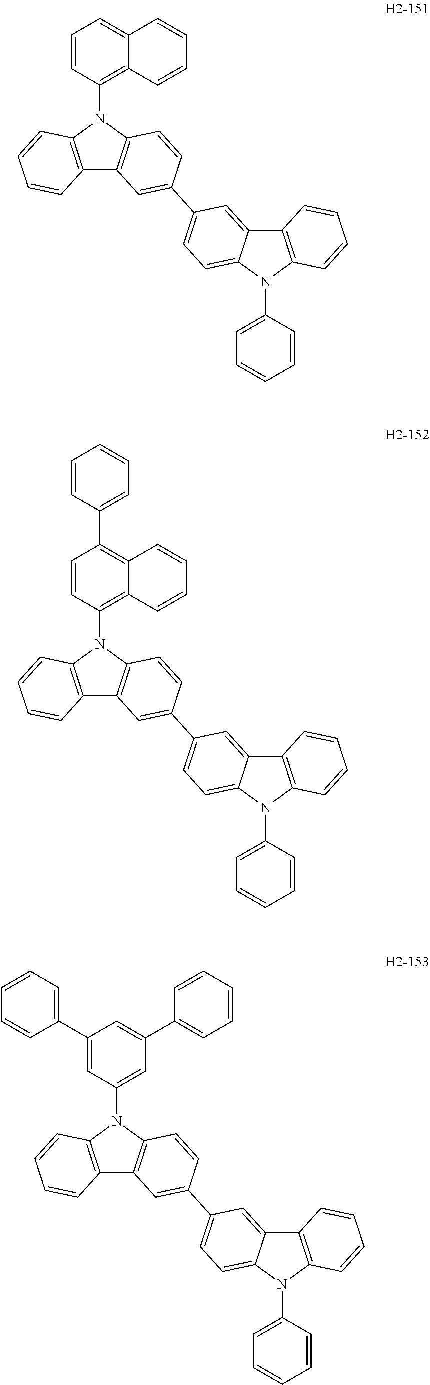 [SMILES: c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1]

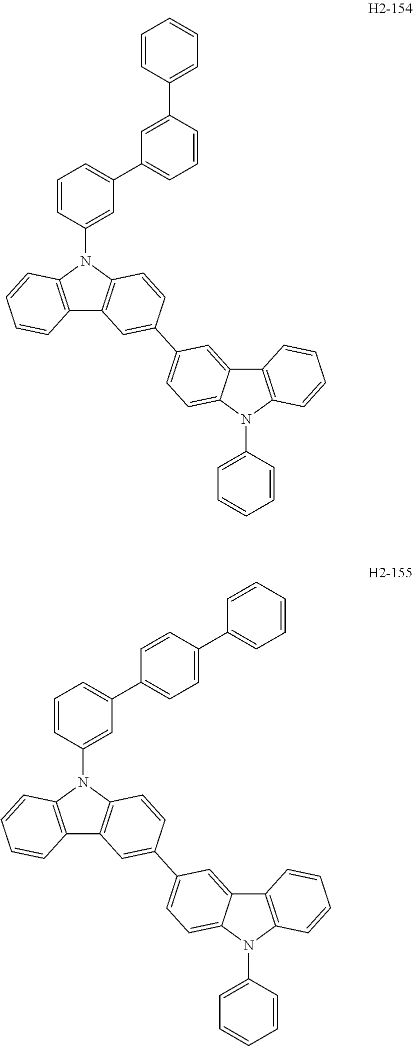 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1]

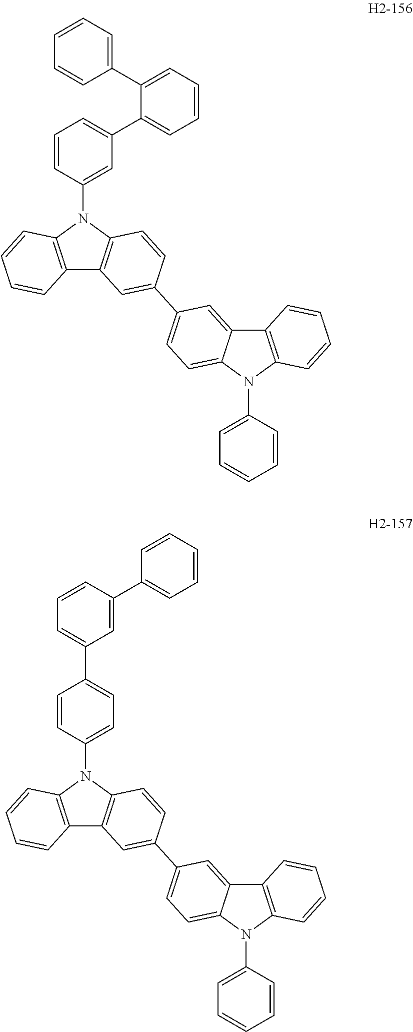 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

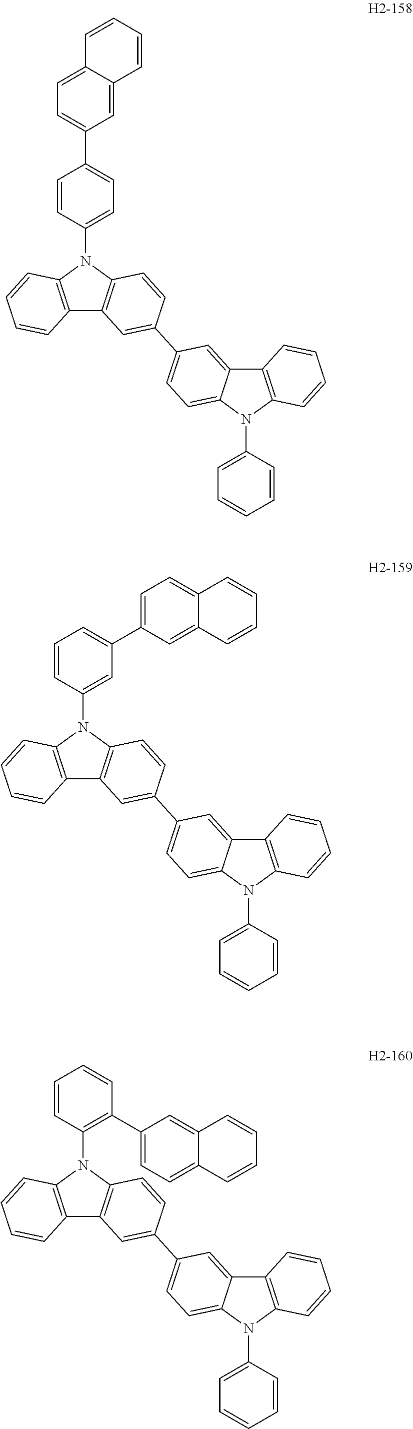 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1]

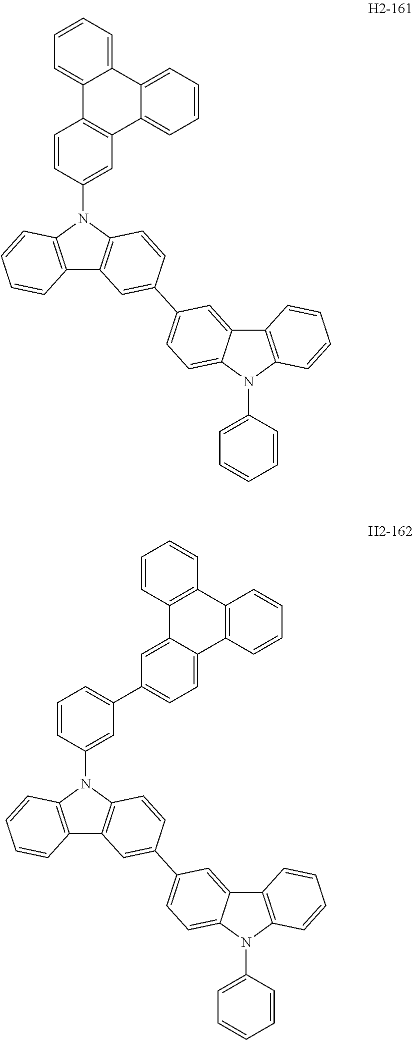 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1]

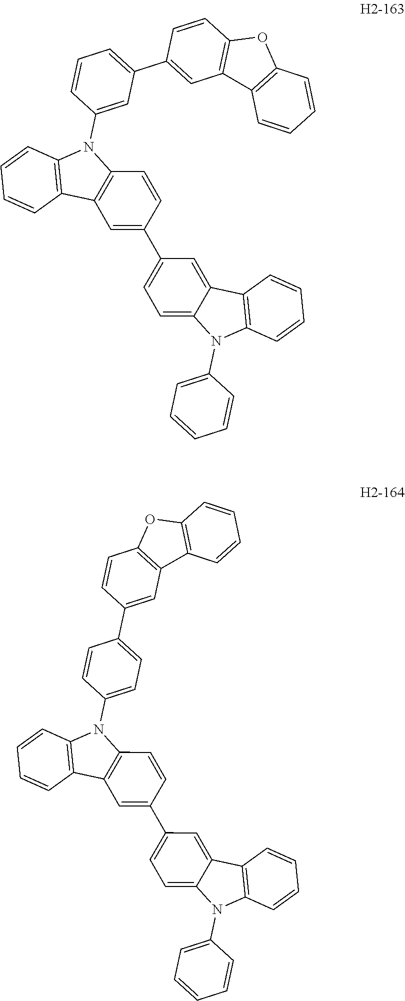 [SMILES: c1ccc(-n2c3ccccc3c3cc(-c4ccc5c(c4)c4ccccc4n5-c4cccc(-c5ccc6oc7ccccc7c6c5)c4)ccc32)cc1 ; c1ccc(-n2c3ccccc3c3cc(-c4ccc5c(c4)c4ccccc4n5-c4ccc(-c5ccc6oc7ccccc7c6c5)cc4)ccc32)cc1]

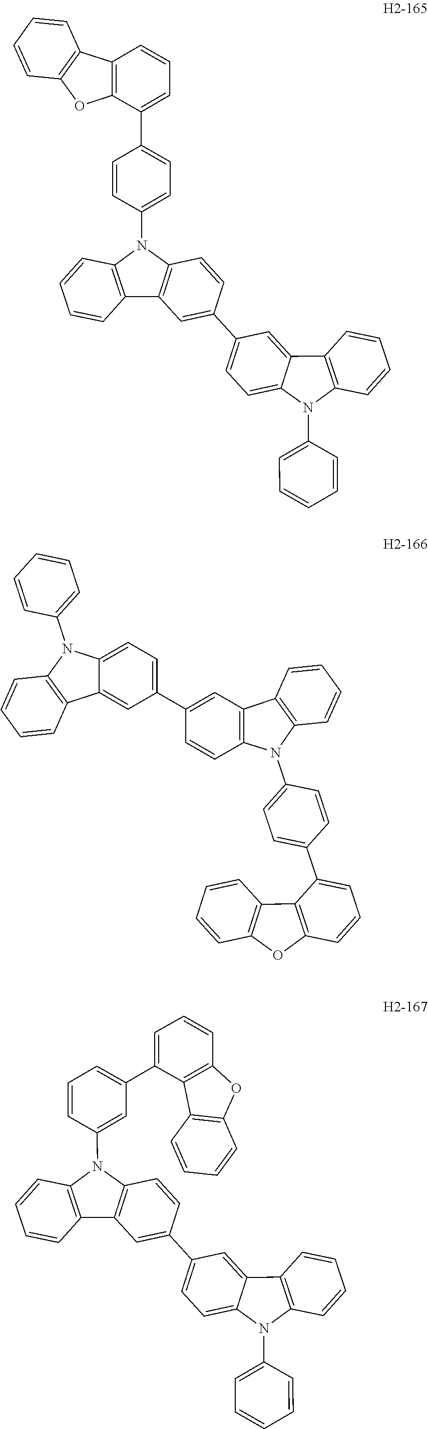 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1]

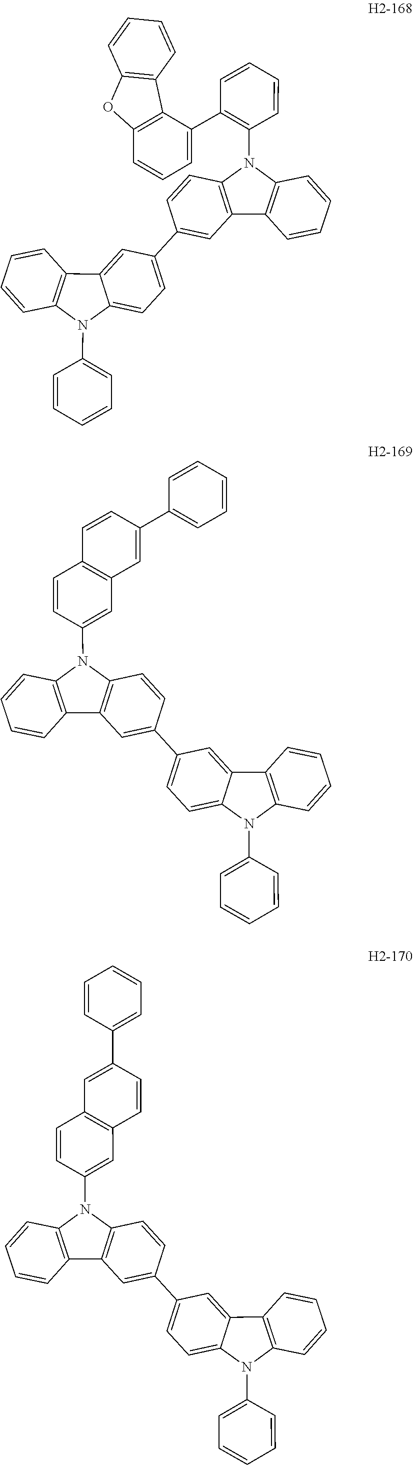 [SMILES: c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1]

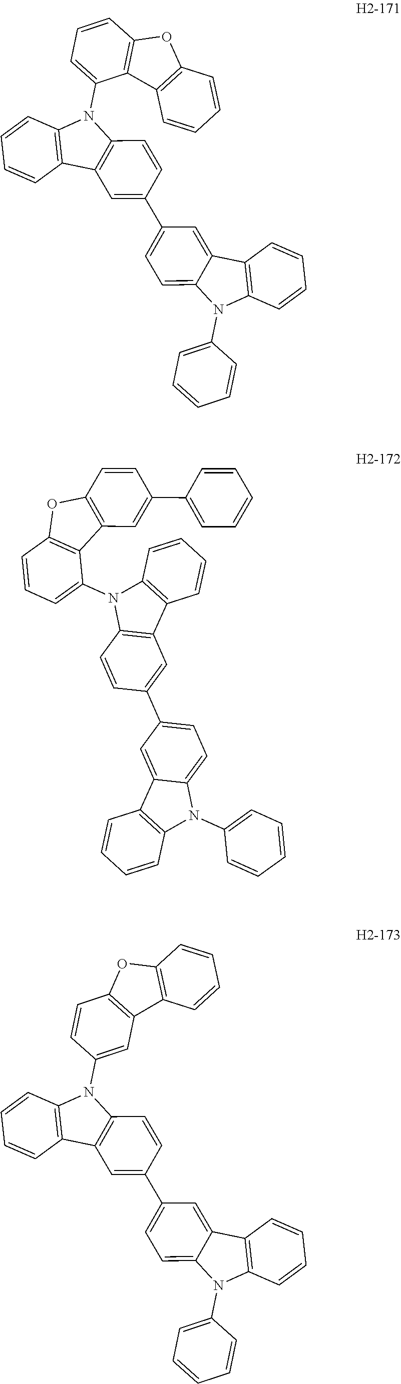 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5oc6ccccc6c5c4)ccc32)cc1]

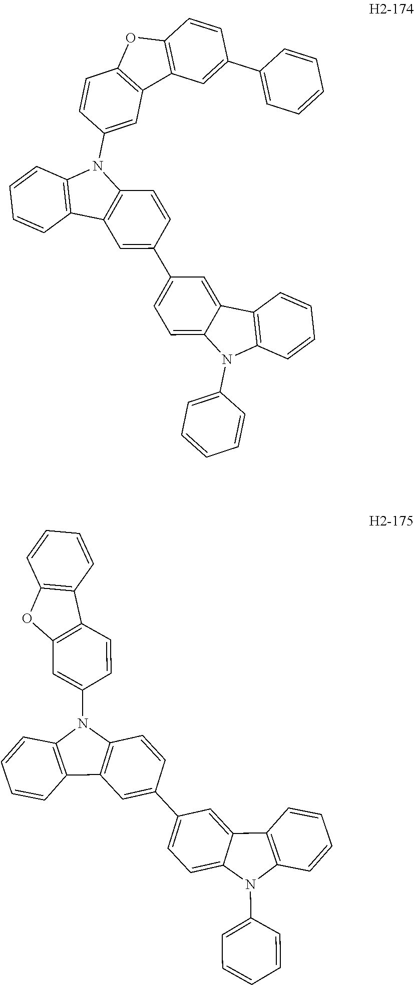 [SMILES: c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1]

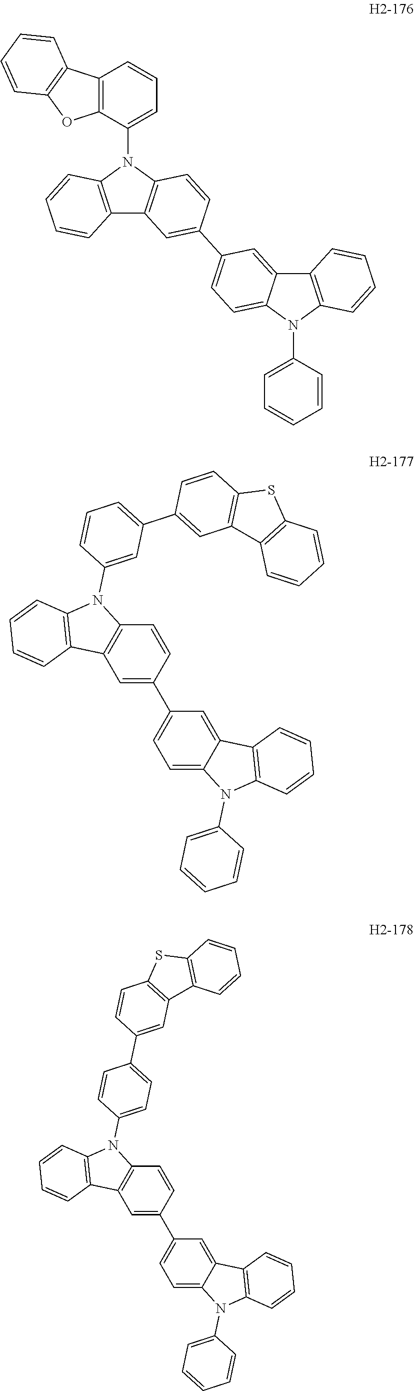 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

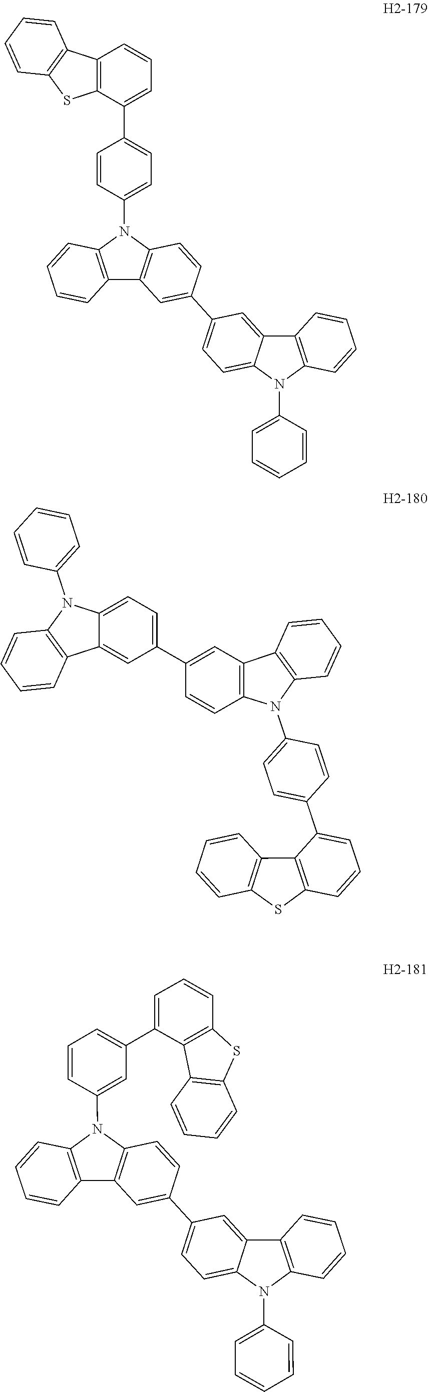 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1]

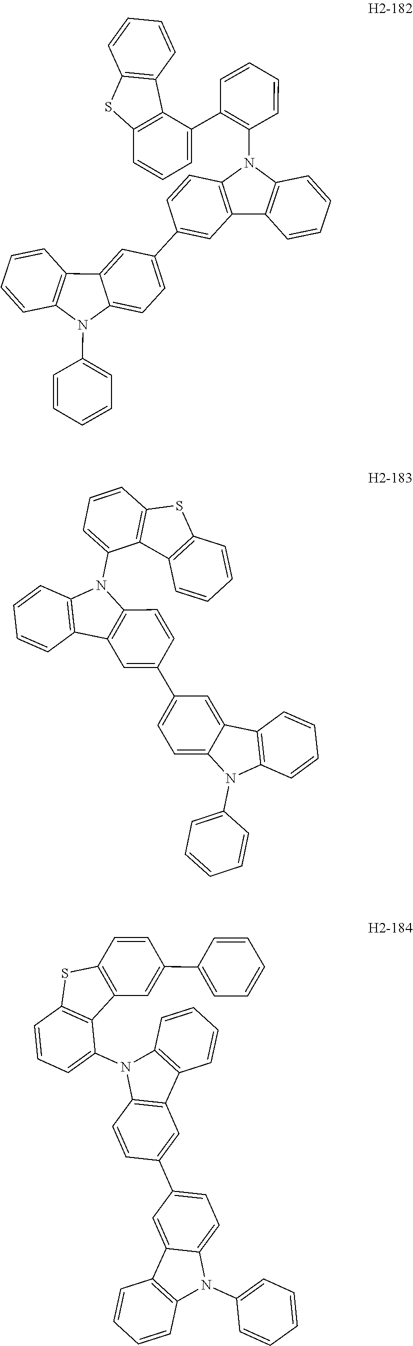 [SMILES: c1ccc(-n2c3ccccc3c3cc(-c4ccc5c(c4)c4ccccc4n5-c4ccccc4-c4cccc5sc6ccccc6c45)ccc32)cc1 ; c1ccc(-n2c3ccccc3c3cc(-c4ccc5c(c4)c4ccccc4n5-c4cccc5sc6ccccc6c45)ccc32)cc1 ; c1ccc(-c2ccc3sc4cccc(-n5c6ccccc6c6cc(-c7ccc8c(c7)c7ccccc7n8-c7ccccc7)ccc65)c4c3c2)cc1]

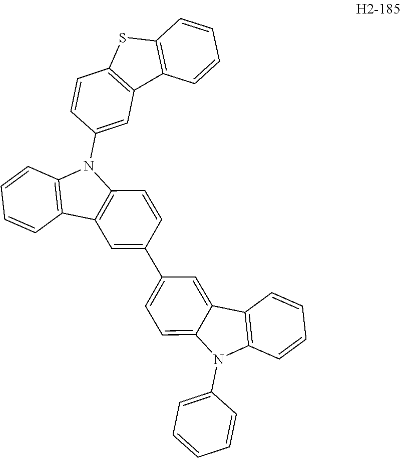 [SMILES: c1ccc(-n2c3ccccc3c3cc(-c4ccc5c(c4)c4ccccc4n5-c4ccc5sc6ccccc6c5c4)ccc32)cc1]

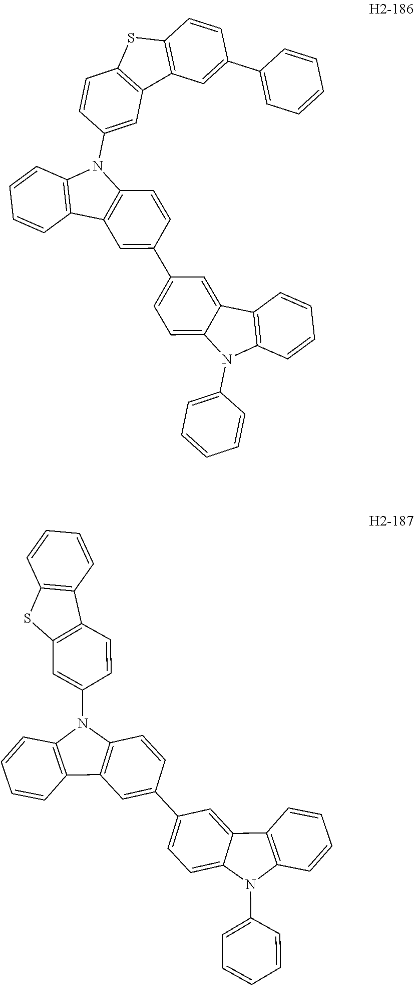 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1]

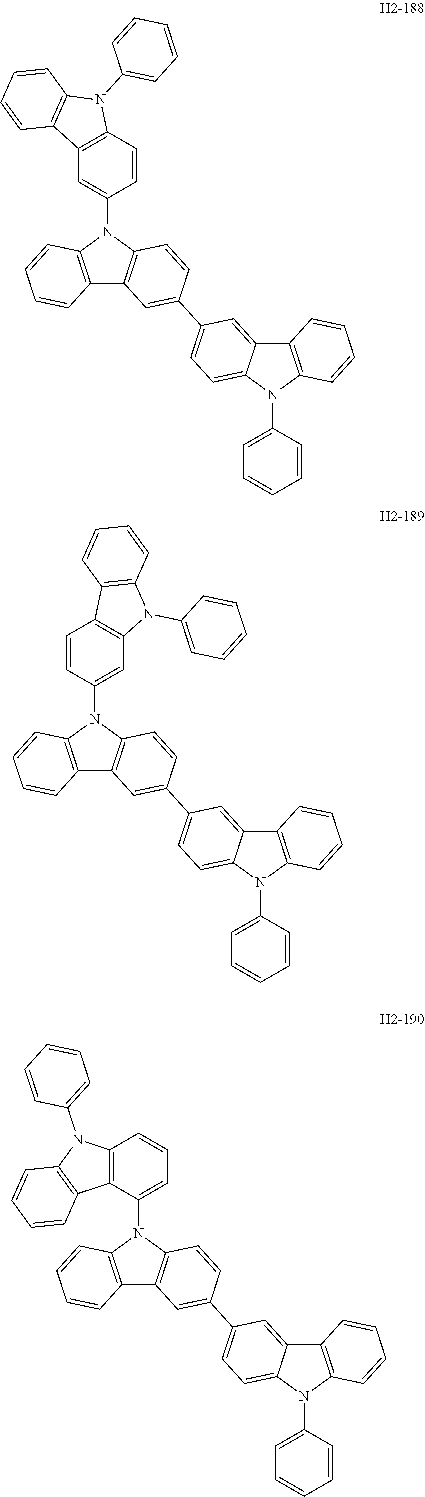 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1]

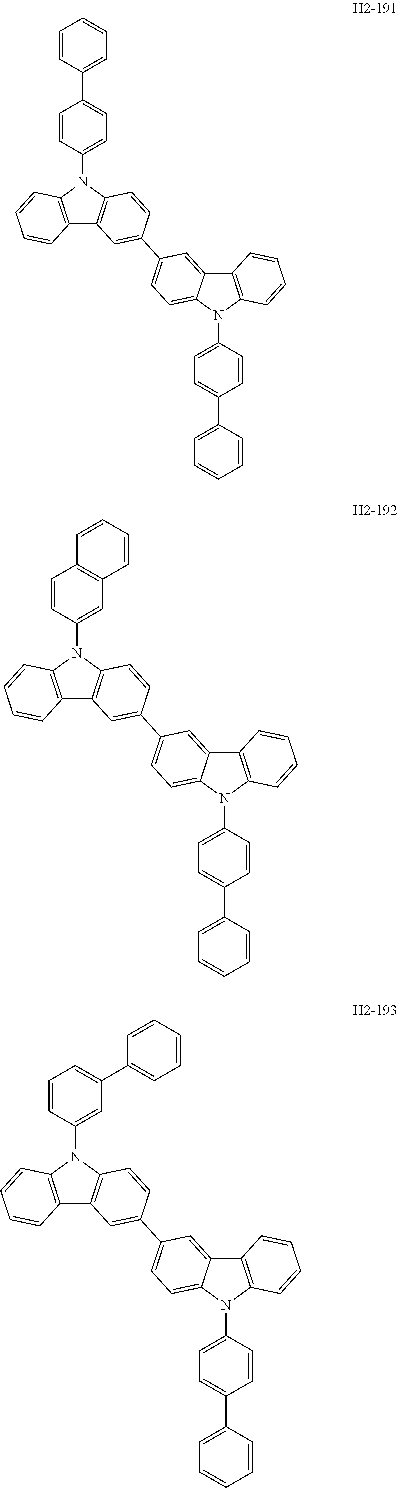 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

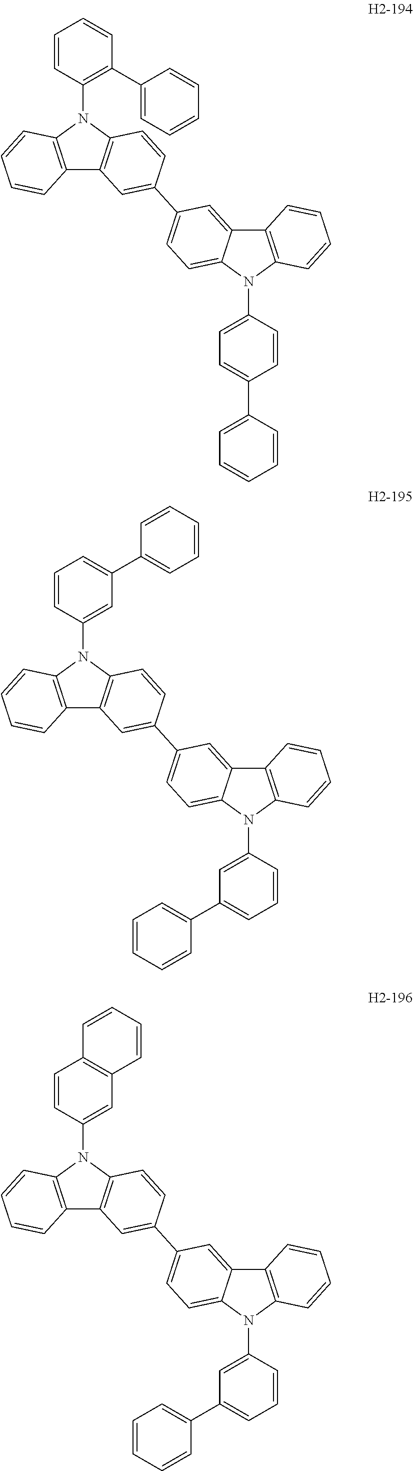 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1]

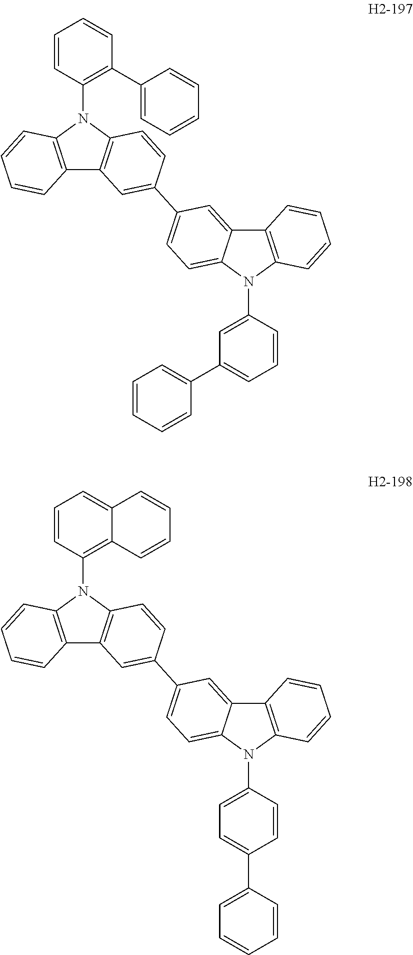 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1]

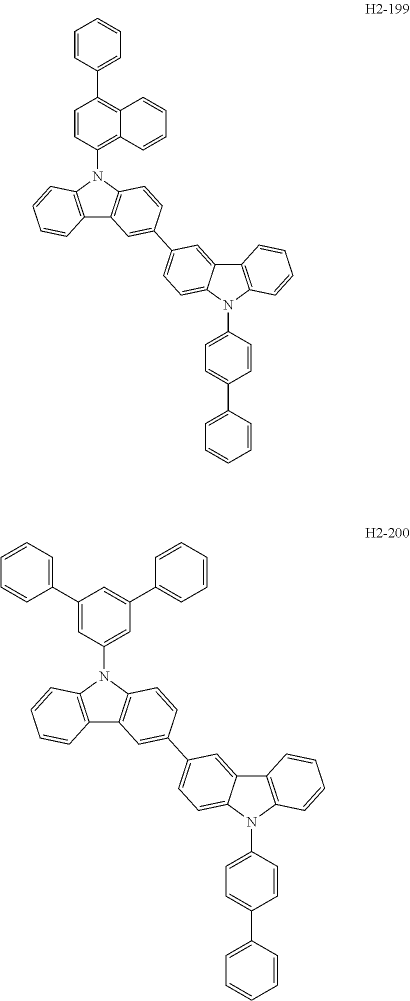 [SMILES: c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

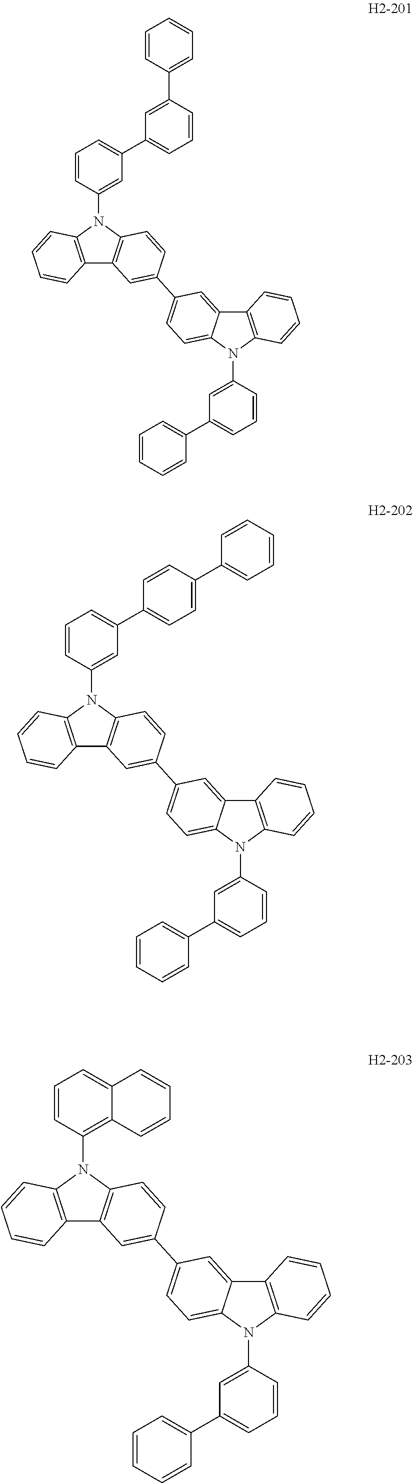 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2cccc(-n3c4ccccc4c4cc(-c5ccc6c(c5)c5ccccc5n6-c5cccc6ccccc56)ccc43)c2)cc1]

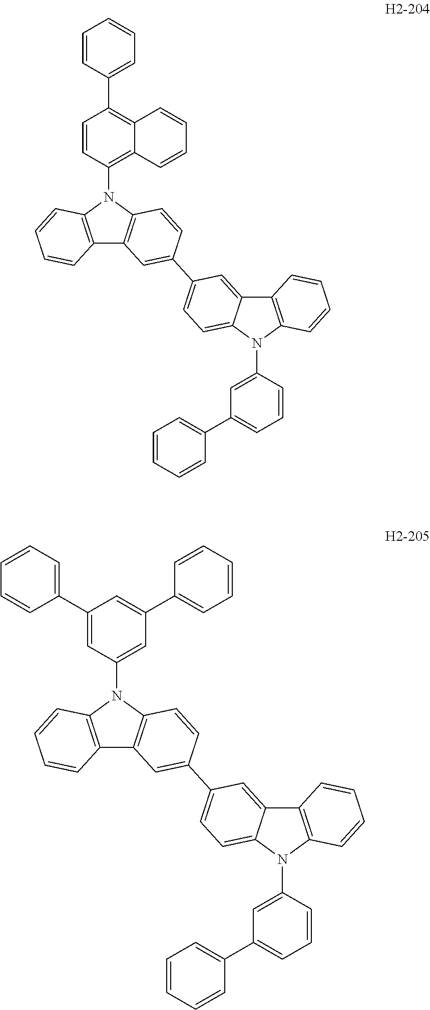 [SMILES: c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

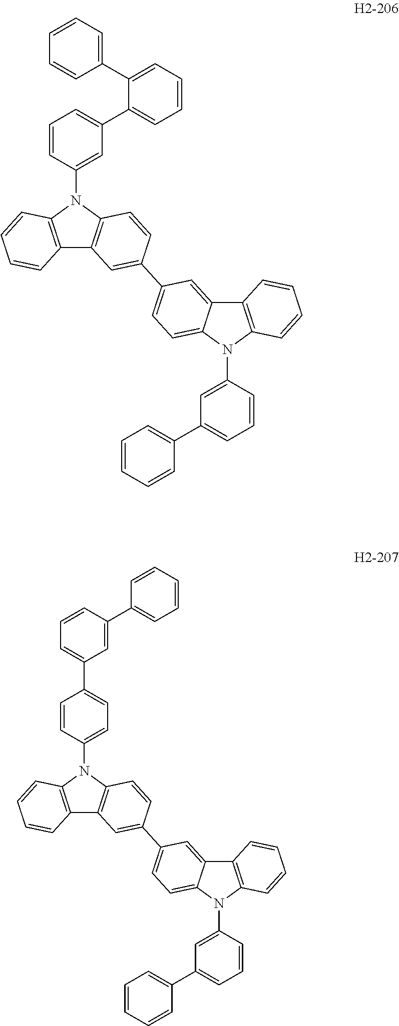 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1]

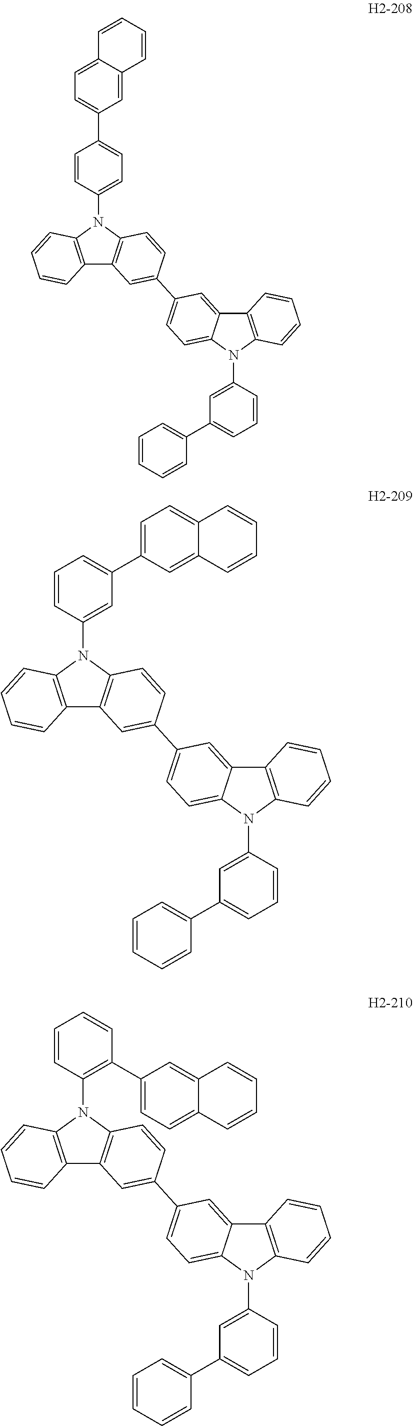

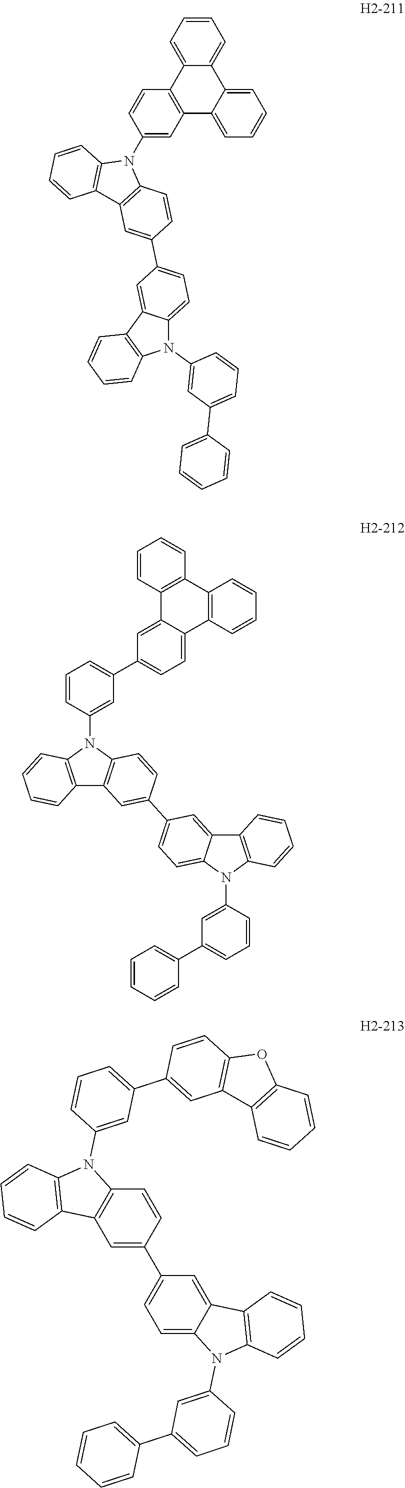 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1]

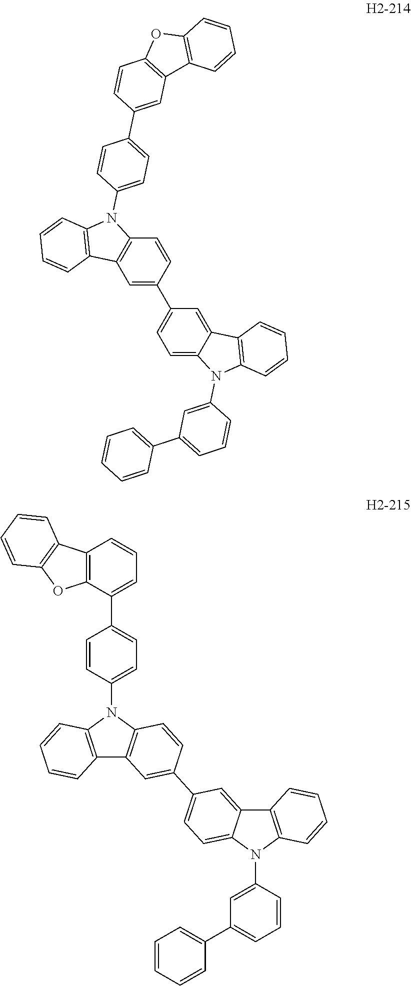 [SMILES: c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

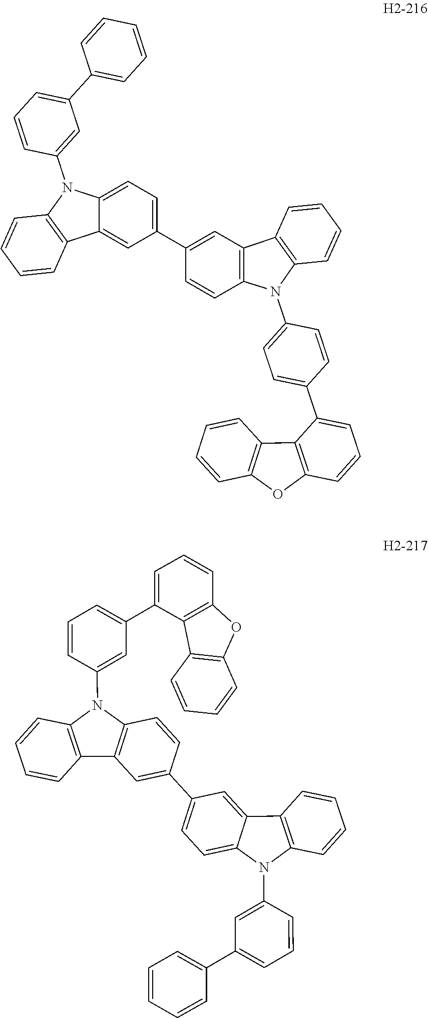 [SMILES: c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

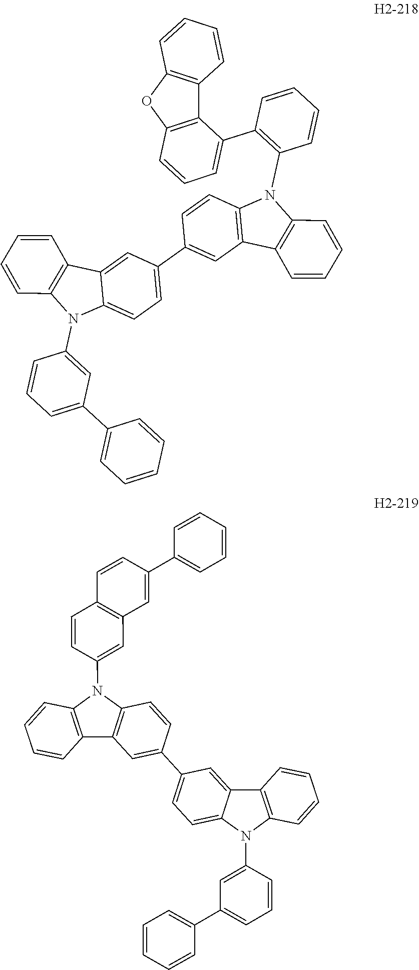 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1]

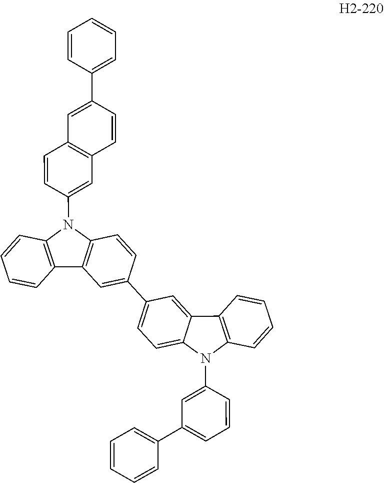 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1]

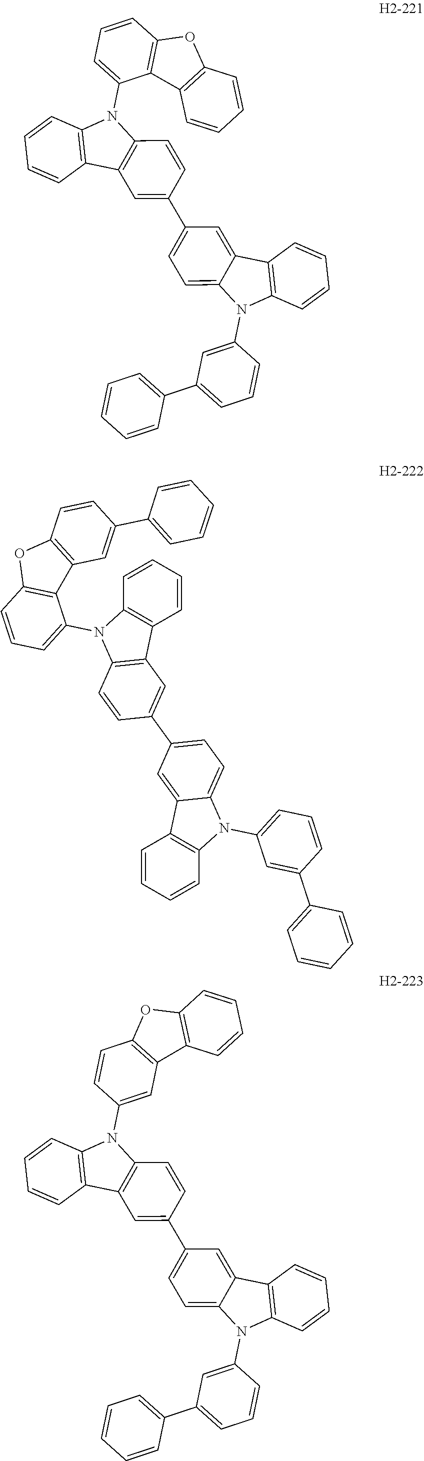 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

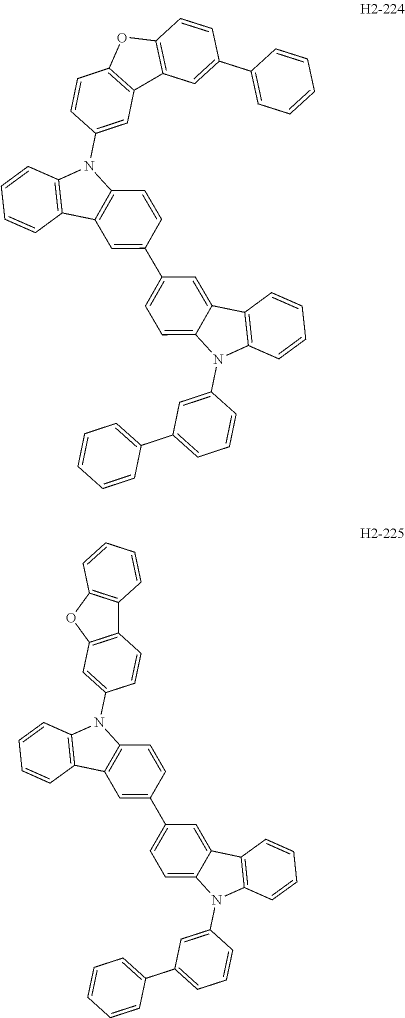 [SMILES: c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)oc5ccccc56)ccc43)c2)cc1]

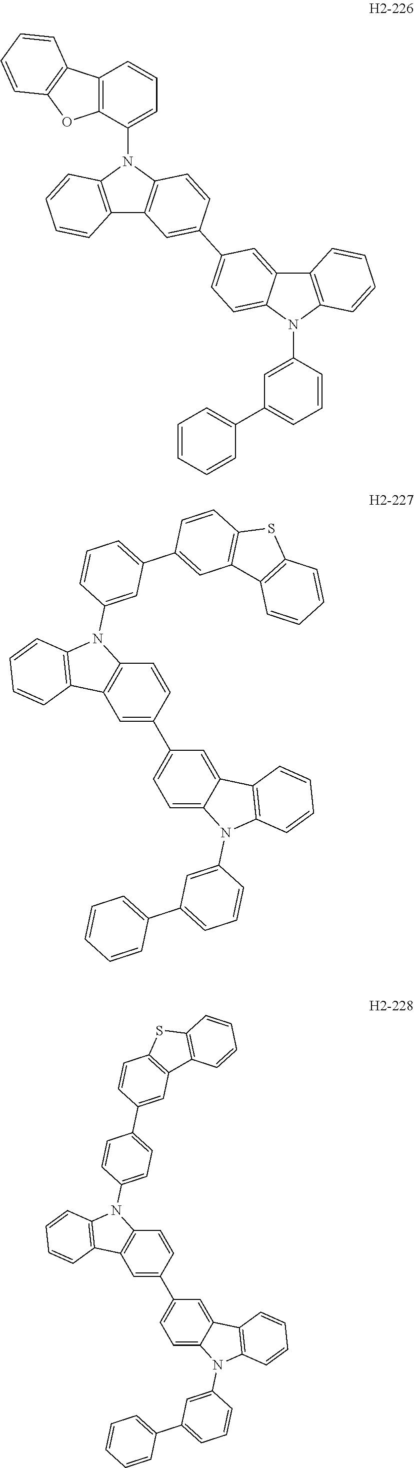

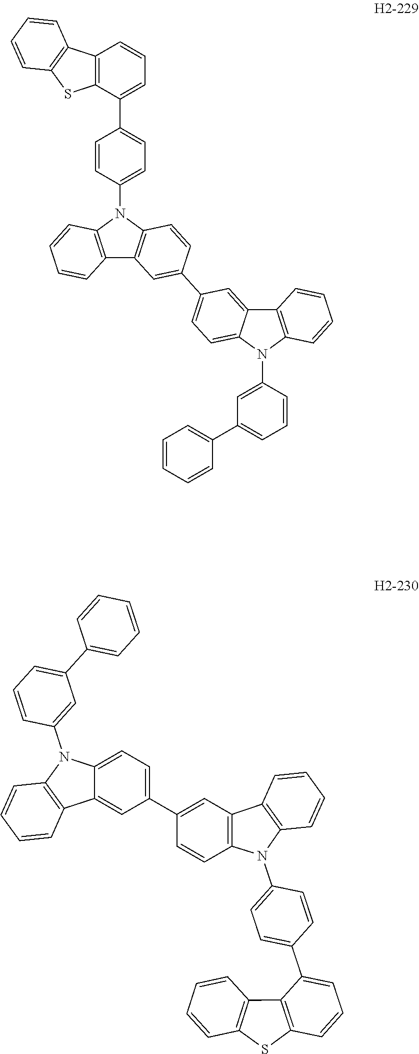 [SMILES: c1ccc(-c2cccc(-n3c4ccccc4c4cc(-c5ccc6c(c5)c5ccccc5n6-c5ccc(-c6cccc7c6sc6ccccc67)cc5)ccc43)c2)cc1 ; c1ccc(-c2cccc(-n3c4ccccc4c4cc(-c5ccc6c(c5)c5ccccc5n6-c5ccc(-c6cccc7sc8ccccc8c67)cc5)ccc43)c2)cc1]

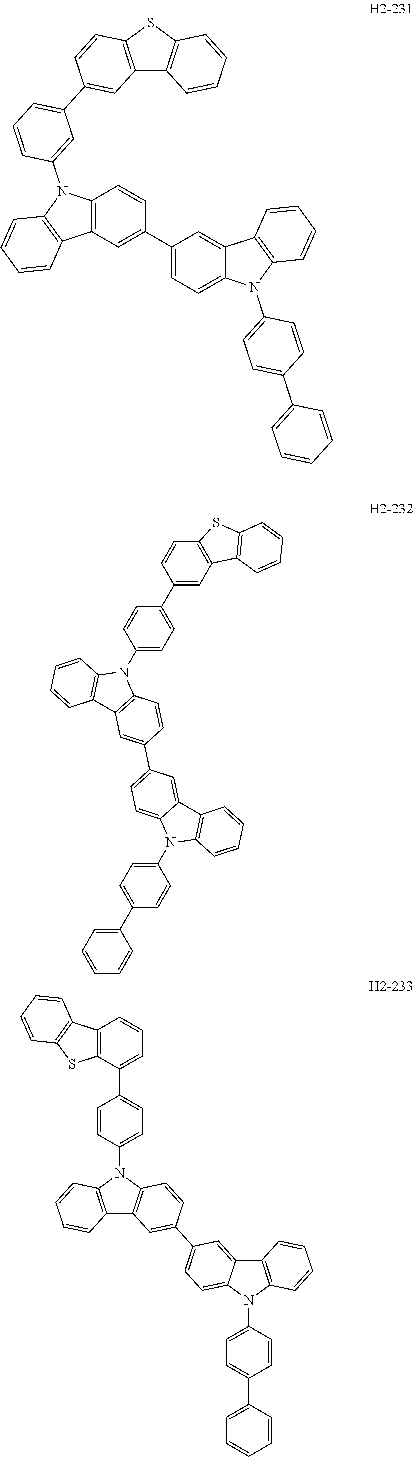 [SMILES: c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1 ; c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

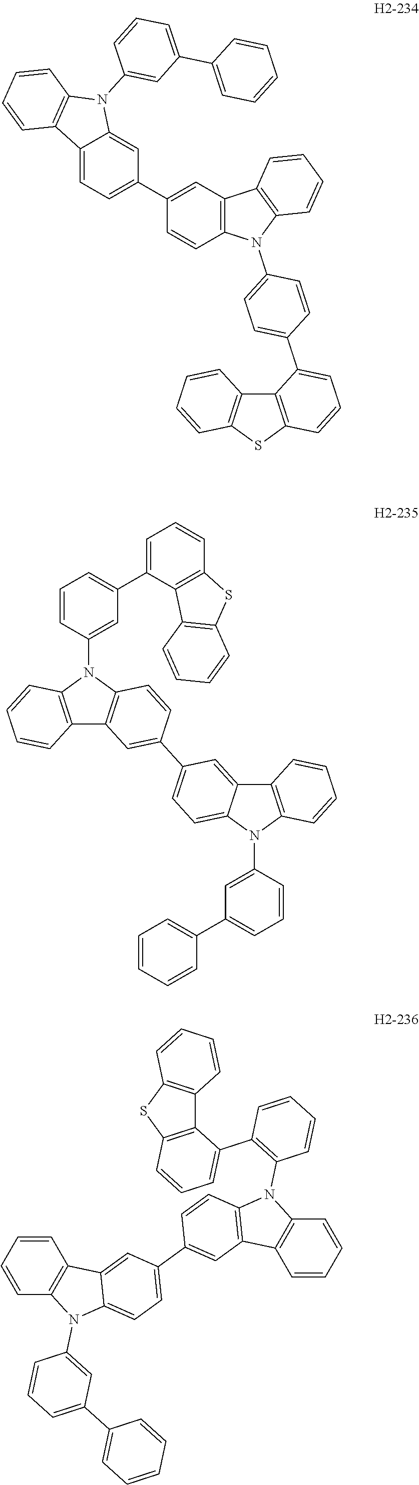

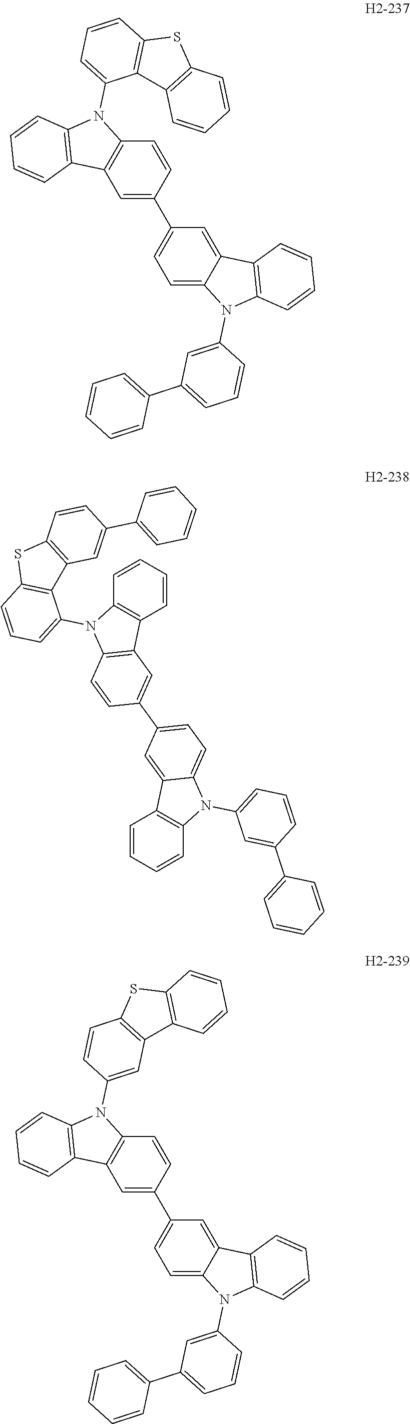 [SMILES: c1ccc(-c2cccc(-n3c4ccccc4c4cc(-c5ccc6c(c5)c5ccccc5n6-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

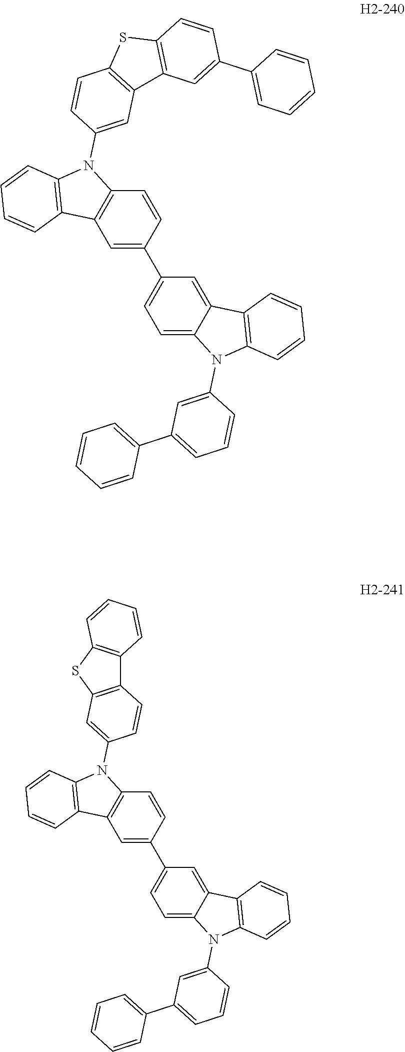 [SMILES: c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)sc5ccccc56)ccc43)c2)cc1]

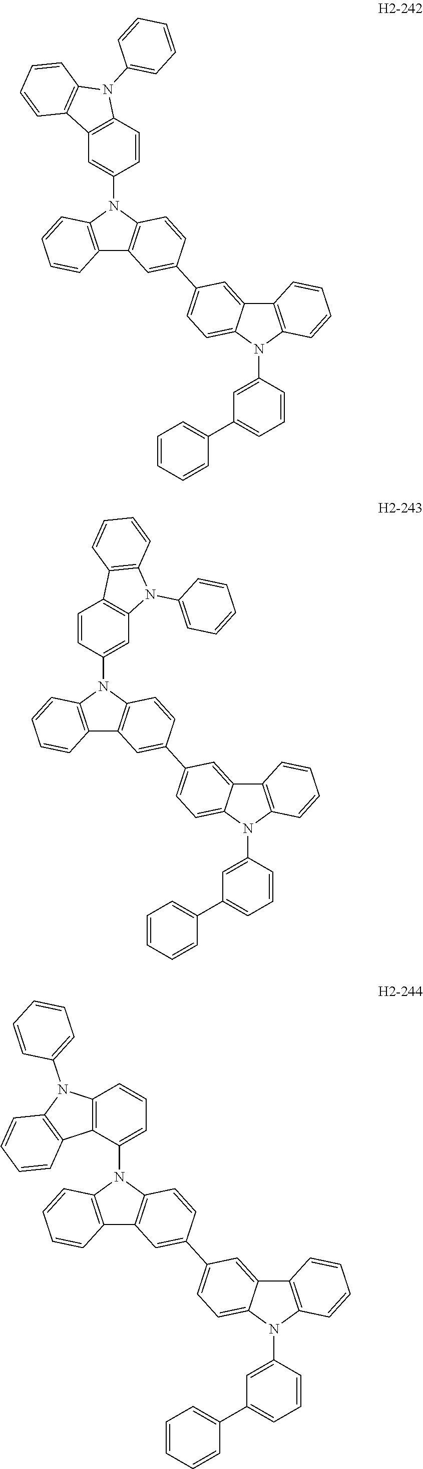 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1]

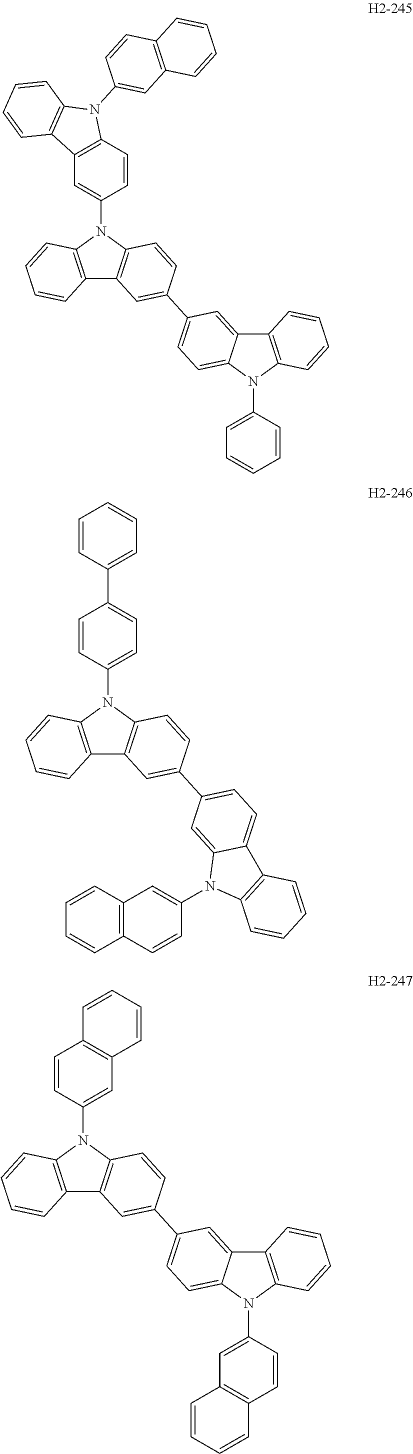

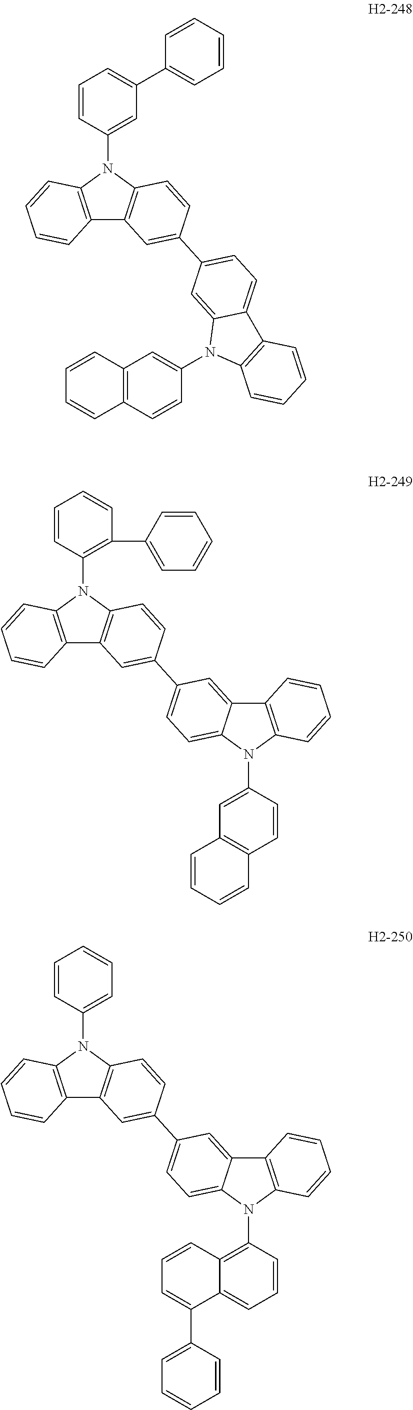 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1]

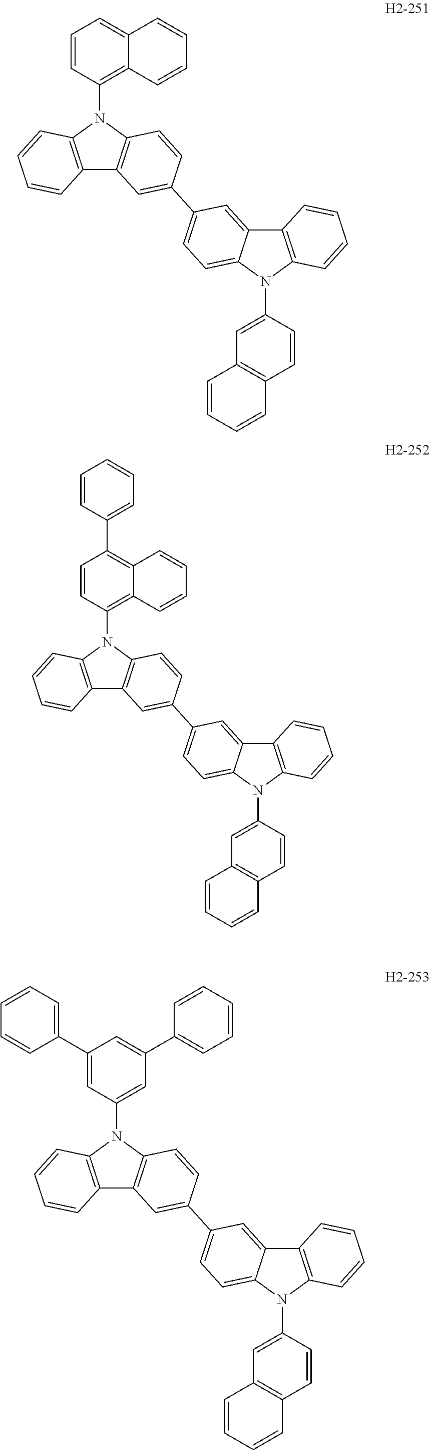 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

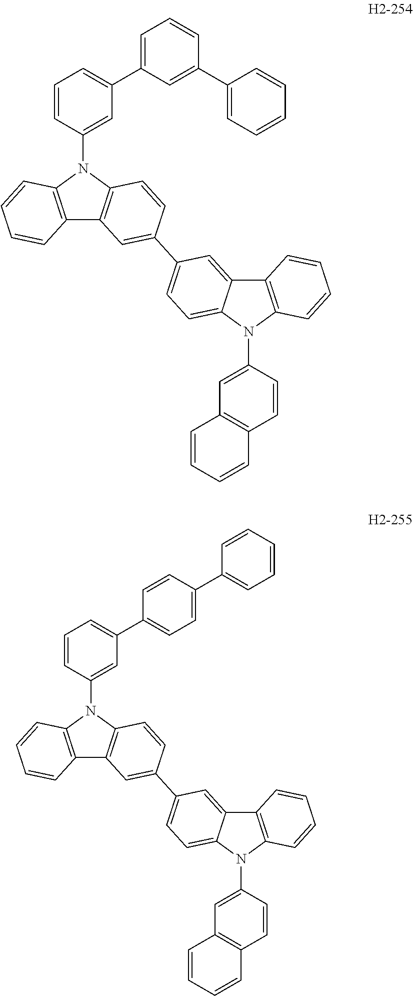 [SMILES: c1ccc(-c2cccc(-c3cccc(-n4c5ccccc5c5cc(-c6ccc7c(c6)c6ccccc6n7-c6ccc7ccccc7c6)ccc54)c3)c2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

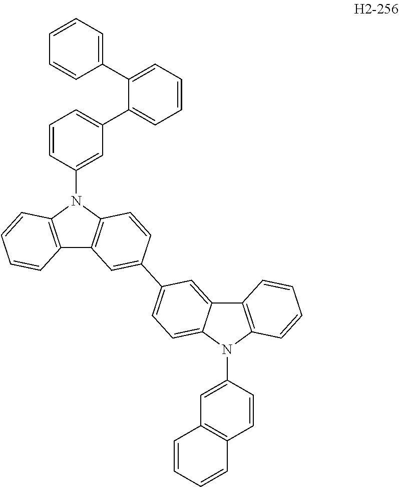 [SMILES: c1ccc(-c2ccccc2-c2cccc(-n3c4ccccc4c4cc(-c5ccc6c(c5)c5ccccc5n6-c5ccc6ccccc6c5)ccc43)c2)cc1]

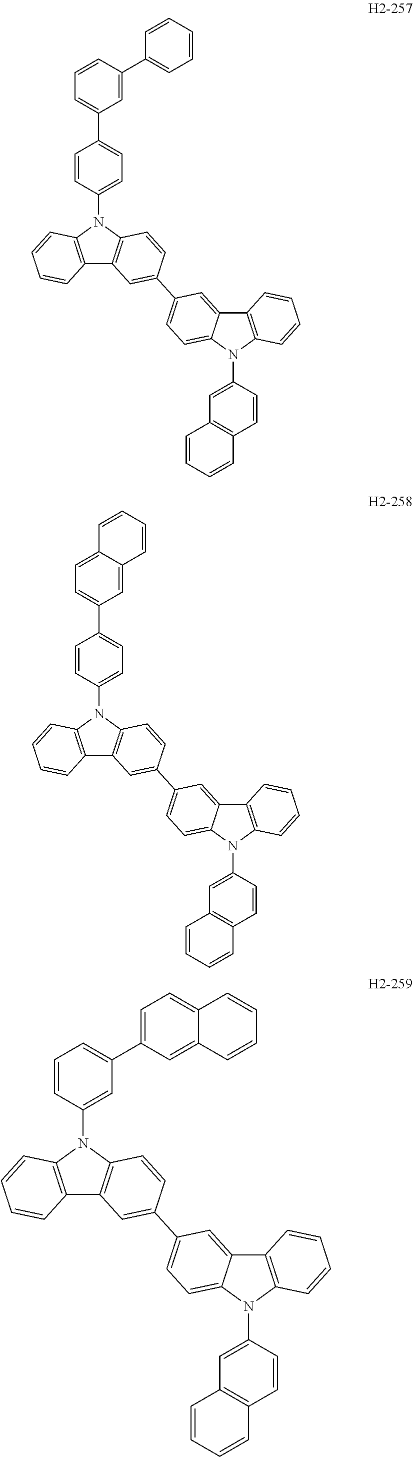

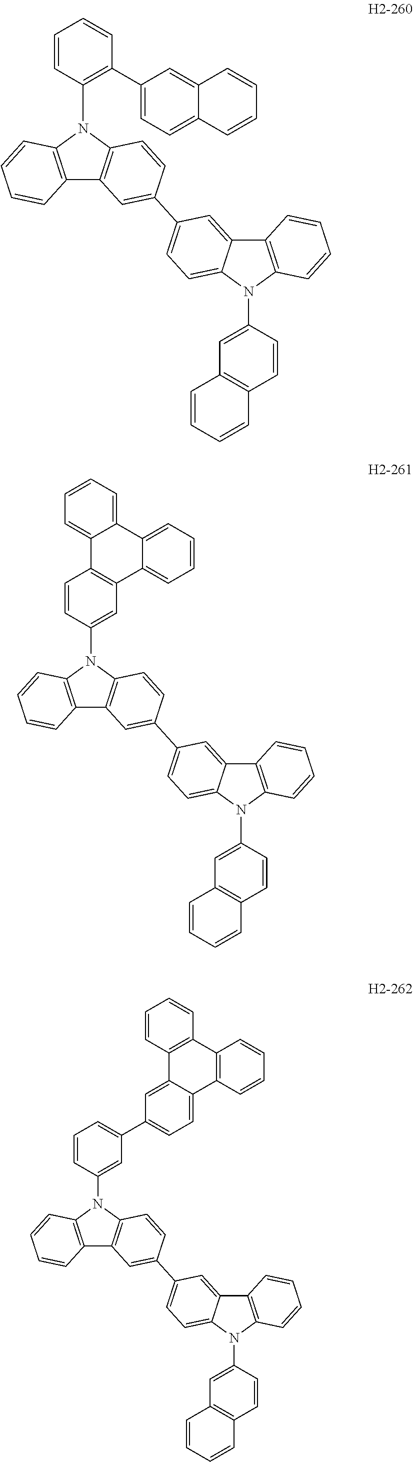 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1]

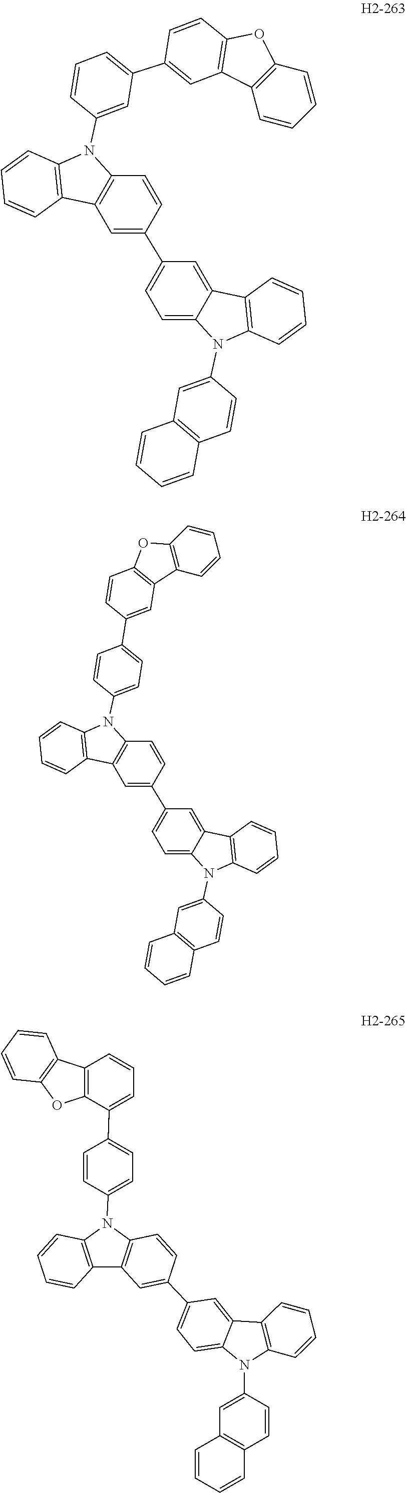 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1]

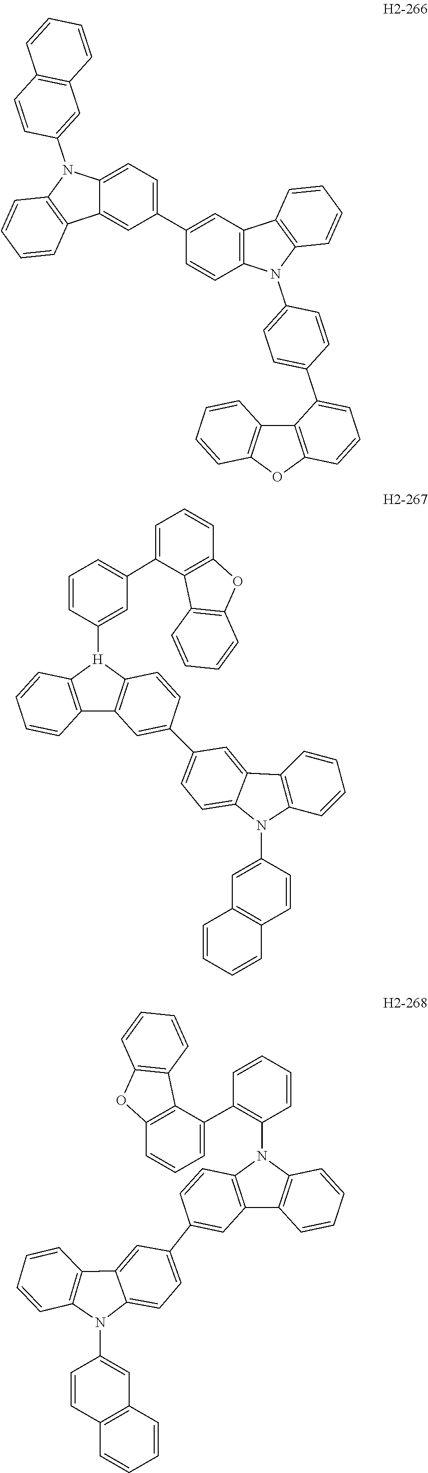

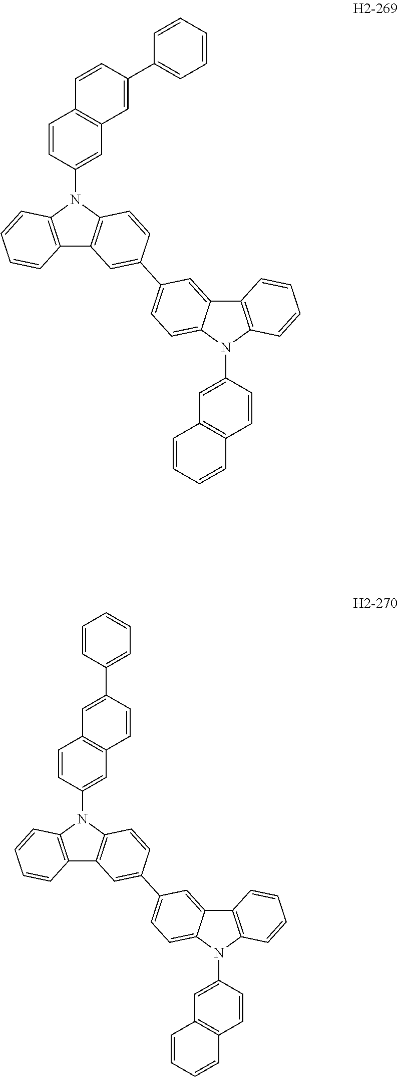 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1]

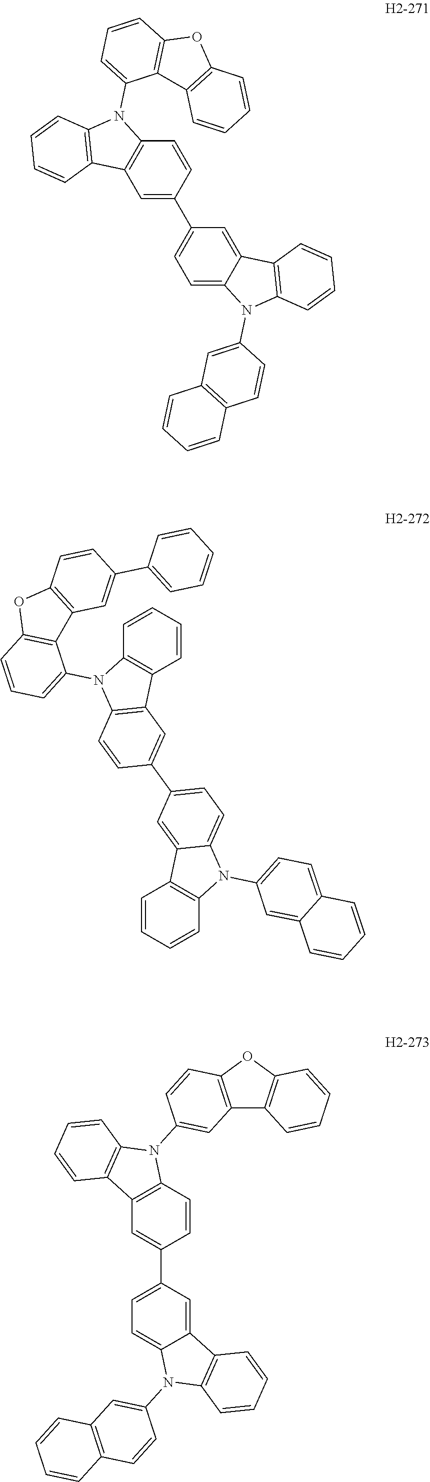 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1]

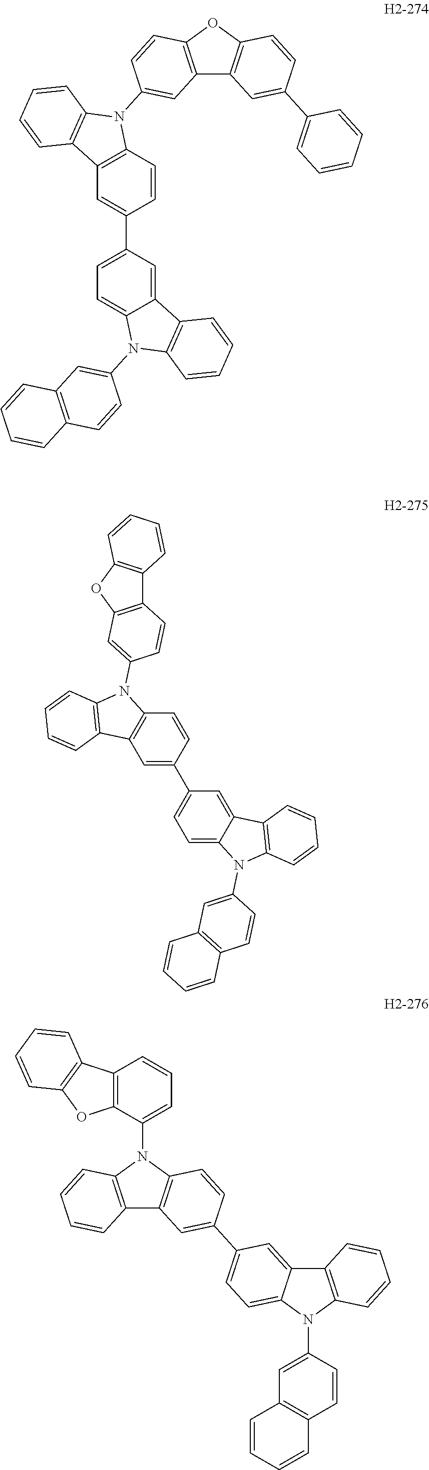 [SMILES: c1ccc(-c2ccc3oc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1]

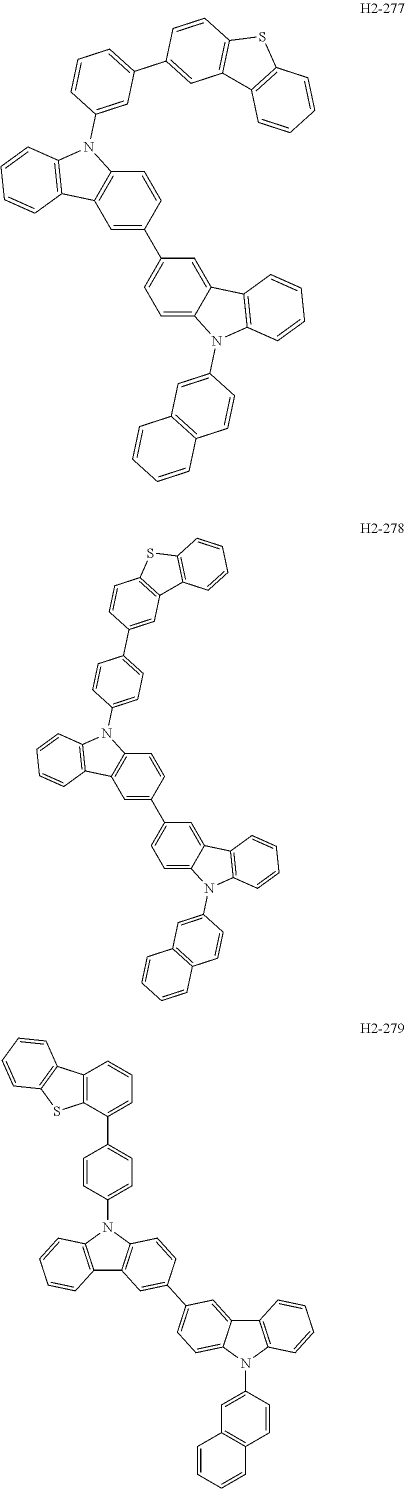 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1]

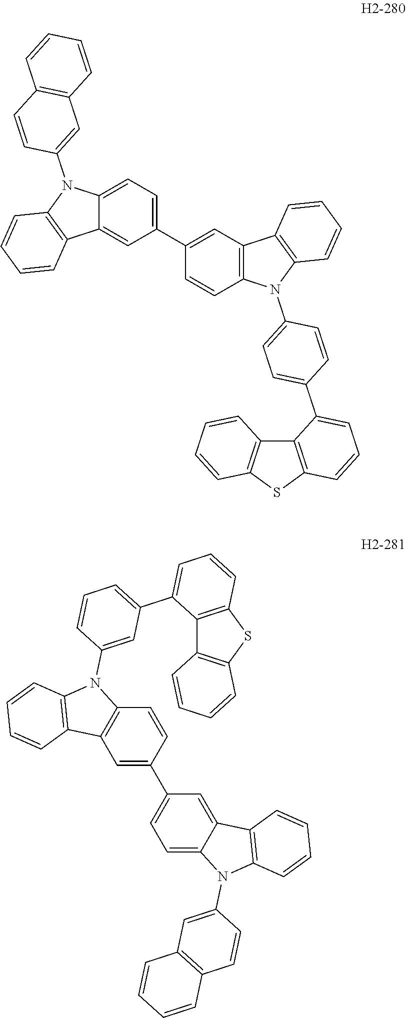 [SMILES: c1ccc2cc(-n3c4ccccc4c4cc(-c5ccc6c(c5)c5ccccc5n6-c5ccc(-c6cccc7sc8ccccc8c67)cc5)ccc43)ccc2c1 ; c1cc(-c2cccc3sc4ccccc4c23)cc(-n2c3ccccc3c3cc(-c4ccc5c(c4)c4ccccc4n5-c4ccc5ccccc5c4)ccc32)c1]

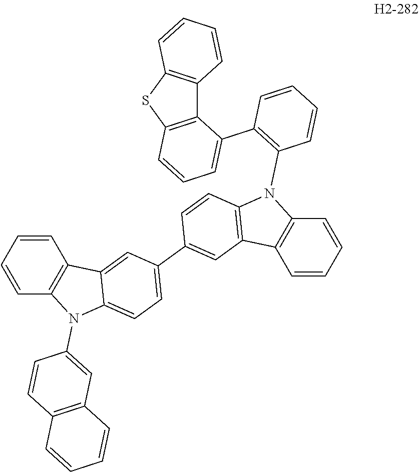 [SMILES: c1ccc(-n2c3ccccc3c3cc(-c4ccc5c(c4)c4ccccc4n5-c4ccc5ccccc5c4)ccc32)c(-c2cccc3sc4ccccc4c23)c1]

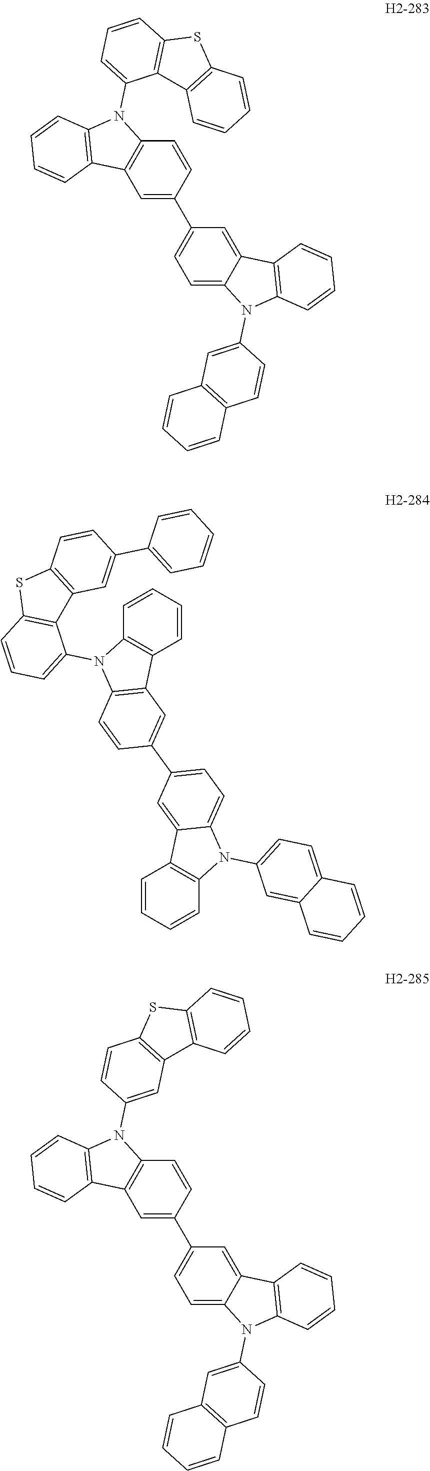 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6sc7ccccc7c6c5)ccc43)ccc2c1]

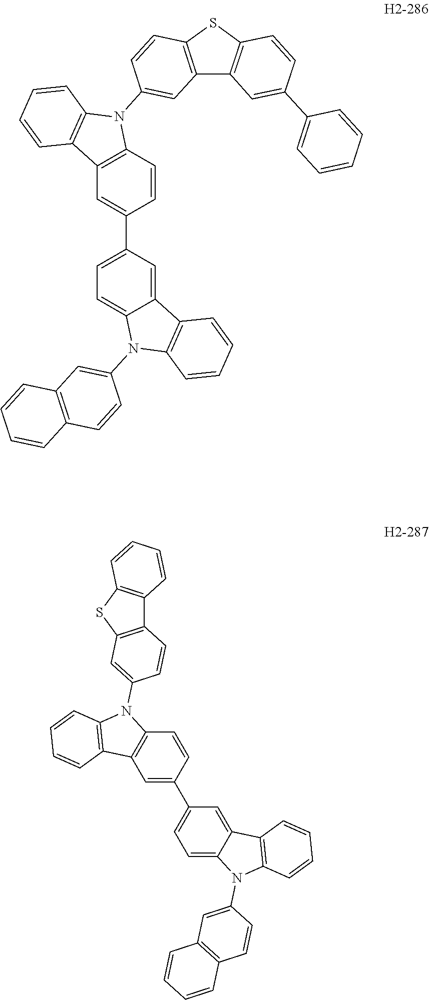 [SMILES: c1ccc(-c2ccc3sc4ccc(-n5c6ccccc6c6cc(-c7ccc8c(c7)c7ccccc7n8-c7ccc8ccccc8c7)ccc65)cc4c3c2)cc1 ; c1ccc2cc(-n3c4ccccc4c4cc(-c5ccc6c(c5)c5ccccc5n6-c5ccc6c(c5)sc5ccccc56)ccc43)ccc2c1]

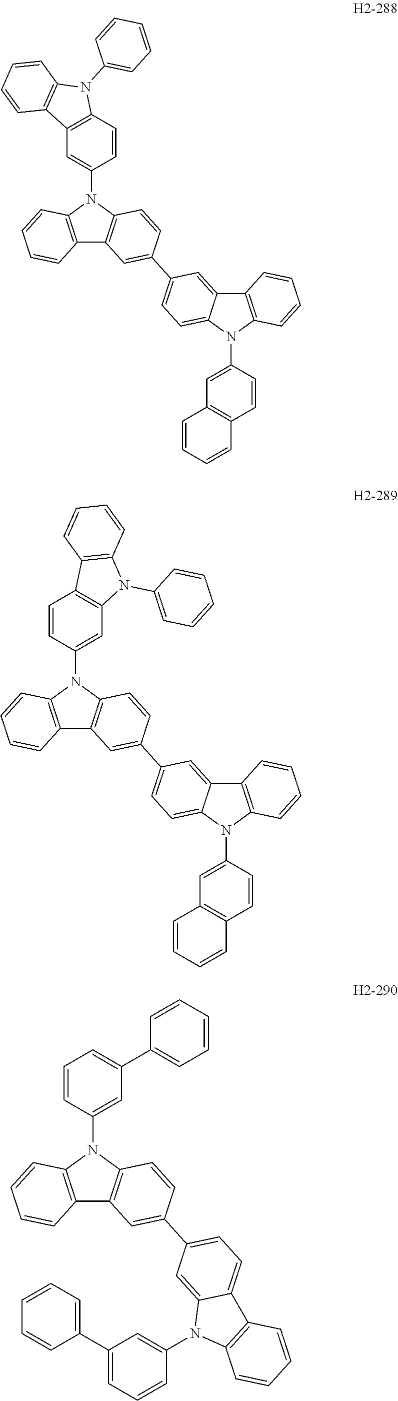

Among the above compounds, Dn signifies that n hydrogens are replaced with deuterium.

The compound represented by Formula 2 according to the present disclosure may be produced by referring to Japanese Patent No. 3139321, etc. Further, the compound represented by Formula 2 according to the present disclosure may be produced by the following Reaction Scheme 2, but is not limited thereto.

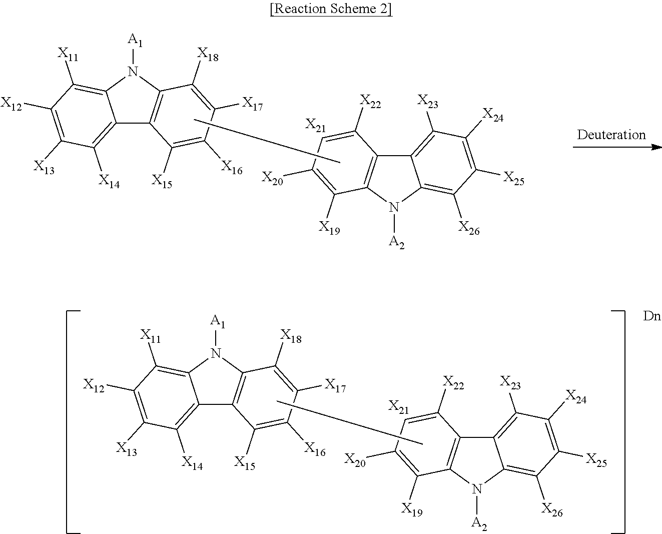

In reaction scheme 2, A₁, A₂, and X₁₁ to X₂₆ are as defined in Formula 2, and Dn signifies that n hydrogens are replaced with deuterium.

Although illustrative synthesis examples of the compounds represented by Formula 2 of the present disclosure are described above, one skilled in the art will be able to readily understand that all of them are based on a Buchwald-Hartwig cross-coupling reaction, an N-arylation reaction, an H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a cyclic dehydration reaction, an SN₁ substitution reaction, an SN₂ substitution reaction, a phosphine-mediated reductive cyclization reaction, etc., and the reaction above proceeds even when substituents which are defined in Formula 2, but are not specified in the specific synthesis example, are bonded.

In addition, the deuterated compounds of Formulas 1 and 2 may be prepared in a similar manner by using deuterated precursor materials, or more generally may be prepared by treating the non-deuterated compound with a deuterated solvent or D6-benzene in the presence of an H/D exchange catalyst such as a Lewis acid, e.g. aluminum trichloride or ethyl aluminum chloride. In addition, the degree of deuteration can be controlled by changing the reaction conditions such as the reaction temperature. For example, the number of deuterium in Formulas 1 and 2 can be controlled by adjusting the reaction temperature and time, the equivalent of acid, etc.

The organic electroluminescent compound according to the present disclosure is represented by the above Formula 1. According to one embodiment of the present disclosure, the organic electroluminescent device of the present disclosure may comprise an organic electroluminescent compound represented by Formula 1, and the compound may be used as a host material. According to another embodiment of the present disclosure, the present disclosure can provide a host material comprising a compound represented by Formula 1, and the host material may consist of only a single host or may comprise a plurality of host materials.

An organic electroluminescent device according to the present disclosure comprises an anode, a cathode, and at least one organic layer between the anode and the cathode, wherein the organic layer comprises a light-emitting layer(s).

According to one embodiment of the present disclosure, the light-emitting layer may comprise a plurality of host compounds comprising the first and second host compounds. In which, the weight ratio of the first and second host compounds may be about 1:99 to about 99:1, according to another embodiment of the present disclosure, it may be about 10:90 to about 90:10, according to another embodiment of the present disclosure, it may be about 30:70 to about 70:30, according to another embodiment of the present disclosure, it may be about 40:60 to about 60:40, and according to another embodiment of the present disclosure, it may be about 50:50. For example, the plurality of host materials of the present disclosure may comprise at least one compound selected from C-1 to C-140 as the first host compound, and at least one compound selected from H2-1 to H2-290 as the second host compound. The plurality of host materials may be included in the same organic layer, e.g. a light-emitting layer, or may be included in different light-emitting layers.

According to another embodiment of the present disclosure, the light-emitting layer may include an organic electroluminescent compound represented by Formula 1 according to the present disclosure. For example, the organic electroluminescent compound of the present disclosure may include at least one compound selected from C-1 to C-140, and may be comprised in the light-emitting layer as a host material.

The light-emitting layer may further include one or more dopants in addition to the plurality of host materials and/or organic electroluminescent compounds according to the present disclosure. The dopant may be at least one phosphorescent or fluorescent dopant, and for example, a phosphorescent dopant. The phosphorescent dopant material is not particularly limited, but may be a complex compound of metal selected from the group consisting of iridium (Ir), osmium (Os), copper (Cu), and platinum (Pt). According to one embodiment of the present disclosure, the complex compound of metal may be an ortho-metallated complex compound of metal selected from the group consisting of iridium (Ir), osmium (Os), copper (Cu), and platinum (Pt), and according to another embodiment of the present disclosure, it may be an ortho-metallated iridium complex compounds.

The dopant may be a compound represented by the following Formula 101, but is not limited thereto.

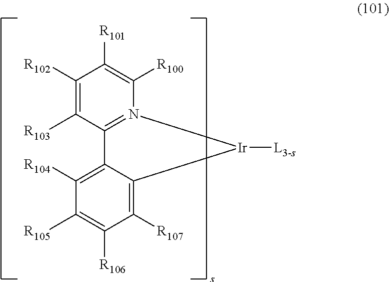

In Formula 101,
L is selected from the following structures 1 to 3:

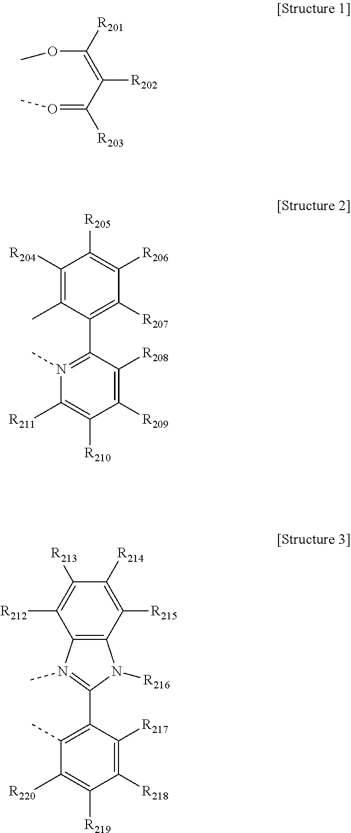

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g. a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), e.g. a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and
s represents an integer of 1 to 3.

The specific examples of the dopant compound are as follows, but are not limited thereto.

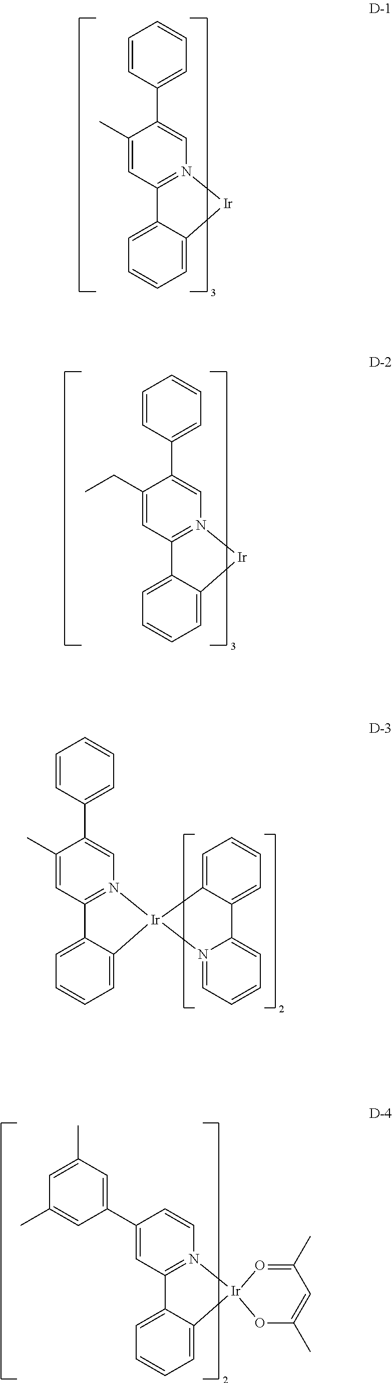

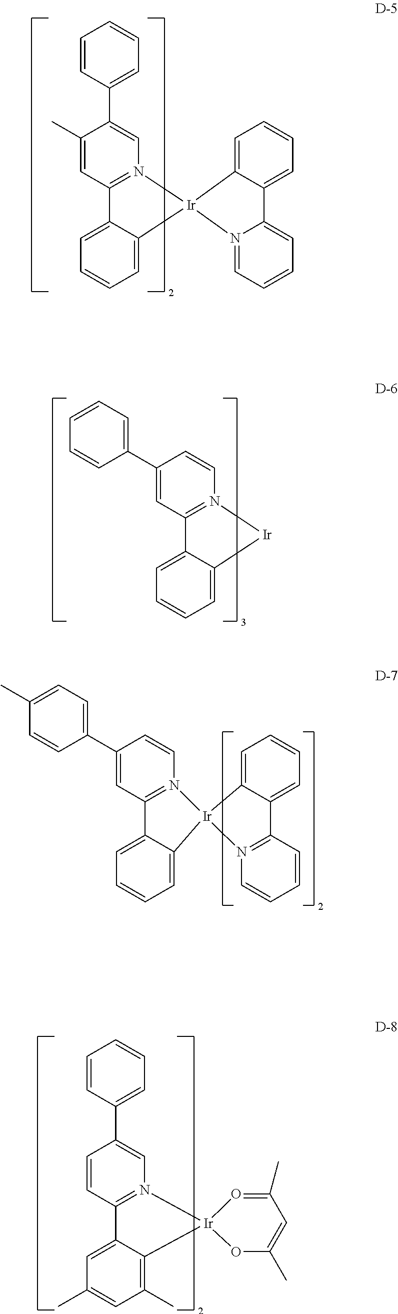

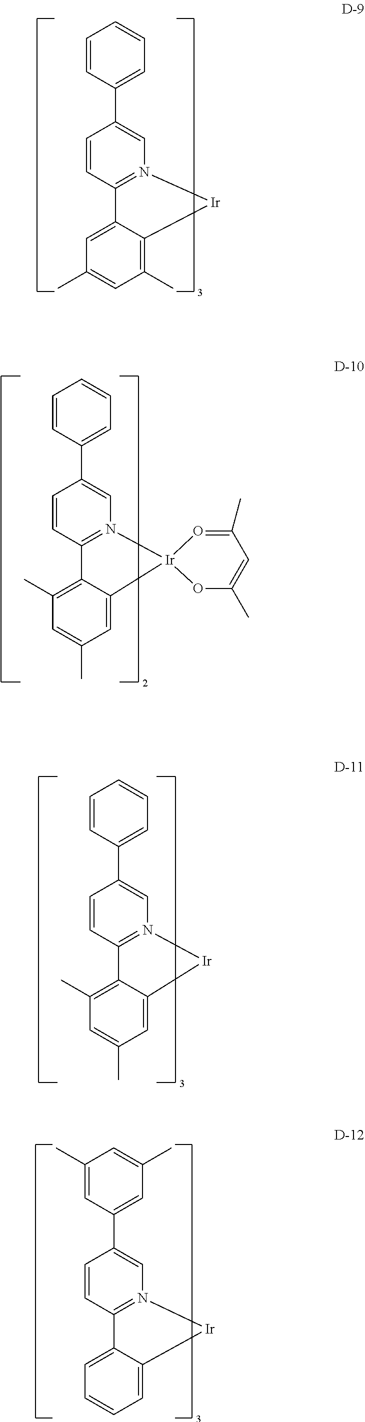

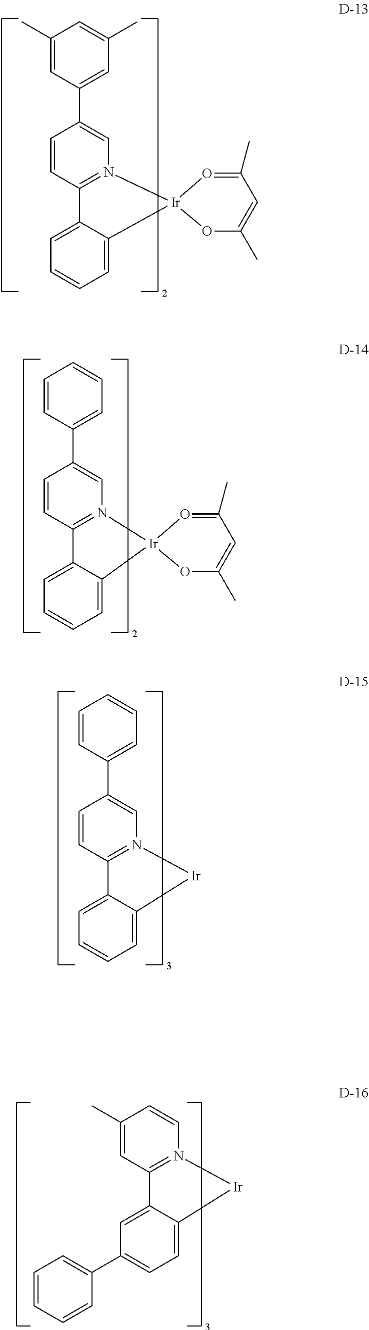

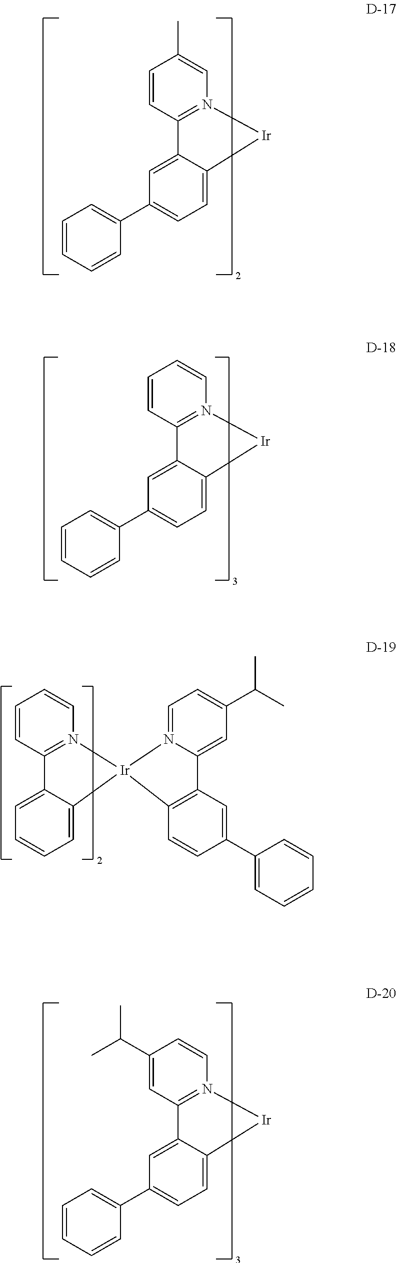

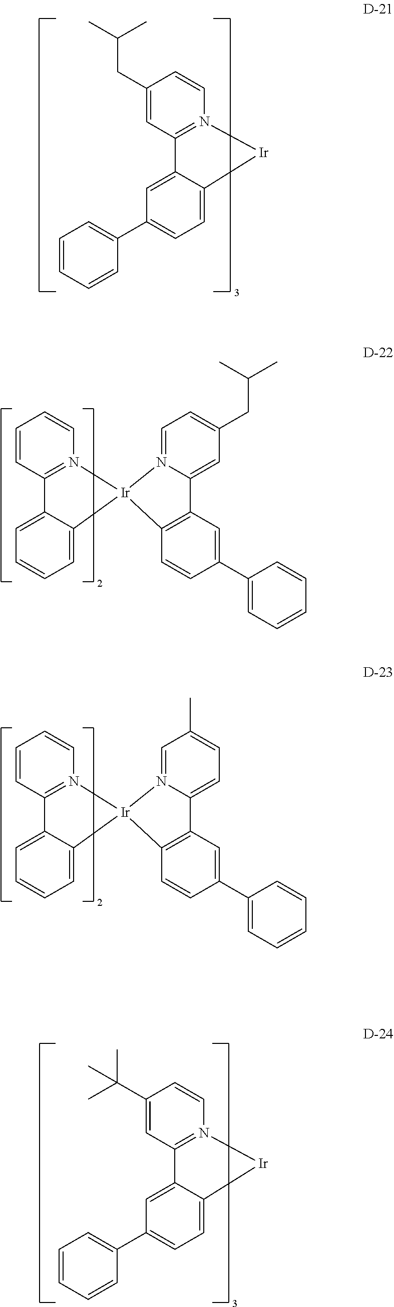

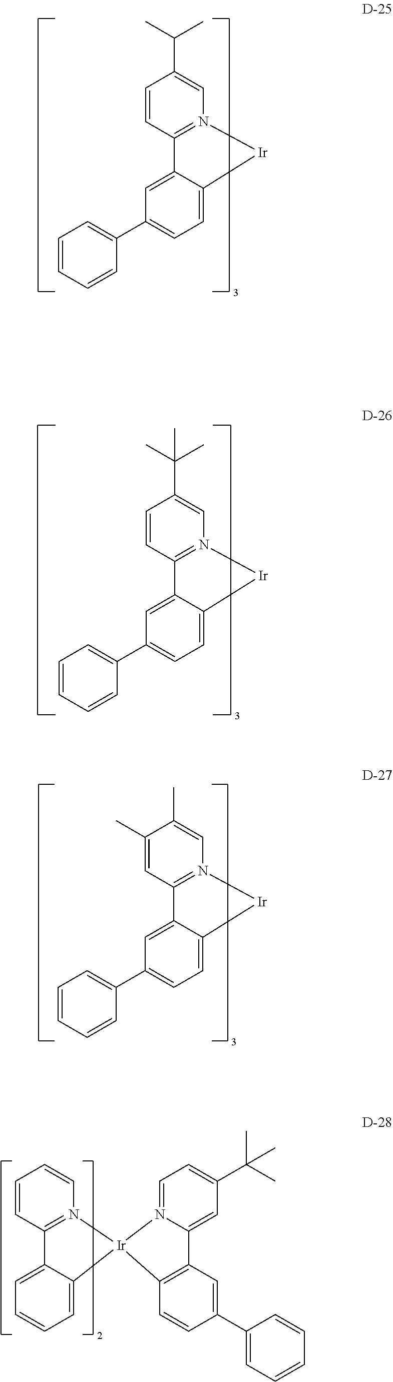

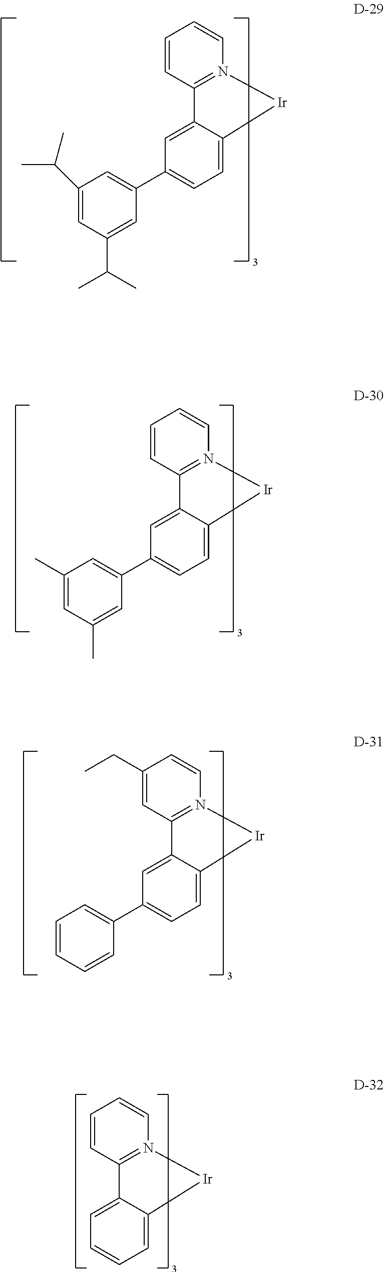

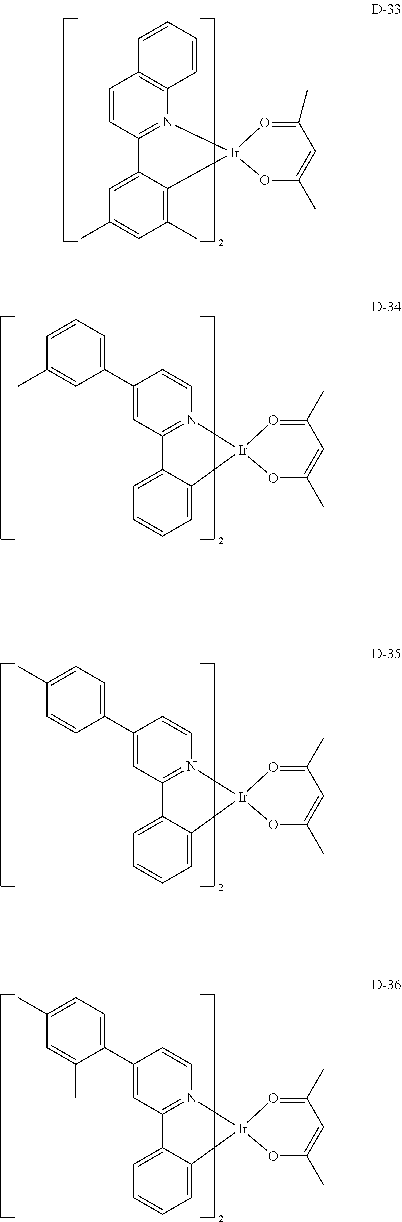

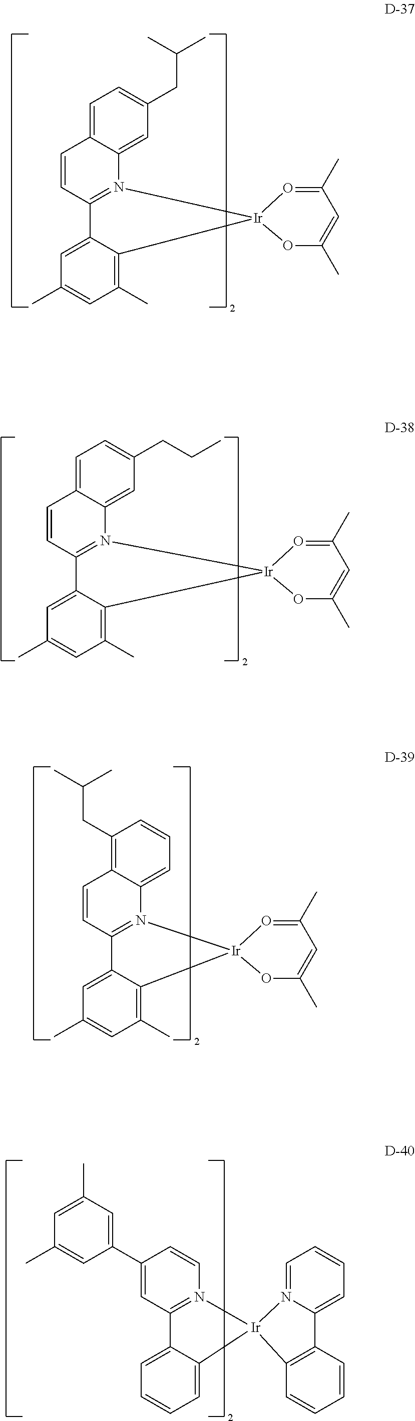

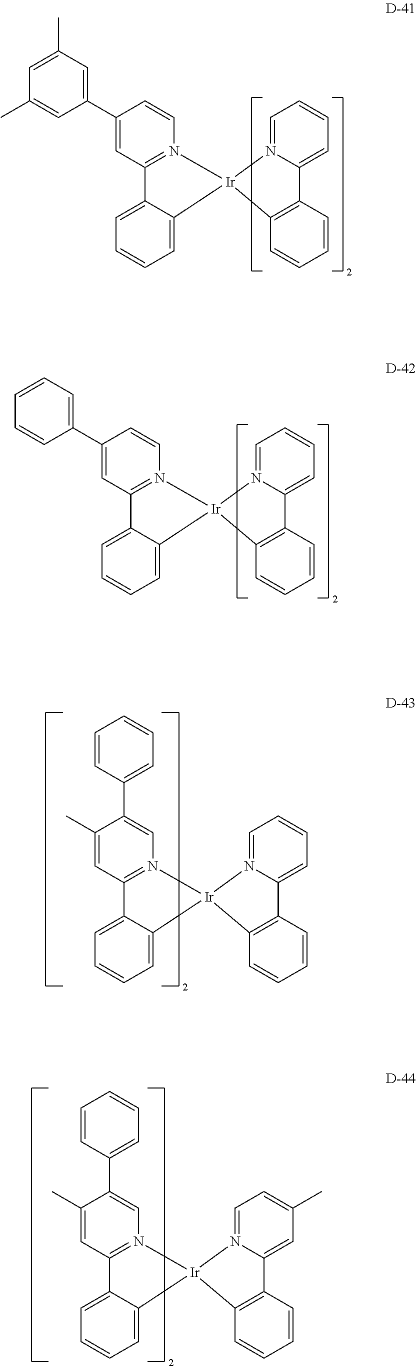

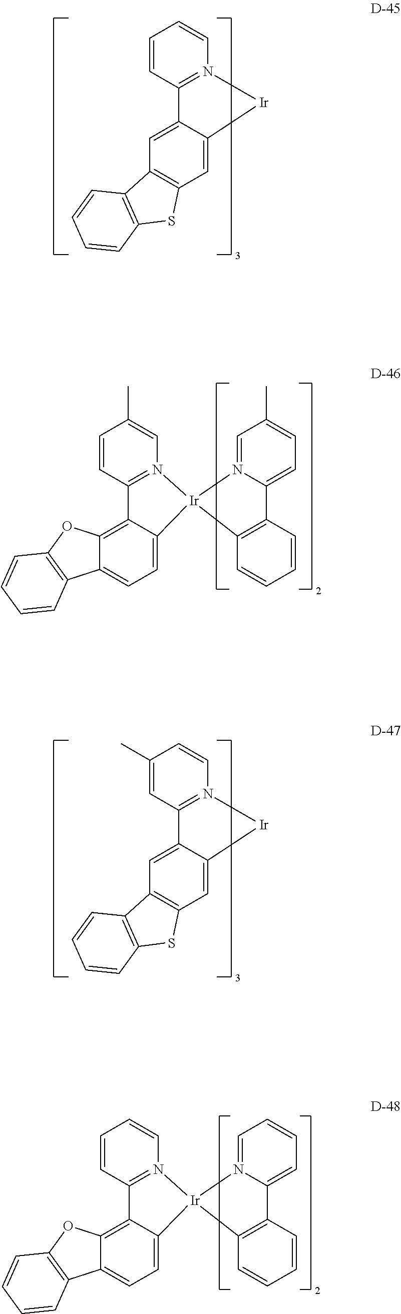

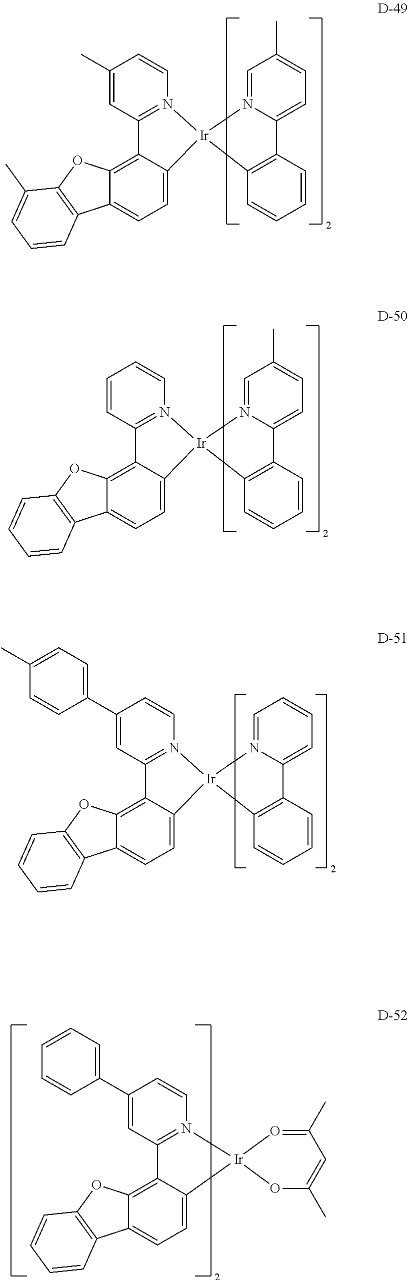

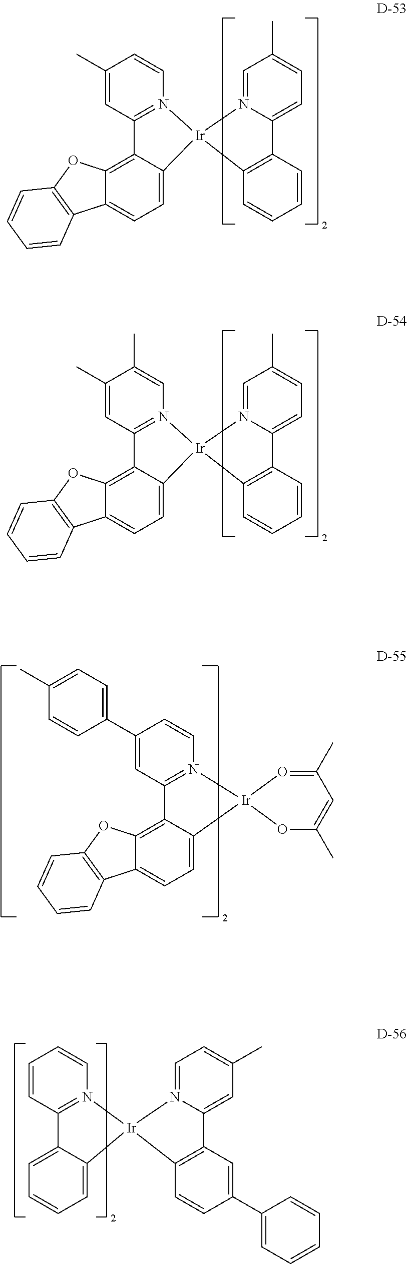

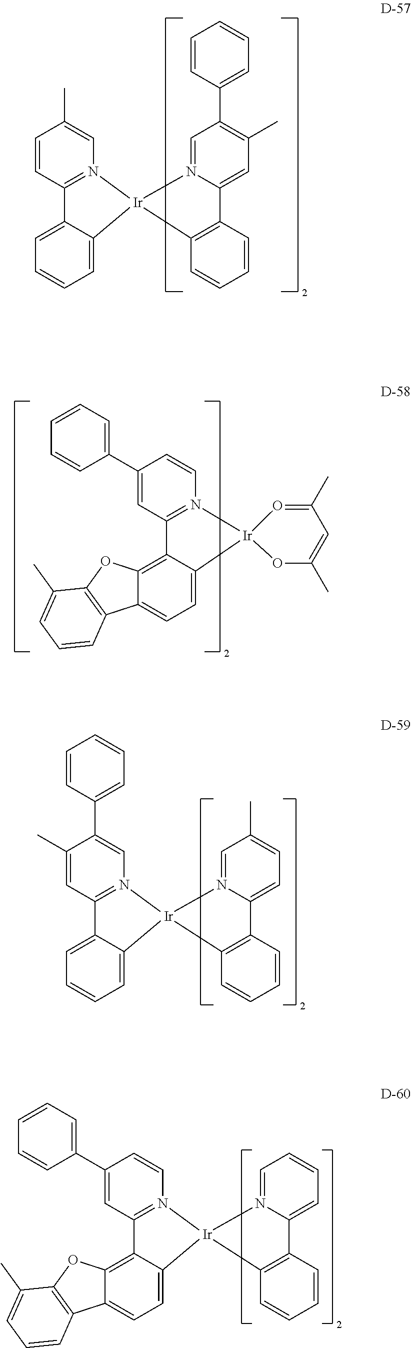

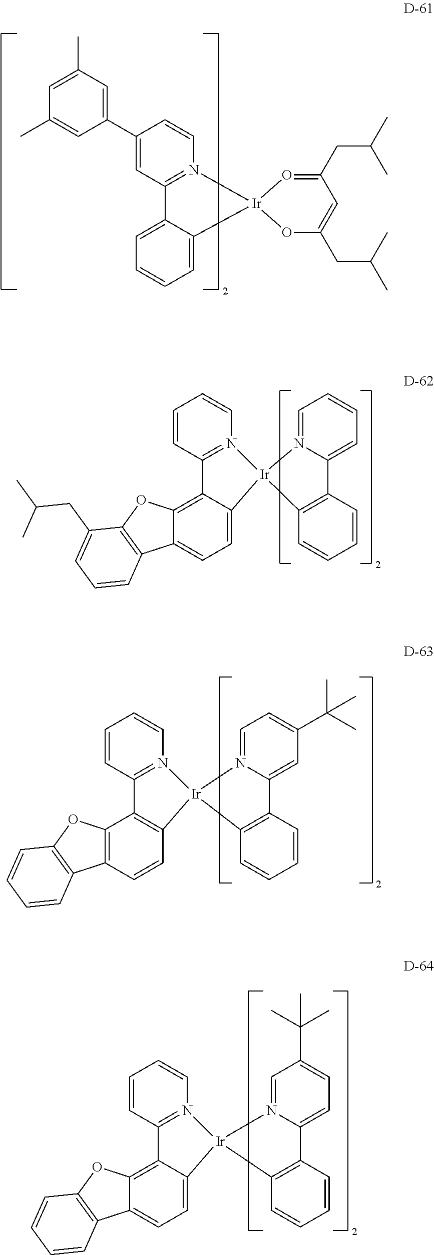

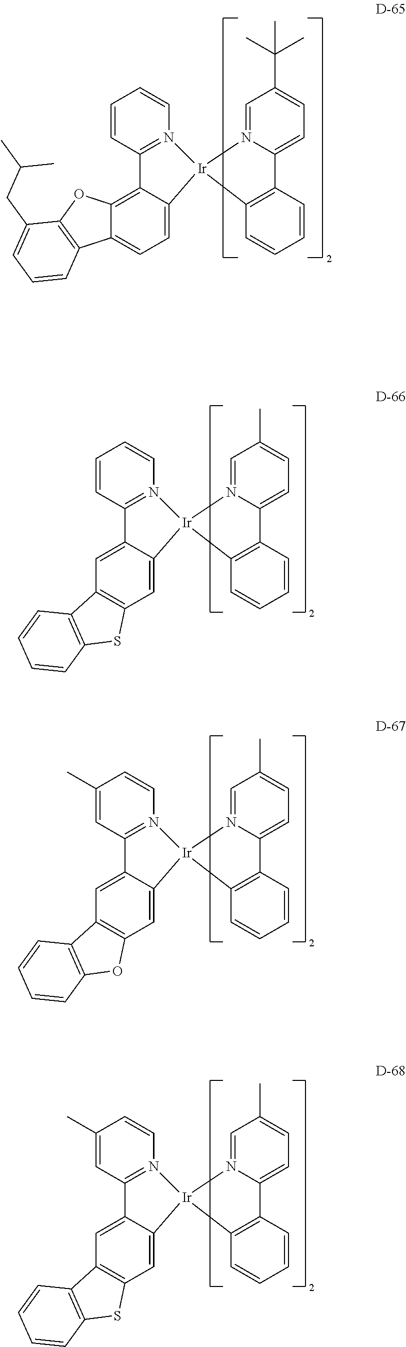

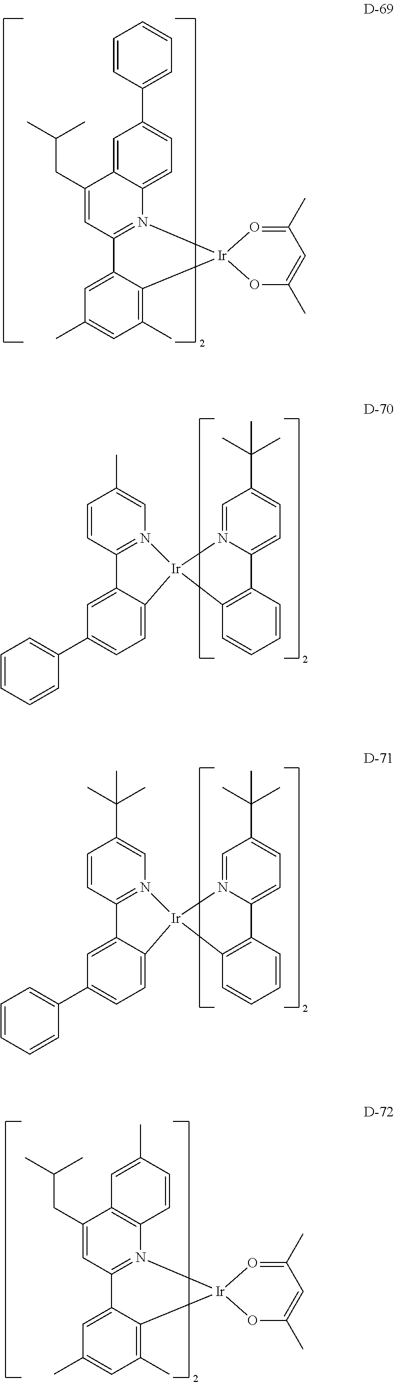

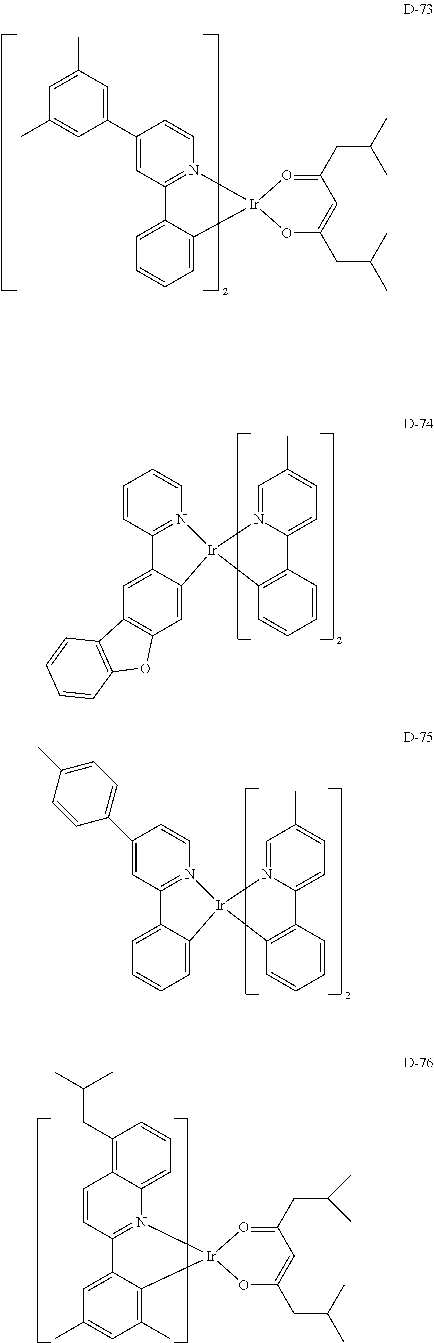

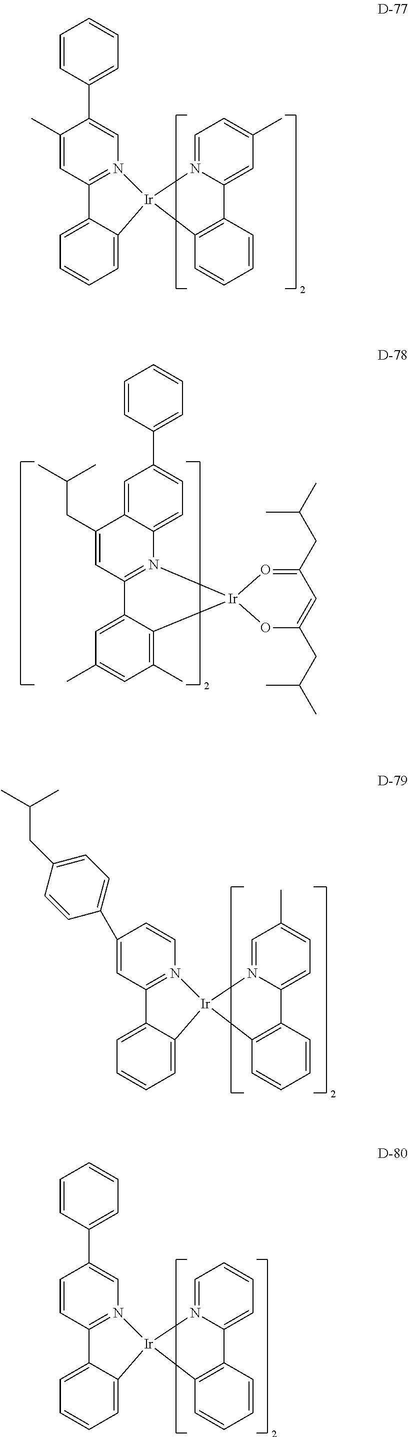

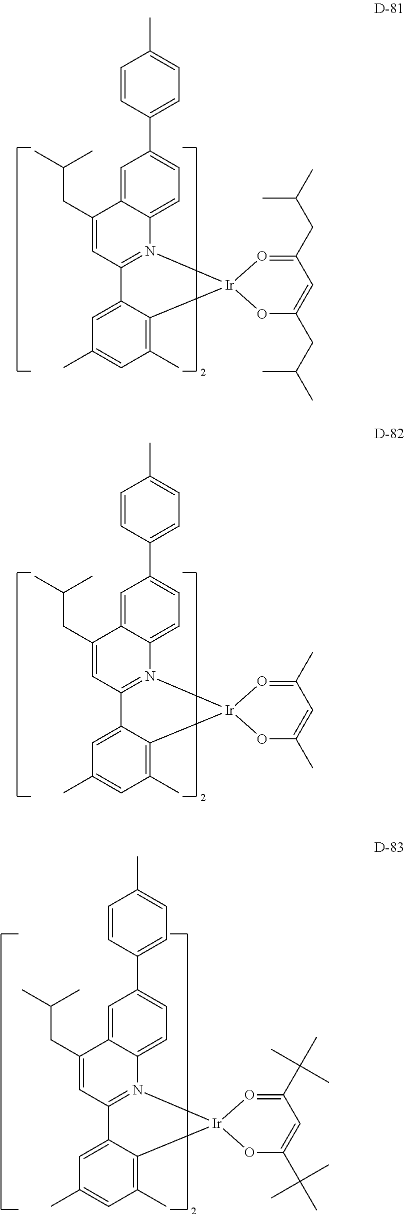

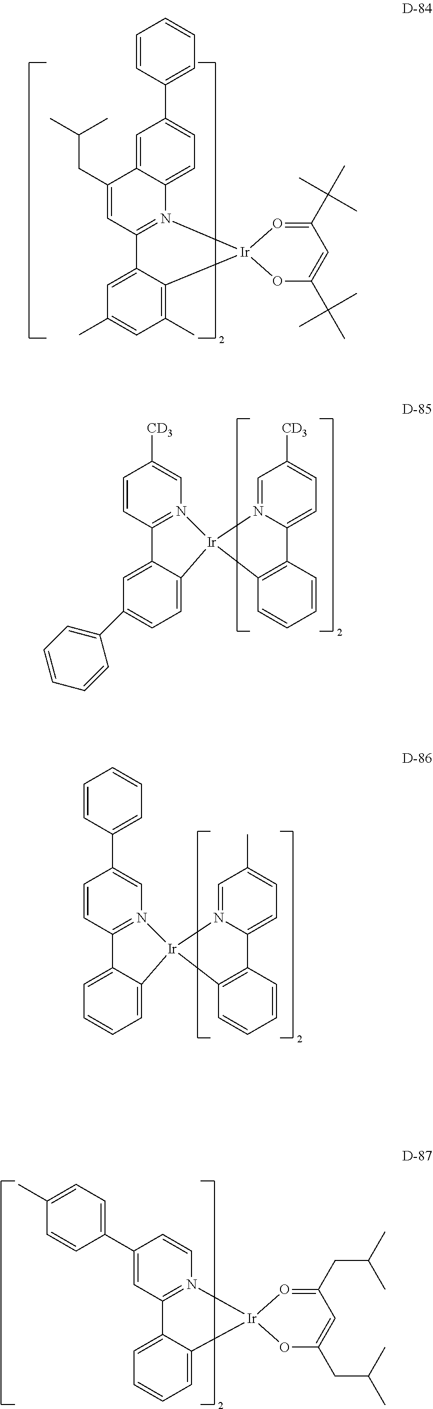

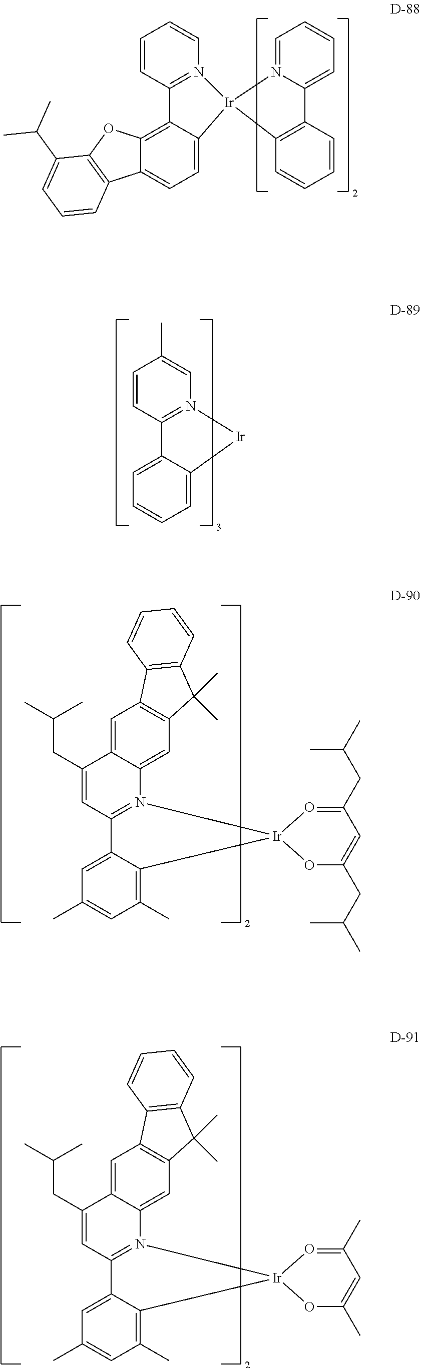

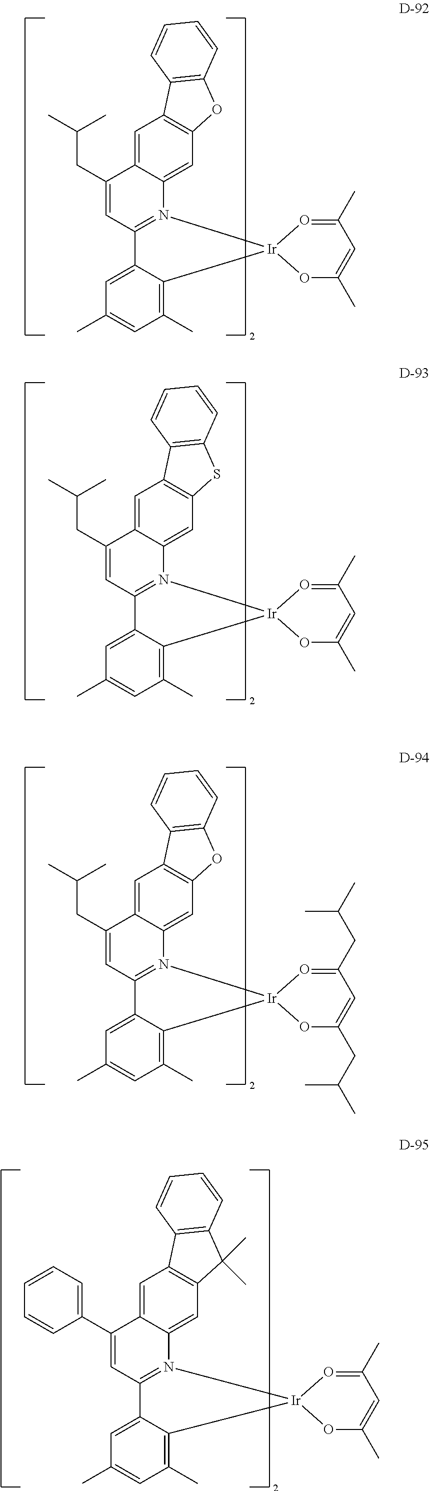

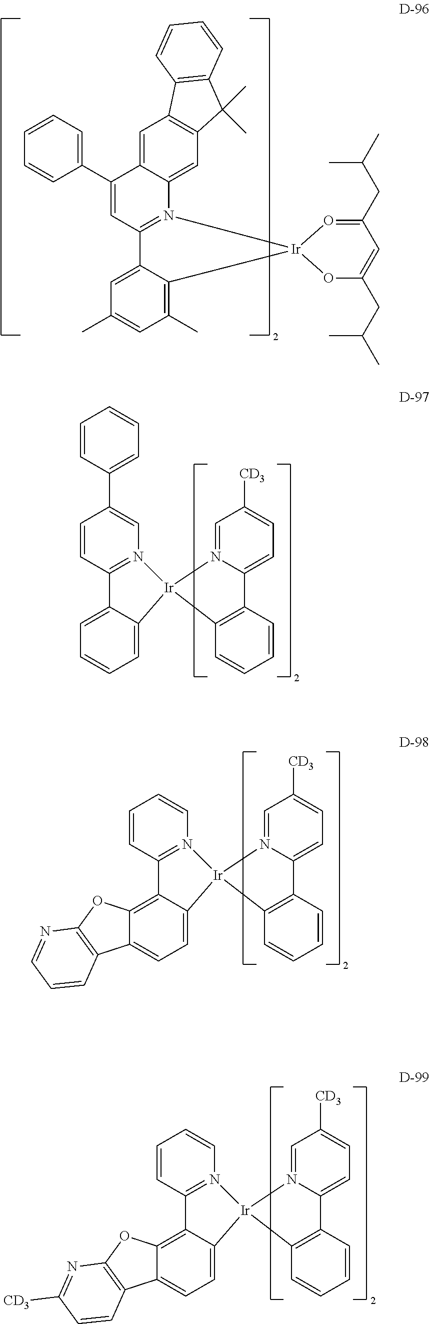

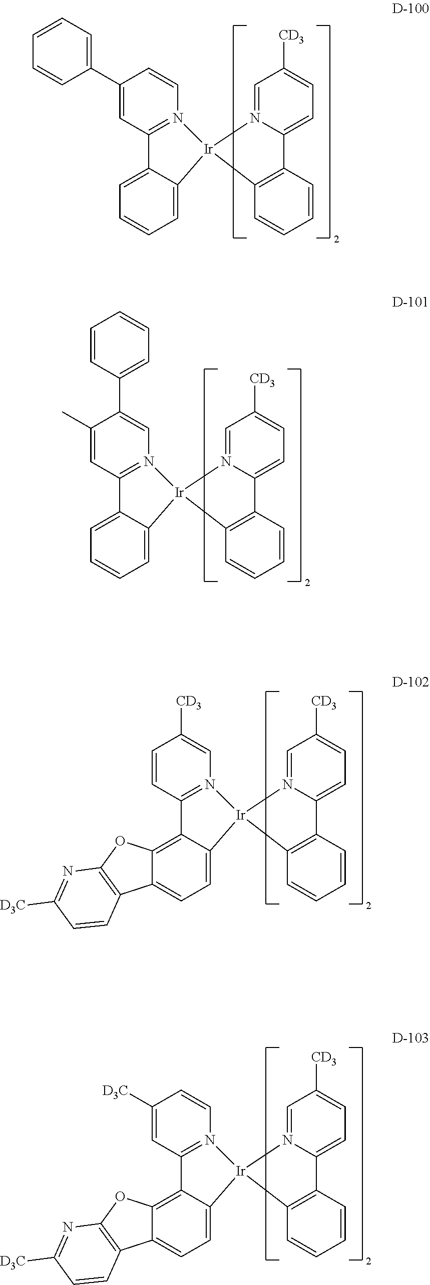

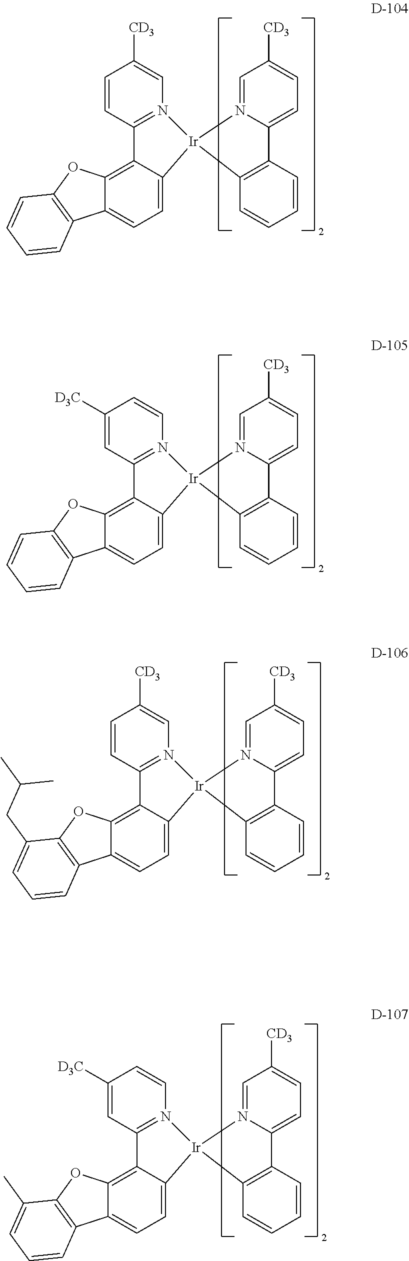

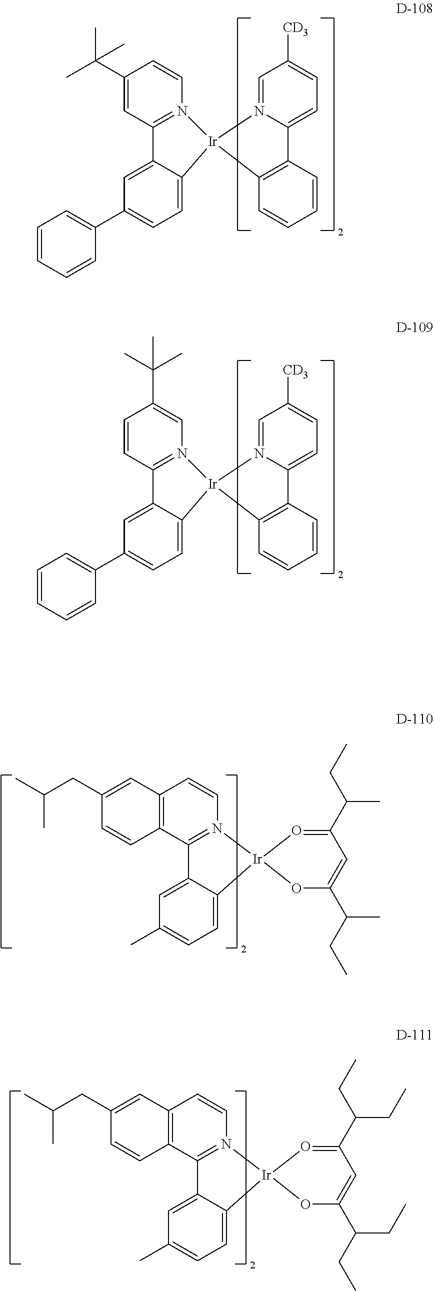

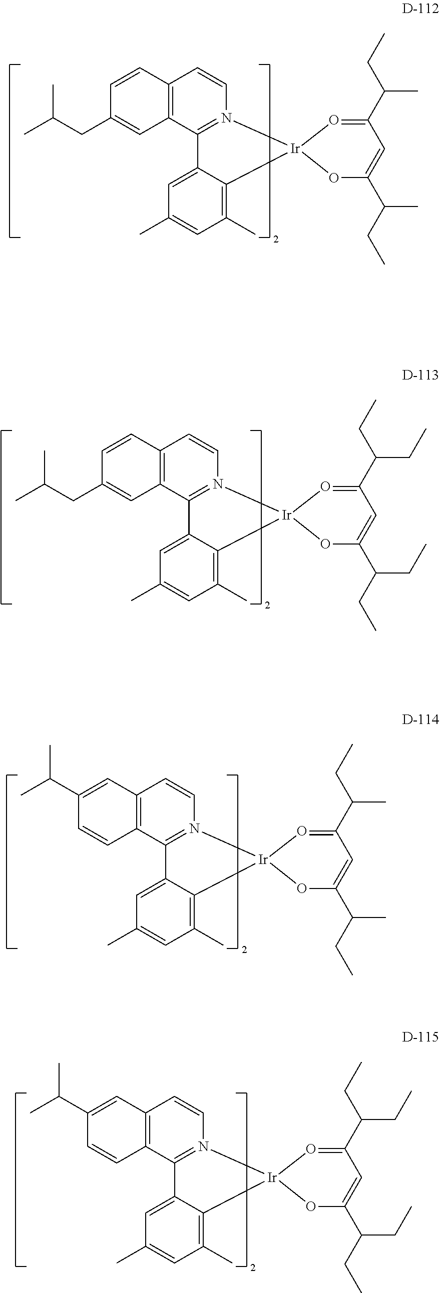

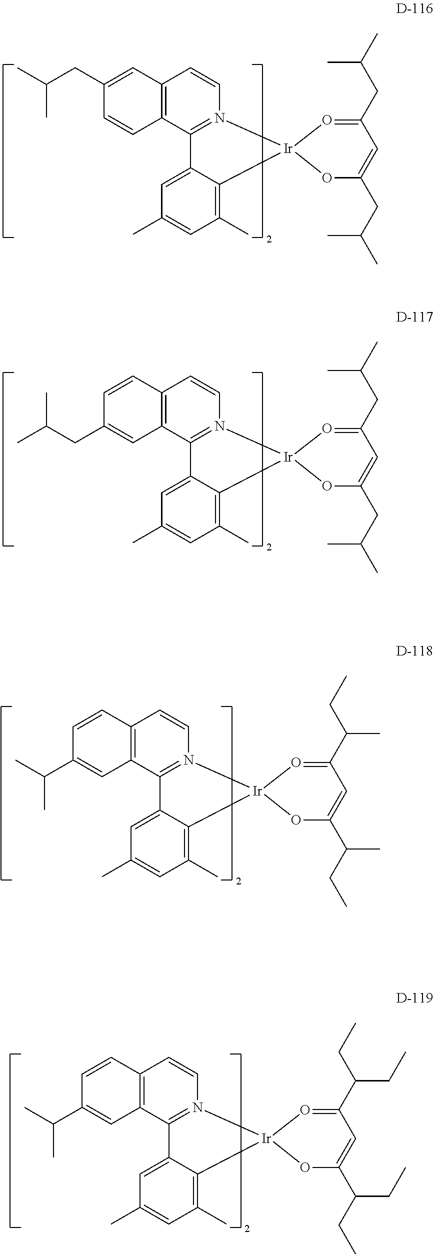

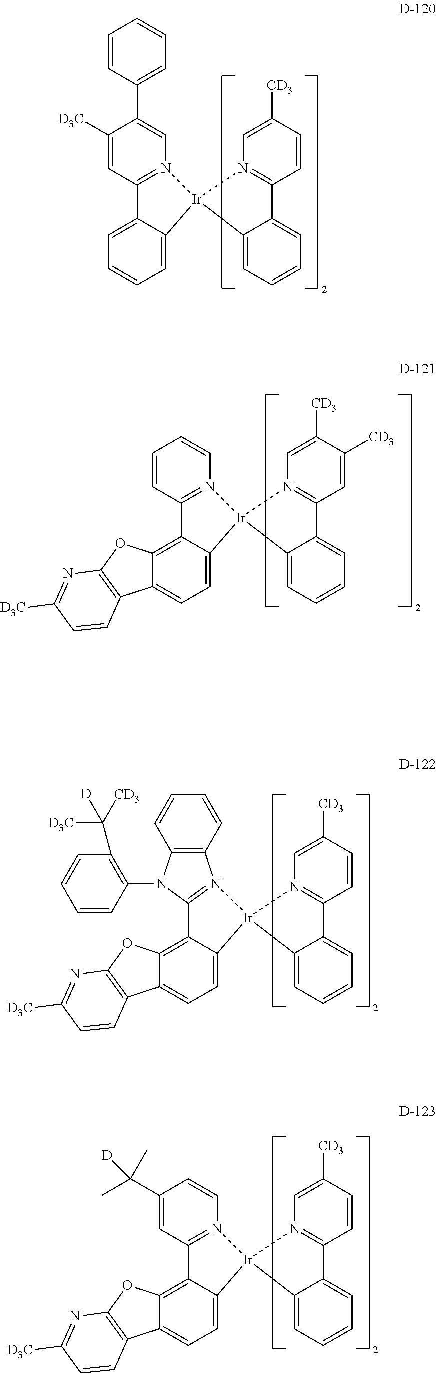

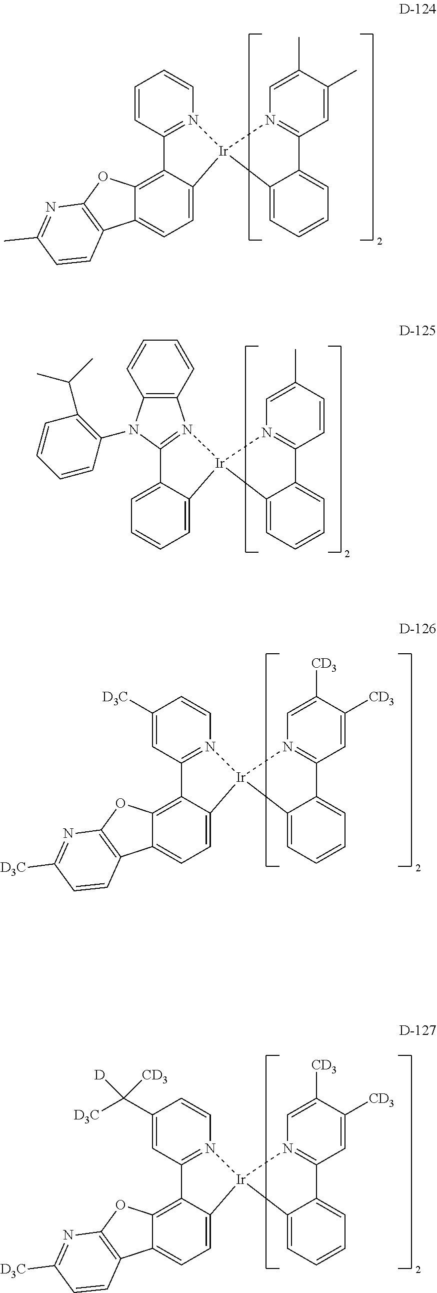

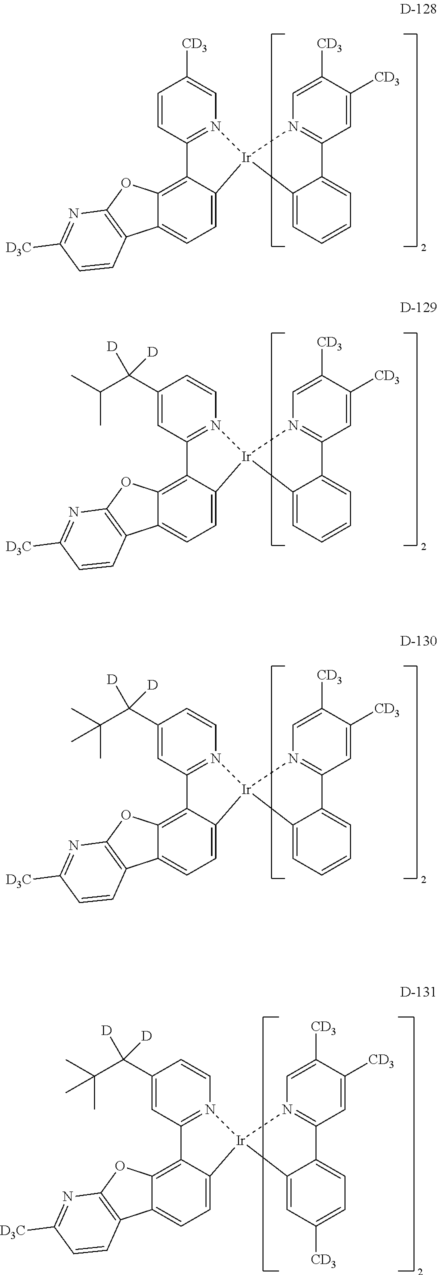

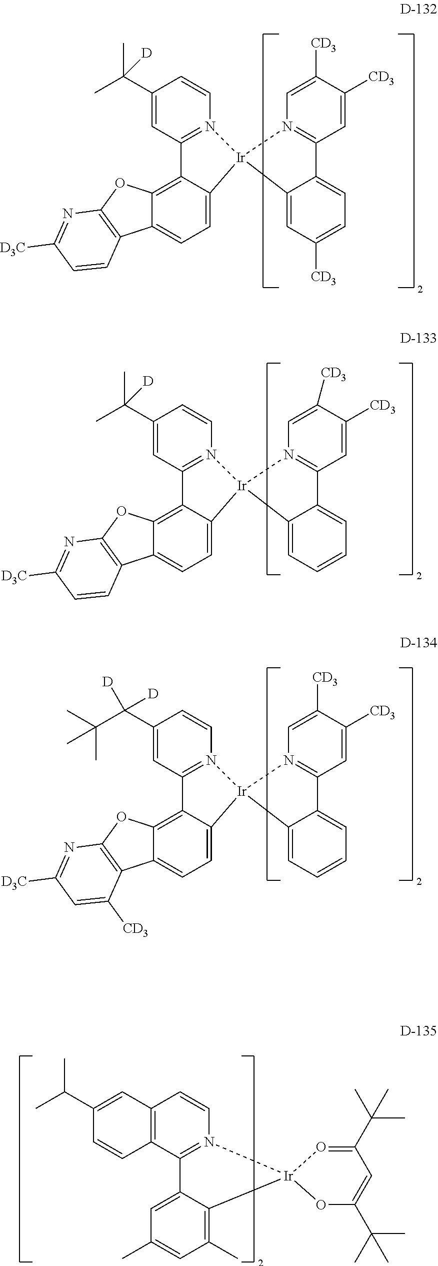

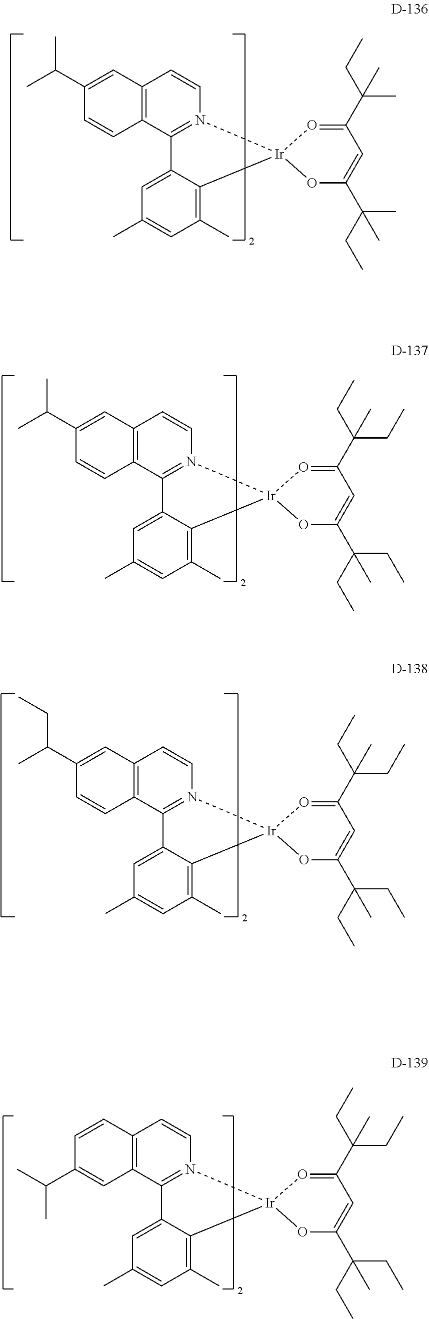

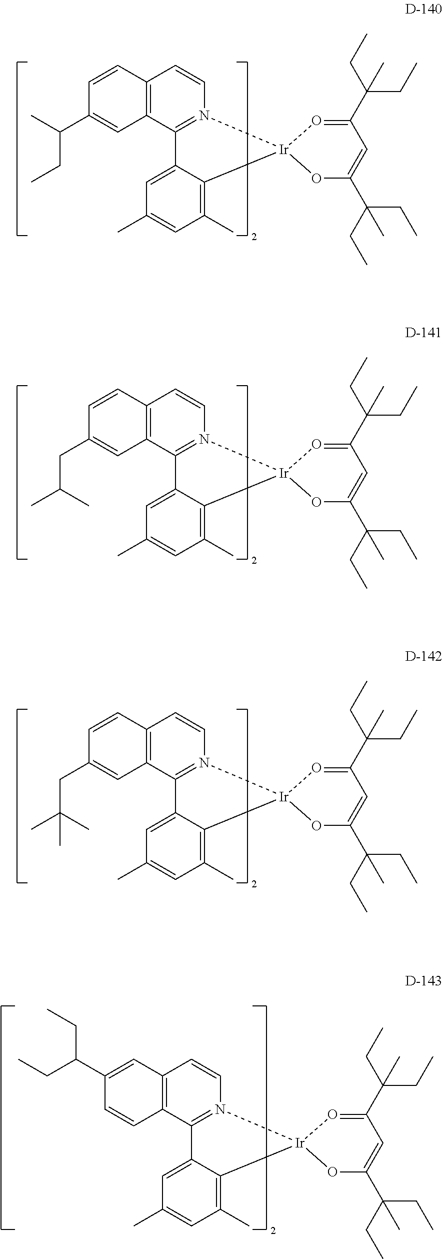

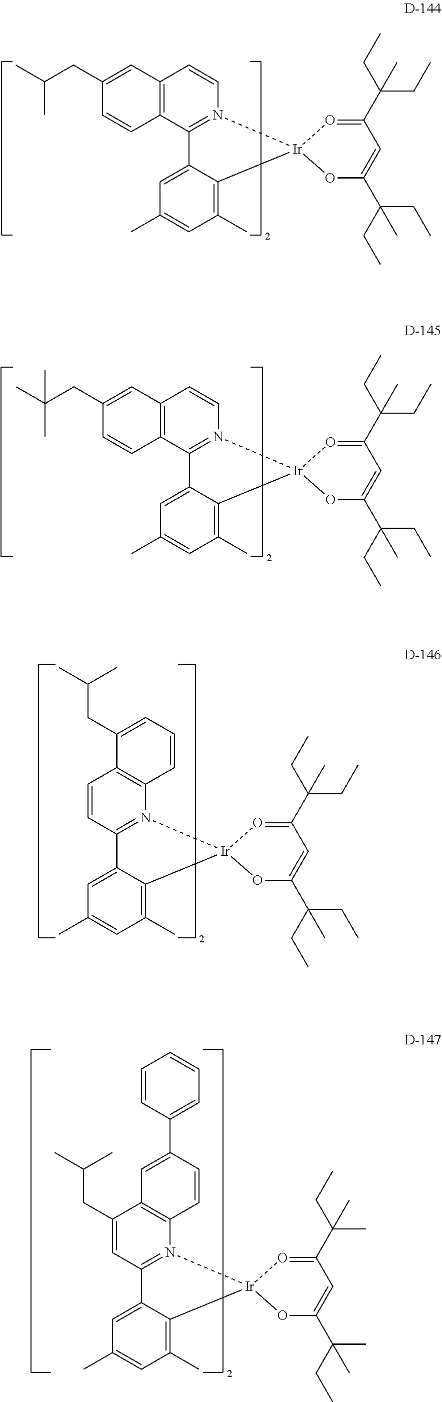

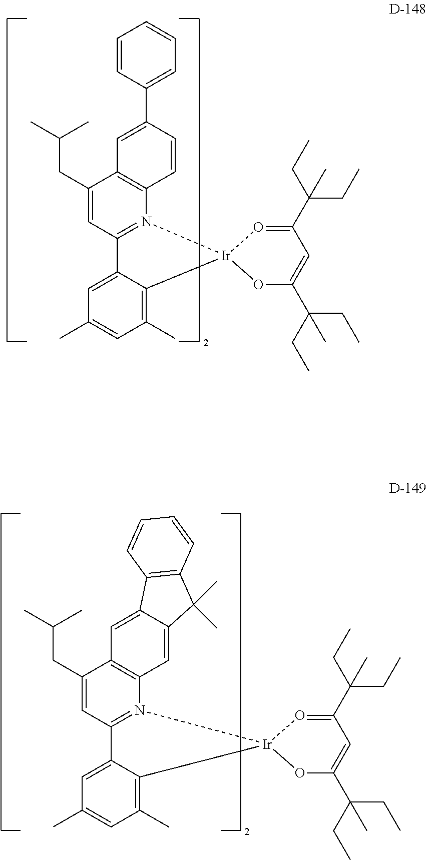

The organic layer comprises a light-emitting layer and may further comprise at least one layer selected from the group consisting of a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer. The organic layer may further include an amine-based compound and/or azine-based compound in addition to the light-emitting material of the present disclosure. Specifically, the hole injection layer, the hole transport layer, the hole auxiliary layer, the light-emitting layer, the light-emitting auxiliary layer, or the electron blocking layer may comprise an amine-based compound, e.g. an arylamine-based compound, a styrylarylamine-based compound, etc., as a hole injection material, a hole transport material, a hole auxiliary material, a light-emitting material, a light-emitting auxiliary material, and an electron blocking material. In addition, the electron transport layer, electron injection layer, electron buffer layer, and hole blocking layer may include an azine-based compound as an electron transport material, an electron injection material, an electron buffer material, and a hole blocking material. In addition, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising a metal.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped with a p-dopant. The electron blocking layer is located between the hole transport layer (or hole injection layer) and the light-emitting layer, and can prevent light leakage by blocking the overflow of electrons from the light-emitting layer and confining excitons within the light-emitting layer. The hole transport layer or the electron blocking layer may be multi-layers, wherein a plurality of compounds may be used in each of the multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer is located between the electron transport layer (or electron injection layer) and the light-emitting layer, and can improve the probability of recombination of electrons and holes in the light-emitting layer by preventing holes from reaching the cathode. The hole blocking layer or the electron transport layer may be multi-layers, wherein a plurality of compounds may be used in each of the multi-layers. In addition, the electron injection layer may be doped with an n-dopant.

The light-emitting auxiliary layer, the hole auxiliary layer, and the electron blocking layer may be provided to improve the efficiency and/or lifetime properties of the organic electroluminescent device.

The light-emitting auxiliary layer may be placed between the anode and the light-emitting layer, or between the cathode and the light-emitting layer. In one embodiment of the present disclosure, when the light-emitting auxiliary layer is placed between the anode and the light-emitting layer, it can be used for promoting the injection and/or transport of hole, or for preventing the overflow of electrons. In another embodiment of the present disclosure, when the light-emitting auxiliary layer is placed between the cathode and the light-emitting layer, it can be used for promoting the injection and/or transport of electron, or for preventing the overflow of holes.

The hole auxiliary layer may be placed between the hole transport layer (or hole injection layer) and the light-emitting layer, and may be effective to promote or block the hole transport rate (or hole injection rate), thereby enabling the charge balance to be controlled. When an organic electroluminescent device includes two or more hole transport layers, the hole transport layer, which is further included, may be used as a hole auxiliary layer or an electron blocking layer.

In the organic electroluminescent device of the present disclosure, at least one layer selected from the group consisting of a chalcogenide layer, a metal halide layer, and a metal oxide layer (hereinafter, “a surface layer”) may be placed on an inner surface(s) of one or both electrode(s). For example, a chalcogenide (including oxides) layer of silicon or aluminum may be placed on an anode surface of an electroluminescent medium layer, and a metal halide layer or a metal oxide layer may be placed on a cathode surface of an electroluminescent medium layer. Such a surface layer provides operation stability for the organic electroluminescent device. Specifically, the chalcogenide includes SiO_{X}(1≤X≤2), AlO_{X}(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this way, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Specifically, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

In order to form each layer of the organic electroluminescent device of the present disclosure, dry film-forming methods such as vacuum deposition, sputtering, plasma, ion plating methods, etc., or wet film-forming methods such as spin coating, dip coating, flow coating methods, etc., can be used. When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

According to one embodiment of the present disclosure, when forming a layer of the first host compound and the second host compound, the layer can be formed by the methods listed above, and often can be formed by a co-deposition or mixed deposition process. Co-deposition is a method for mixing two or more materials into each individual crucible source and applying current to both cells simultaneously to evaporate the materials. Mixed deposition is a method for mixing two or more materials in one crucible source before deposition and then applying current to one cell to evaporate the materials.

According to one embodiment of the present disclosure, when the first host compound and the second host compound are present in the same layer or different layers in the organic electroluminescent device, the two host compounds may be individually formed into films. For example, the second host compound may be deposited after depositing the first host compound.

The plurality of host materials and the organic electroluminescent compound according to one embodiment of the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The plurality of host materials and the organic electroluminescent compound according to one embodiment of the present disclosure may also be used in an organic electroluminescent device comprising a quantum dot (QD).

According to one embodiment of the present disclosure, a display system comprising the plurality of host materials and/or the organic electroluminescent compound of the present disclosure can be provided. In addition, it is possible to produce a display system, e.g. a display system for smart phones, tablets, notebooks, PCs, TVs, or cars; or a lighting system, e.g. an outdoor or indoor lighting system, by using the organic electroluminescent device of the present disclosure. Hereinafter, the preparation method of the plurality of host materials, the organic electroluminescent compound, and the organic electroluminescent device according to the present disclosure and the properties thereof will be explained in detail with reference to the representative compounds of the present disclosure. However, the present disclosure is not limited to the following examples.

### Example 1: Preparation of Compound C-1

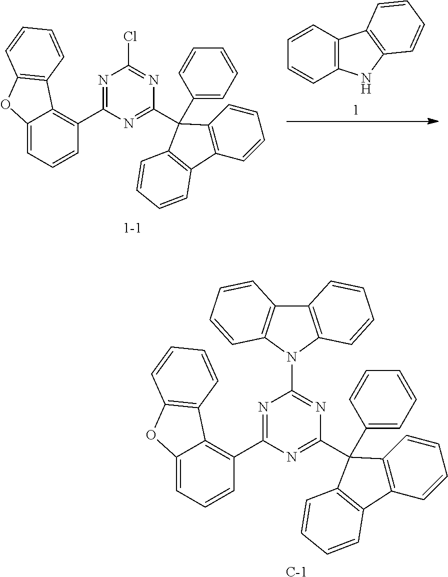

In a flask, compound 1 (carbazole) (0.51 g, 3.03 mmol), compound 1-1 (1.9 g, 3.64 mmol), cesium carbonate (Cs₂CO₃) (0.99 g, 3.03 mmol), and DMAP (0.19 g, 1.52 mmol) were dissolved in 15 mL of DMSO, and the mixture was refluxed at 100° C. for 4 hours. The mixture was cooled to room temperature, and H₂O was added to the solid reaction product, stirred for 30 minutes, filtered, and separated by column chromatography to obtain compound C-1 (1.1 g, yield: 56%).

| | MW | M.P. |
|---|---|---|
| C-1 | 652.76 | 254.1° C. |

### Example 2: Preparation of Compound C-86

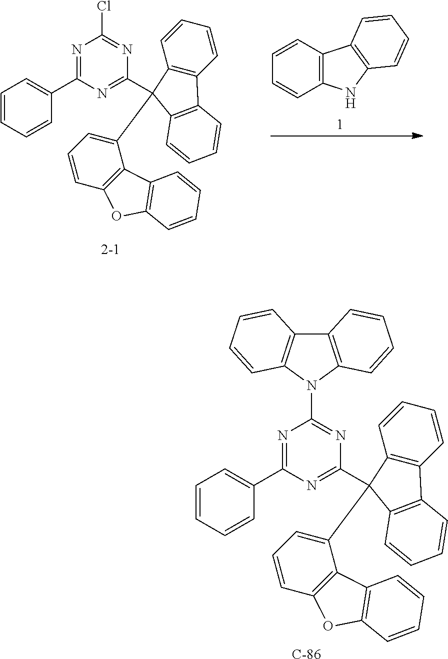

In a flask, compound 1 (carbazole) (1.4 g, 8.37 mmol), compound 2-1 (5.3 g, 10.05 mmol), cesium carbonate (Cs₂CO₃) (2.73 g, 8.37 mmol), and DMAP (0.51 g, 4.19 mmol) were dissolved in 42 mL of DMSO, and the mixture was refluxed at 100° C. for 4 hours. The mixture was cooled to room temperature, and H₂O was added to the solid reaction product, stirred for 30 minutes, filtered, and separated by column chromatography to obtain compound C-86 (2.3 g, yield: 42%).

| | MW | M.P. |
|---|---|---|
| C-86 | 652.76 | 305.8° C. |

### Example 3: Preparation of Compound C-87

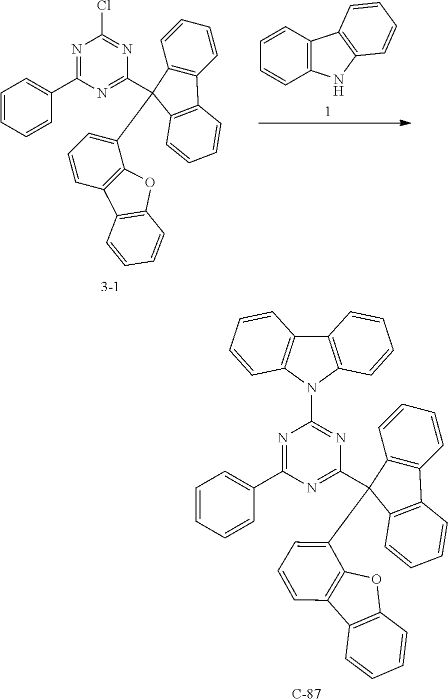

In a flask, compound 1 (carbazole) (1.9 g, 11.13 mmol), compound 3-1 (5.8 g, 11.13 mmol), cesium carbonate (Cs₂CO₃) (5.4 g, 16.69 mmol), and DMAP (0.68 g, 5.565 mmol) were dissolved in 60 mL of DMSO, and the mixture was refluxed at 100° C. for 4 hours. The mixture was cooled to room temperature, and H₂O was added to the solid reaction product, stirred for 30 minutes, filtered, and separated by column chromatography to obtain compound C-87 (3.2 g, yield: 44%).

| | MW | M.P. |
|---|---|---|
| C-87 | 652.76 | 246.6° C. |

### Device Examples 1 to 8: Producing Green Light-Emitting OLEDs Comprising the Plurality of Host Materials According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. The compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: each of the first host compound and the second host compound shown in Table 1 below were introduced into two cells of the vacuum vapor deposition apparatus as hosts, and compound D-130 was introduced into another cell as a dopant. The two host materials were evaporated at a rate of 1:2 (first host: second host) and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Compound ETL-1 and compound EIL-1 were evaporated in a weight ratio of 40:60 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Comparative Compound as a Host

An OLED was produced in the same manner as in Device Examples 1 to 8, except that compound T-1 and compound H2-191 were used as the first host compound and the second host compound of the light-emitting layer, respectively.

### Comparative Example 2: Producing an OLED Comprisinq a Comparative Compound as a Host

An OLED was produced in the same manner as in Device Examples 1 to 8, except that compound T-2 and compound H2-191 were used as the first host compound and the second host compound of the light-emitting layer, respectively.

The driving voltage, luminous efficiency, and light-emitting color at a luminance of 1,000 nit, and the time taken for luminance to reduce from 100% to 95% at a luminance of 20,000 nit (lifetime: T95) of the OLEDs produced in Device Examples 1 to 8 and Comparative Examples 1 and 2 are shown in Table 1 below.

TABLE 1

| | | | | | | Life- |
|---|---|---|---|---|---|---|
| | | | Driving | Luminous | Light- | time |
| | First | Second | Voltage | Efficiency | Emitting | (T95) |
| | Host | Host | [V] | [cd/A] | Color | [hr] |
| Device | C-1 | H2-147 | 3.2 | 97.8 | Green | 208.0 |
| Example 1 | | | | | | |
| Device | C-1 | H2-2 | 3.2 | 97.9 | Green | 242.6 |
| Example 2 | | | | | | |
| Device | C-86 | H2-147 | 3.4 | 102.4 | Green | 158.6 |
| Example 3 | | | | | | |
| Device | C-86 | H2-2 | 3.5 | 101.8 | Green | 171.9 |
| Example 4 | | | | | | |
| Device | C-1 | H2-191 | 3.3 | 98.6 | Green | 235.6 |
| Example 5 | | | | | | |
| Device | C-1 | H2-46 | 3.2 | 98.1 | Green | 235.6 |
| Example 6 | | | | | | |
| Device | C-86 | H2-191 | 3.4 | 103.0 | Green | 178.3 |
| Example 7 | | | | | | |
| Device | C-86 | H2-46 | 3.3 | 102.4 | Green | 195.8 |
| Example 8 | | | | | | |
| Comparative | T-1 | H2-191 | 3.0 | 100.5 | Green | 50.3 |
| Example 1 | | | | | | |
| Comparative | T-2 | H2-191 | 3.5 | 99.4 | Green | 70.5 |
| Example 2 | | | | | | |

As shown in Table 1 above, it can be confirmed that the organic electroluminescent device using the plurality of host materials according to the present disclosure (Device Examples 1 to 8) exhibits a driving voltage and luminous efficiency of at least the same or similar level as the organic electroluminescent device comprising the conventional combination of hosts (Comparative Examples 1 and 2), while exhibiting excellent lifetime properties.

The lifetime of green light-emitting organic electroluminescent devices is generally shorter than that of red light-emitting organic electroluminescent devices. In order to improve the lifetime properties of a green light-emitting organic electroluminescent device, a compound with a deuterated moiety was used. It is not limited by theory, but when an organic electroluminescent compound is replaced with deuterium, the stability of the compound can be increased by lowering the zero point vibration energy of the compound and increasing the bond dissociation energy (BDE) in the compound.

### Device Examples 9 to 11: Producing Green OLEDs by Depositing a Single Host Material According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. The compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: the host compound shown in Table 2 below was introduced into one cell of the vacuum vapor deposition apparatus as a host, and compound D-130 was introduced into another cell as a dopant. The dopant material was evaporated at a different rate, and the dopant was deposited in a doping amount of 10 wt % based on the total amount of the host and the dopant to form a light-emitting layer having a thickness of 40 nm on the second hole transport layer. Compound ETL-1 and compound EIL-1 were evaporated in a weight ratio of 40:60 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 3: Producing an OLED Comprisinq a Comparative Compound as a Host

An OLED was produced in the same manner as in Device Examples 9 to 11, except that compound T-1 was used as the host of the light-emitting layer.

### Comparative Example 4: Producing an OLED Comprising a Comparative Compound as a Host

An OLED was produced in the same manner as in Device Examples 9 to 11, except that compound T-2 was used as the host of the light-emitting layer.

The driving voltage, luminous efficiency, and light-emitting color at a luminance of 1,000 nit, and the time taken for luminance to reduce from 100% to 80% at a luminance of 20,000 nit (lifetime: T80) of the OLEDs produced in Device Examples 9 to 11 and Comparative Examples 3 and 4 are shown in Table 2 below.

TABLE 2

| | | Driving | Luminous | Light- | |
|---|---|---|---|---|---|
| | | Voltage | Efficiency | Emitting | Lifetime |
| | Host | [V] | [cd/A] | Color | (T80) [hr] |
| Device | C-1 | 2.7 | 92.5 | Green | 105.8 |
| Example 9 | | | | | |
| Device | C-86 | 3.1 | 107.6 | Green | 88.2 |
| Example 10 | | | | | |
| Device | C-87 | 3.2 | 104.8 | Green | 58.9 |
| Example 11 | | | | | |
| Comparative | T-1 | 2.6 | 93.5 | Green | 14.8 |
| Example 3 | | | | | |
| Comparative | T-2 | 2.8 | 88.1 | Green | 42.0 |
| Example 4 | | | | | |

As shown in Table 2 above, it can be confirmed that the organic electroluminescent device using the single host material according to present disclosure (Device Examples 9 to 11) exhibits a driving voltage and luminous efficiency of at least the same or similar level as the organic electroluminescent device comprising the conventional host (Comparative Examples 3 and 4), while exhibiting excellent lifetime properties.

The compounds used in the Device Examples and the Comparative Examples are shown in Table 3 below.

TABLE 3

| | |
|---|---|
| Hole Injection Layer/ Hole Transport Layer | 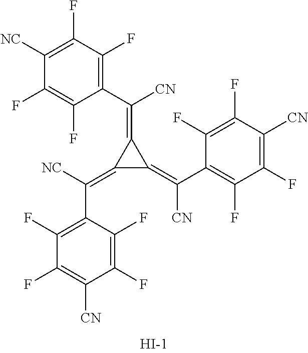 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | 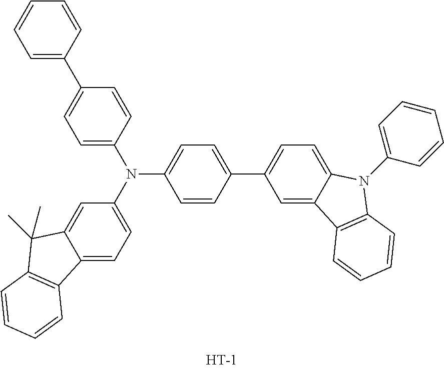 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | 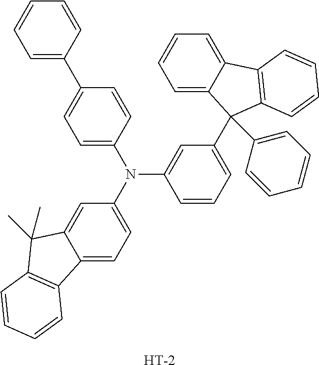 |
| Light-Emitting Layer | 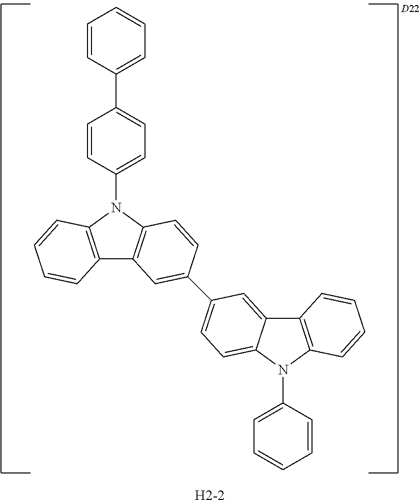 |
| | 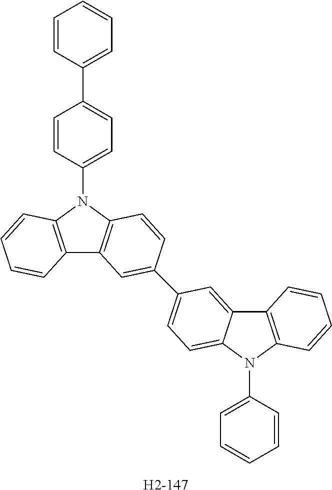 [SMILES: c1ccc(-c2ccc(-n3c4ccccc4c4cc(-c5ccc6c(c5)c5ccccc5n6-c5ccccc5)ccc43)cc2)cc1 ; "H2-147"] |
| | 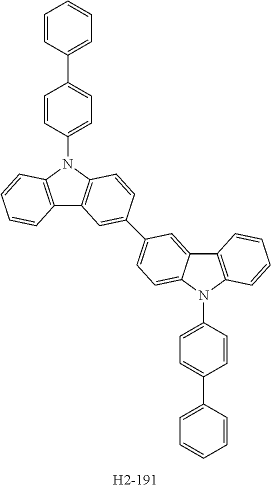 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; "H2-191"] |
| | 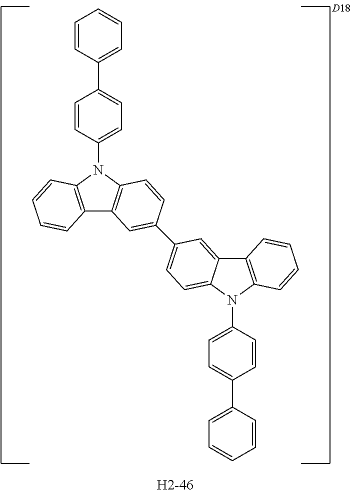 |
| | 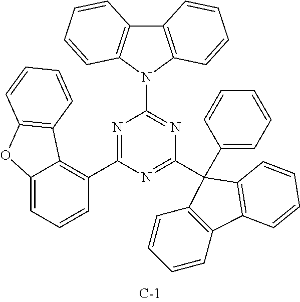 [SMILES: c1ccc(C2(c3nc(-c4cccc5oc6ccccc6c45)nc(-n4c5ccccc5c5ccccc54)n3)c3ccccc3-c3ccccc32)cc1] |
| | 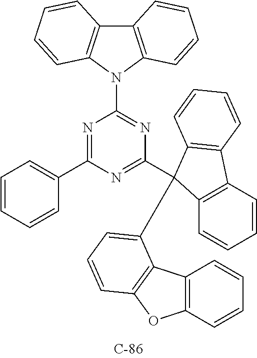 [SMILES: c1ccc(-c2nc(-n3c4ccccc4c4ccccc43)nc(C3(c4cccc5oc6ccccc6c45)c4ccccc4-c4ccccc43)n2)cc1] |
| | 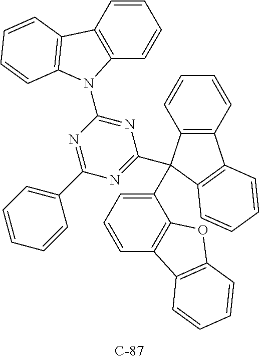 [SMILES: c1ccc(-c2nc(-n3c4ccccc4c4ccccc43)nc(C3(c4cccc5c4oc4ccccc45)c4ccccc4-c4ccccc43)n2)cc1] |
| | 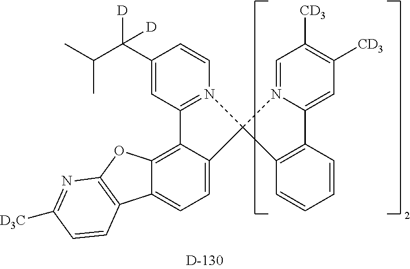 |
| | 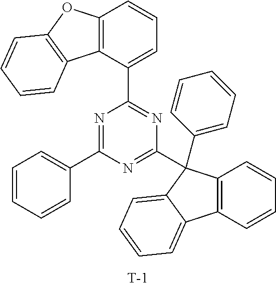 [SMILES: c1ccc(-c2nc(-c3cccc4oc5ccccc5c34)nc(C3(c4ccccc4)c4ccccc4-c4ccccc43)n2)cc1] |
| | 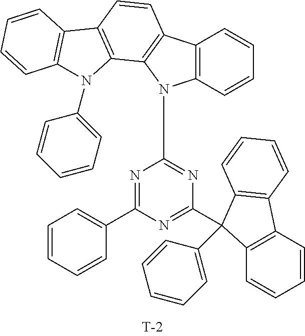 [SMILES: c1ccc(-c2nc(-n3c4ccccc4c4ccc5c6ccccc6n(-c6ccccc6)c5c43)nc(C3(c4ccccc4)c4ccccc4-c4ccccc43)n2)cc1] |
| Electron Transport Layer/ Electron Injection Layer | 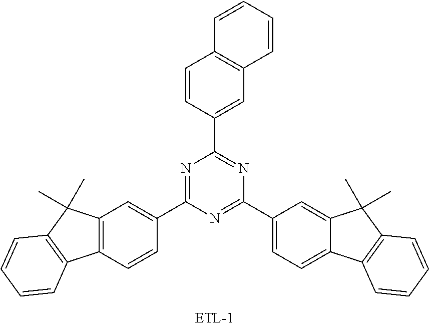 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 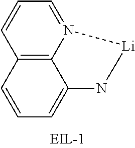 |

1. A plurality of host materials comprising at least one first host compound and at least one second host compound, wherein the first host compound is represented by the following Formula 1, and the second host compound is represented by the following Formula 2: 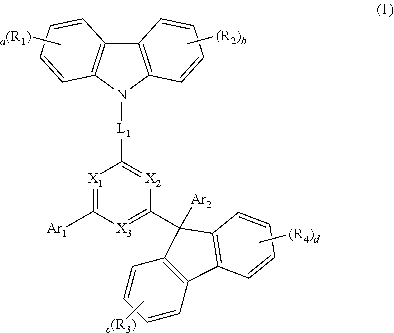
in Formula 1,
X₁ to X₃, each independently, represent N or CRₐ, with a proviso that at least two of X₁ to X₃ represent N;
L₁ represents a single bond, or a substituted or unsubstituted (C6-C30)arylene;
Rₐ, and R₁ to R₄, each independently, represent hydrogen, deuterium, a halogen, a cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di- (C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
Ar₁ and Ar₂, each independently, represent hydrogen, deuterium, a substituted or unsubstituted (C6-C30)aryl, or a substituted or unsubstituted (3- to 30-membered)heteroaryl;
a to d, each independently, represent an integer of 1 to 4; and
if a to d represent an integer of 2 or more, each of R₁ to each of R₄ may be the same as or different from each other; 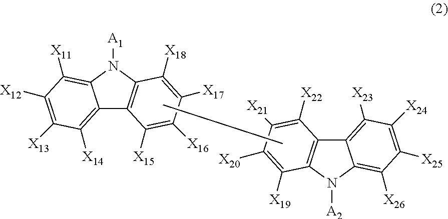
in Formula 2,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;
one of X₁₅ to X₁₈ links to one of X₁₉ to X₂₂ to form a single bond; and
X₁₁ to X₁₄, X₂₃ to X₂₆, and X₁₅ to X₂₂ not forming a single bond, each independently, represent hydrogen, deuterium, a substituted or unsubstituted (C6-C30)aryl, or a substituted or unsubstituted (3- to 30-membered)heteroaryl, or may be linked to an adjacent substituent(s) to form a ring(s).

2. The plurality of host materials according to claim 1, wherein Rₐ, and R₁ to R₄ in Formula 1, each independently, represent hydrogen, deuterium, a phenyl unsubstituted or substituted with deuterium, a biphenyl unsubstituted or substituted with deuterium, a phenanthrenyl unsubstituted or substituted with deuterium, a terphenyl unsubstituted or substituted with deuterium, a quaterphenyl unsubstituted or substituted with deuterium, a naphthyl unsubstituted or substituted with deuterium, a phenylnaphthyl unsubstituted or substituted with deuterium, a naphthylphenyl unsubstituted or substituted with deuterium, a dibenzofuranyl unsubstituted or substituted with deuterium, a carbazolyl unsubstituted or substituted with deuterium, a dibenzothiophenyl unsubstituted or substituted with deuterium, or a combination thereof.

3. The plurality of host materials according to claim 1, wherein Ar₁ and Ar₂ in Formula 1, each independently, represent a phenyl unsubstituted or substituted with deuterium, a biphenyl unsubstituted or substituted with deuterium, a terphenyl unsubstituted or substituted with deuterium, a naphthyl unsubstituted or substituted with deuterium, a dibenzofuranyl unsubstituted or substituted with deuterium, a dibenzothiophenyl unsubstituted or substituted with deuterium, a carbazolyl unsubstituted or substituted with deuterium, or a combination thereof.

4. The plurality of host materials according to claim 1, wherein at least one of X₁₁, X₁₈, X₁₉, and X₂₆ in Formula 2 represents deuterium.

5. The plurality of host materials according to claim 1, wherein the substitution rate of deuterium of X₁₁ to X₂₆ in Formula 2 is 25% to 100%.

6. The plurality of host materials according to claim 1, wherein the substitution rate of deuterium of the compound represented by Formula 2 is 40% to 100%.

7. The plurality of host materials according to claim 1, wherein Formula 2 is represented by any one of the following Formulas 2-1 to 2-8: 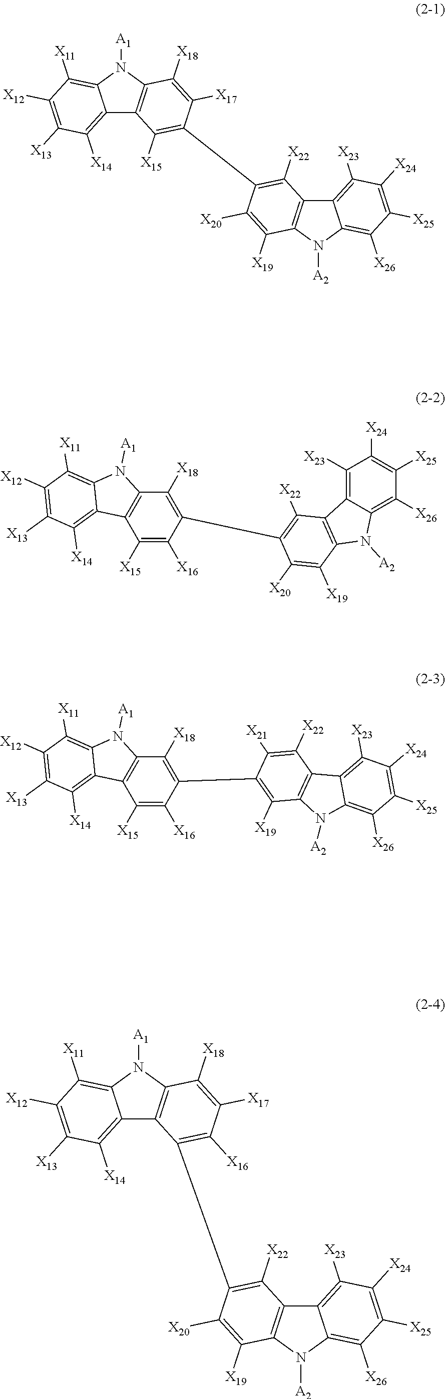 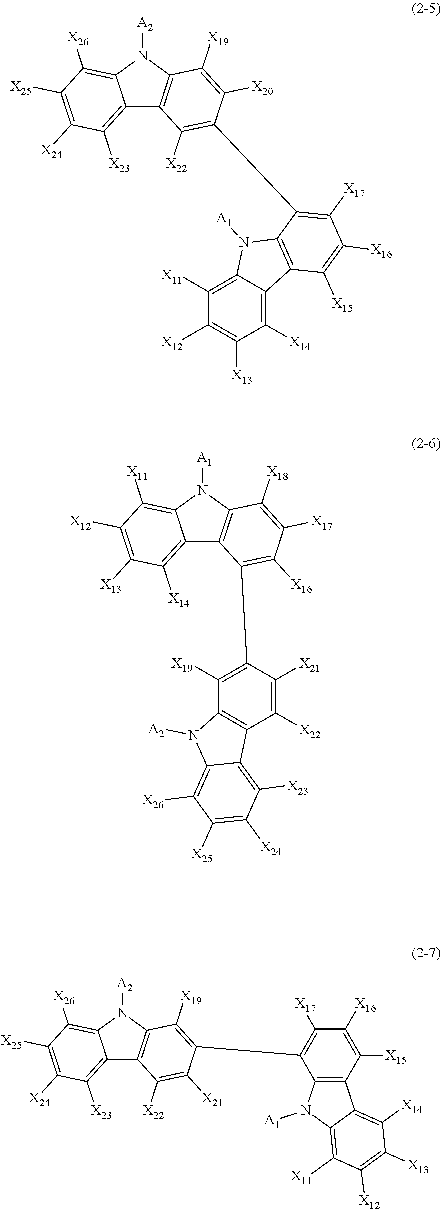 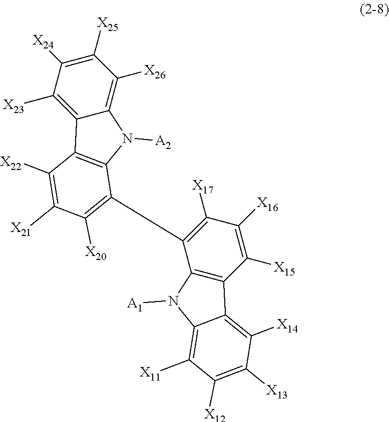
in Formulas 2-1 to 2-8, A₁, A₂, and X₁₁ to X₂₆ are as defined in claim 1.

8. The plurality of host materials according to claim 1, wherein A₁ and A₂ in Formula 2, each independently, represent a phenyl unsubstituted or substituted with deuterium; a biphenyl unsubstituted or substituted with deuterium; a terphenyl unsubstituted or substituted with deuterium; a naphthyl unsubstituted or substituted with deuterium; a fluorenyl unsubstituted or substituted with at least one of deuterium, a (C1-C30)alkyl(s), and a (C6-C30)aryl(s); a benzofluorenyl unsubstituted or substituted with at least one of deuterium, a (C1-C30)alkyl(s), and a (C6-C30)aryl(s); a triphenylenyl unsubstituted or substituted with deuterium; a fluoranthenyl unsubstituted or substituted with deuterium; a phenanthrenyl unsubstituted or substituted with deuterium; a dibenzofuranyl unsubstituted or substituted with deuterium; a dibenzothiophenyl unsubstituted or substituted with deuterium; a carbazolyl unsubstituted or substituted with deuterium; or a combination thereof.

9. The plurality of host materials according to claim 1, wherein the compound represented by Formula 1 is selected from the following compounds: 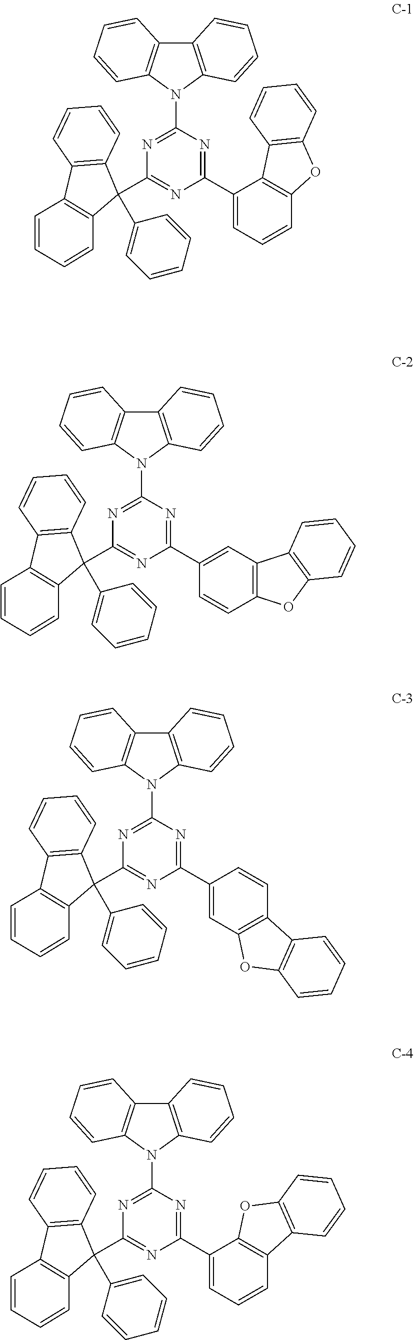 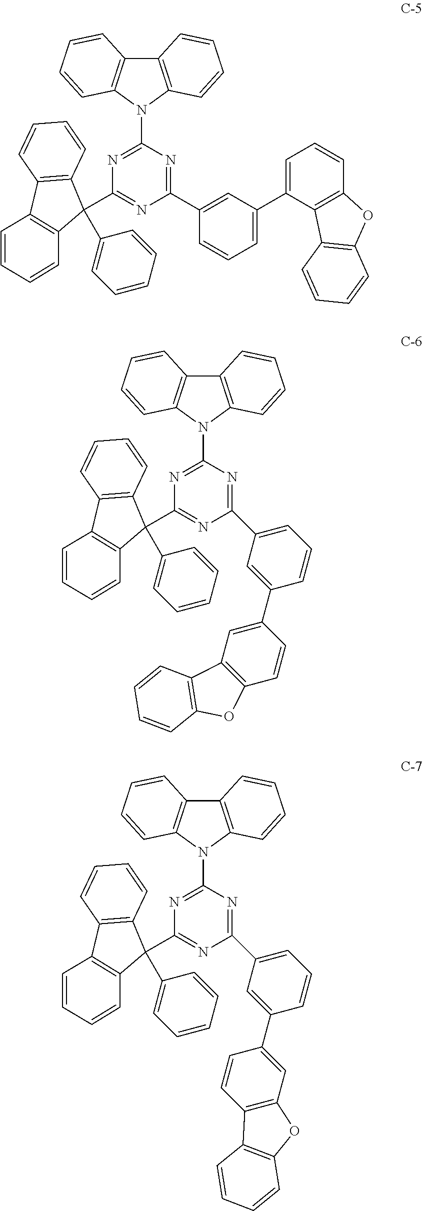 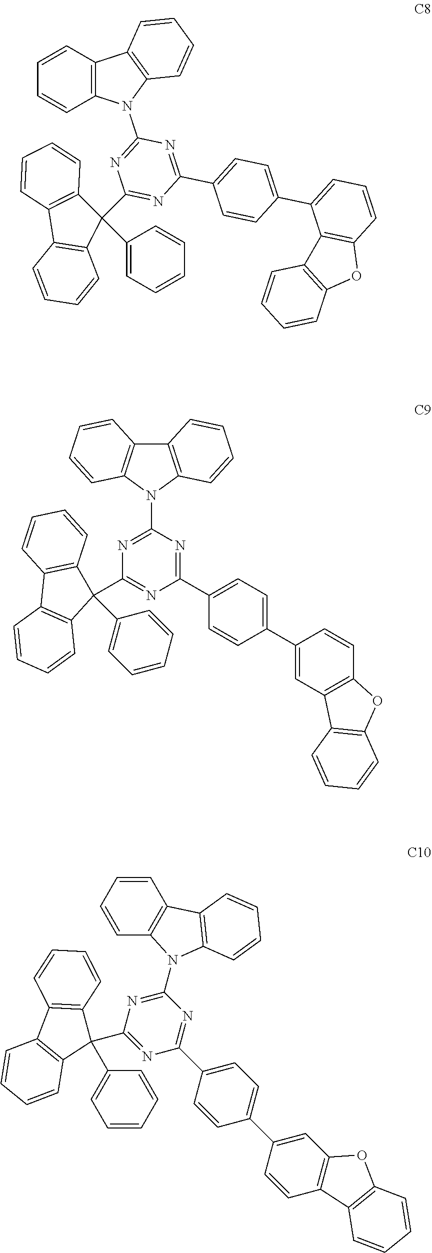 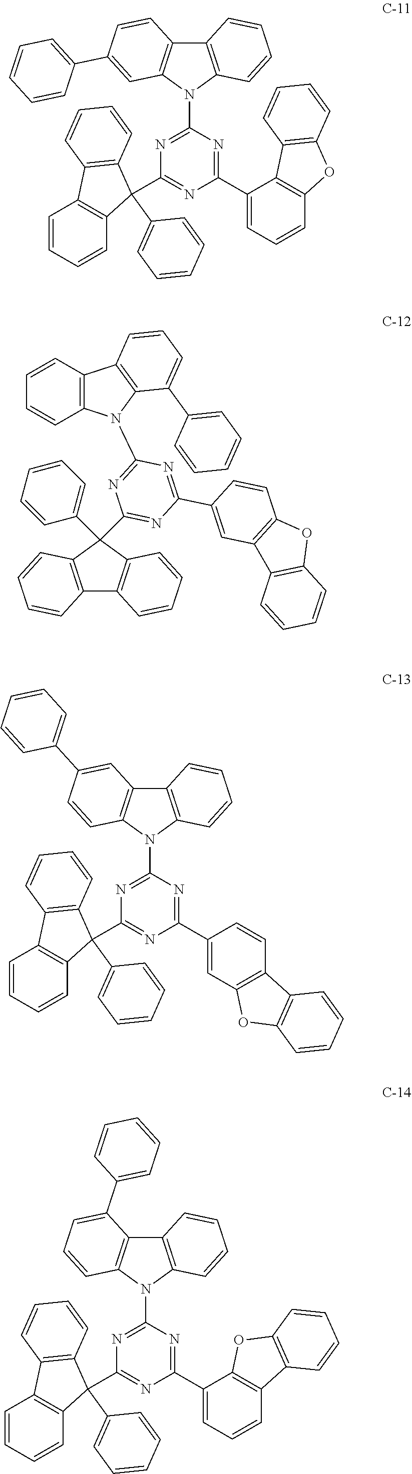 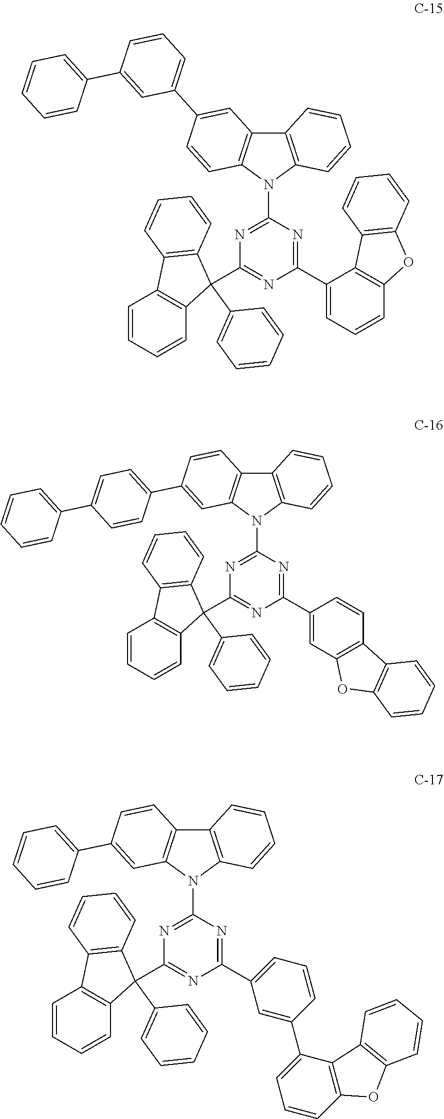 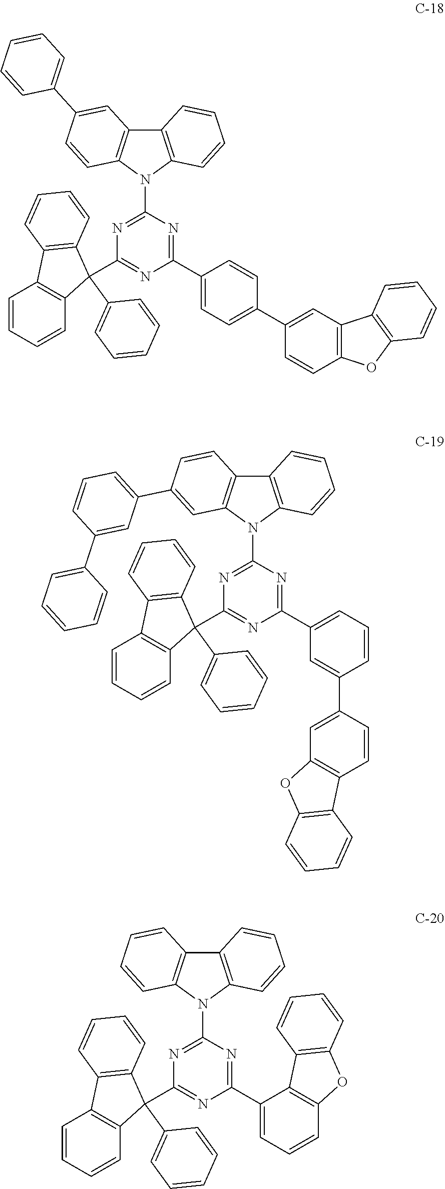 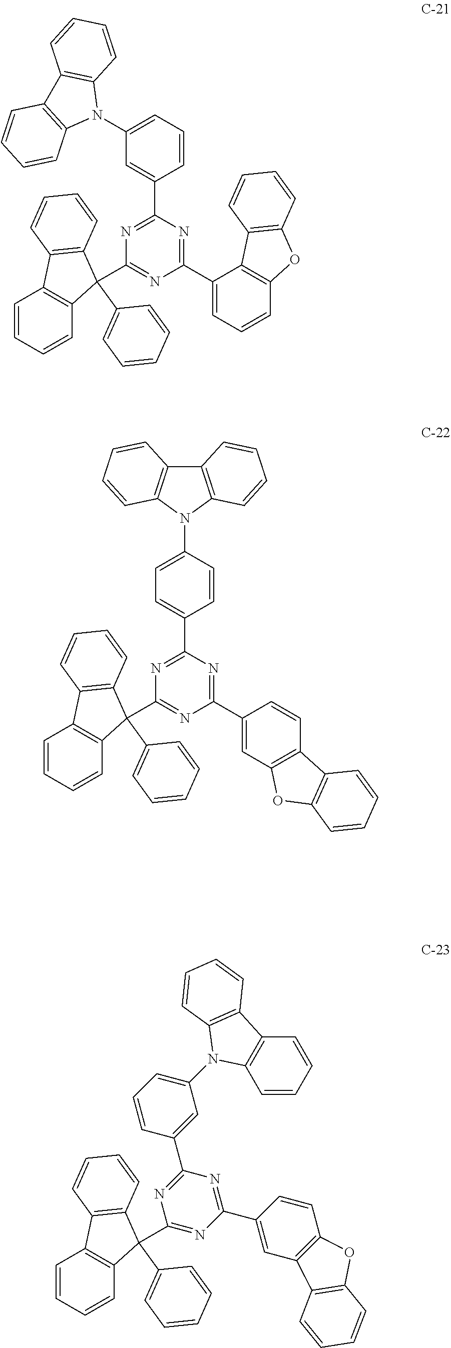 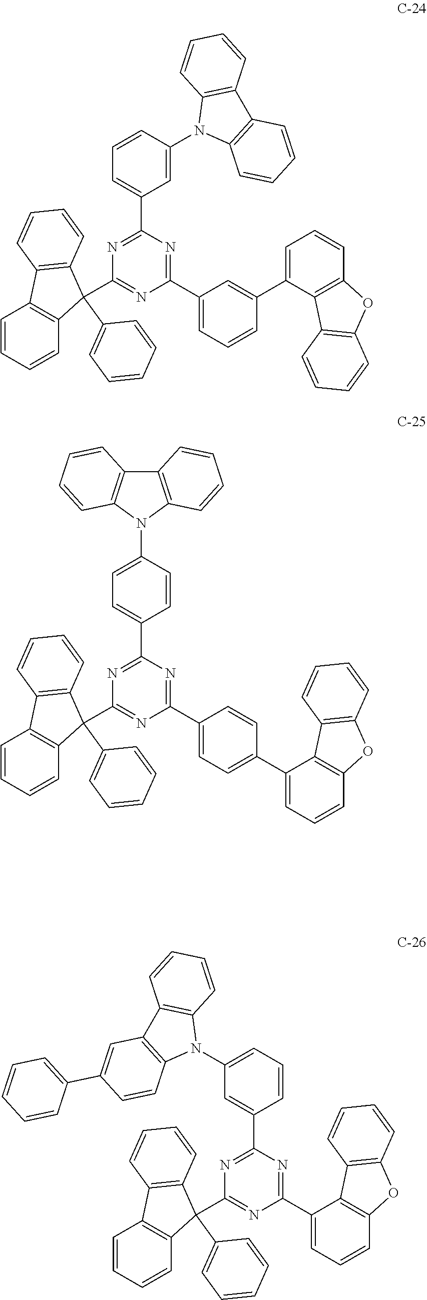 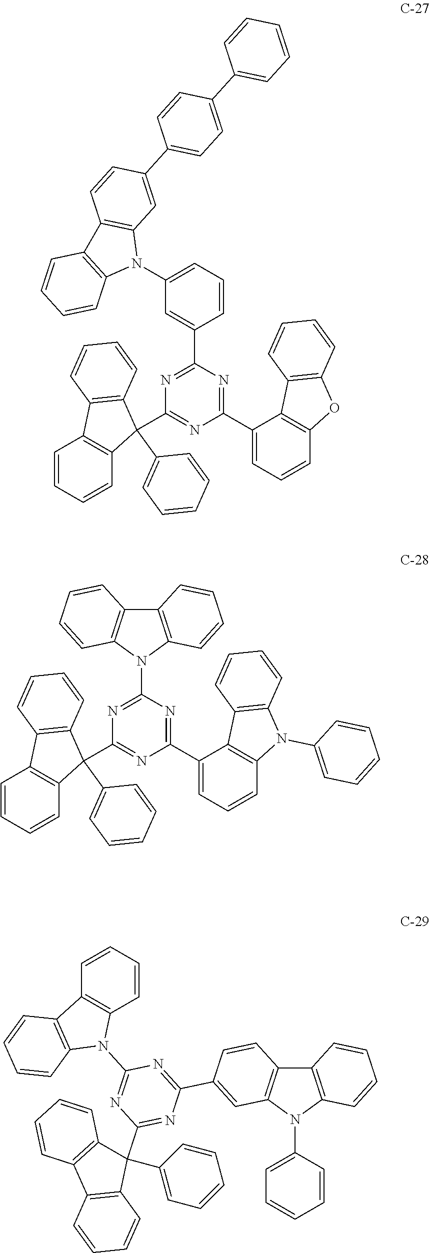 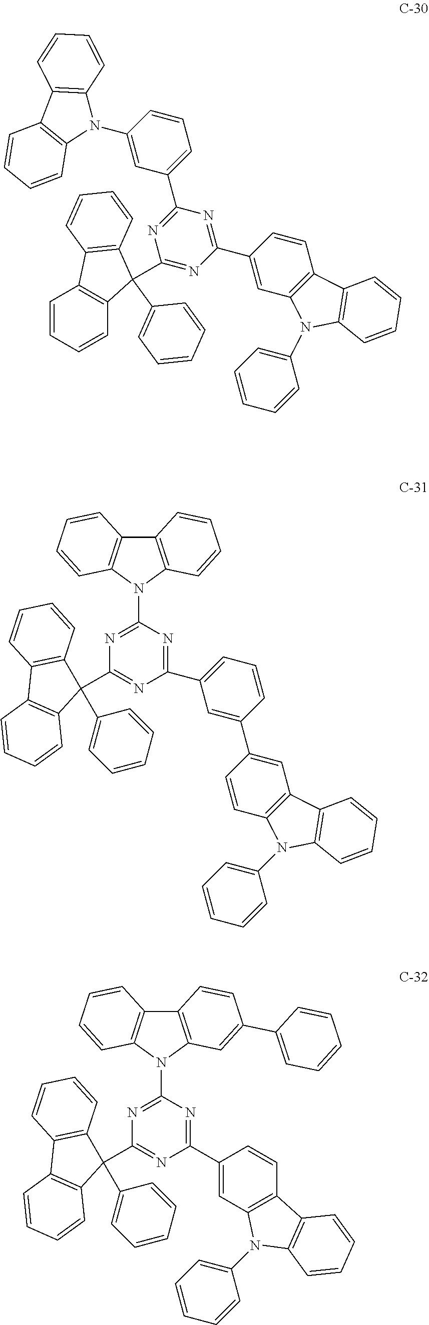 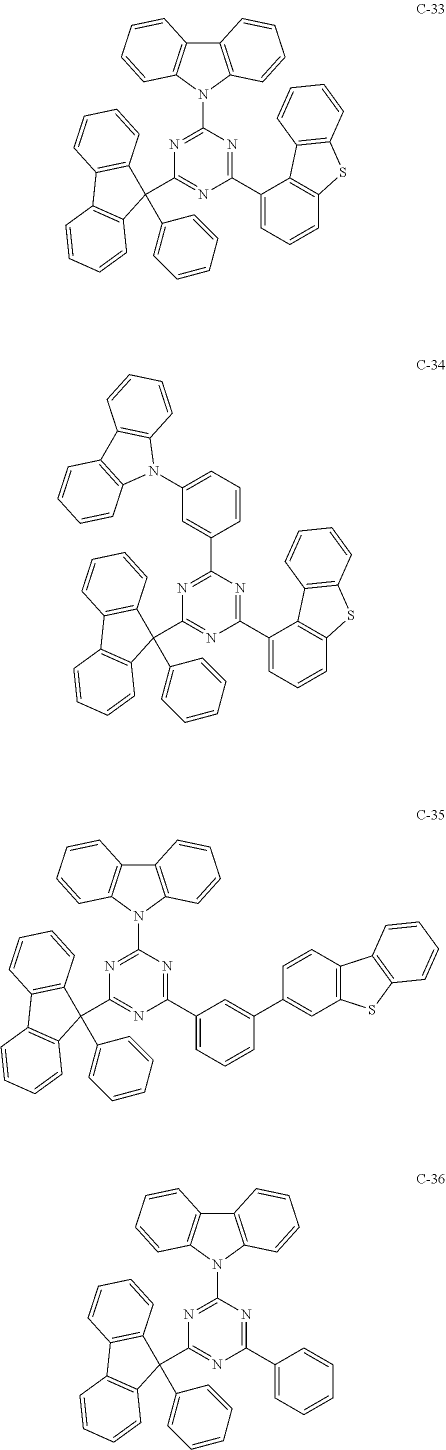 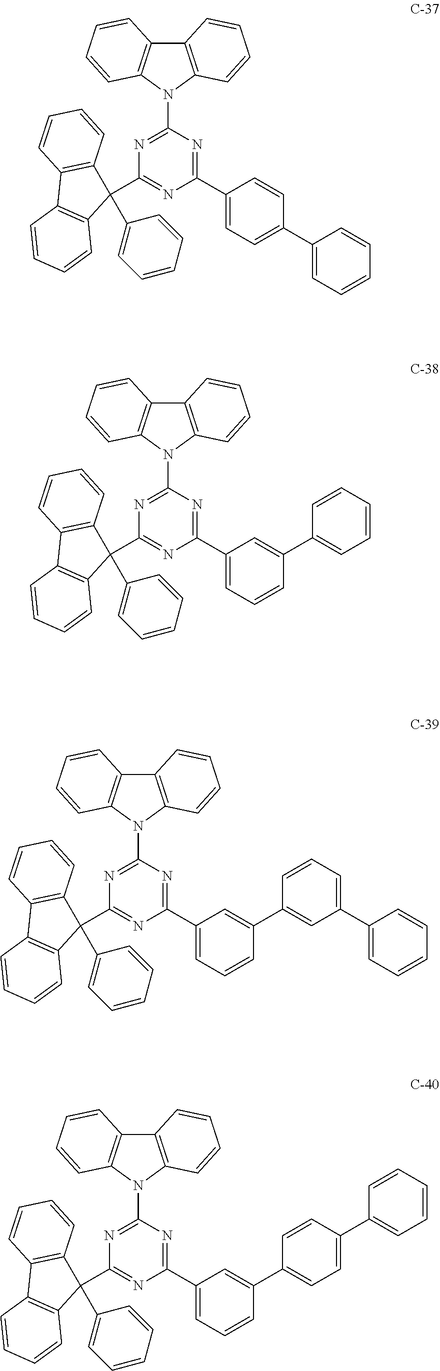 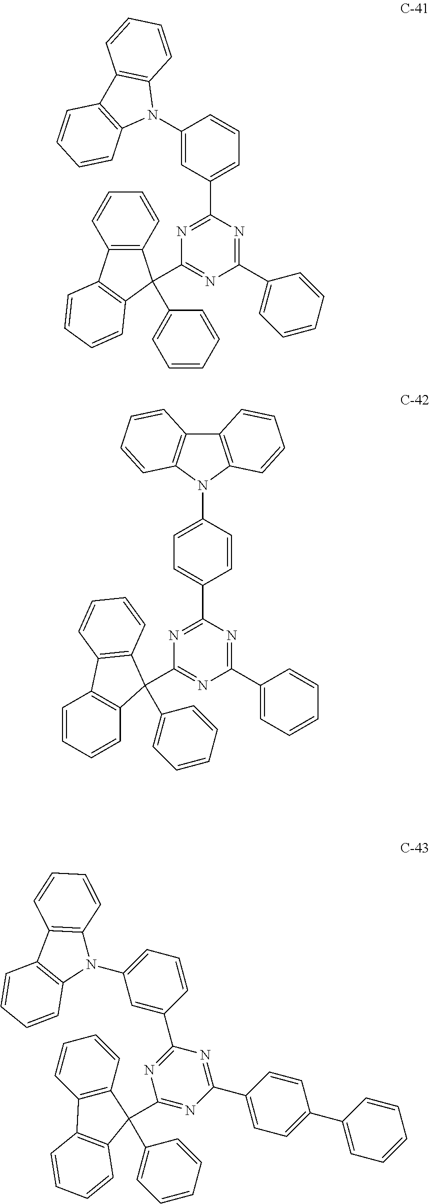 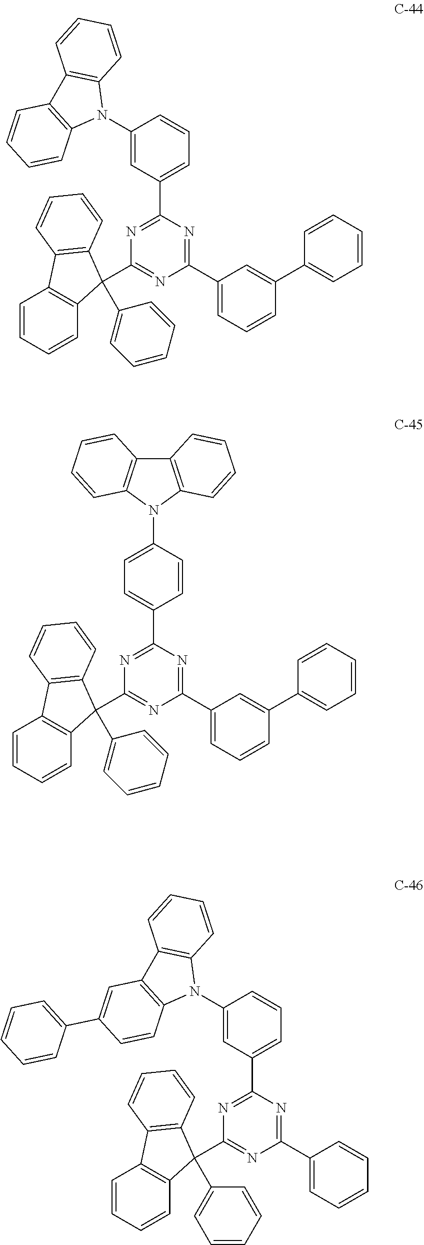 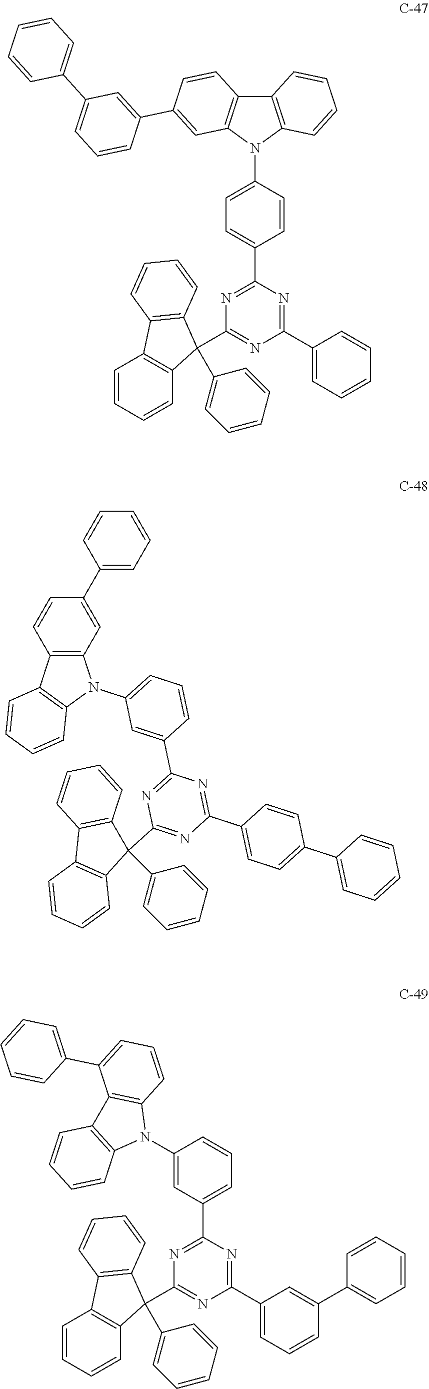 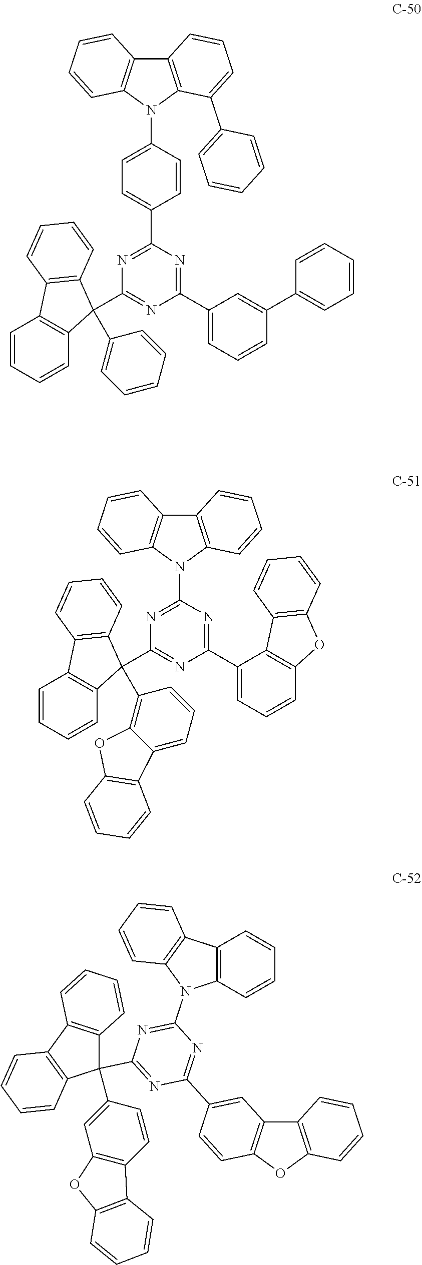 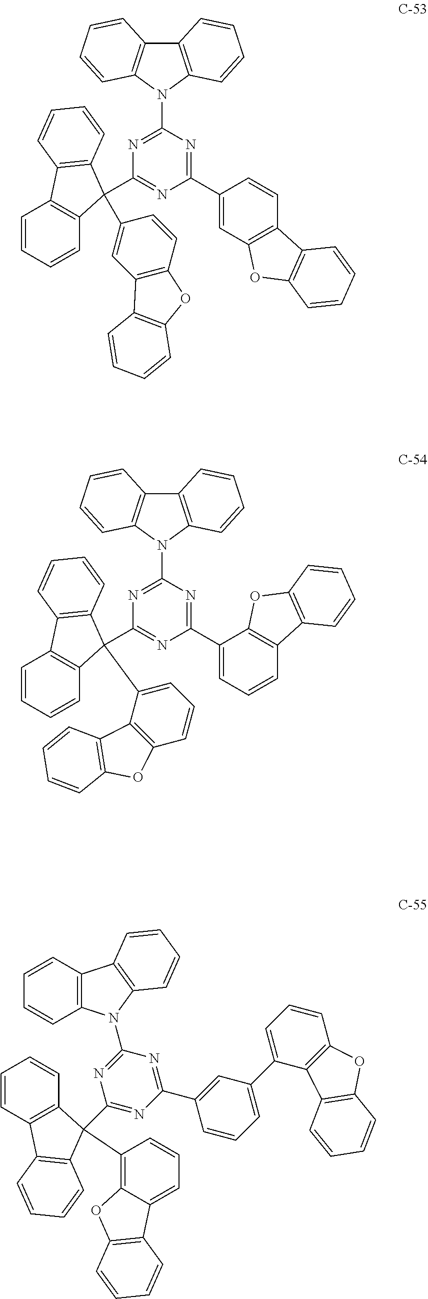 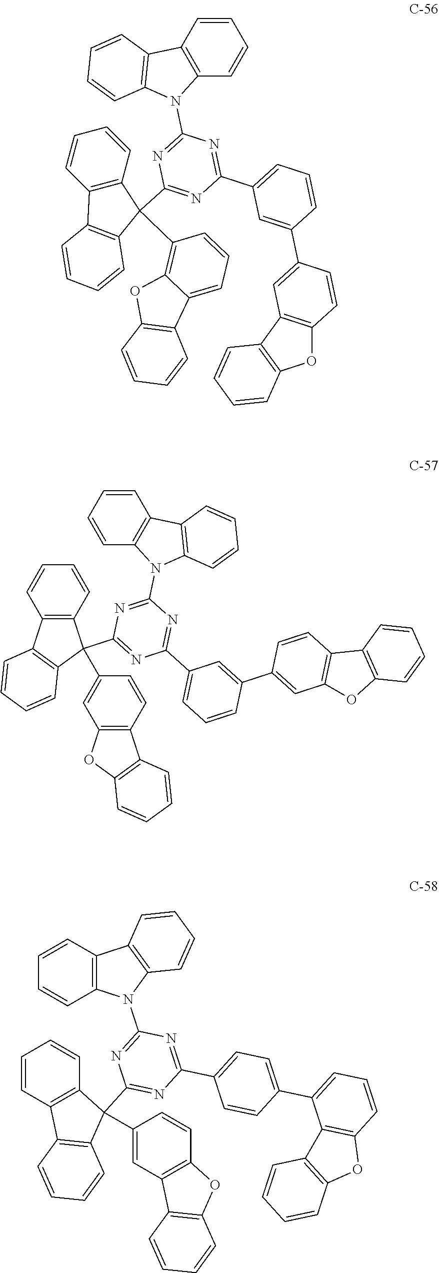 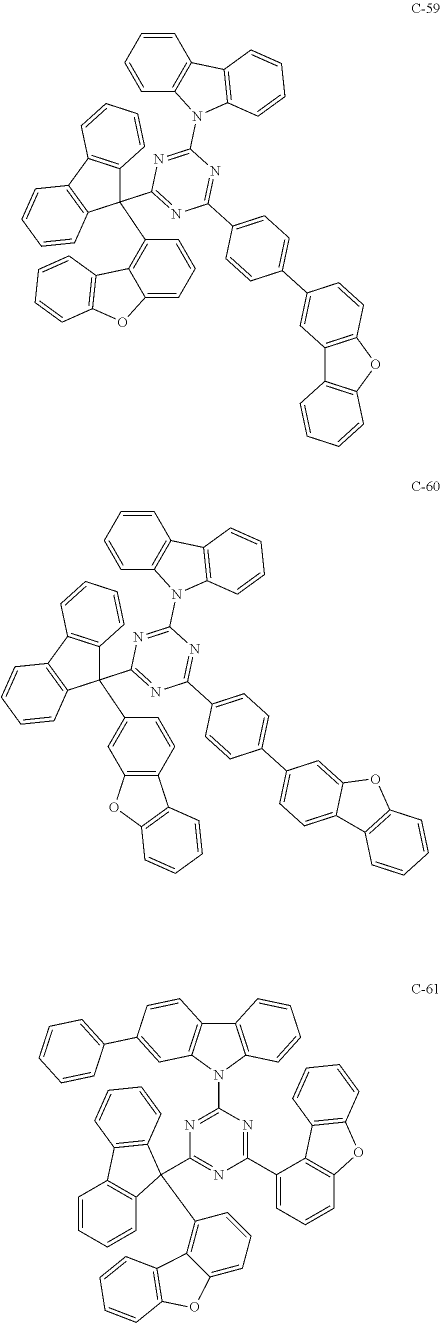 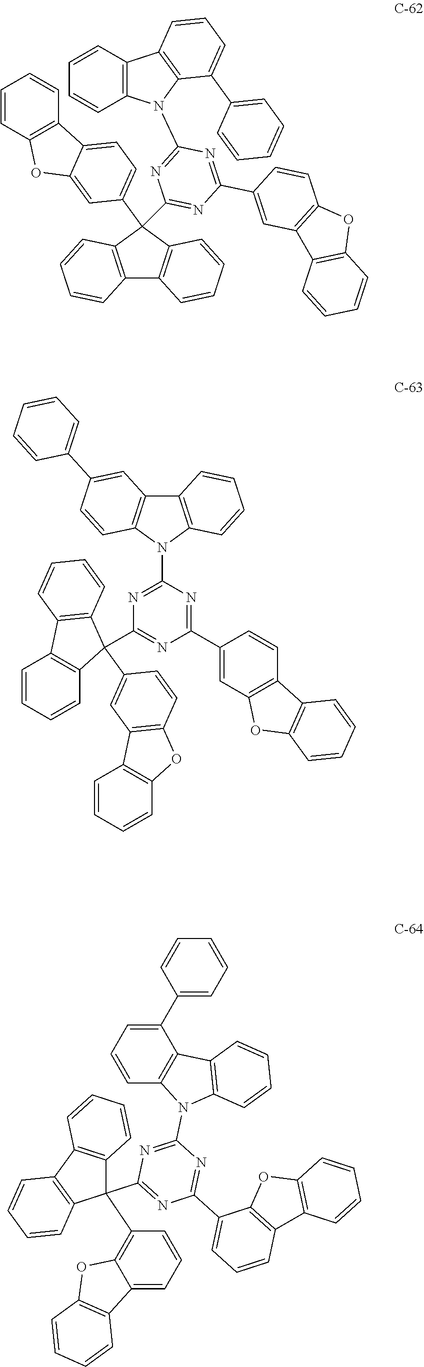 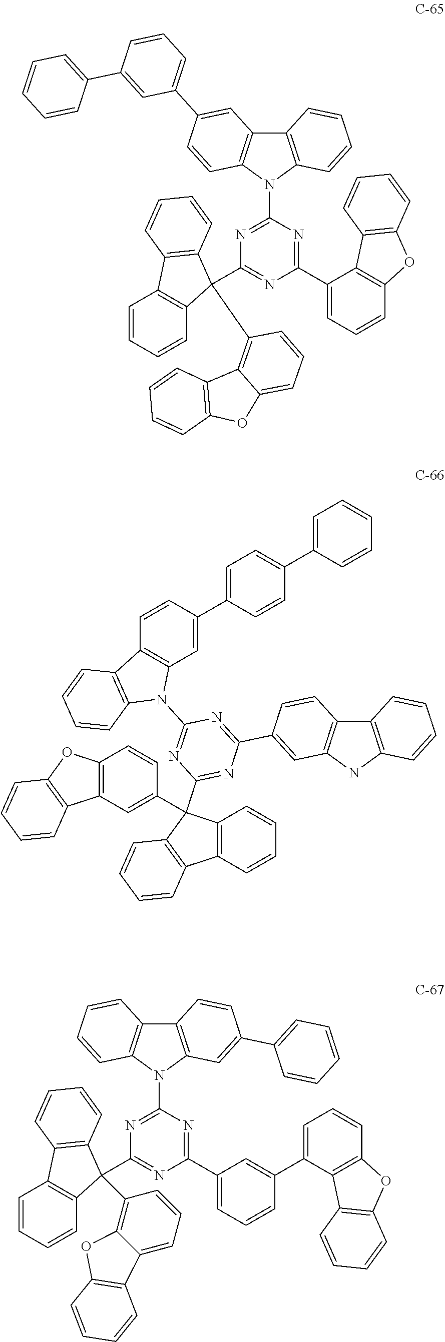 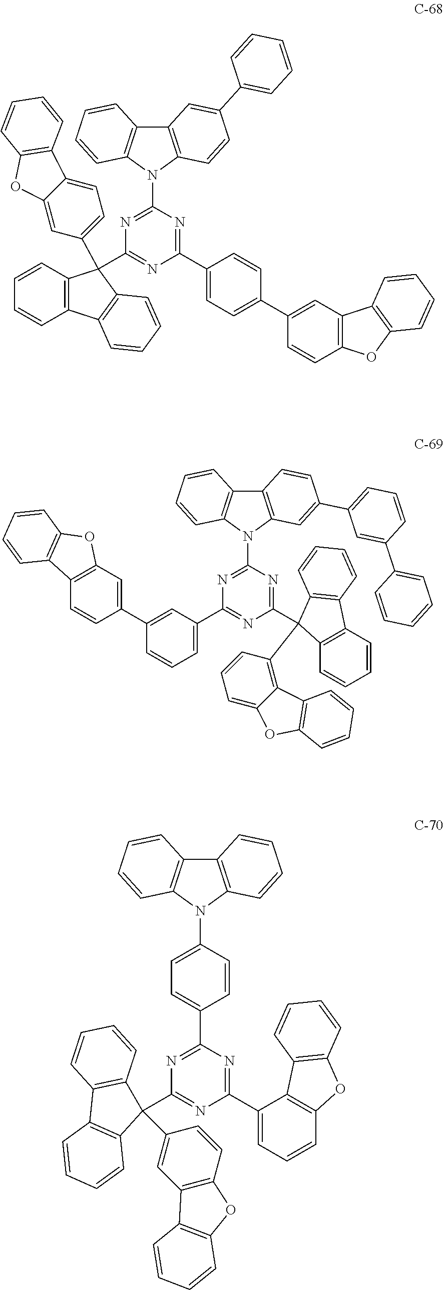 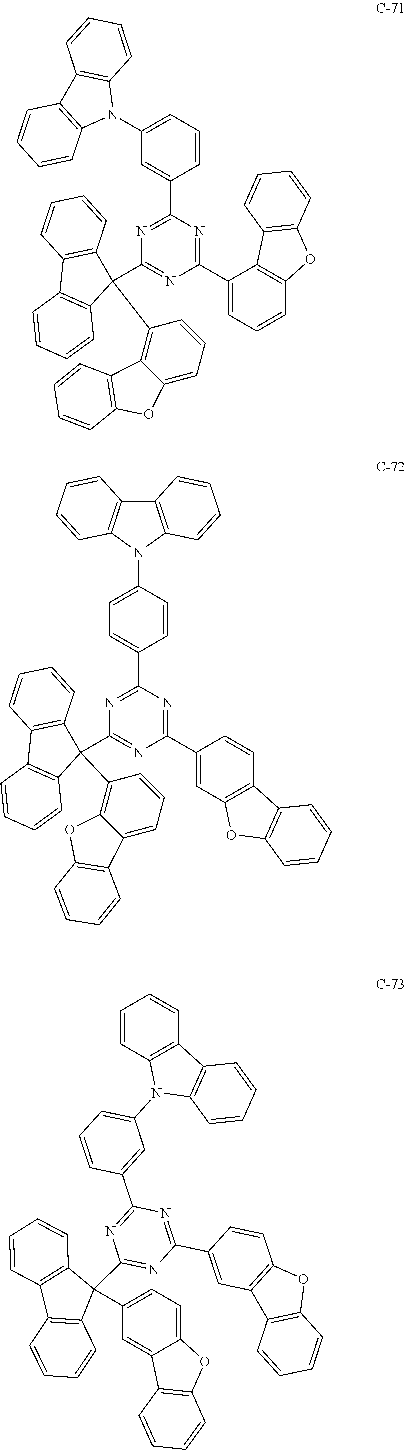 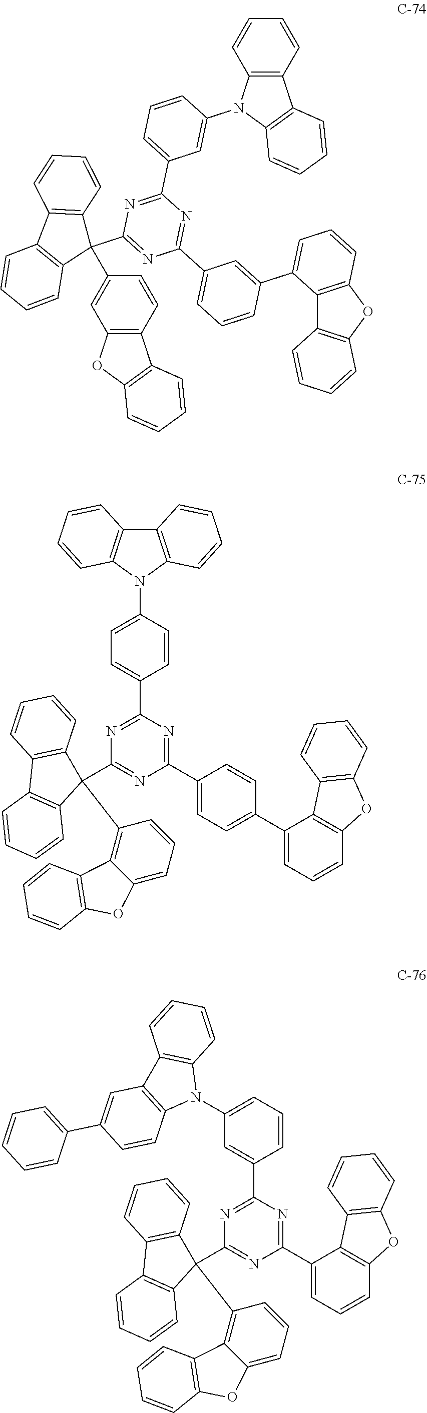 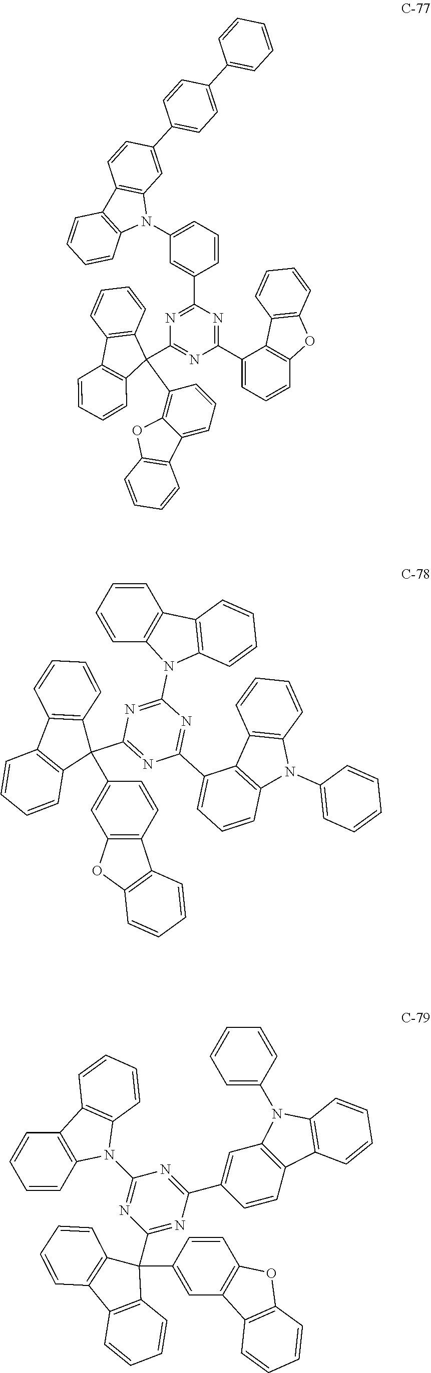 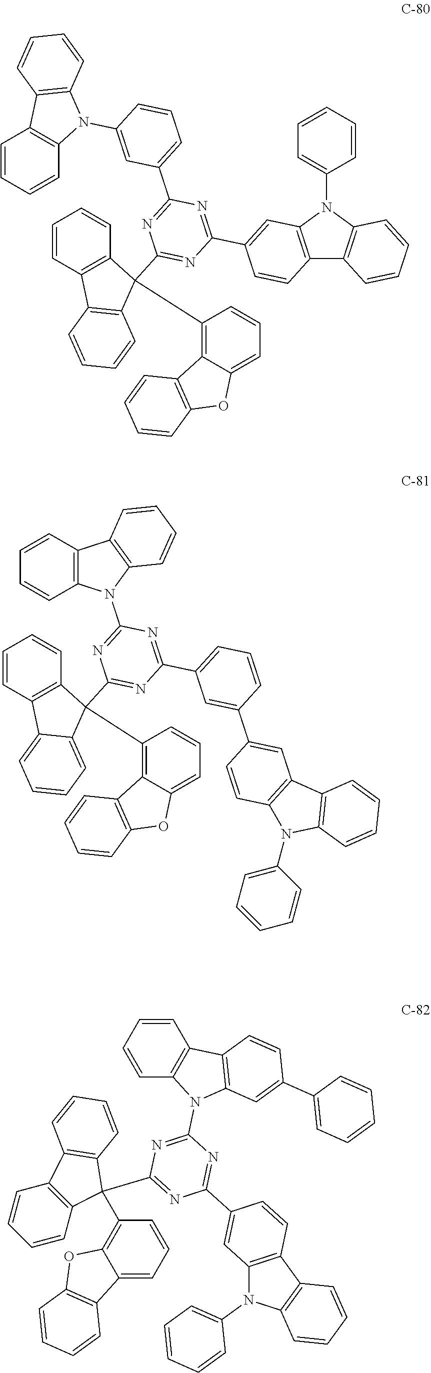 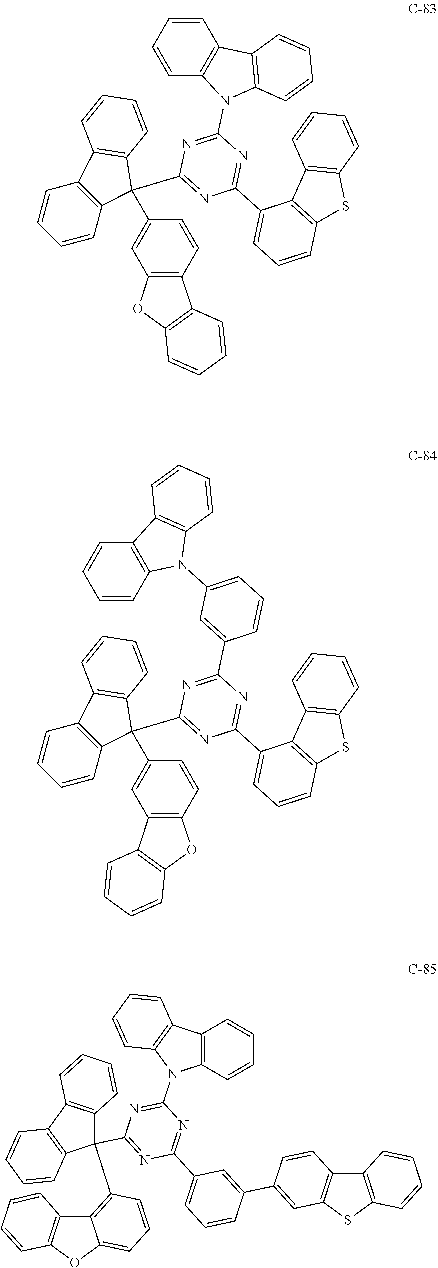 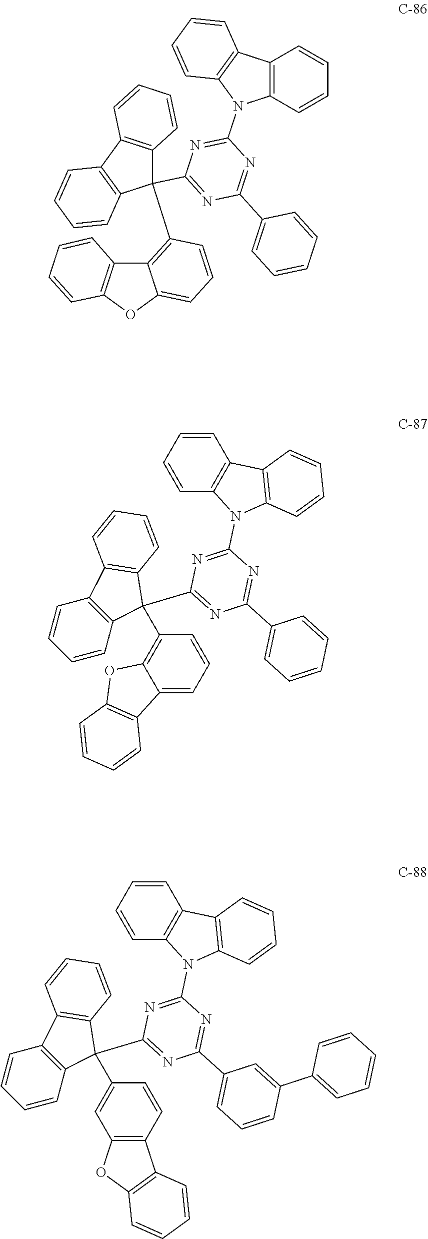 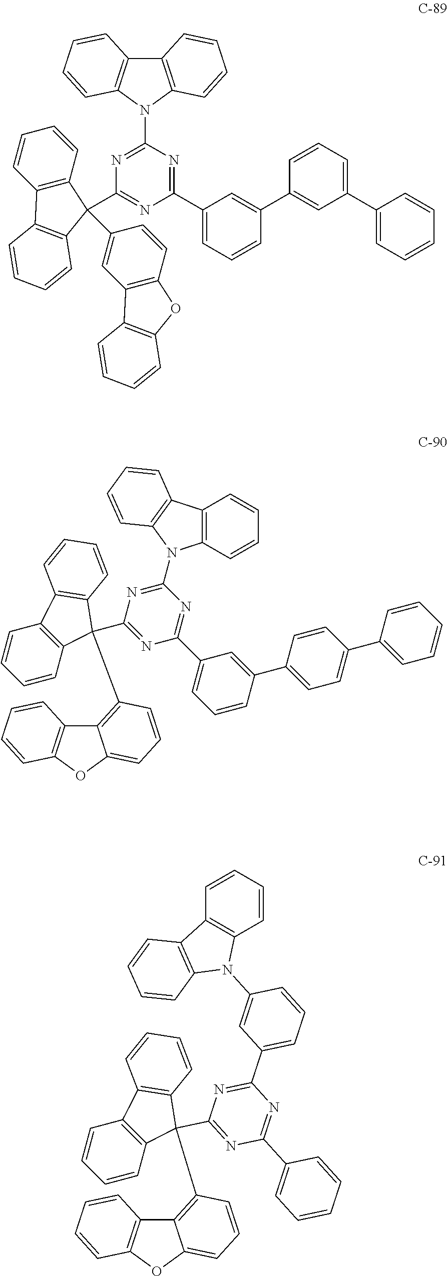 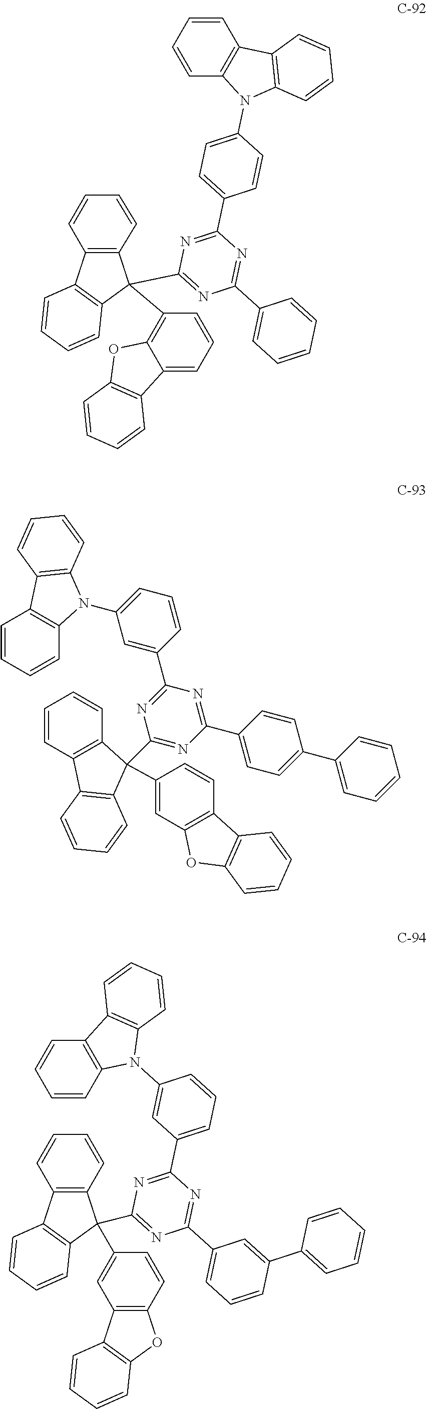 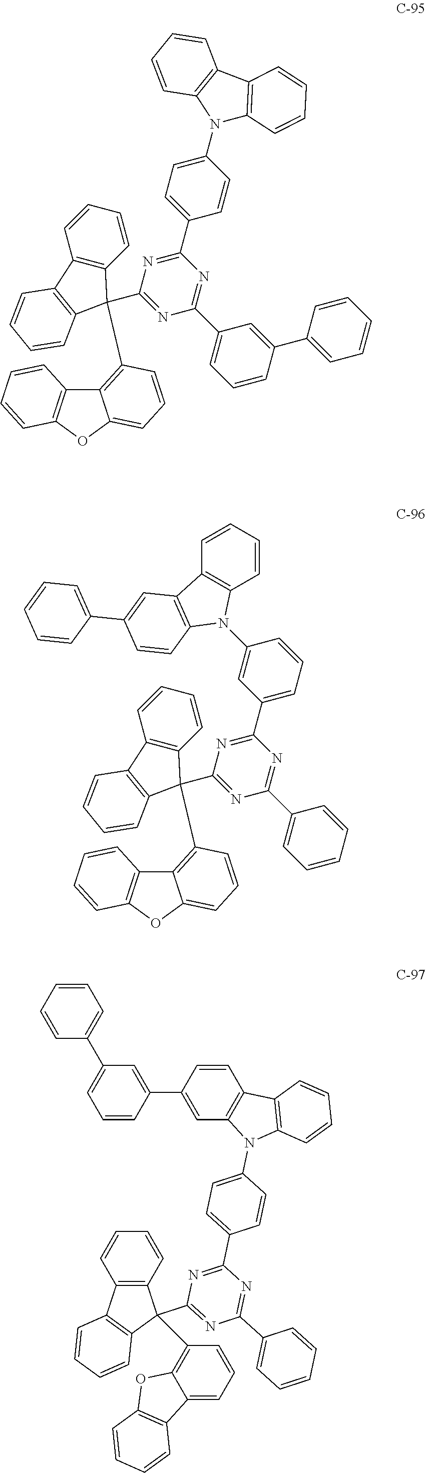 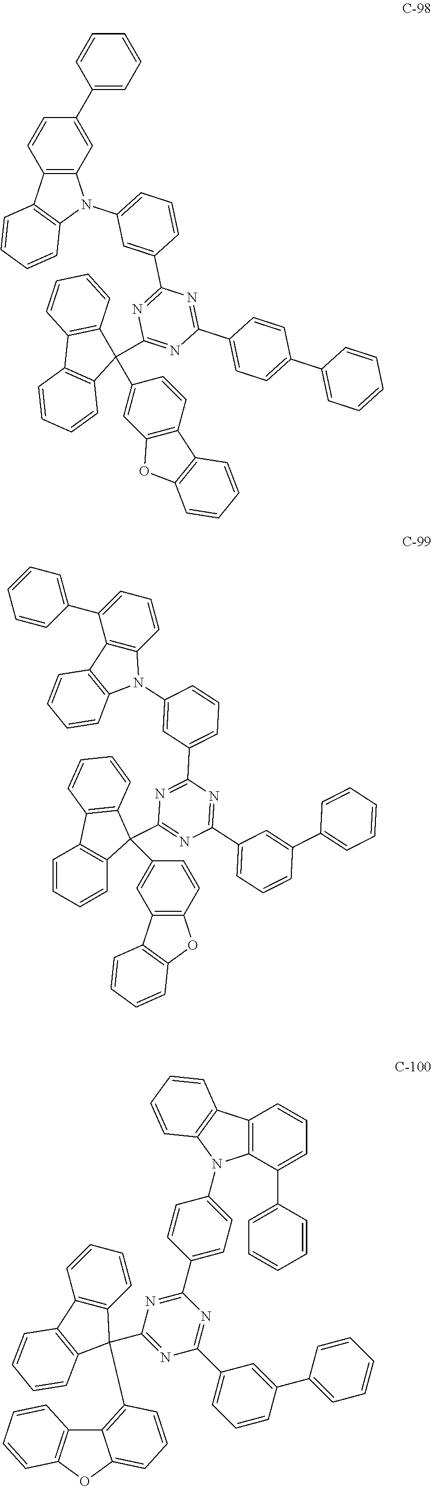 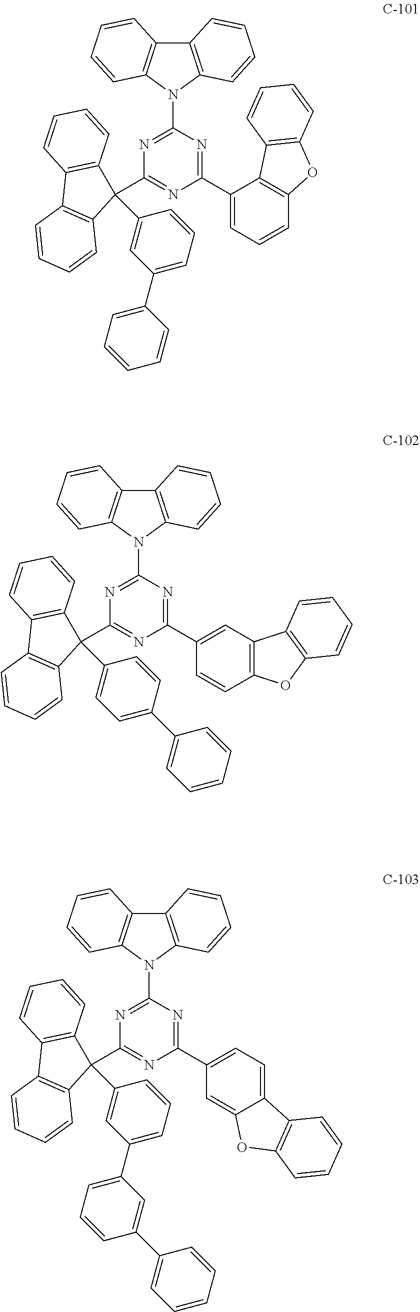 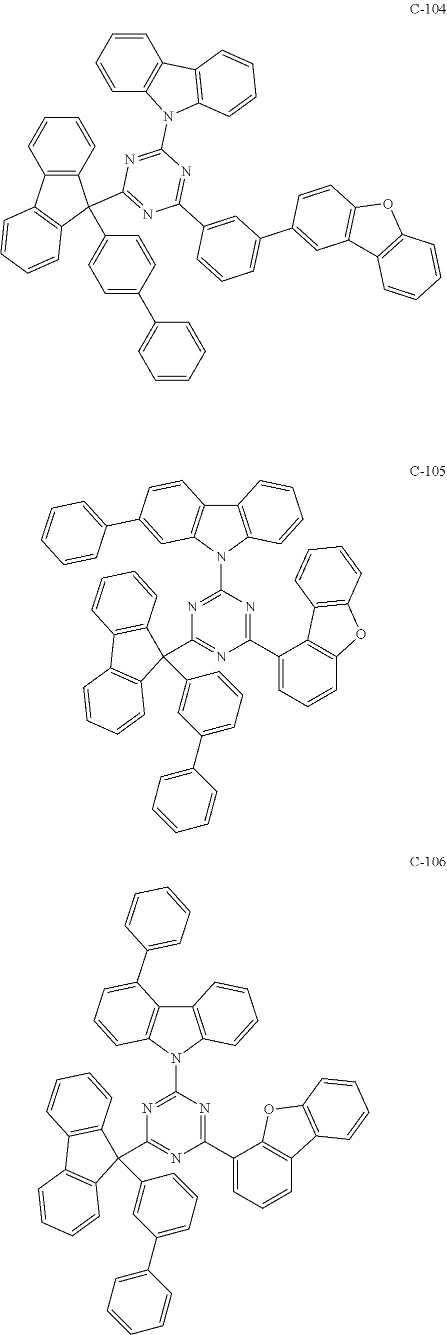 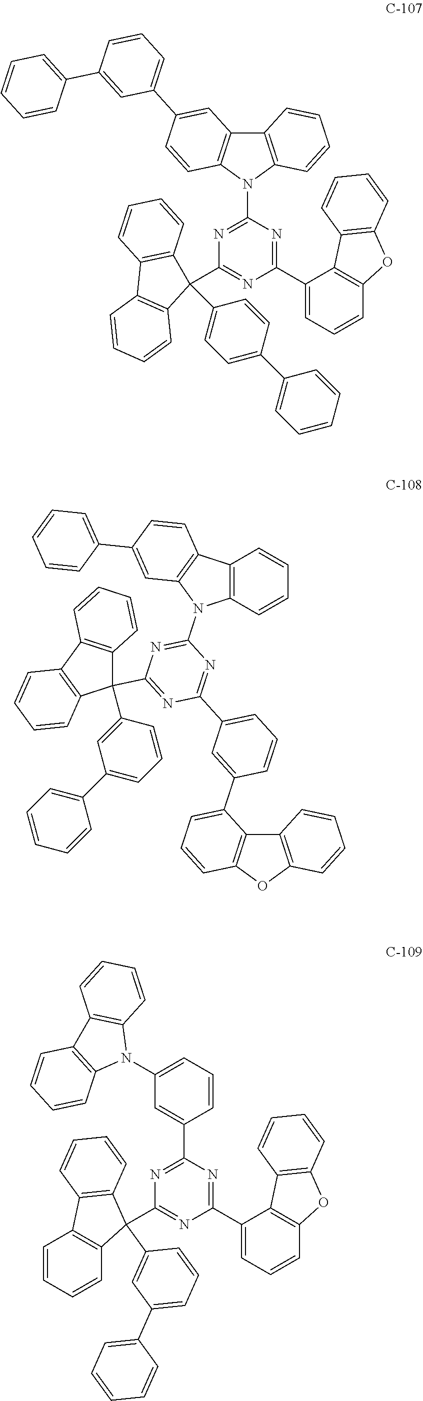 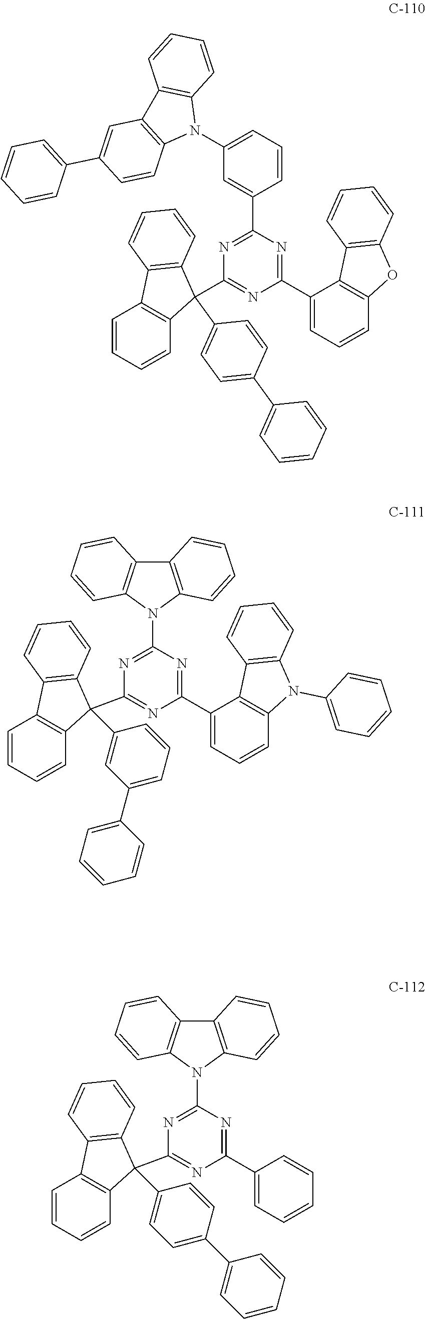 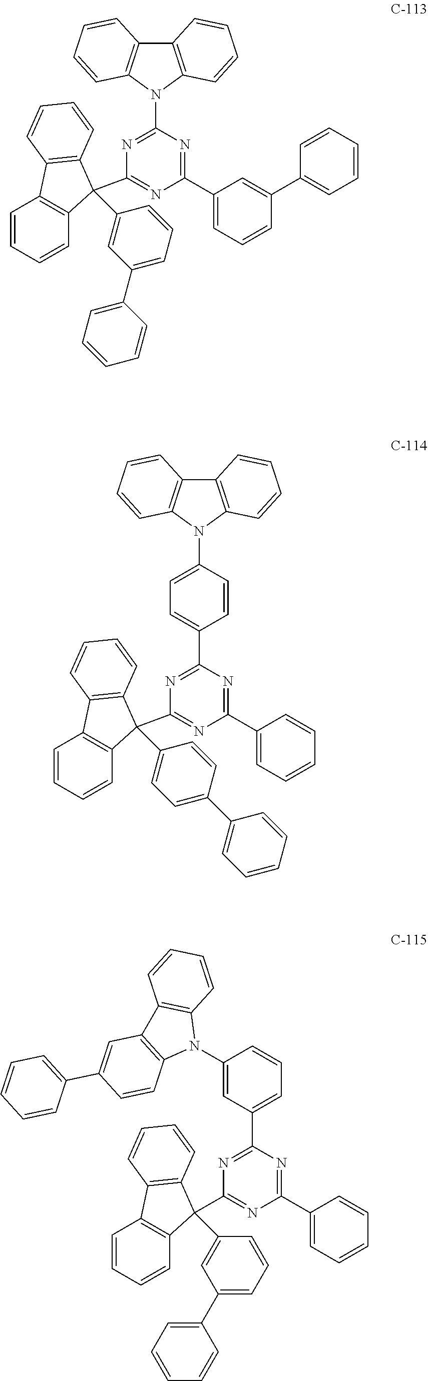 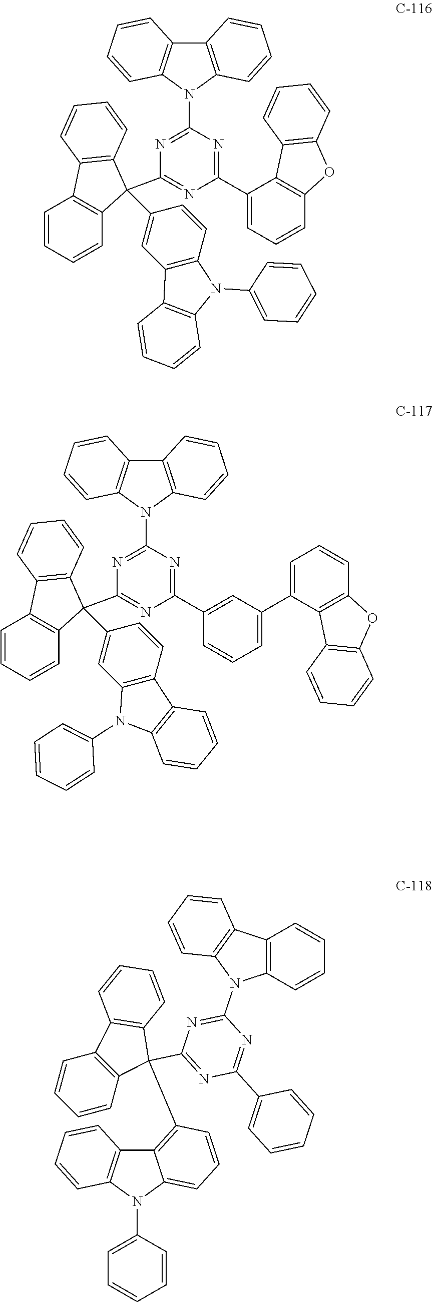 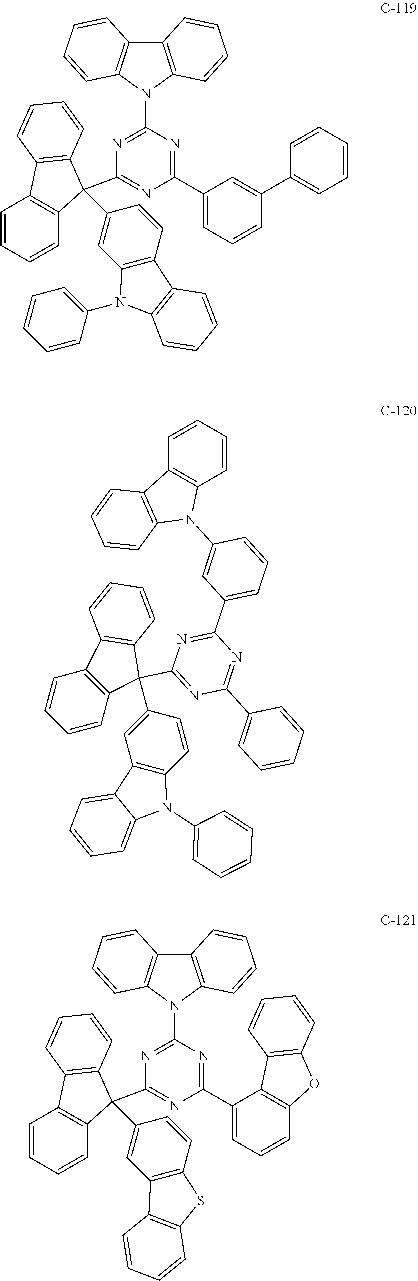 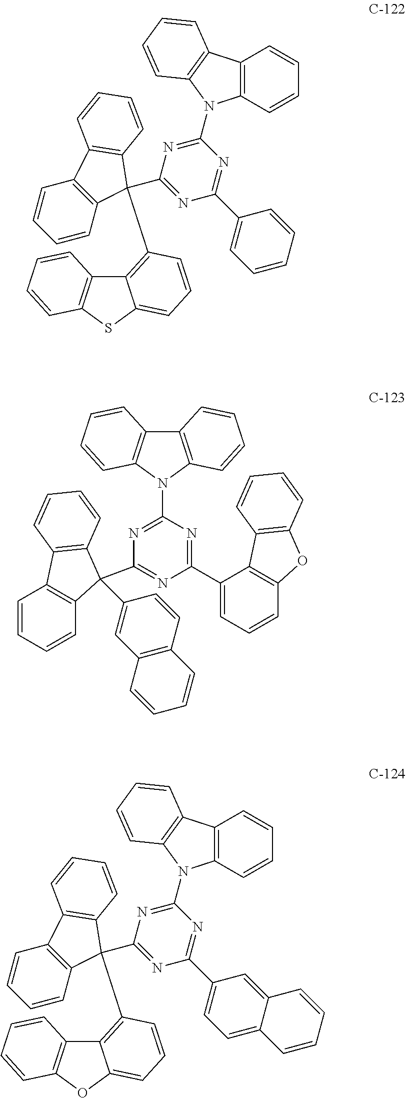 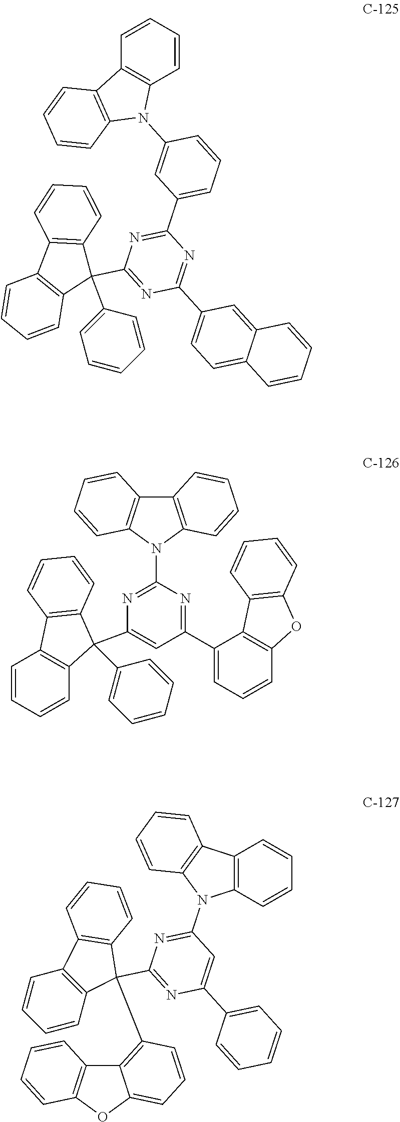 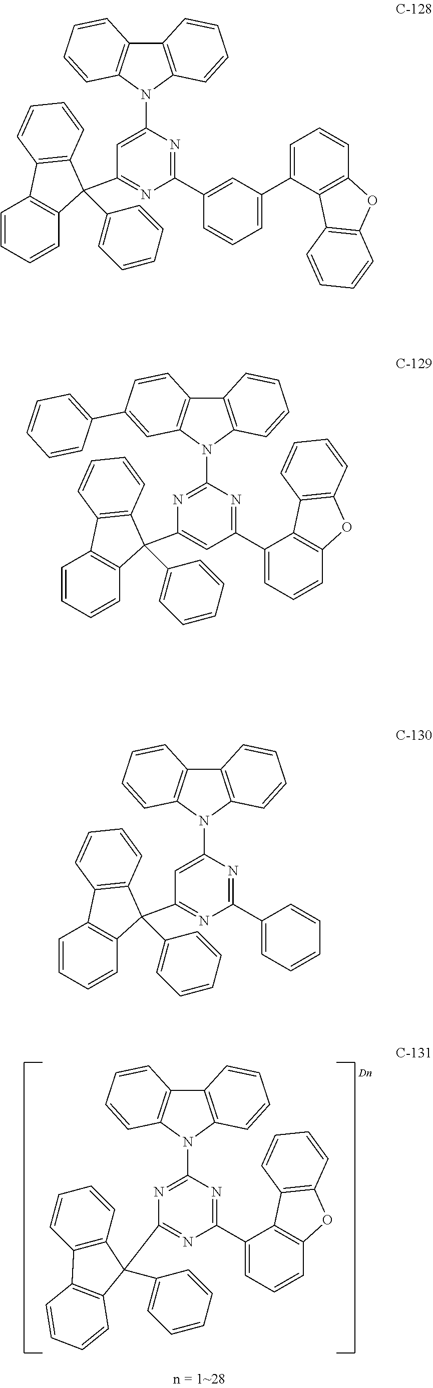 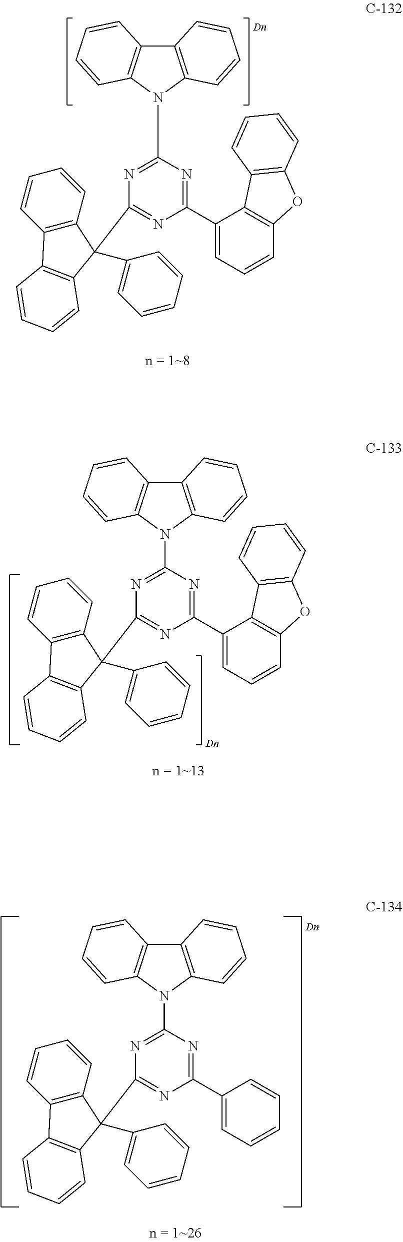 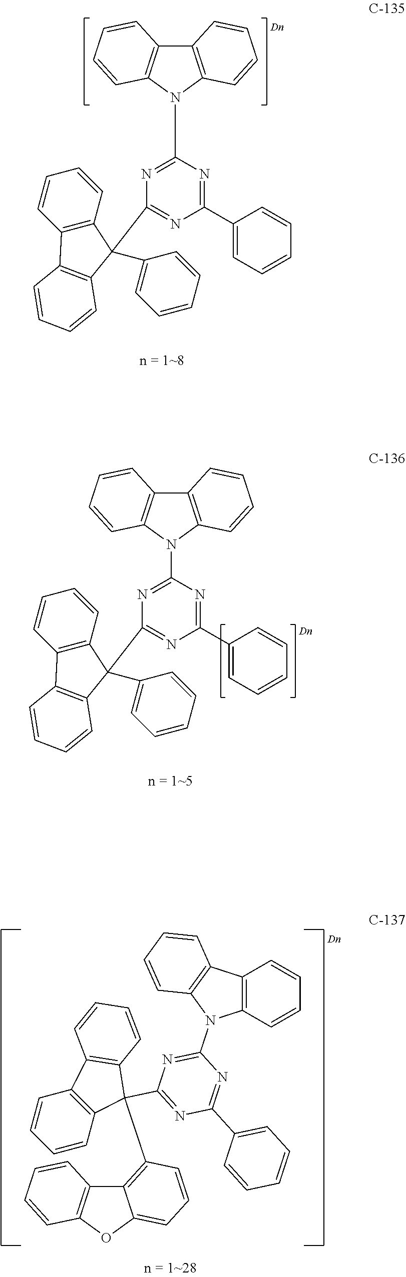 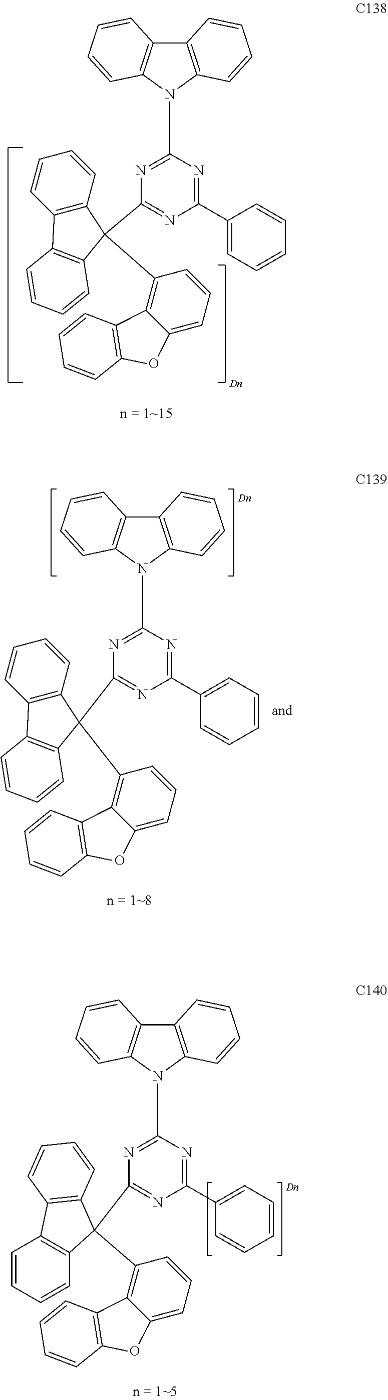
wherein, Dn signifies that n hydrogens are replaced with deuterium.

10. The plurality of host materials according to claim 1, wherein the compound represented by Formula 2 is selected from the following compounds: 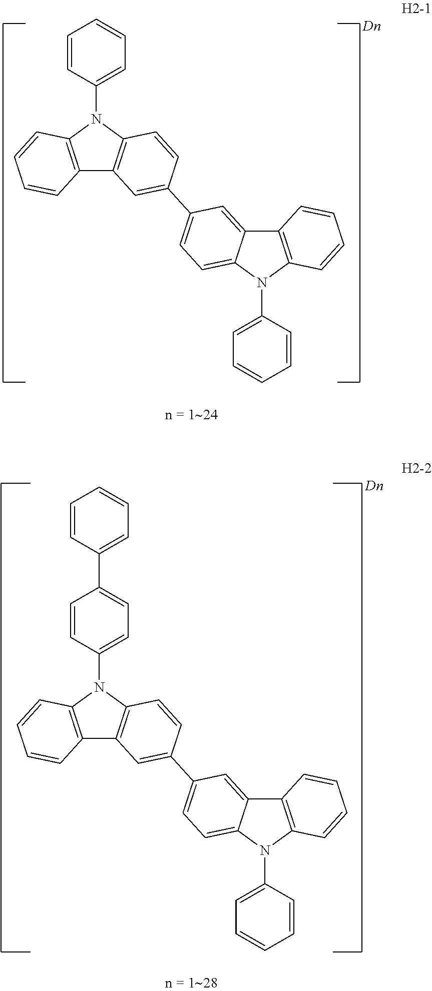 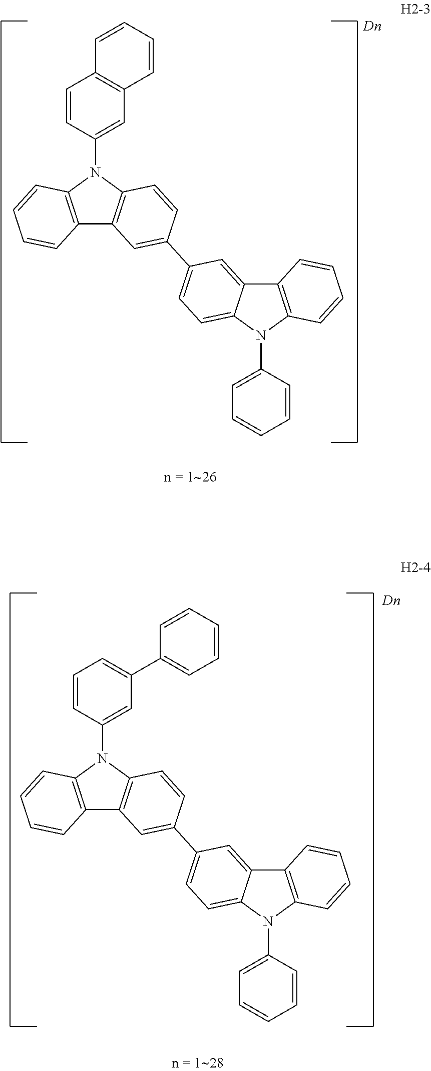 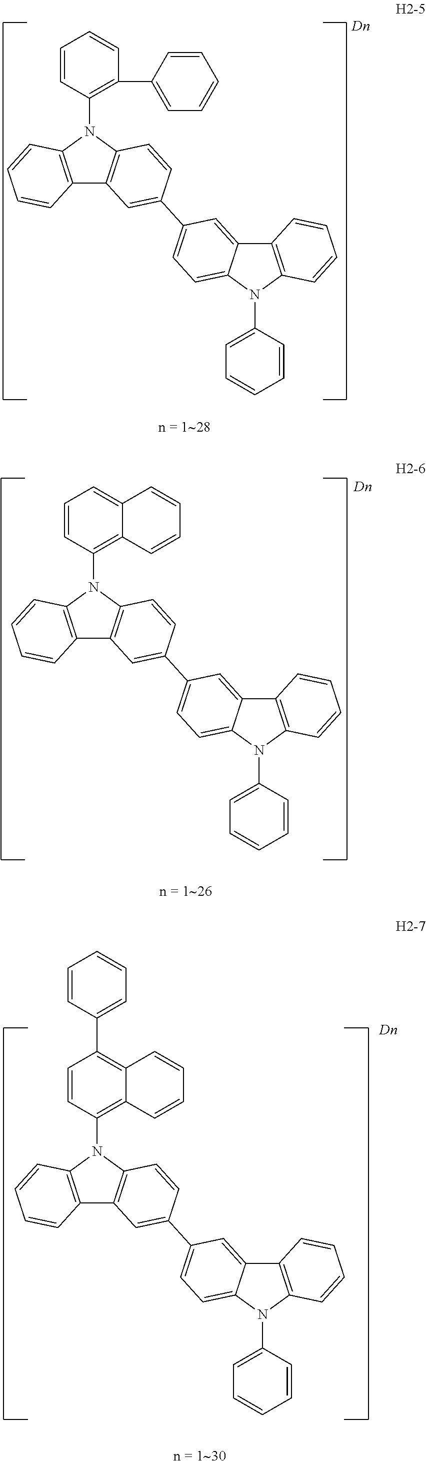 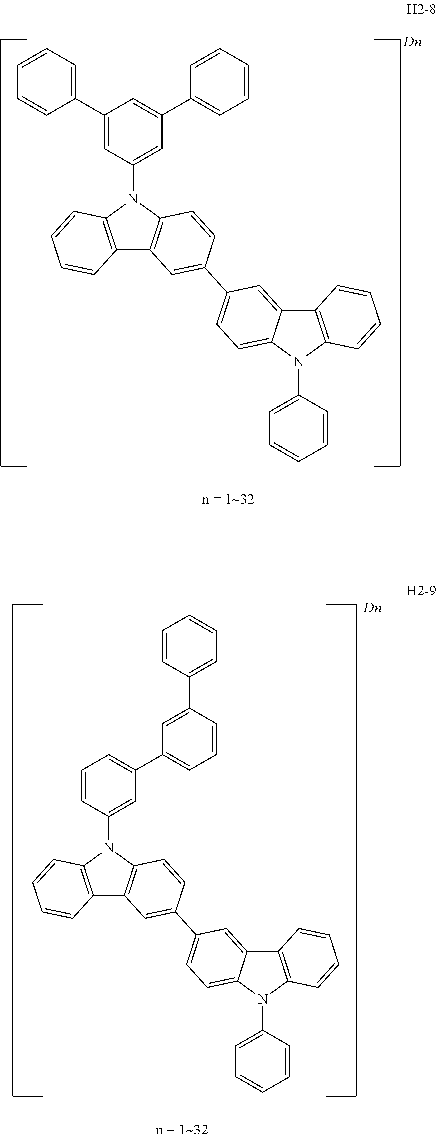 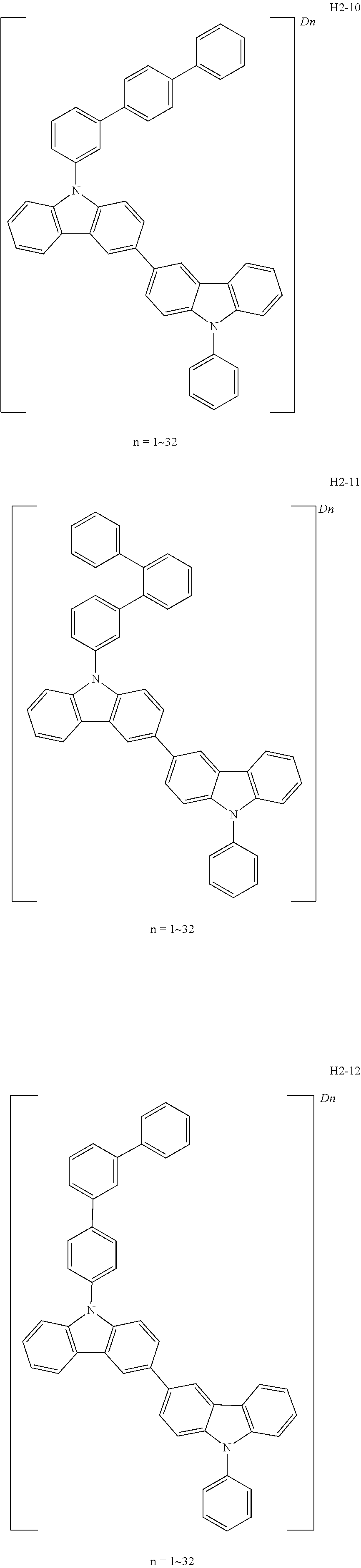 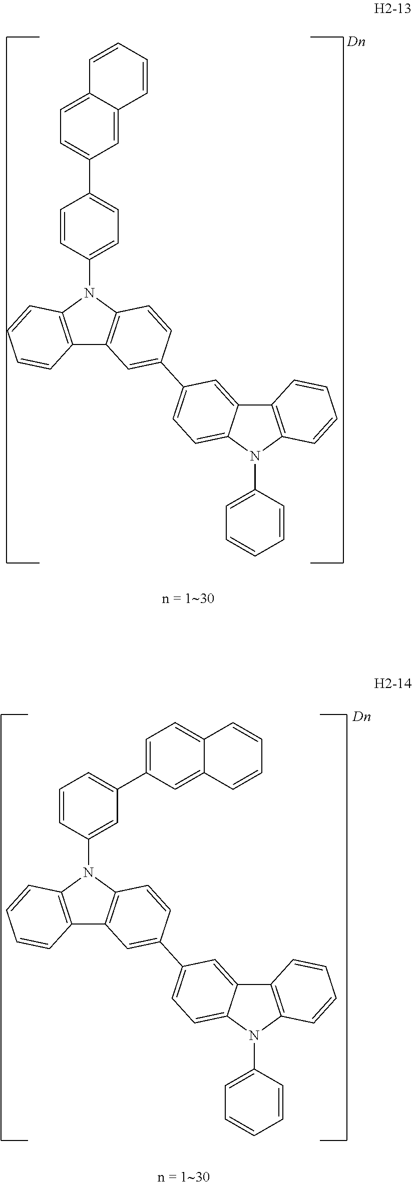 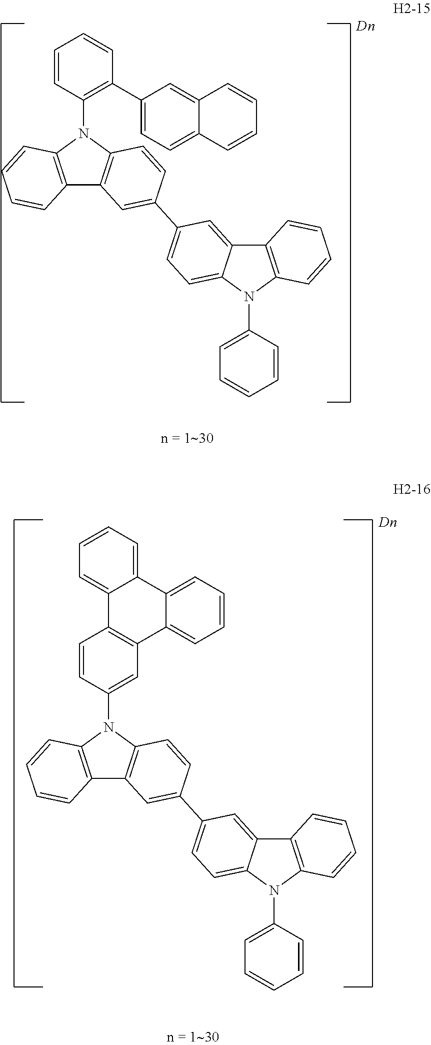 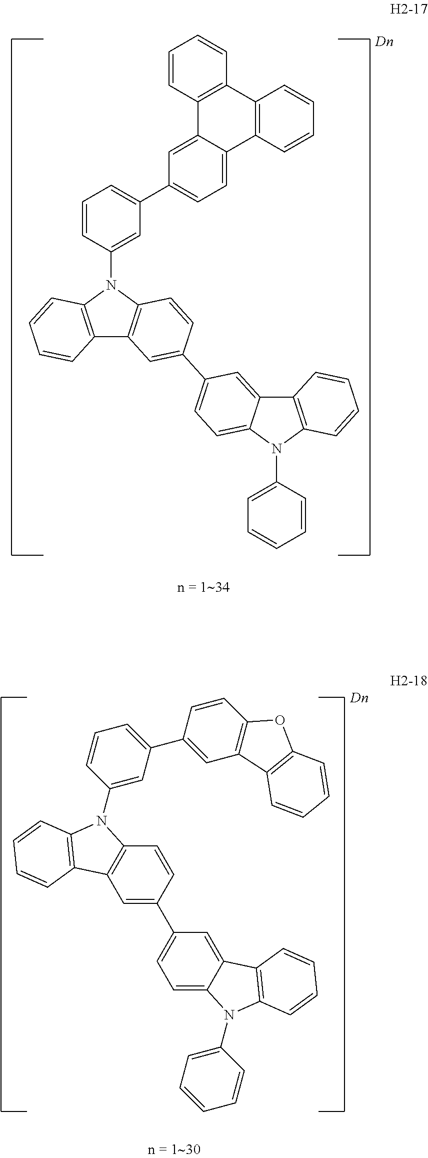 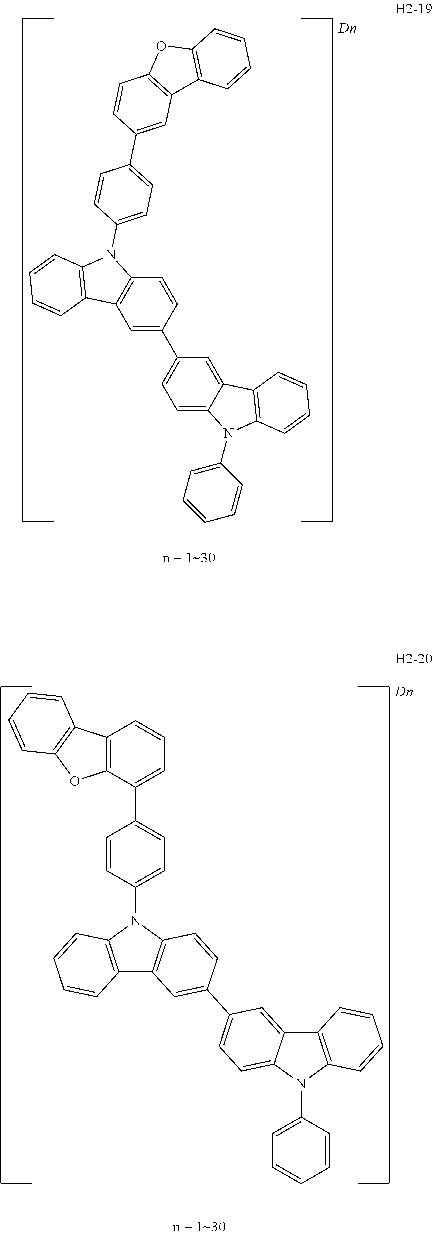 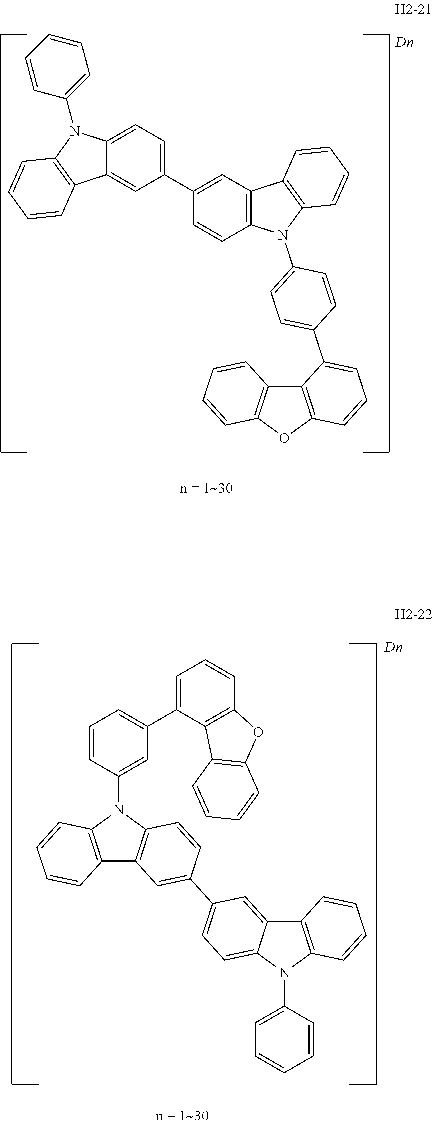 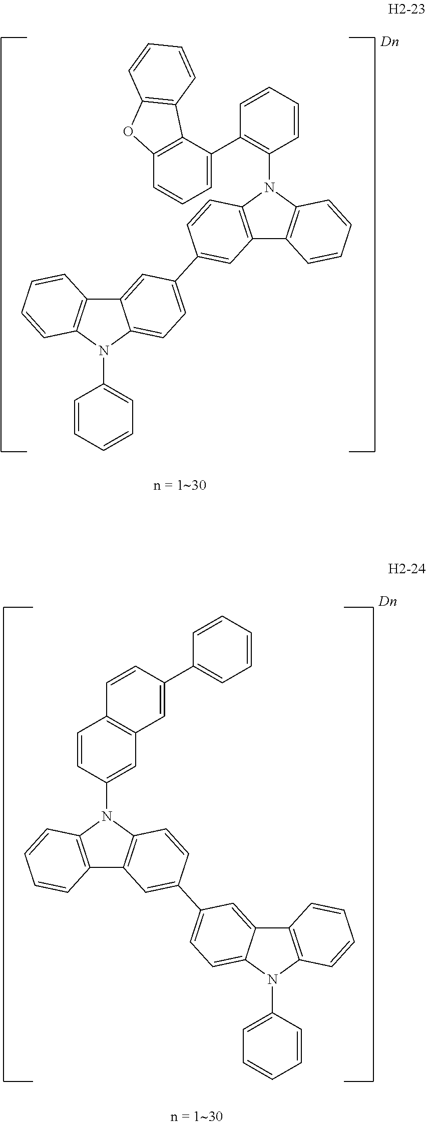 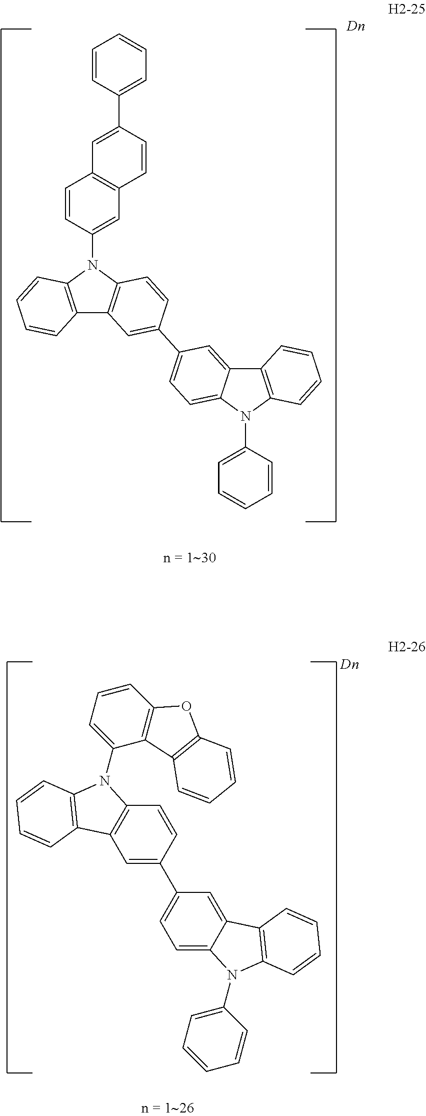 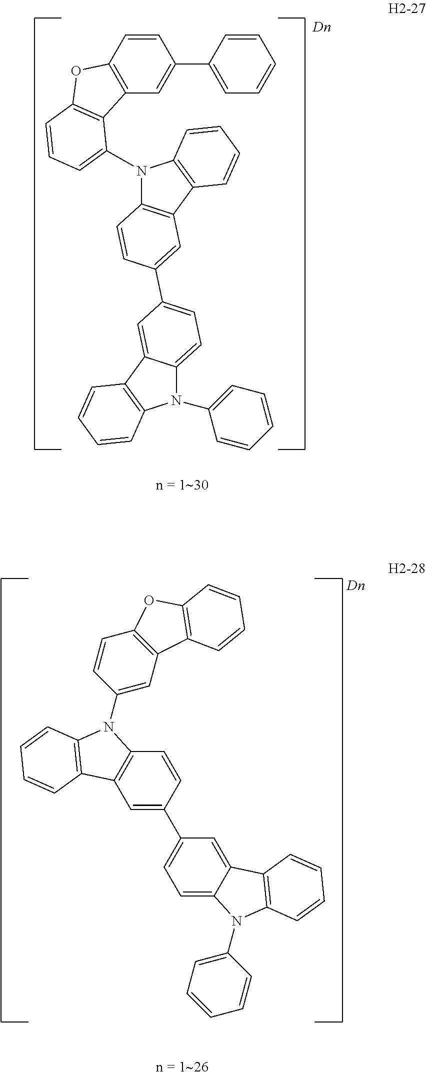 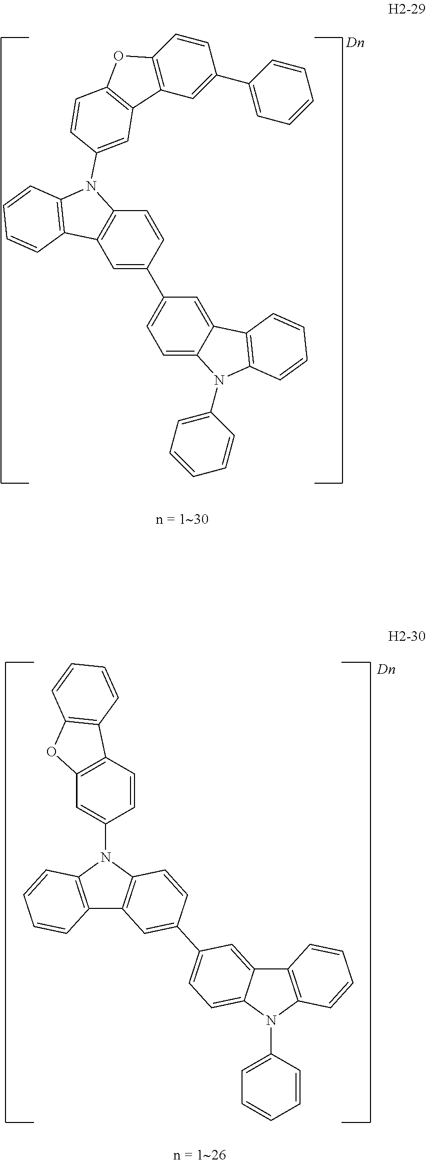 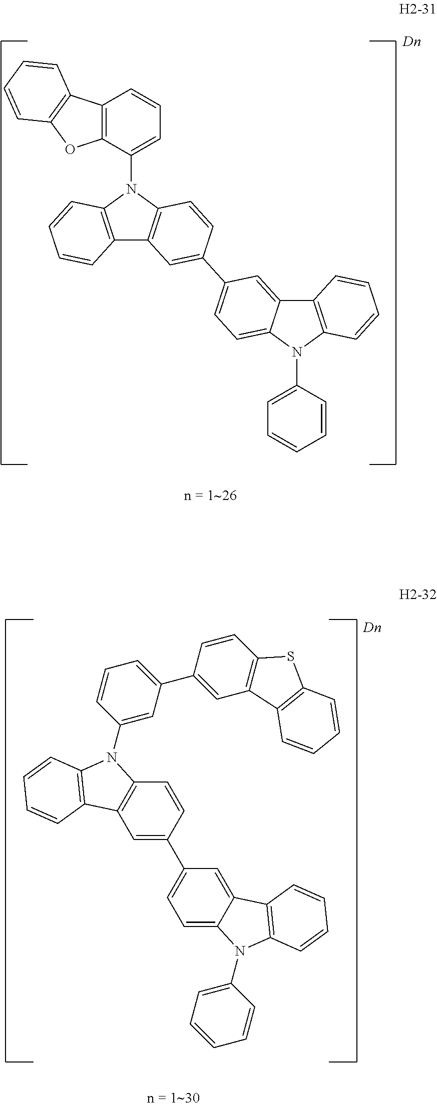 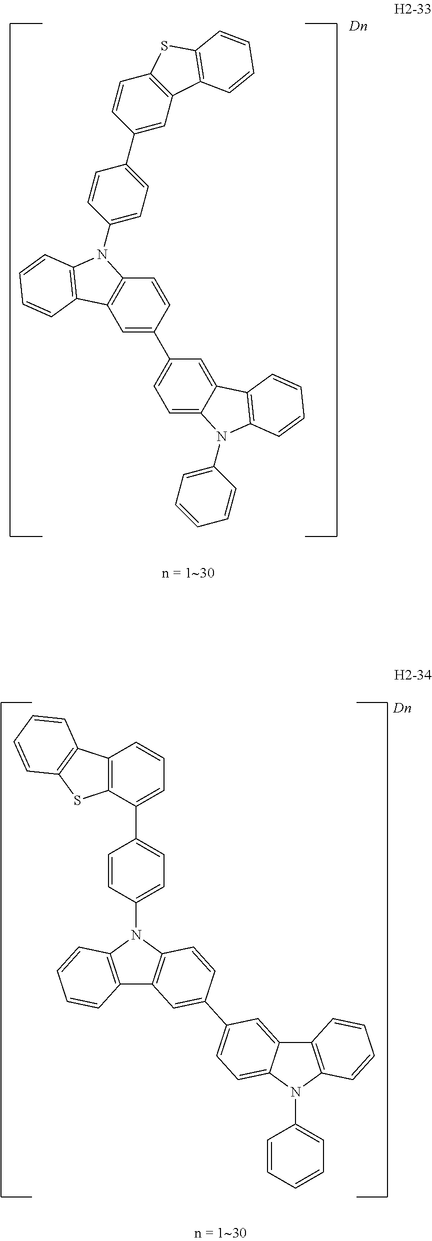 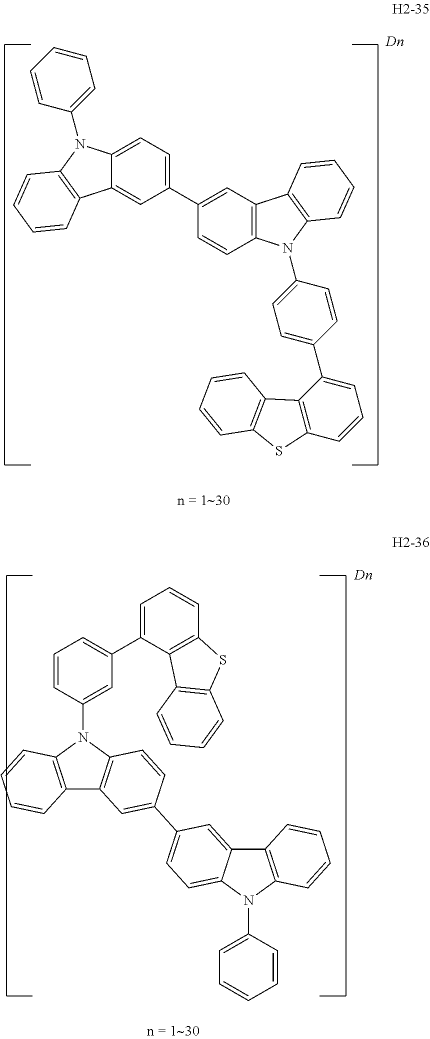 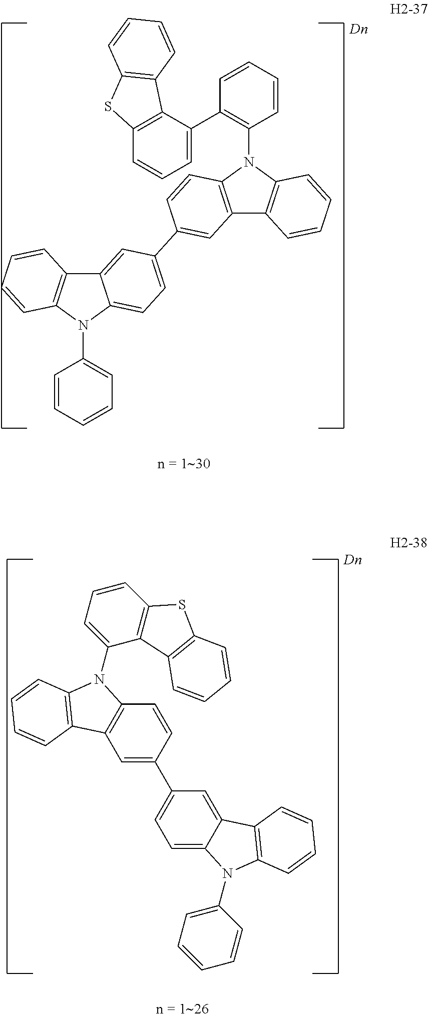 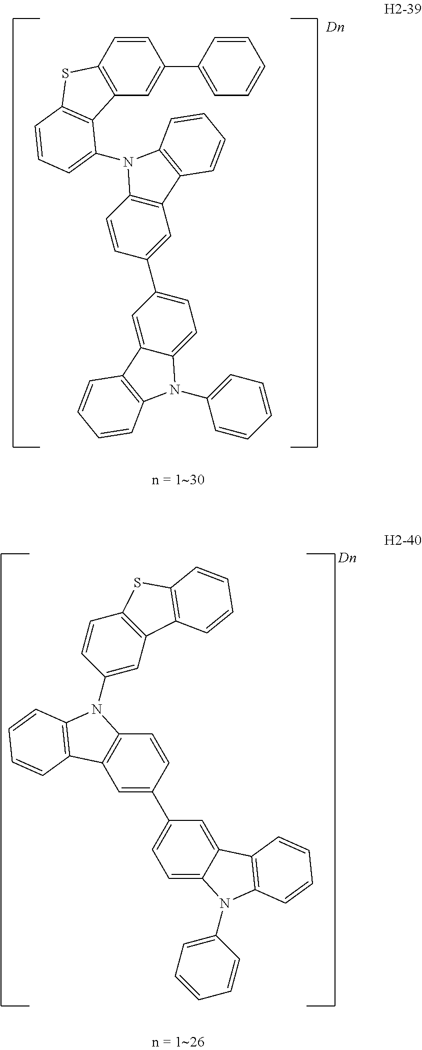 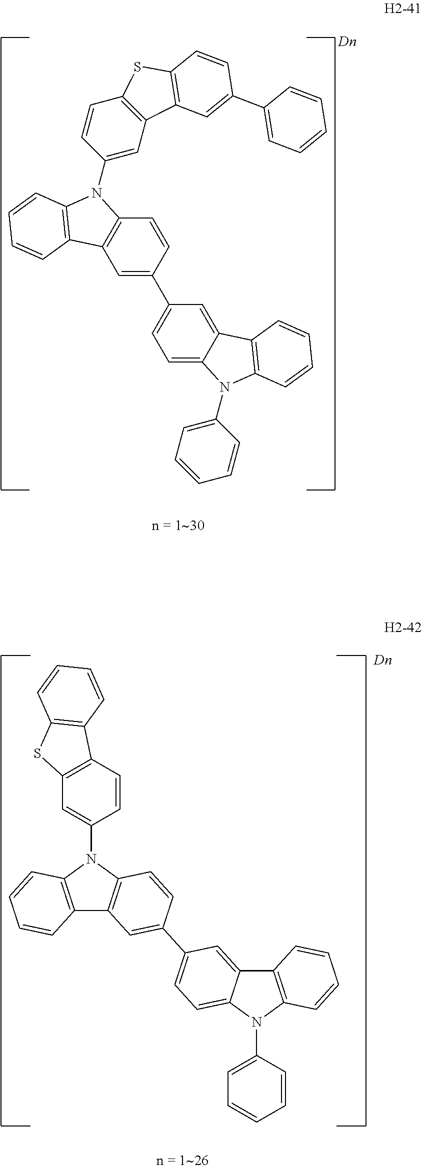 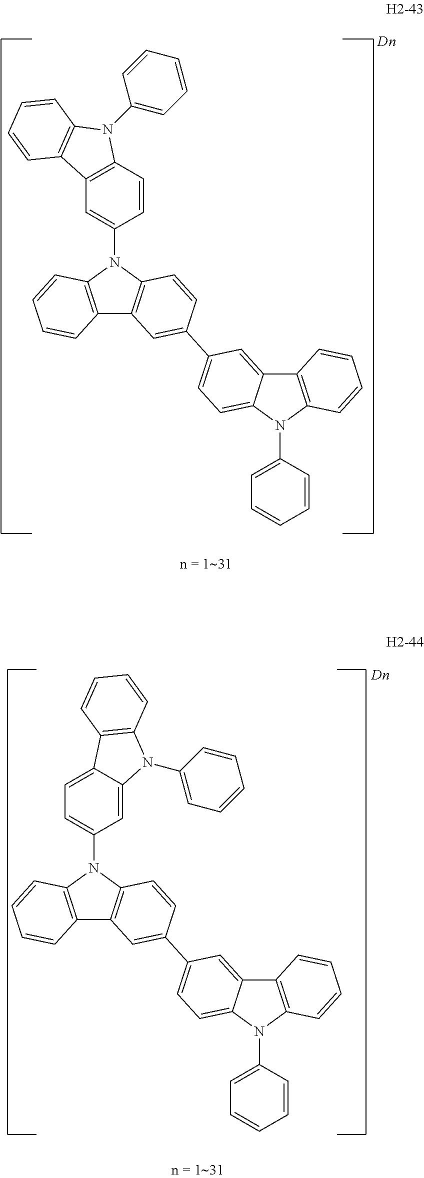 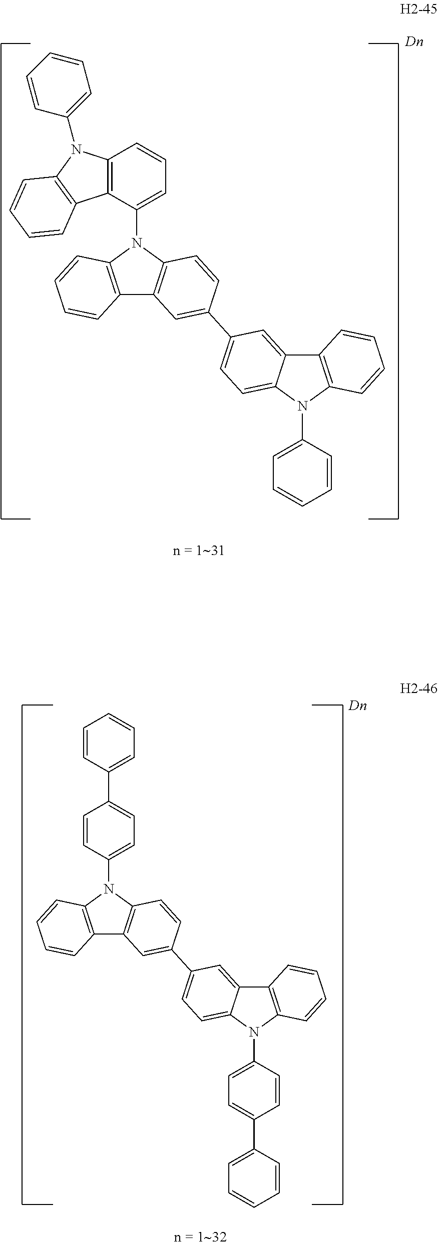 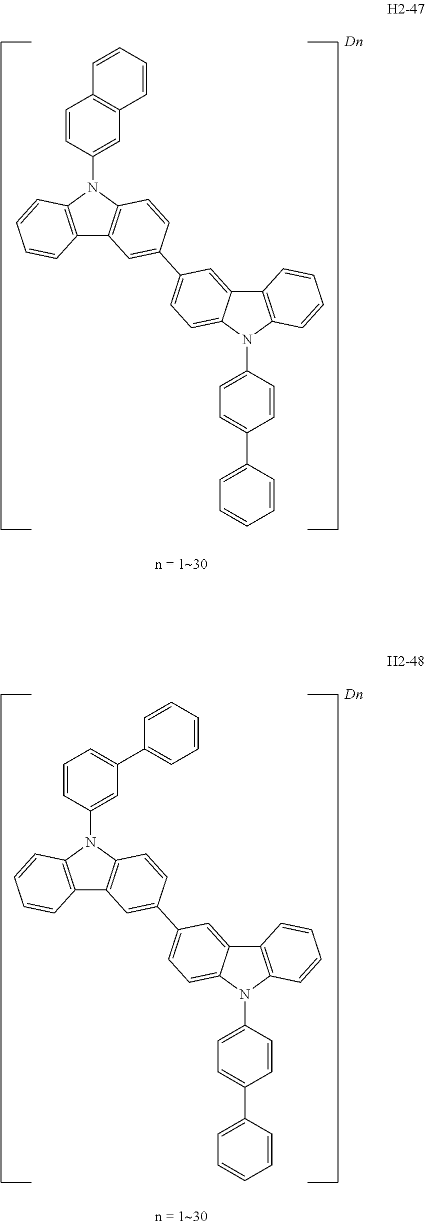 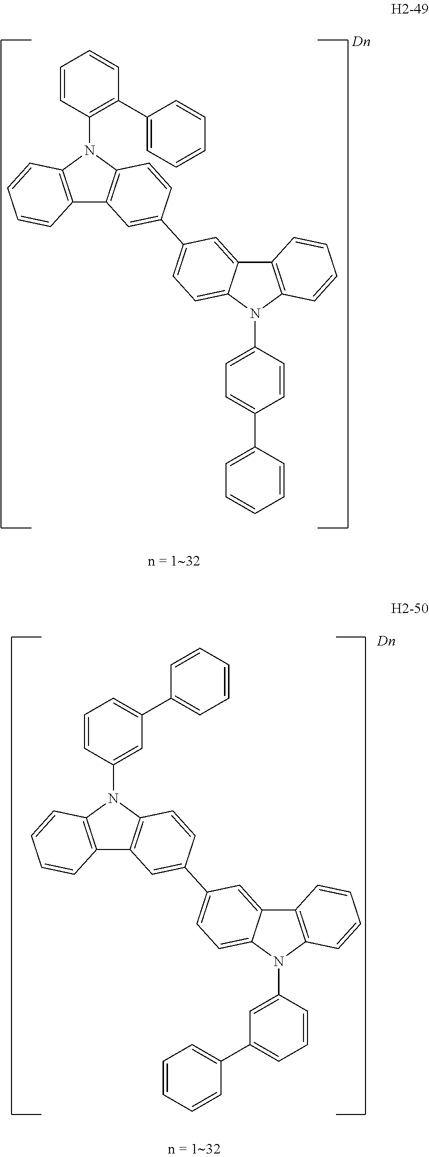 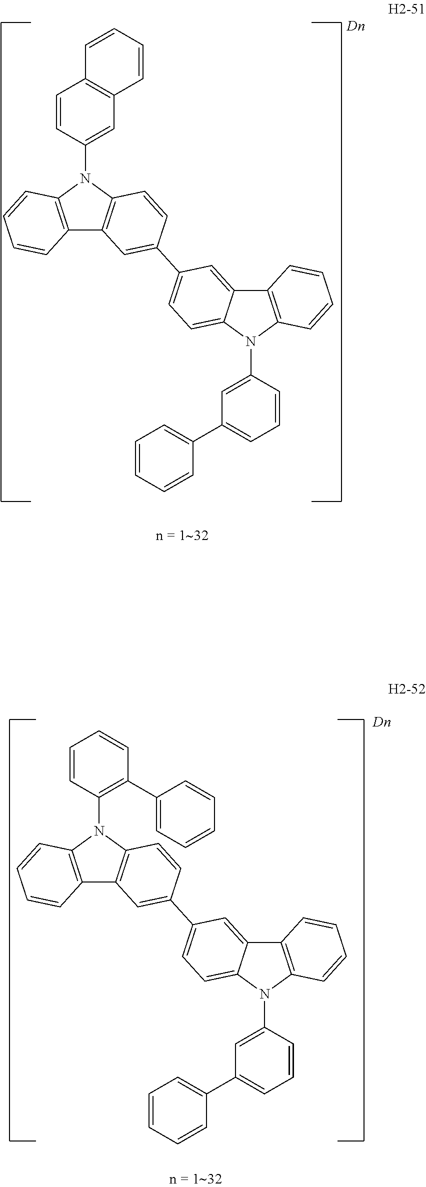 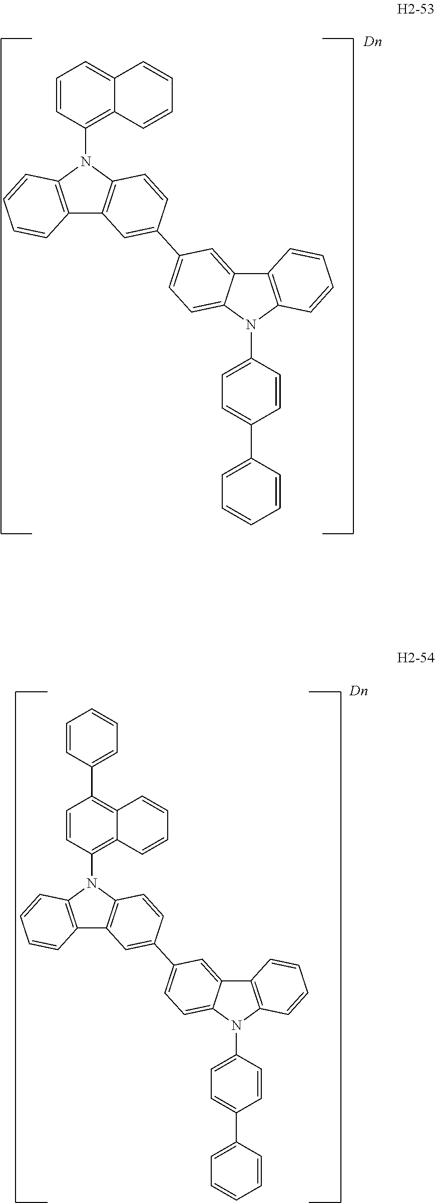 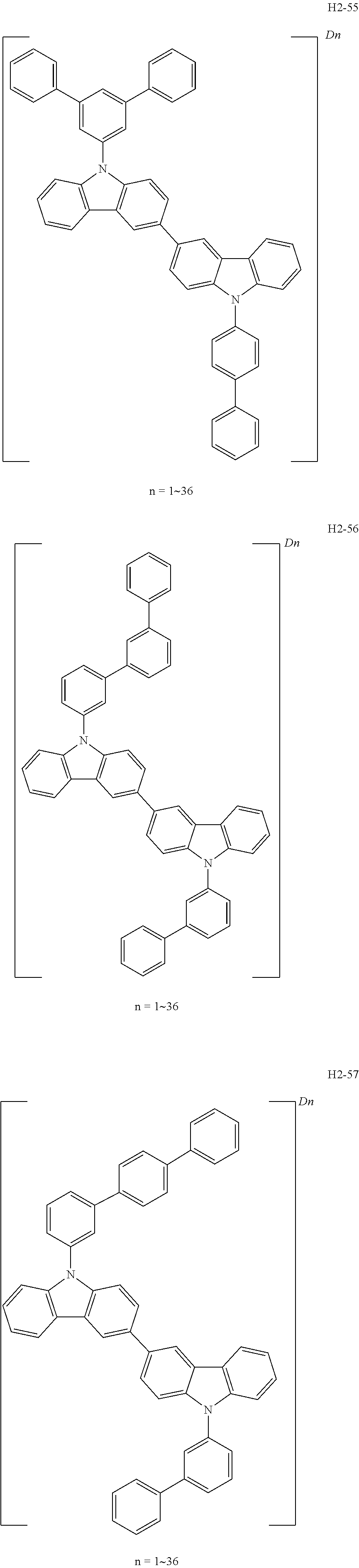 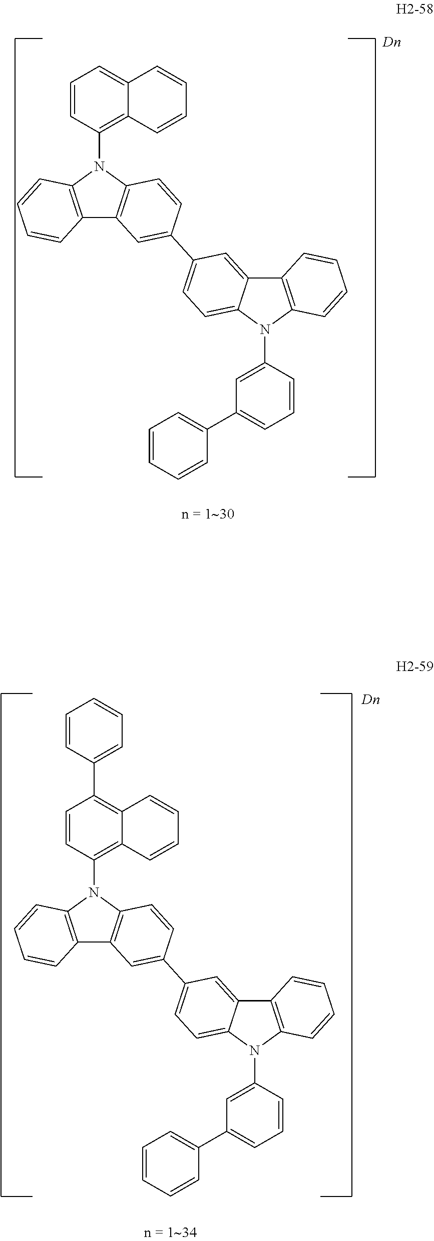 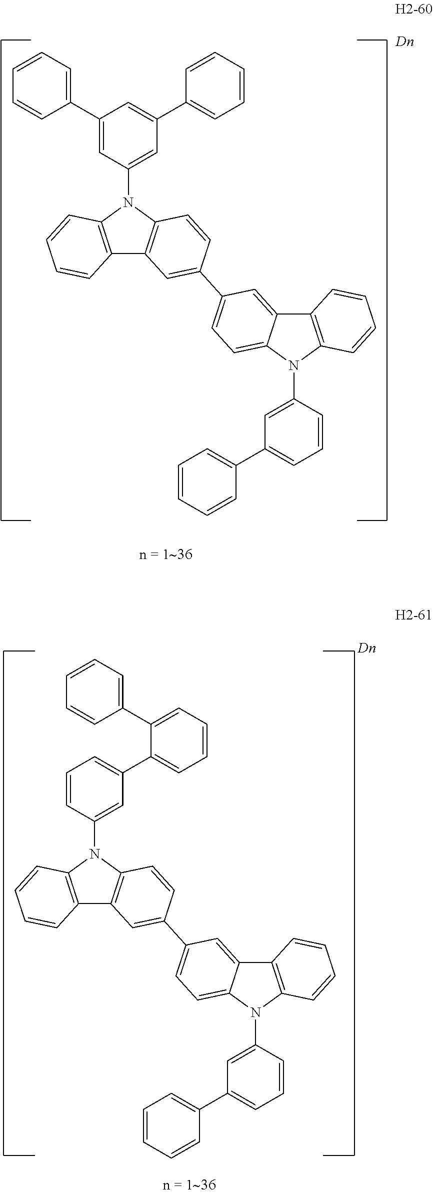 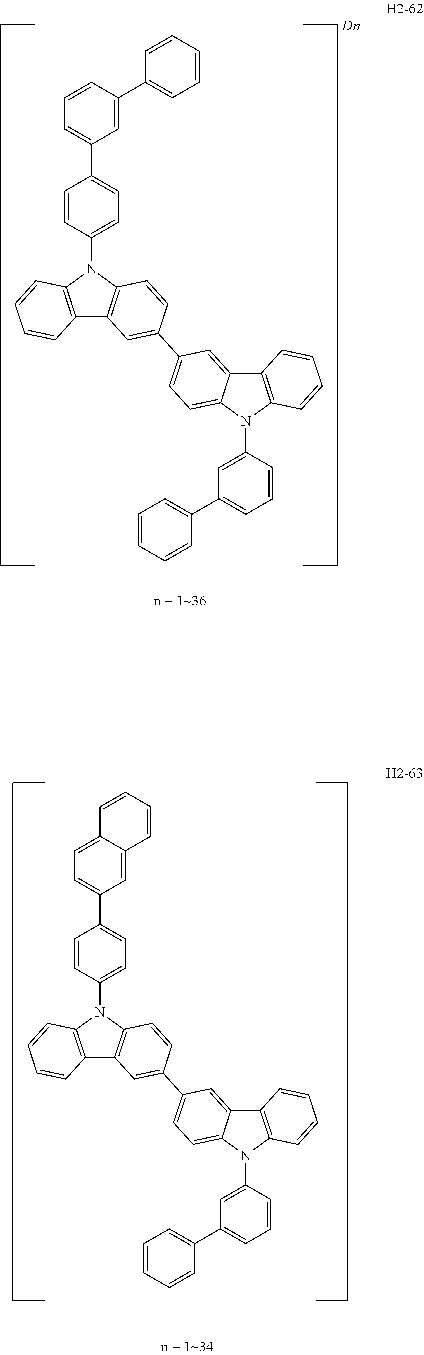 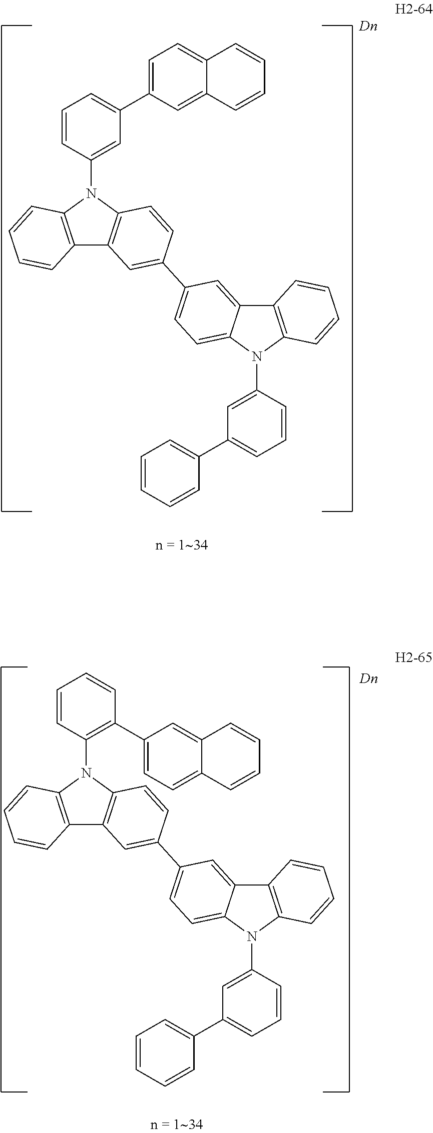 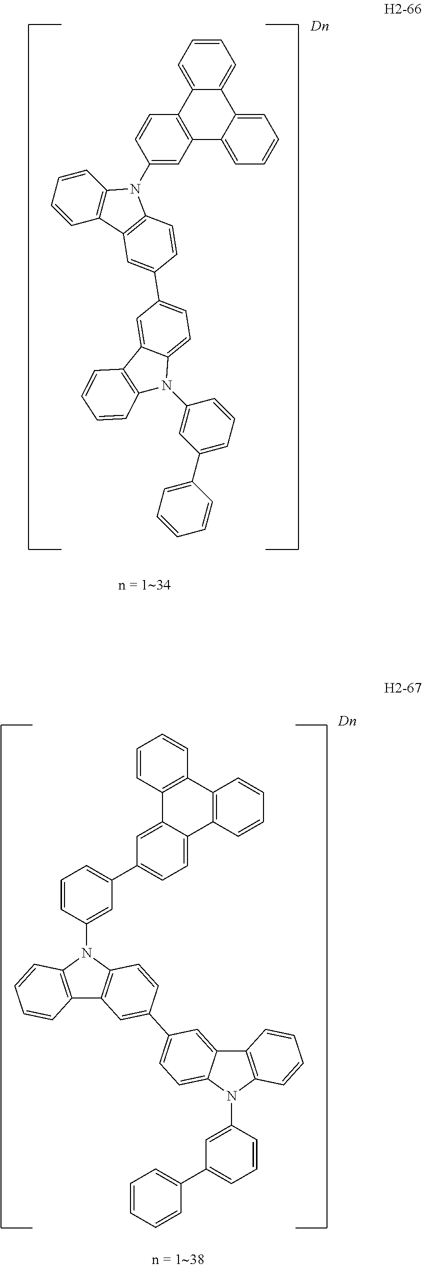 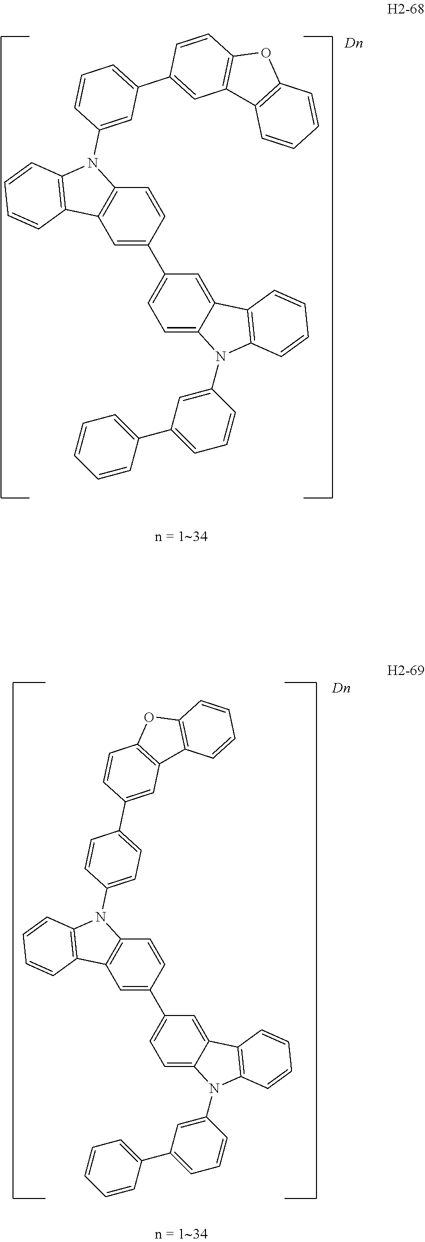 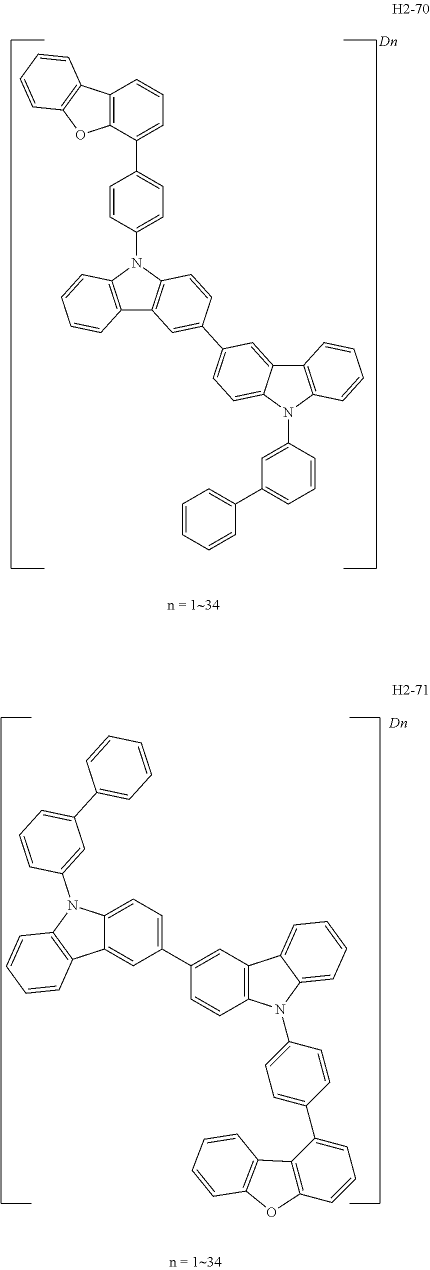 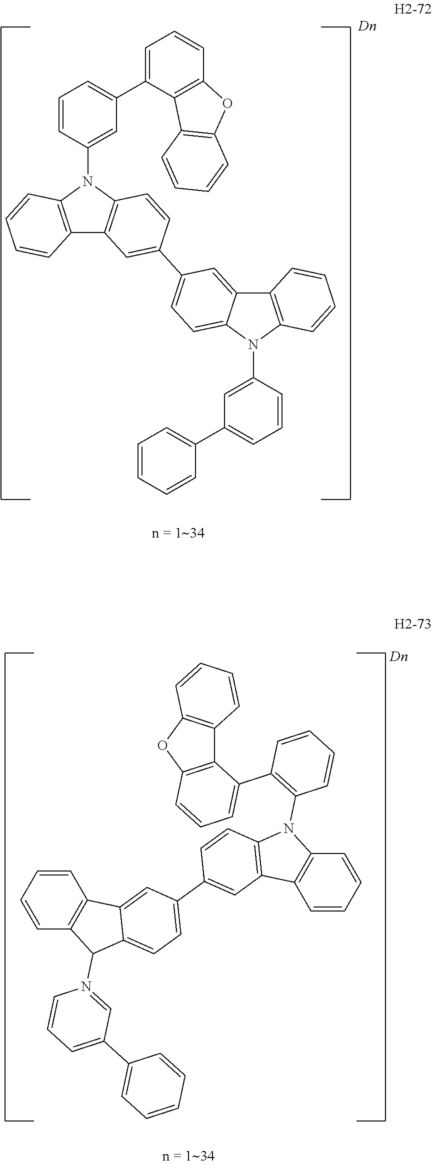 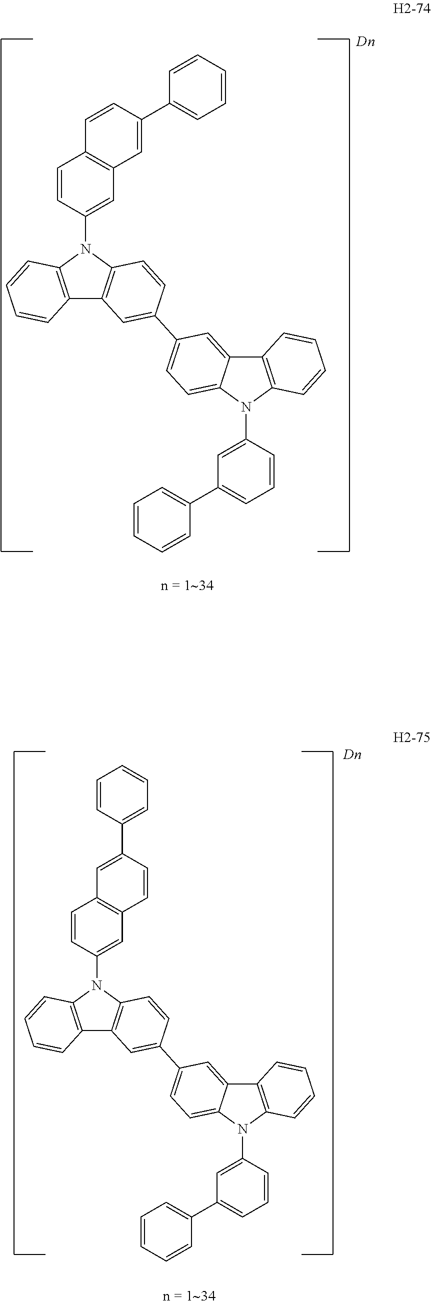 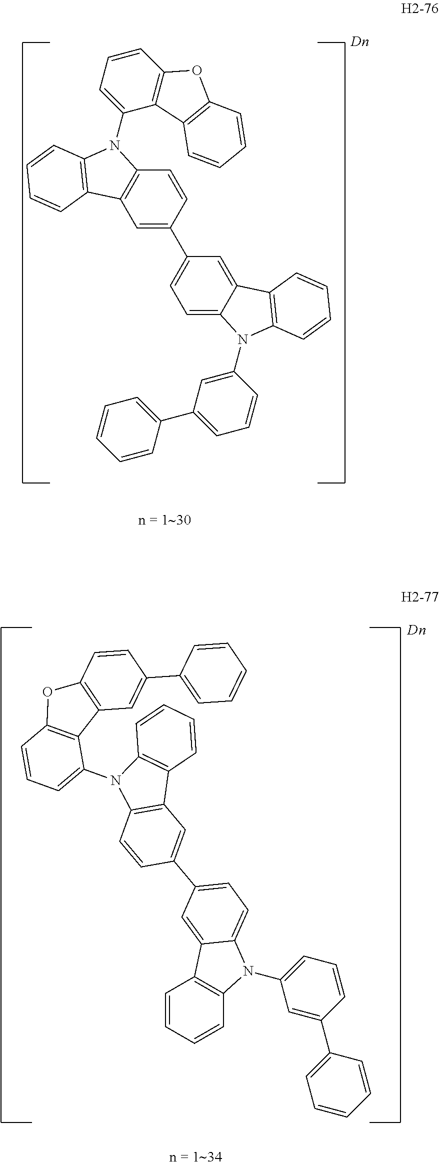 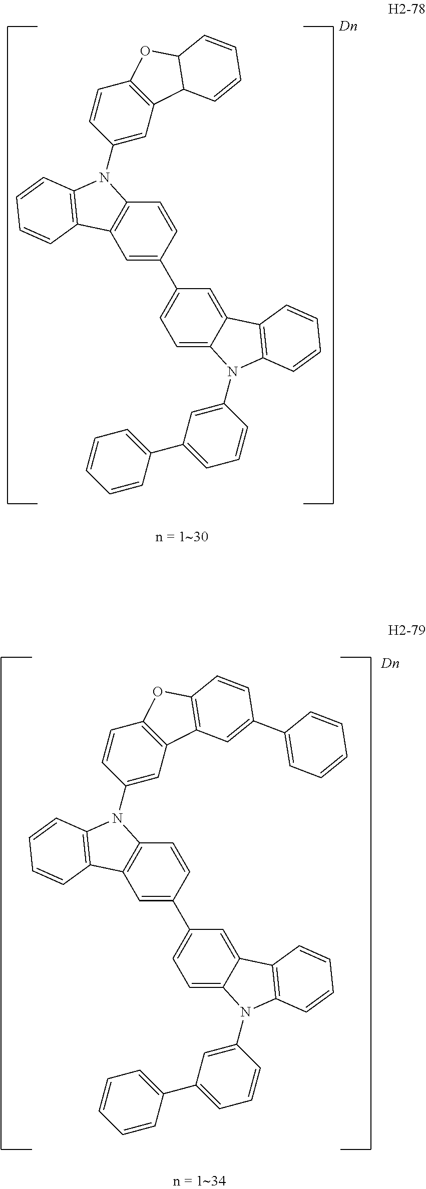 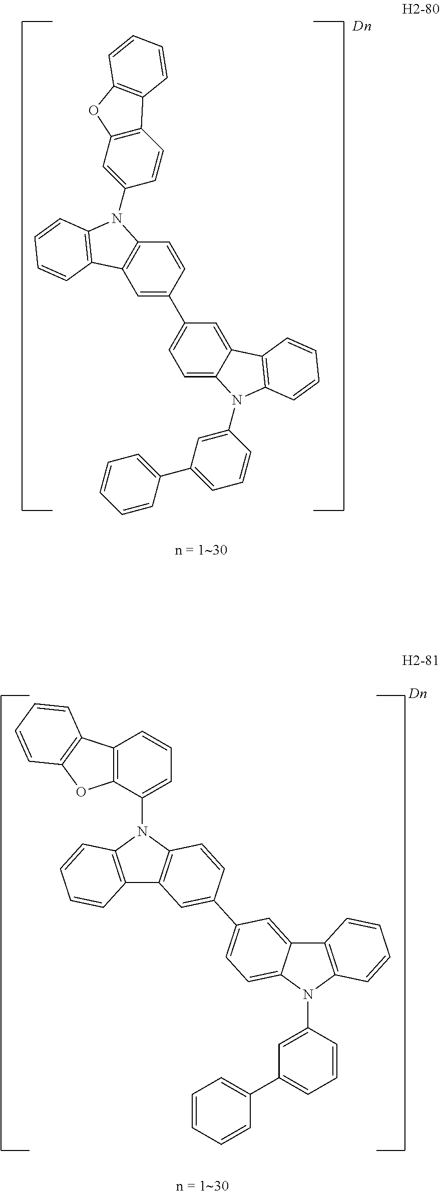 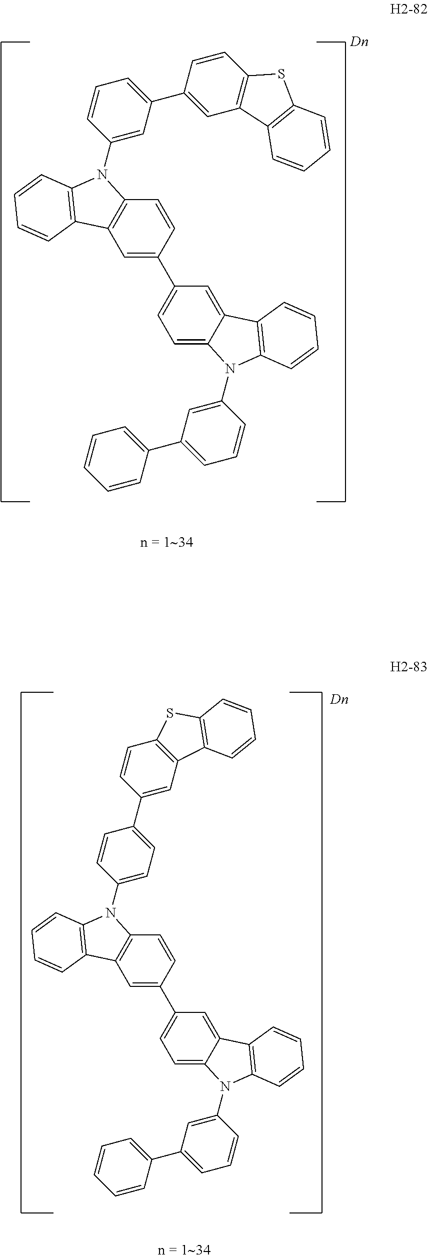 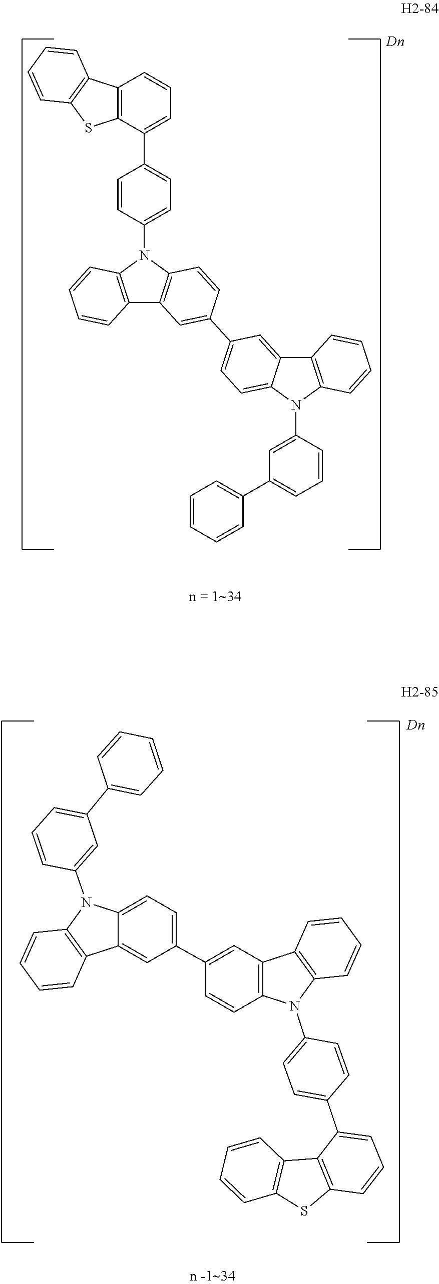 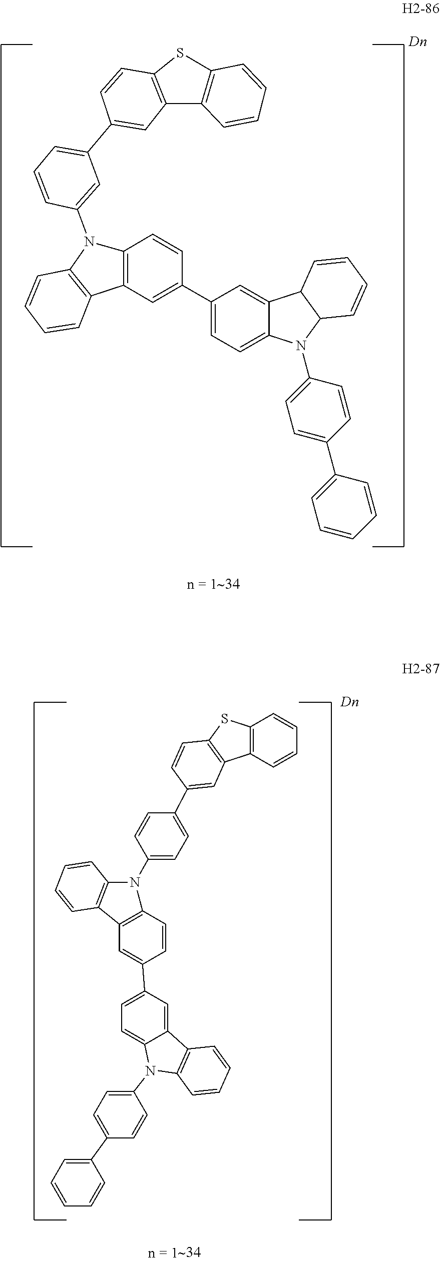 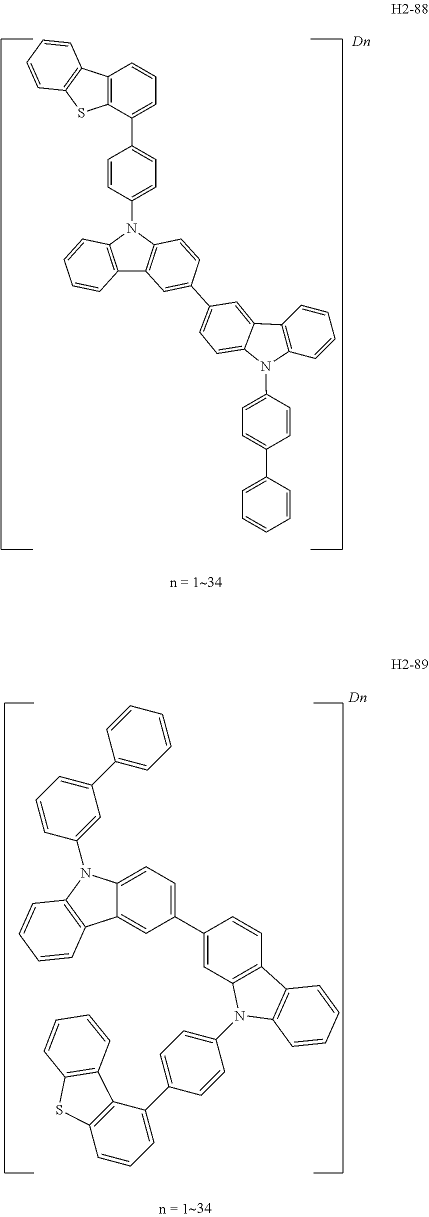 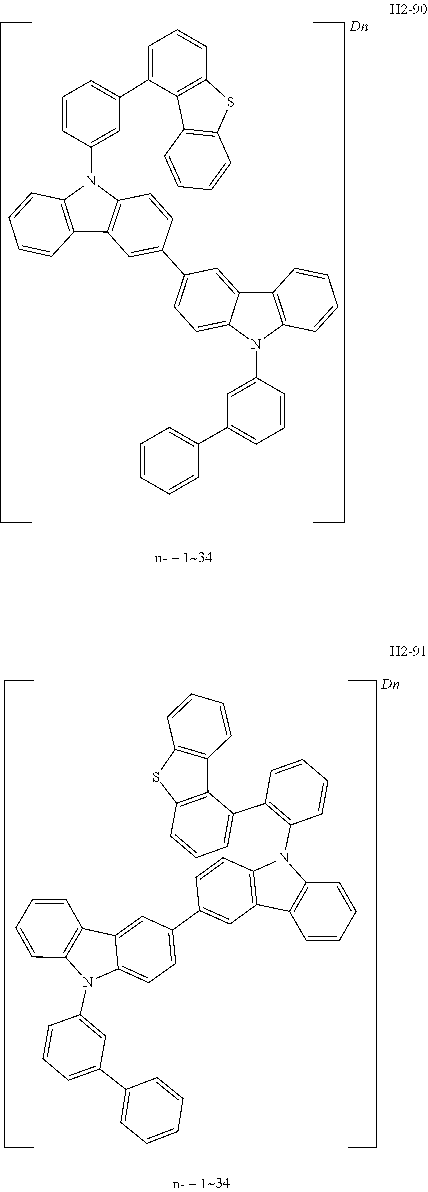 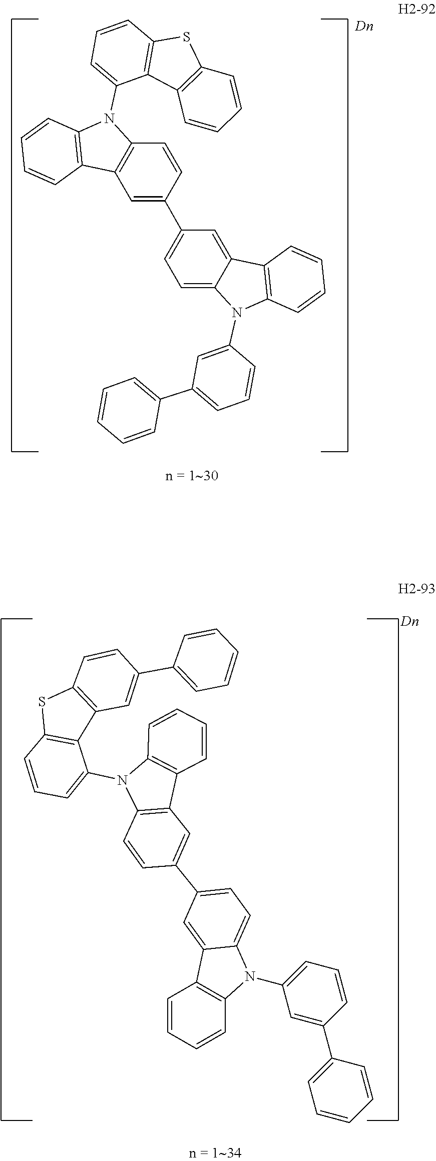 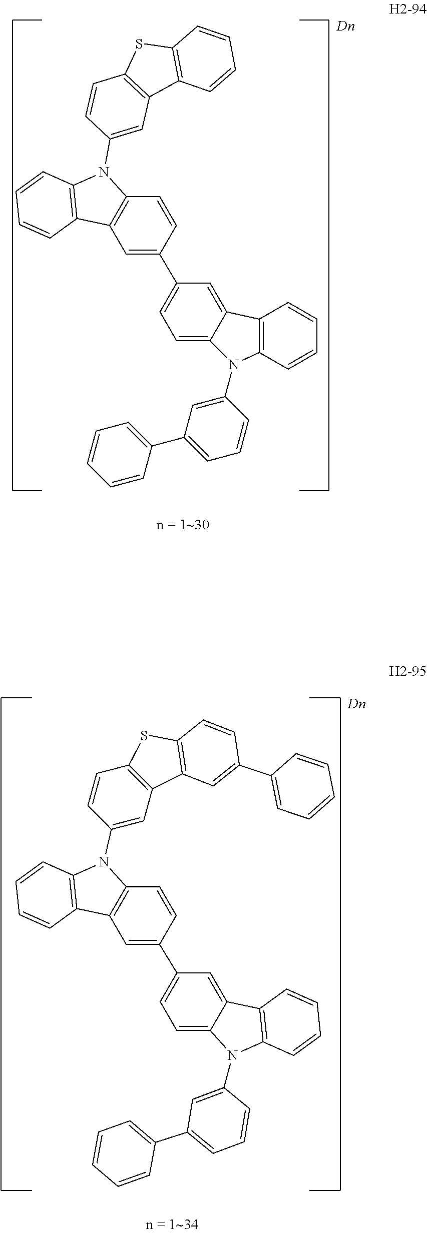 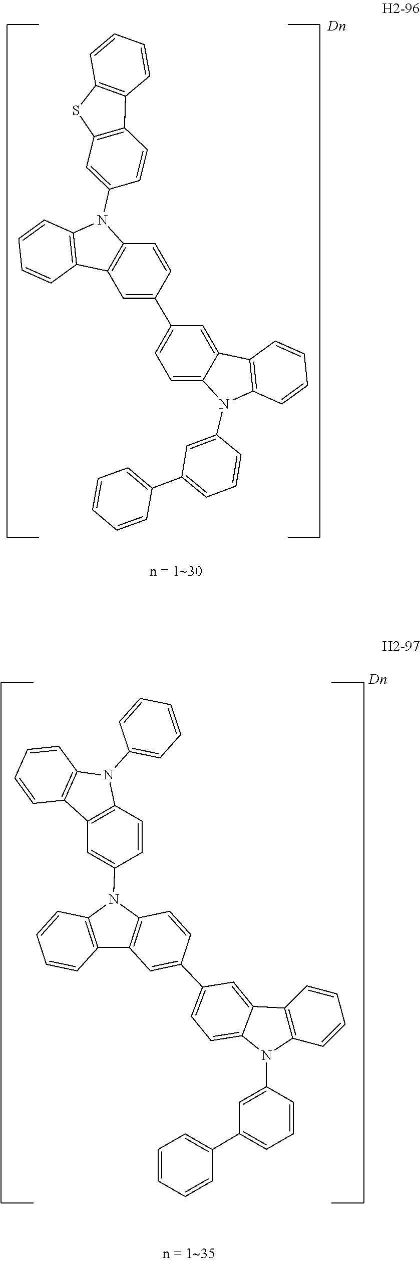 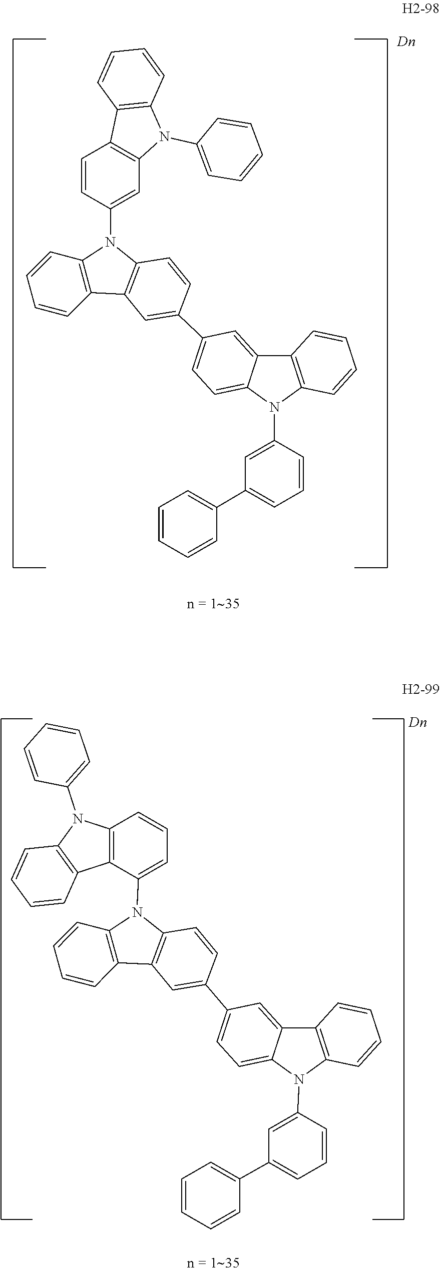 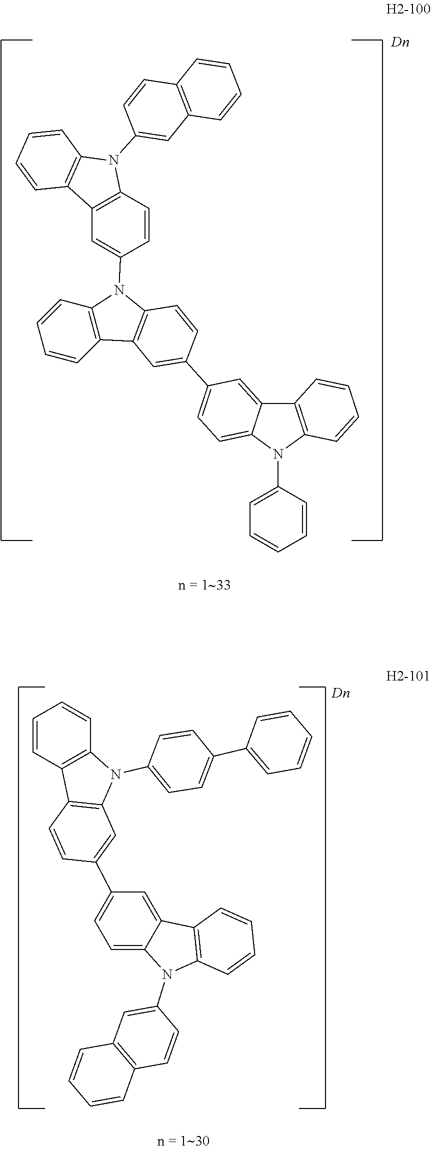 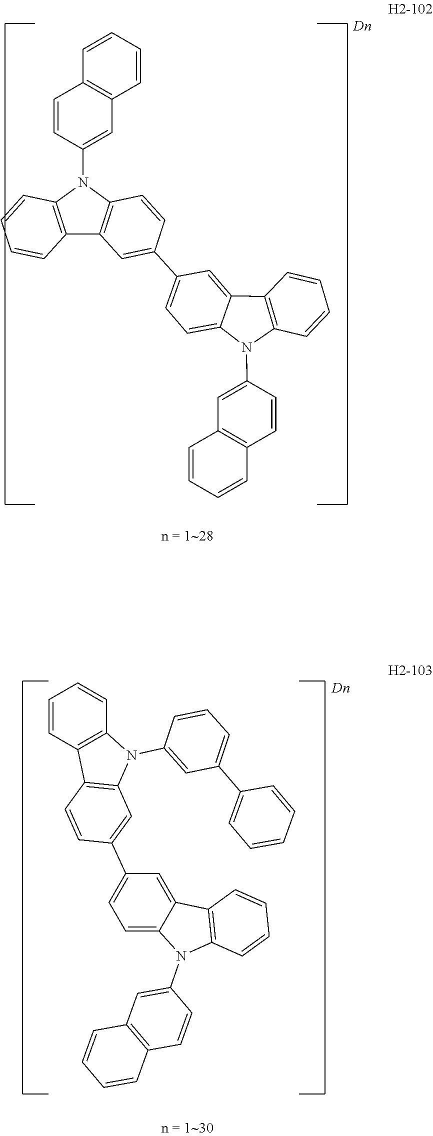 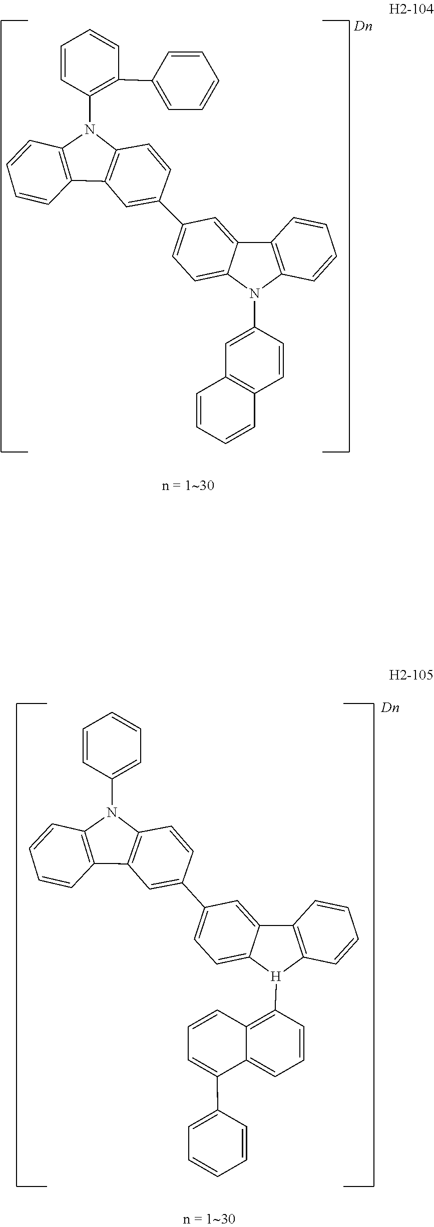 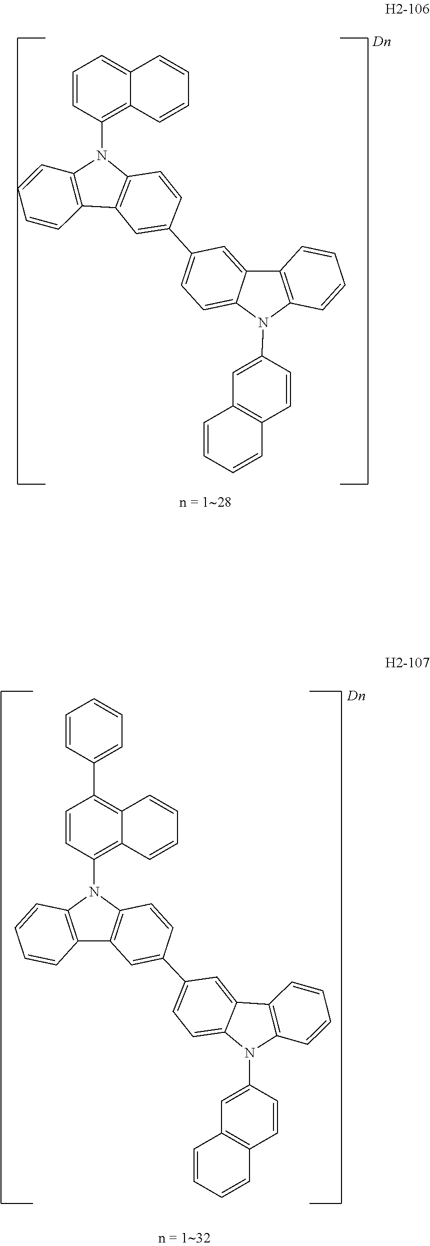 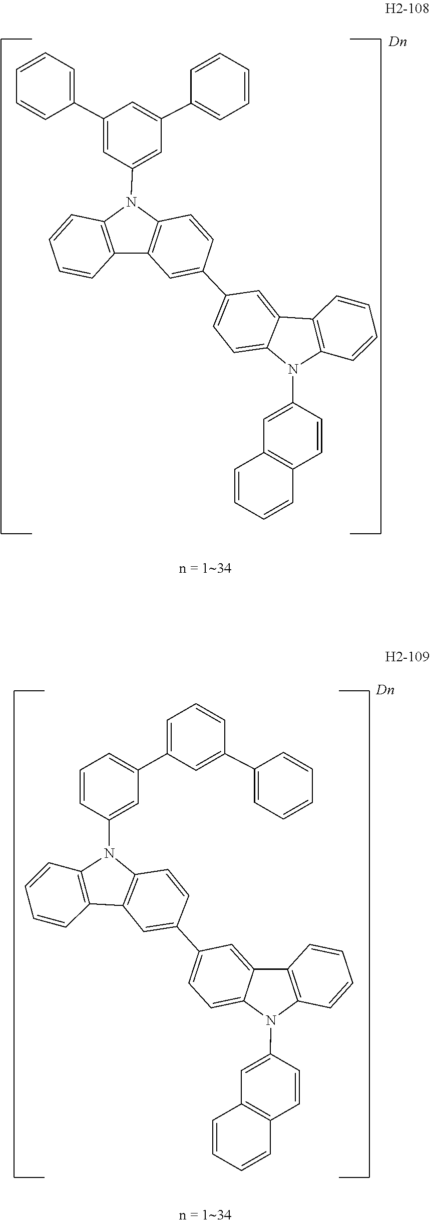 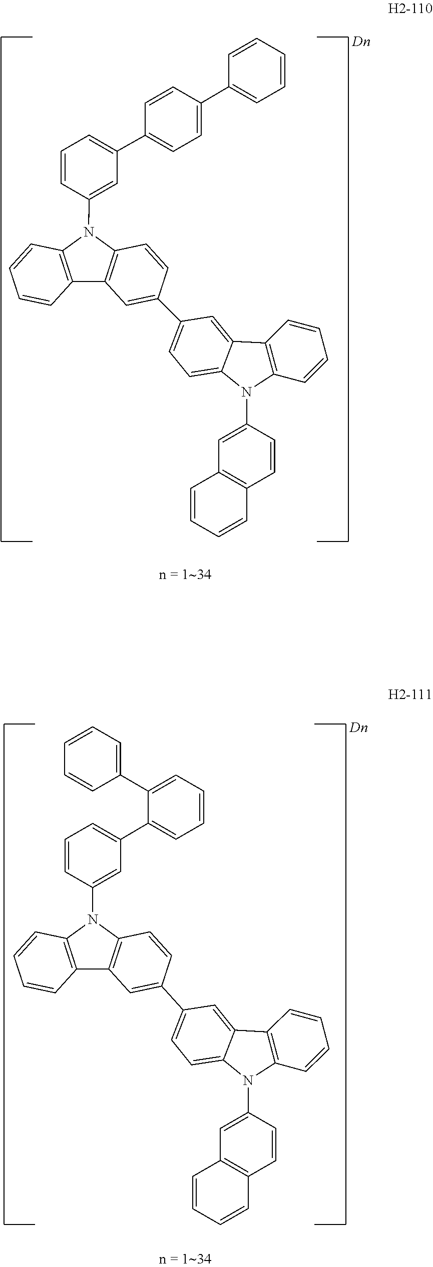 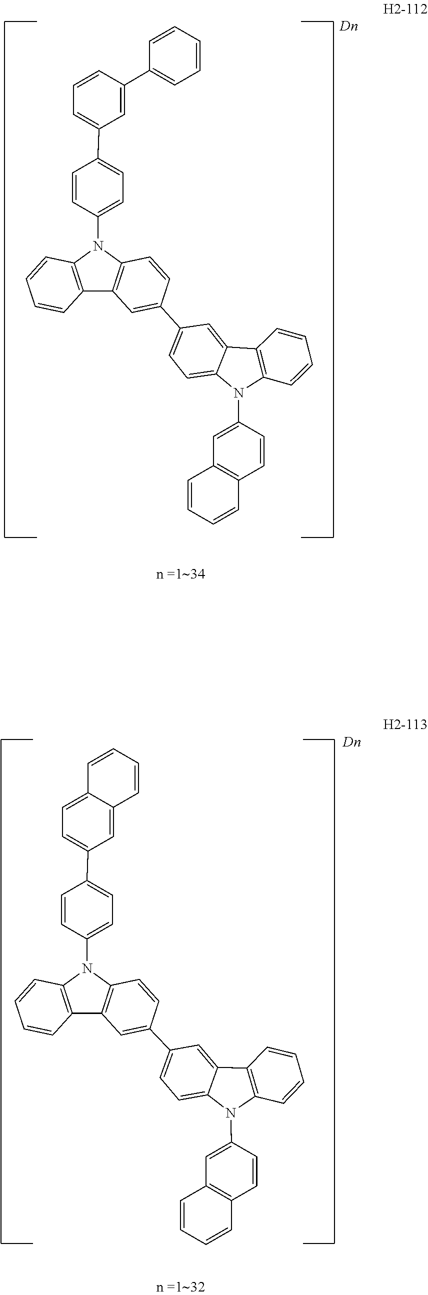 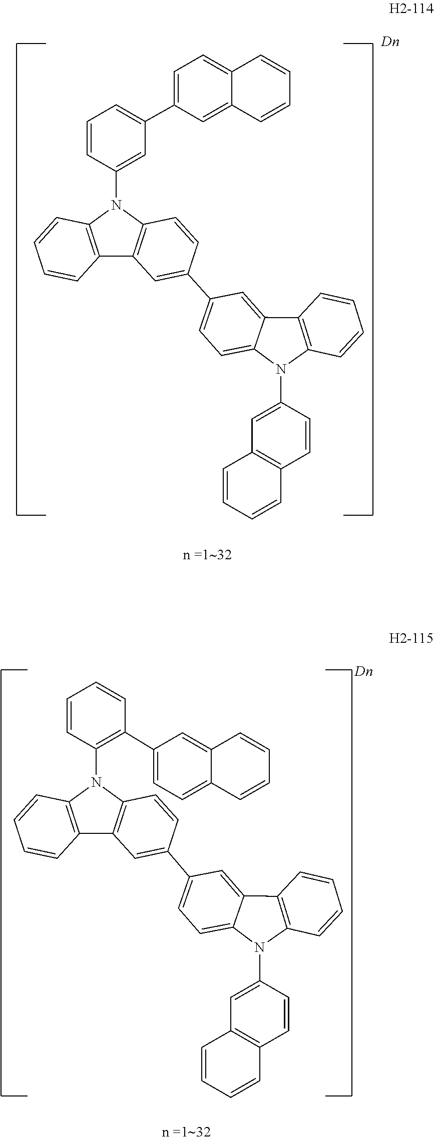 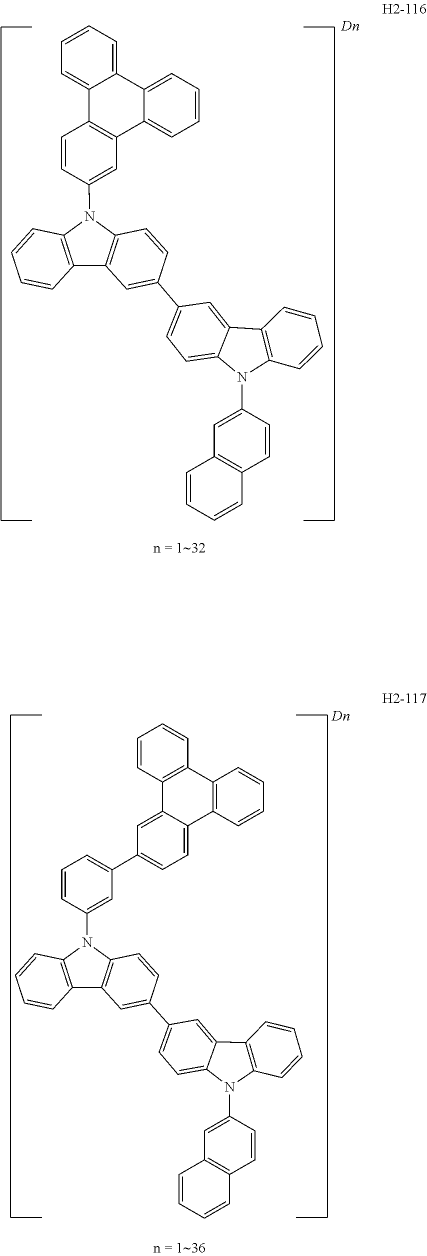 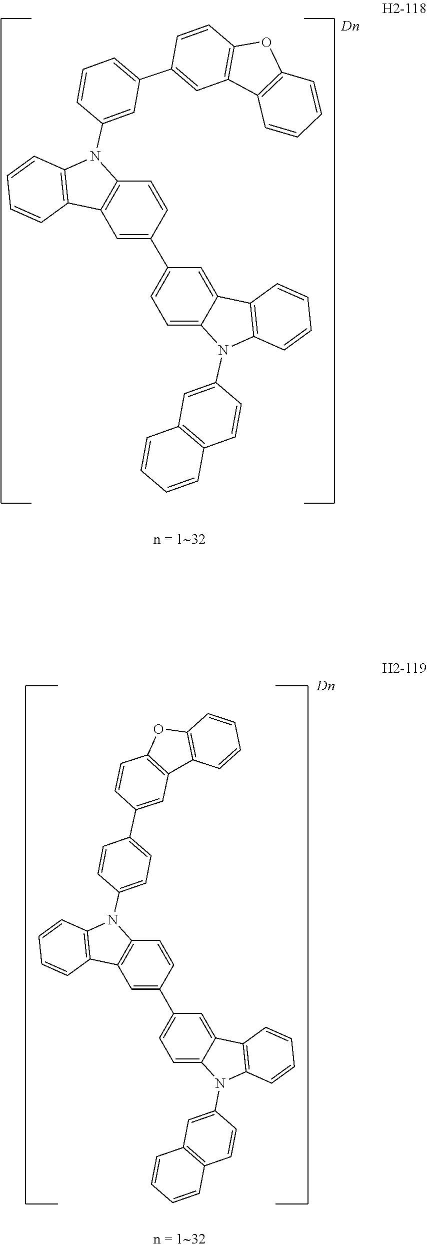 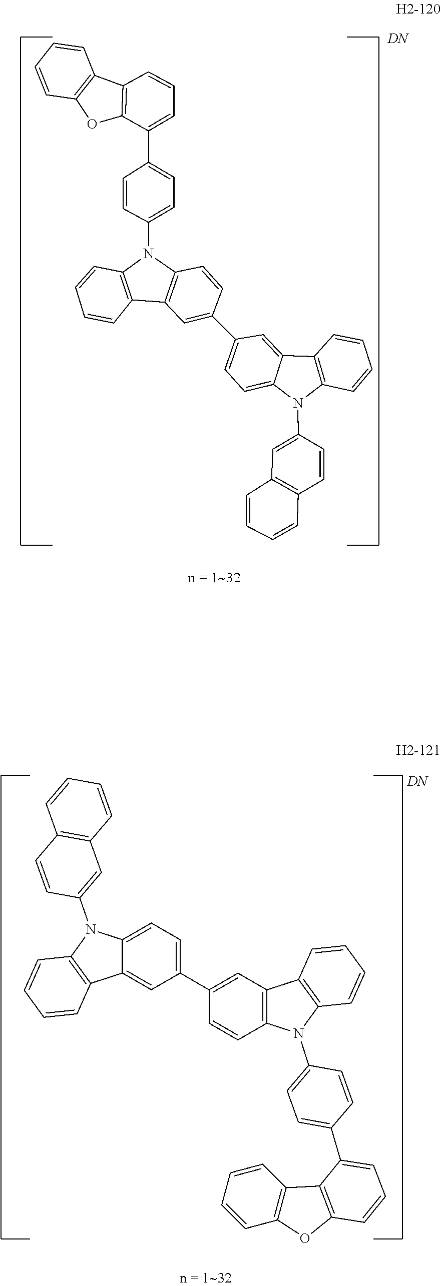 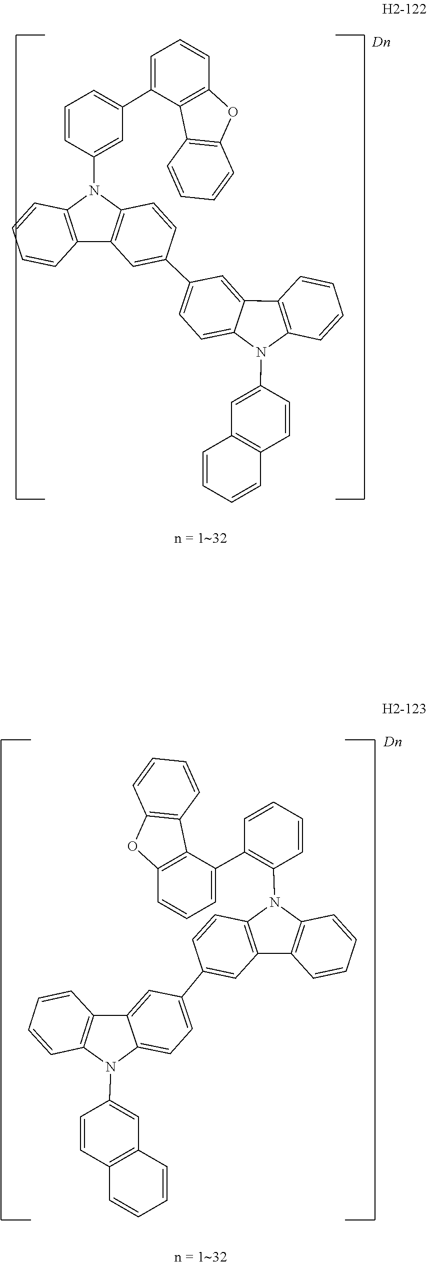 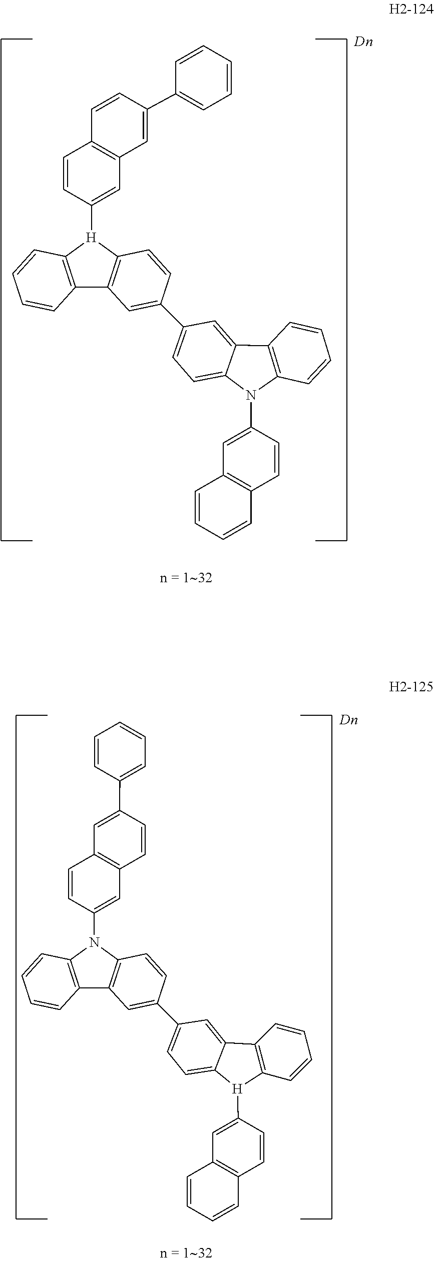 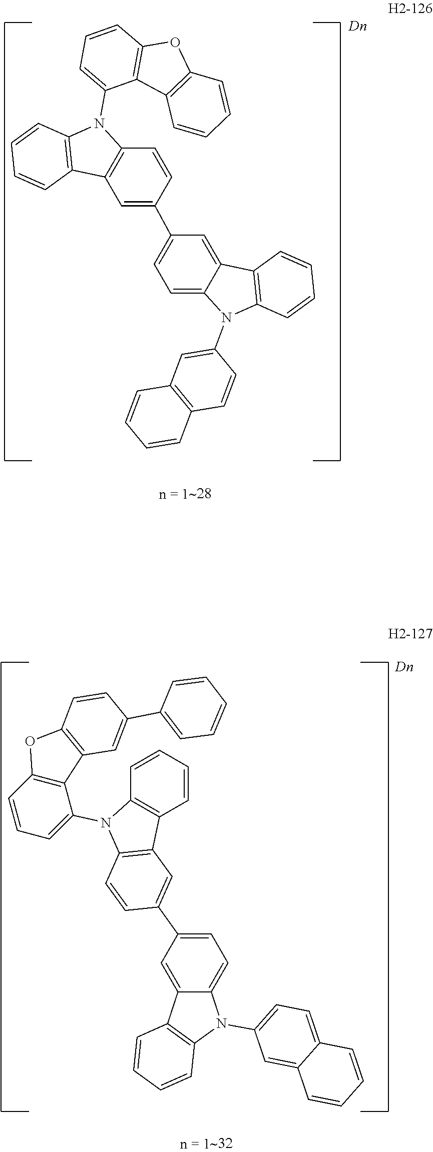 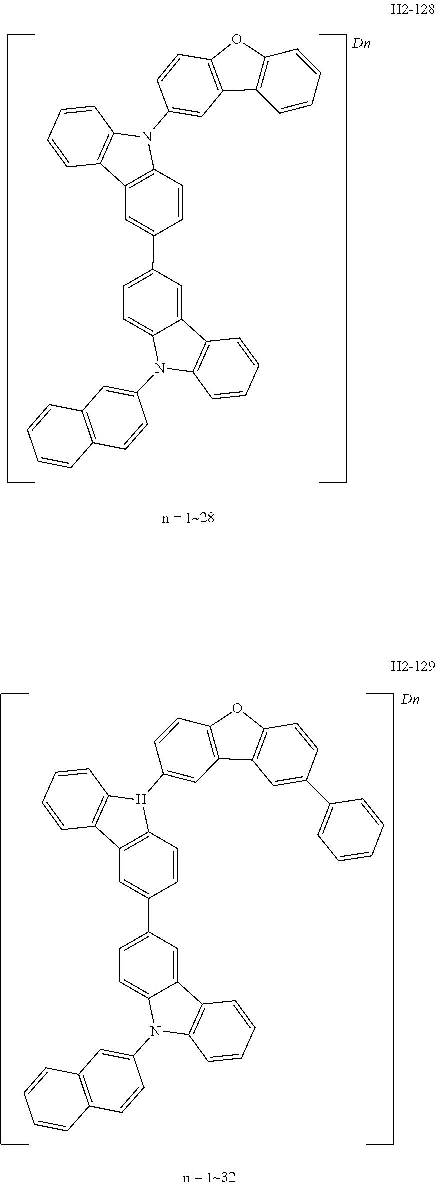 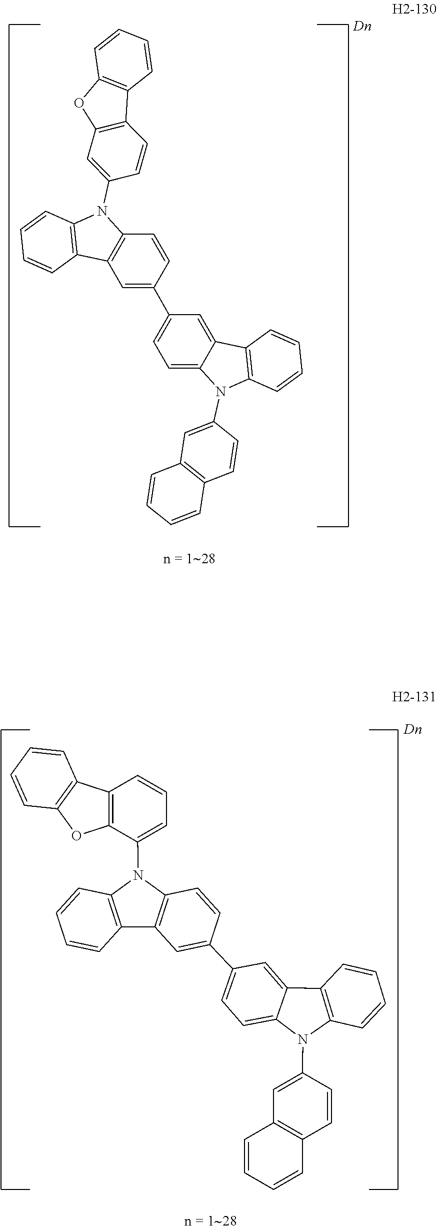 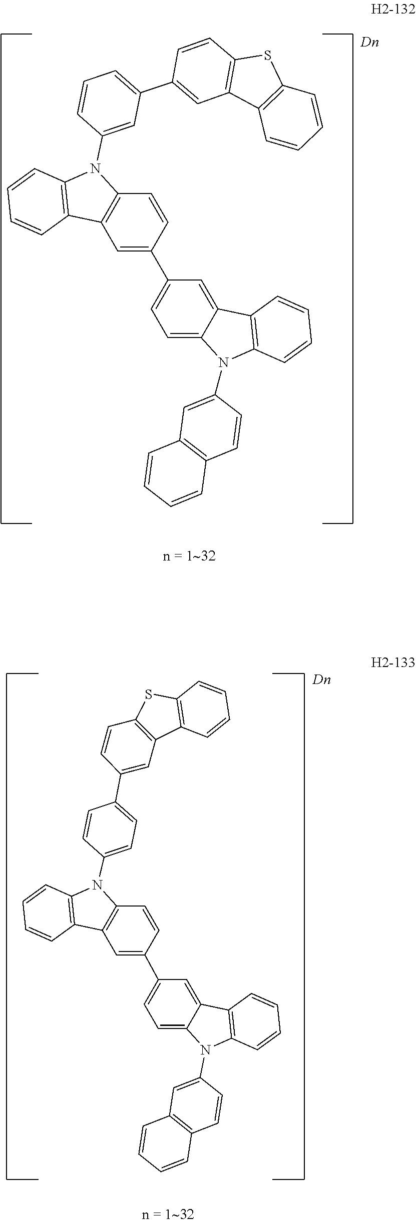 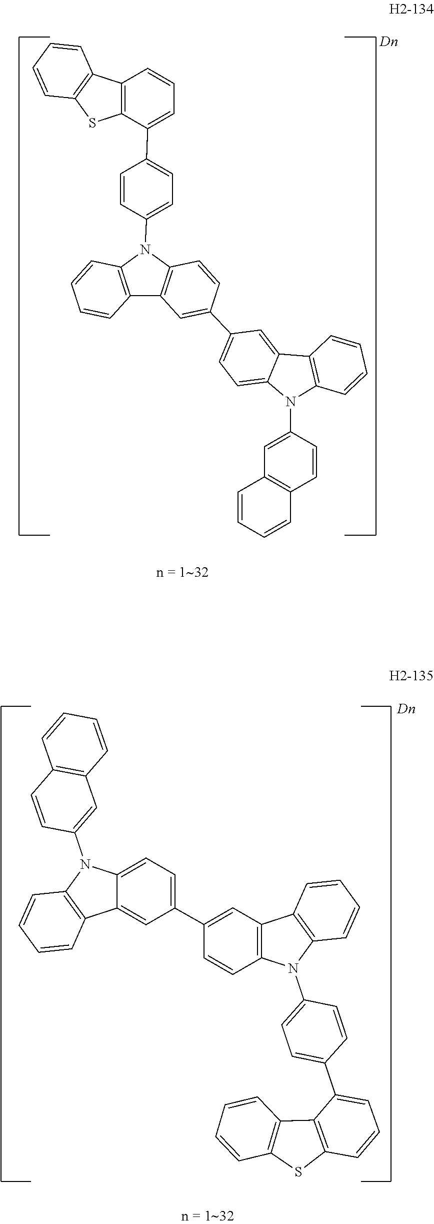 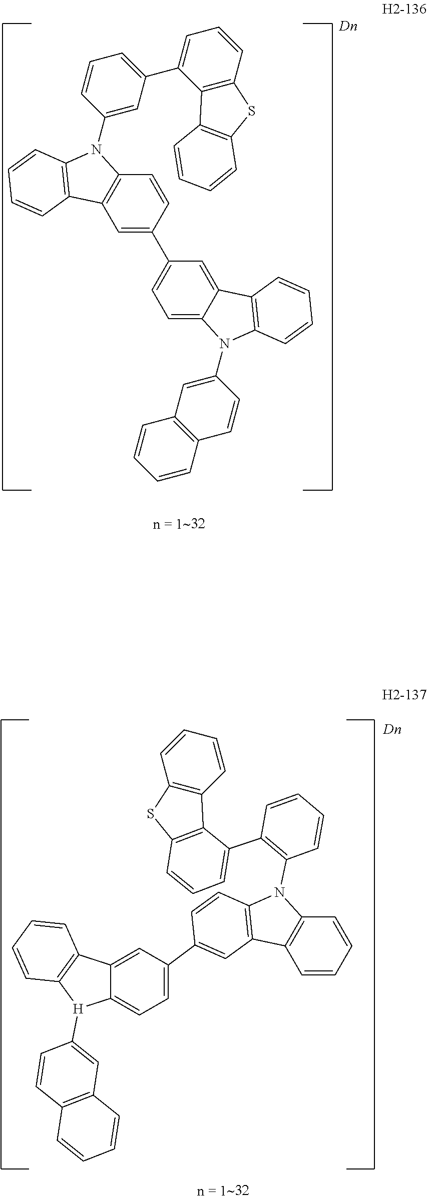 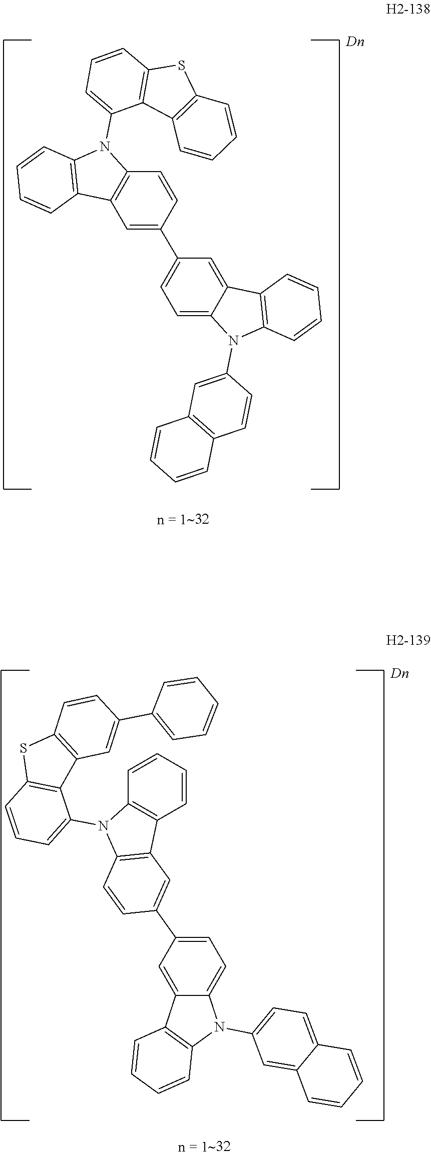 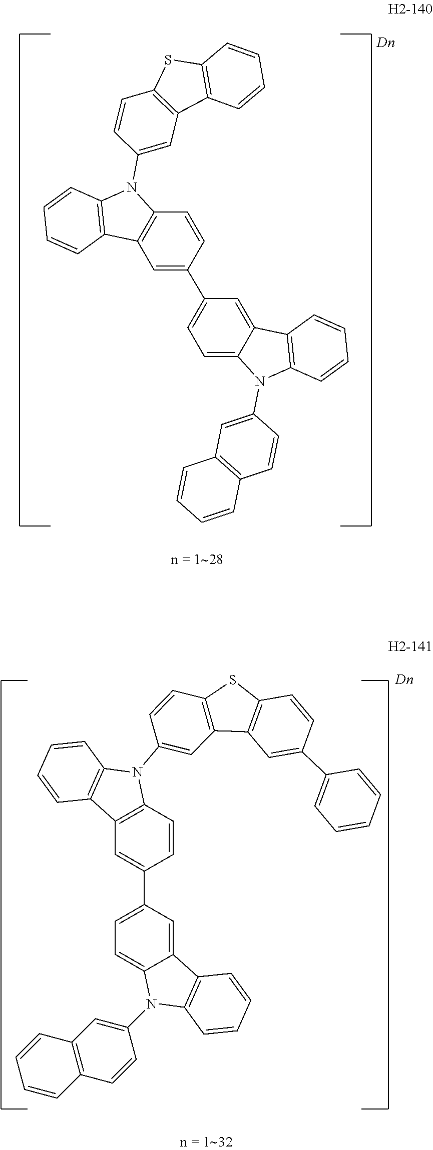 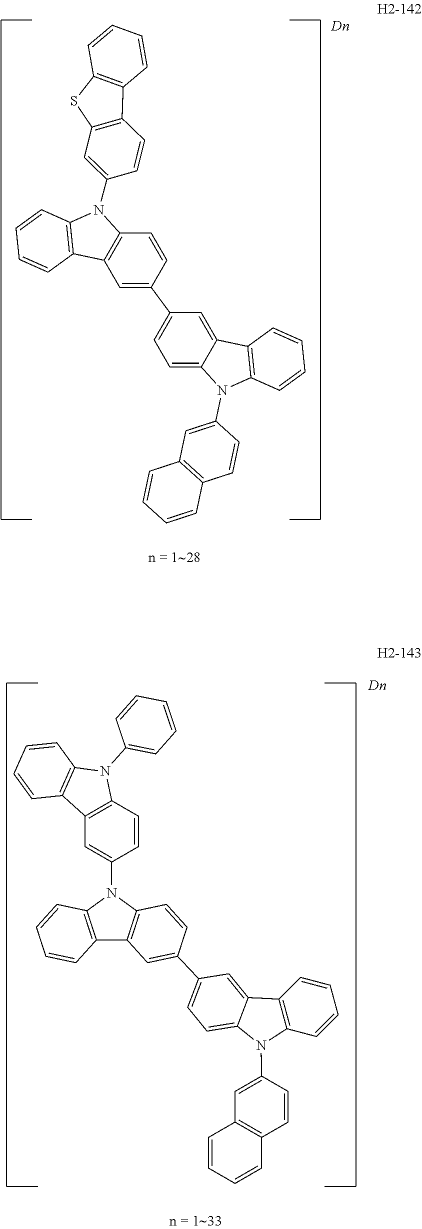 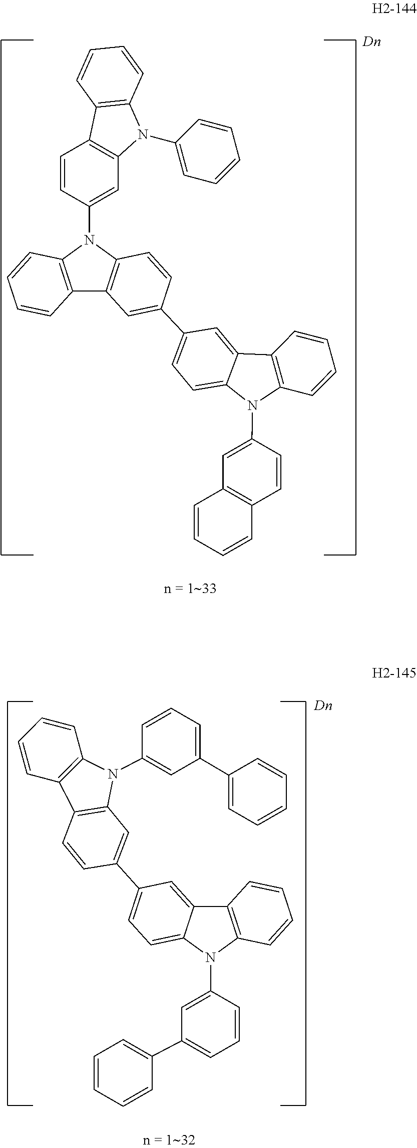 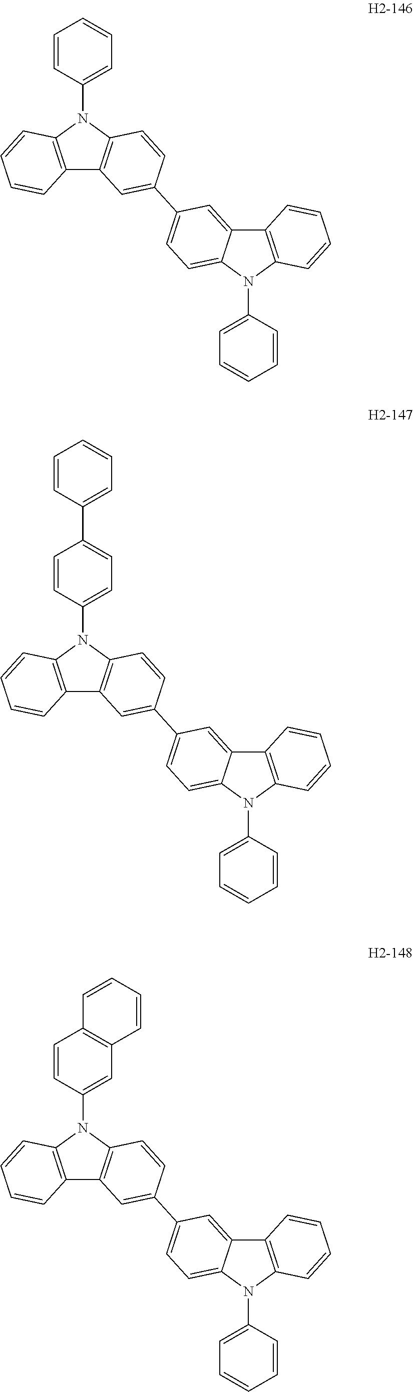 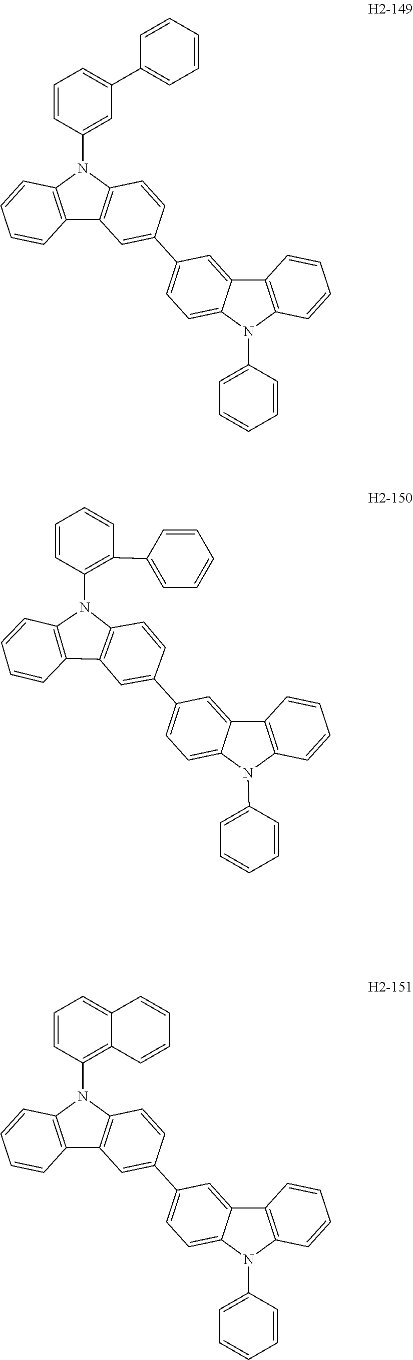 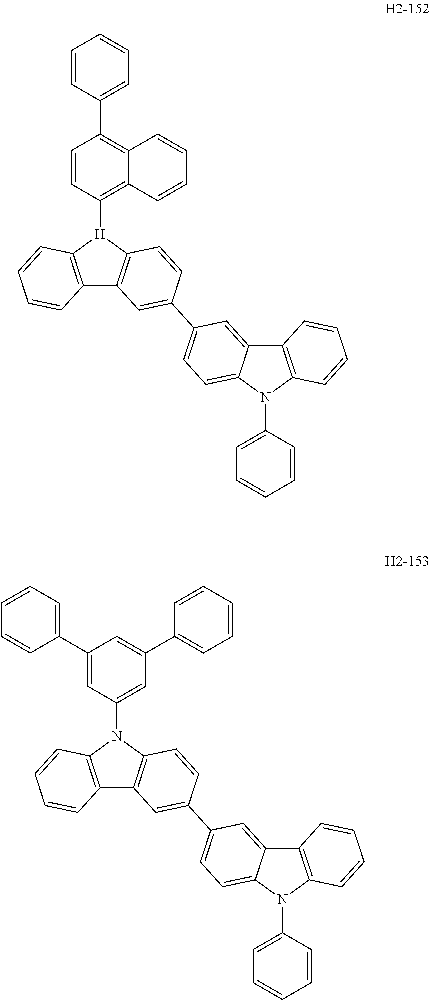 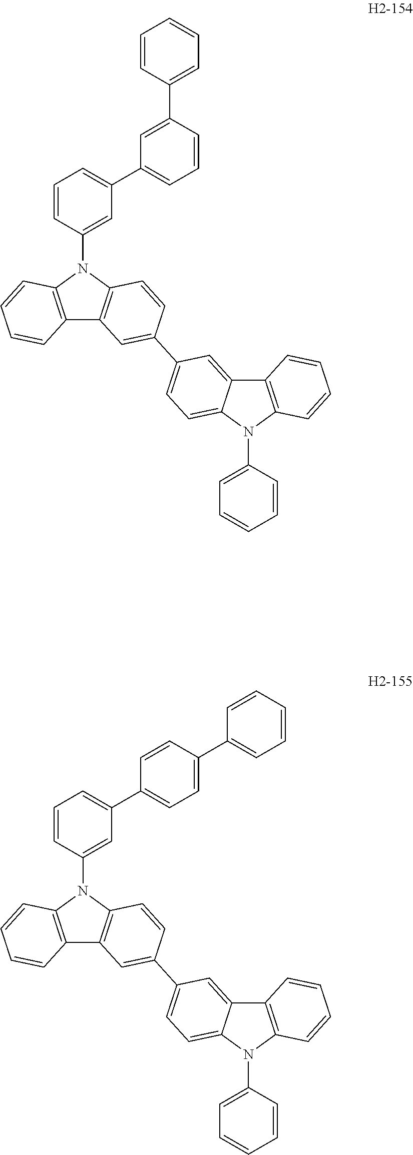 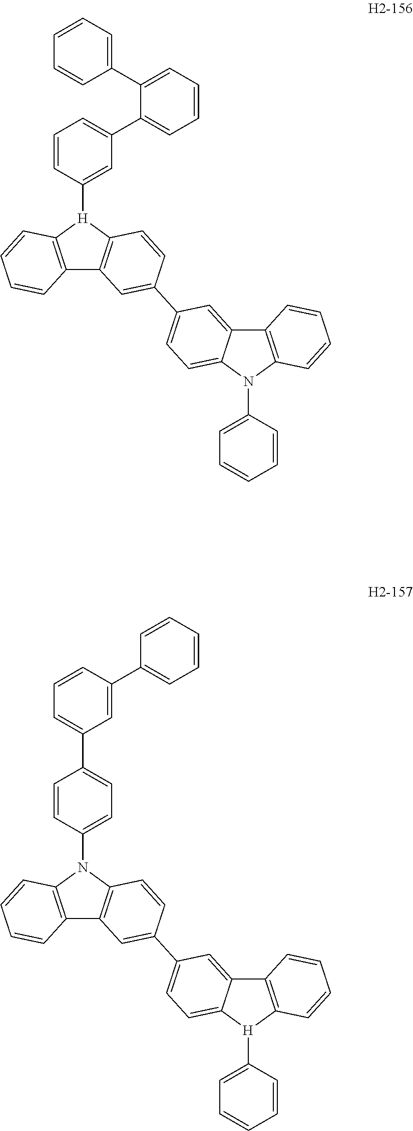 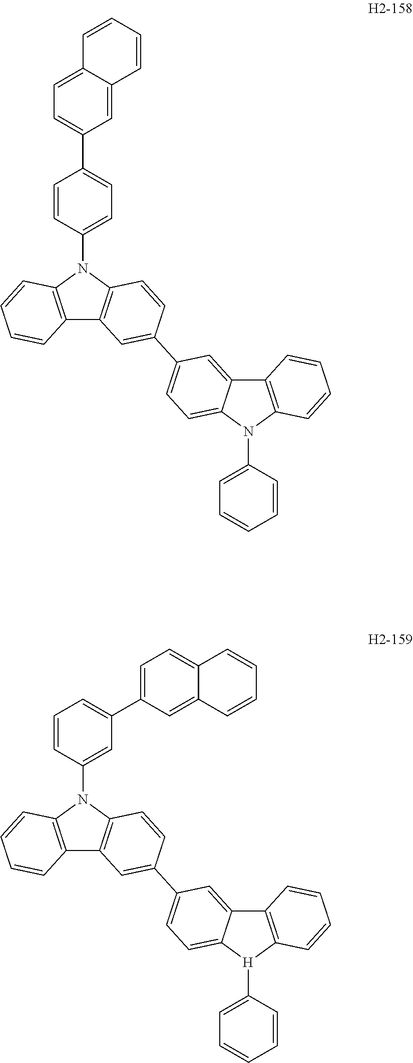 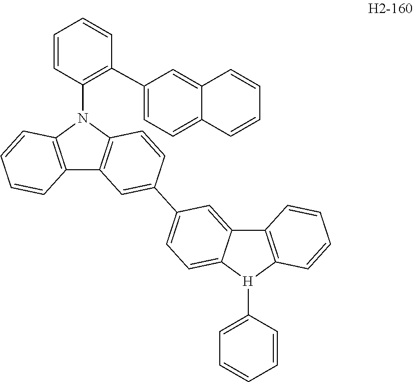 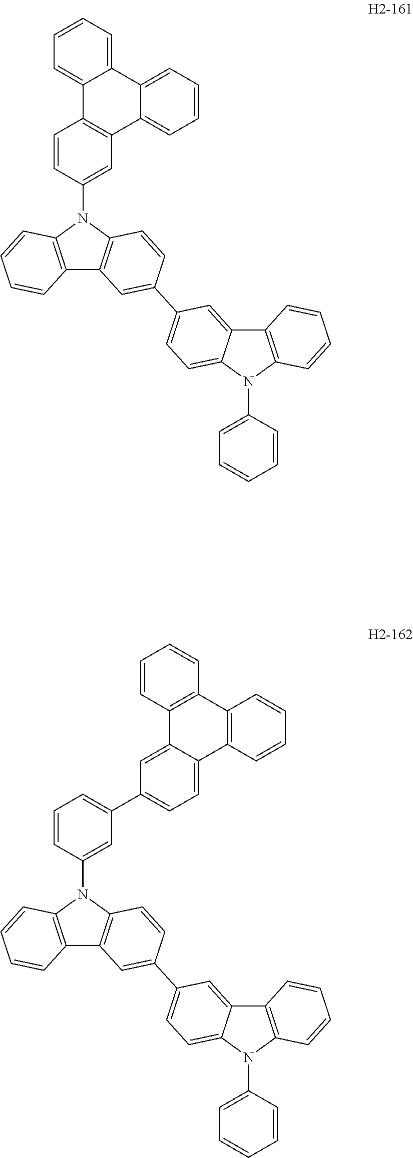 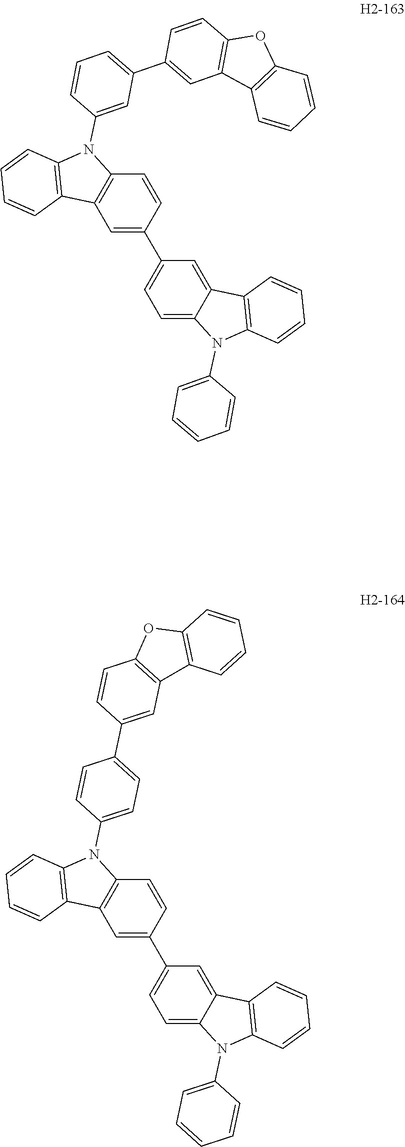 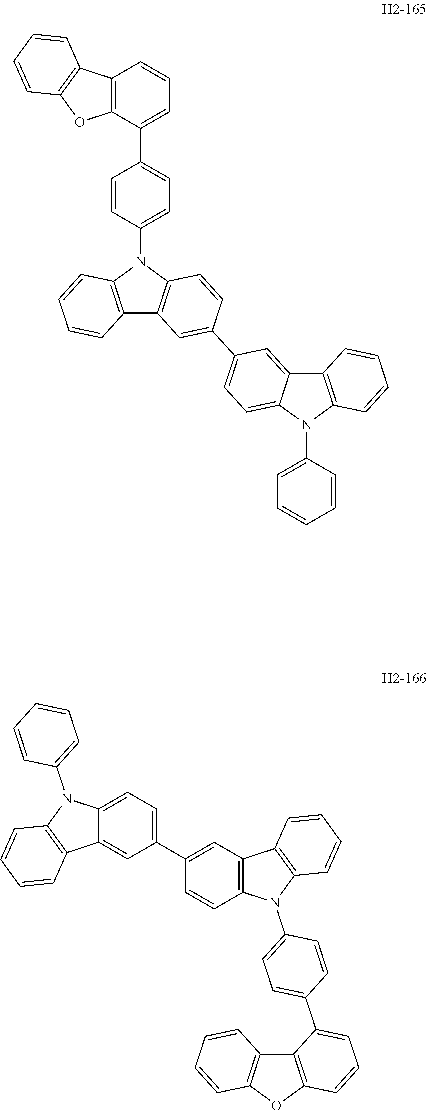 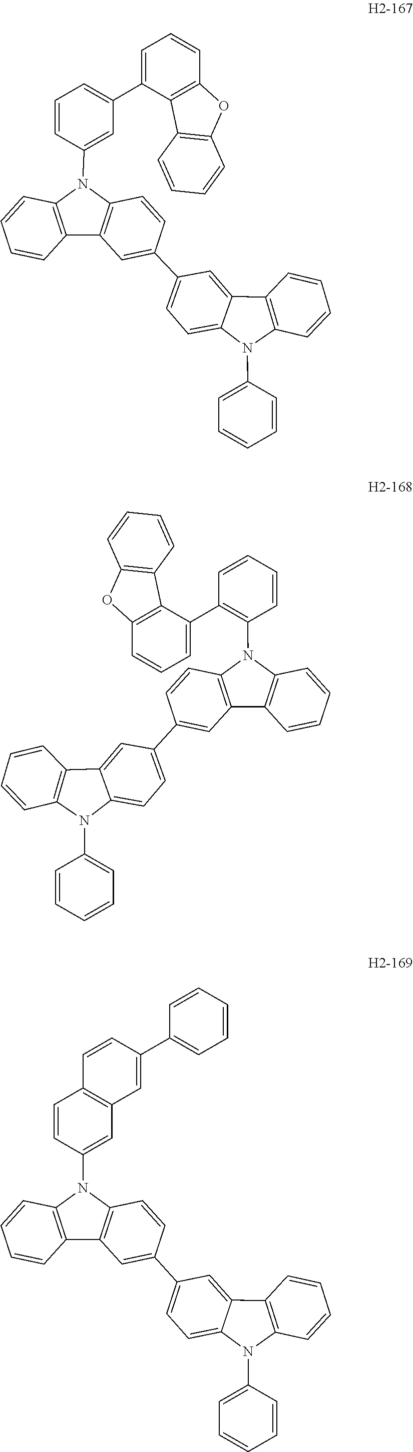 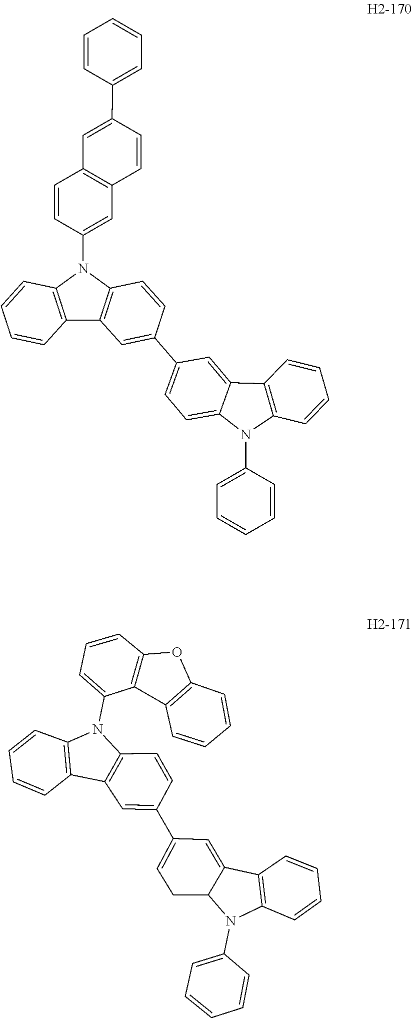 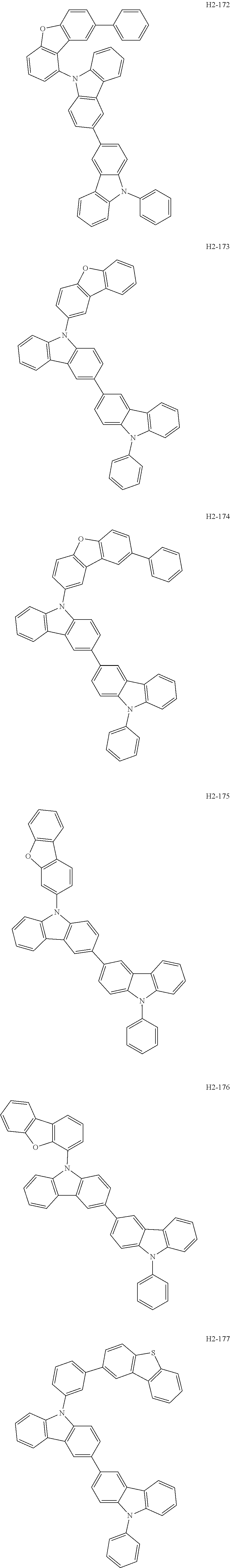 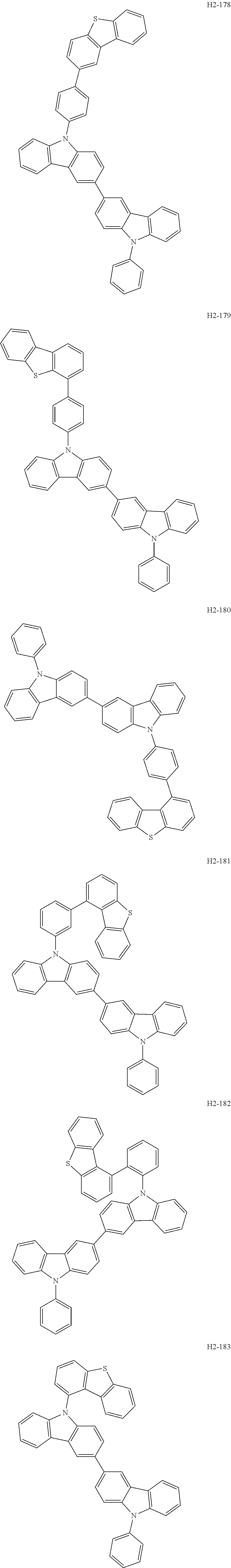 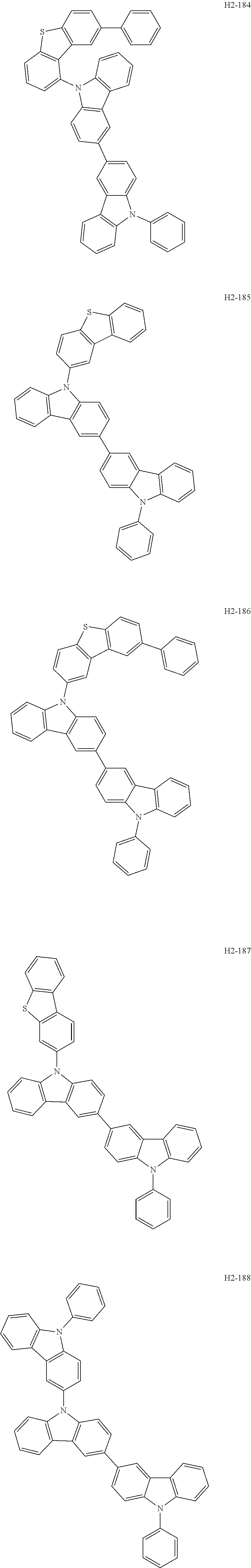 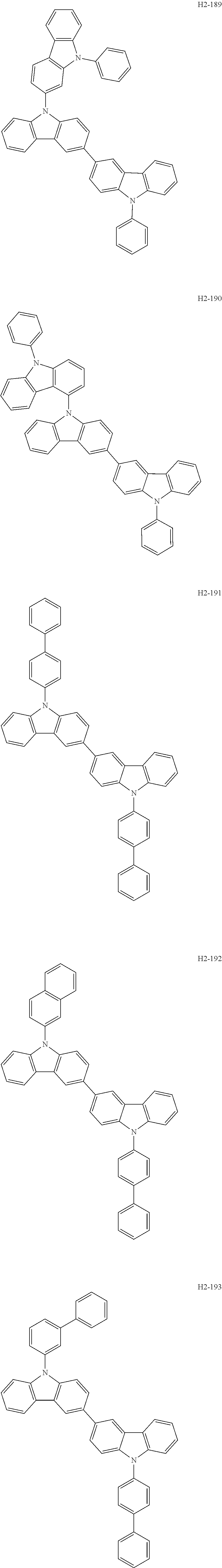 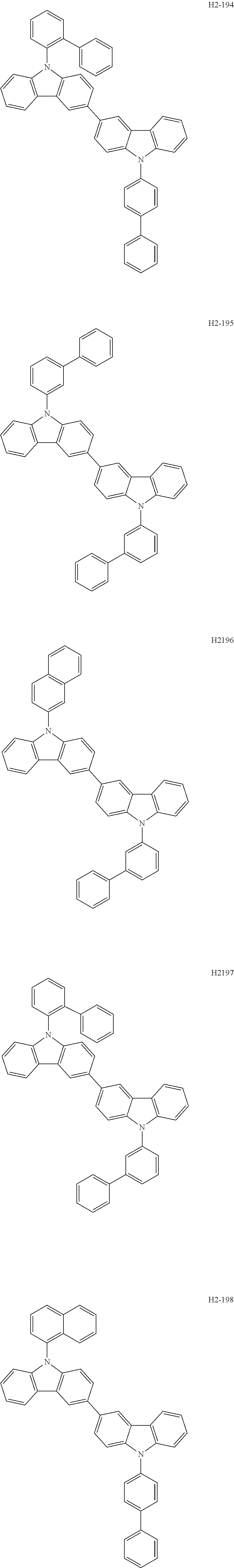 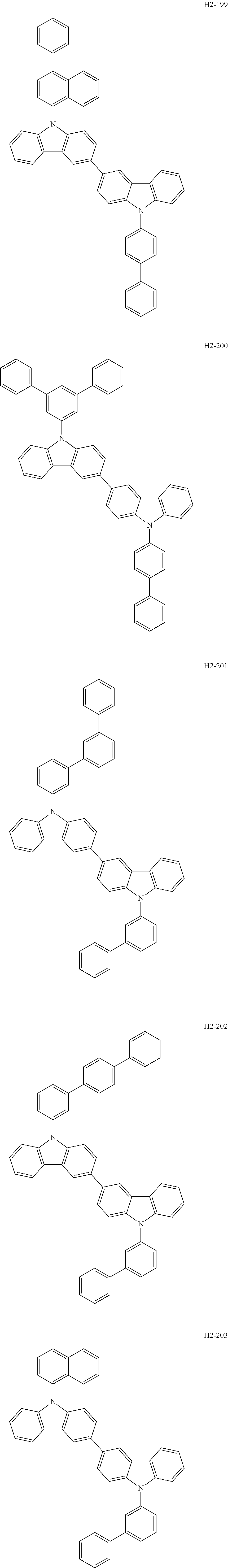 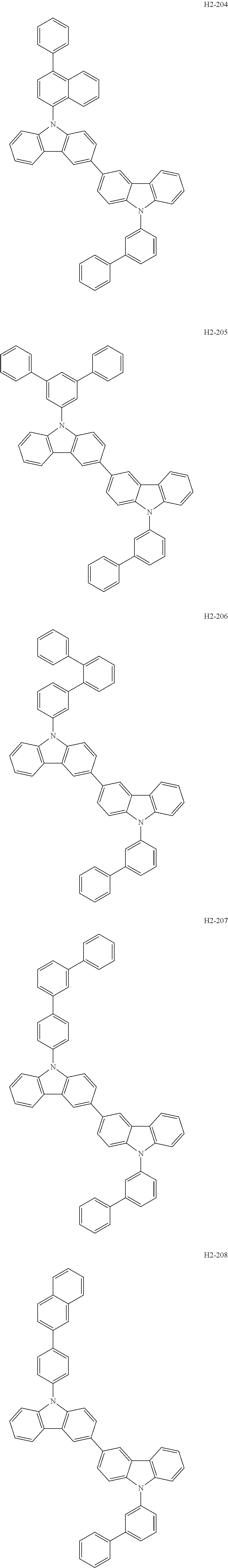 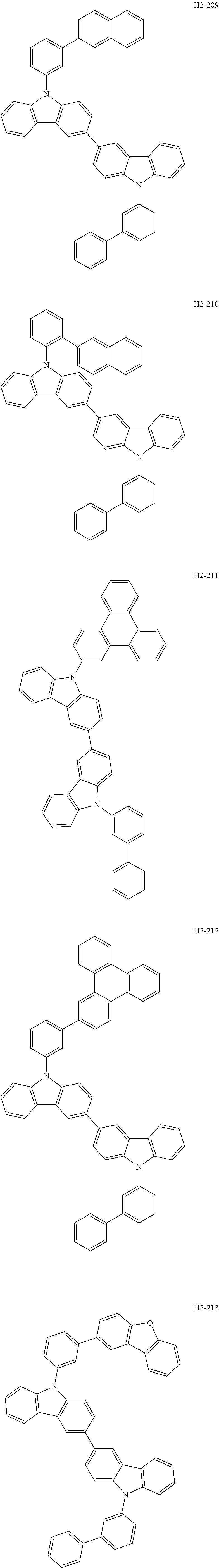 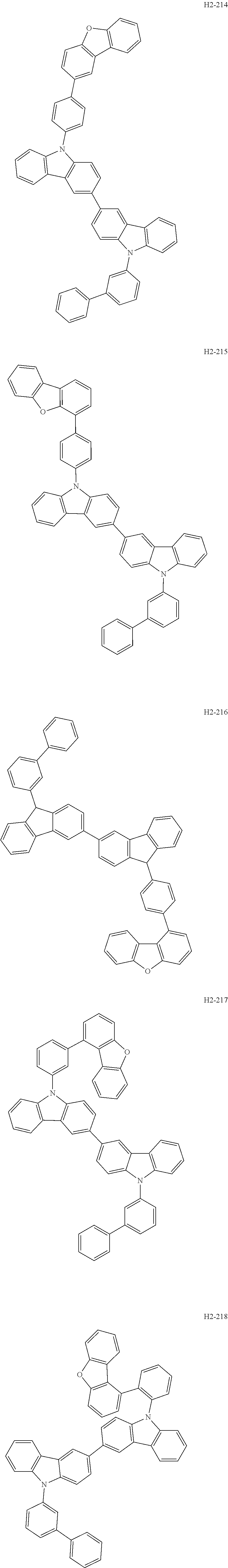 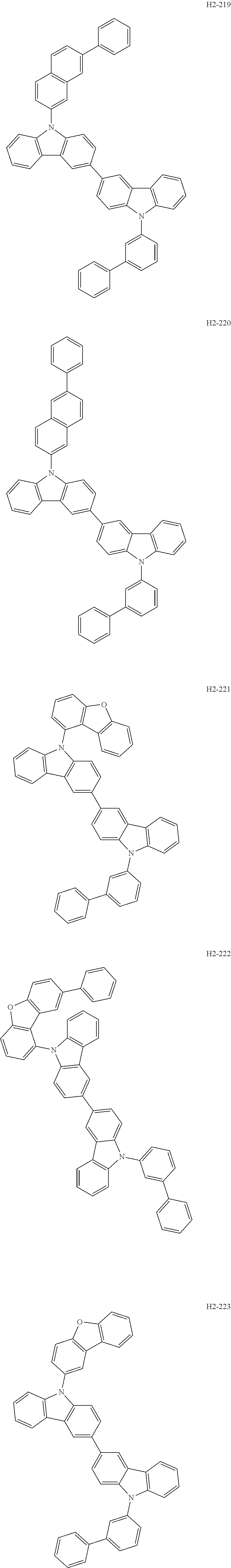 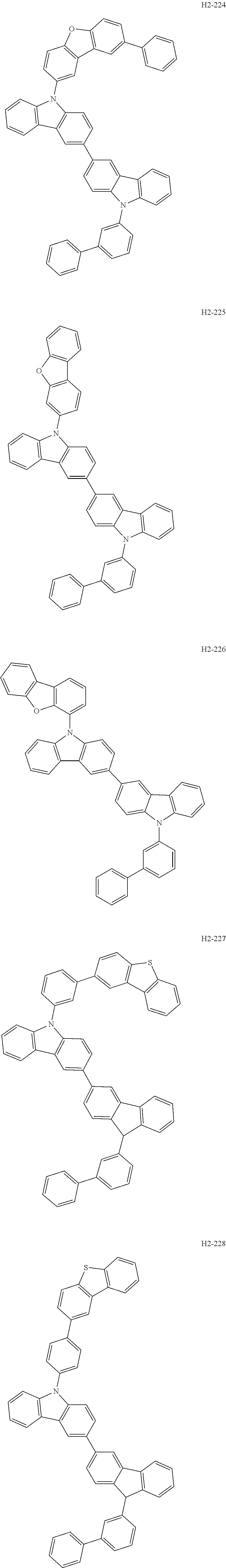 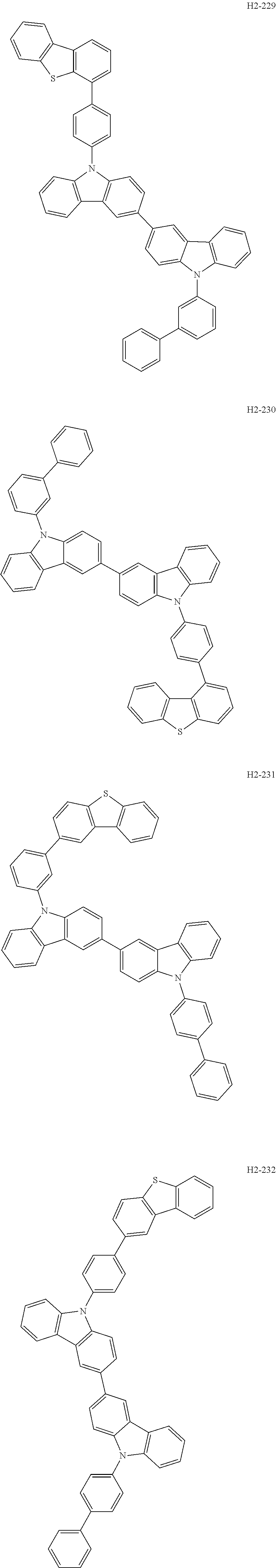 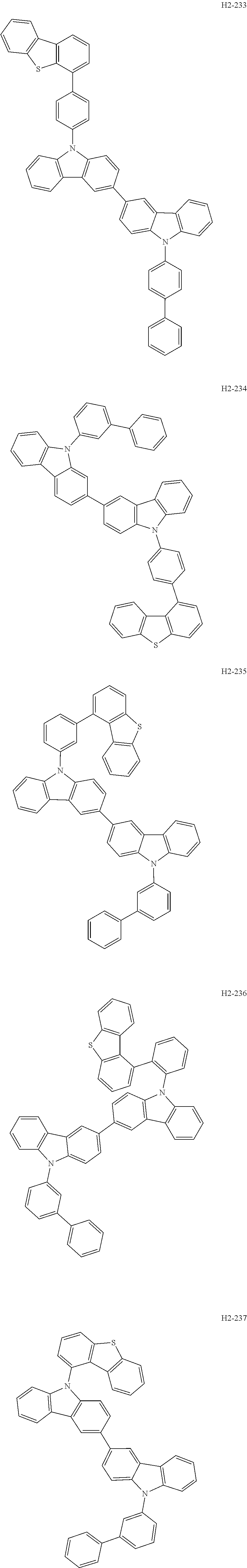 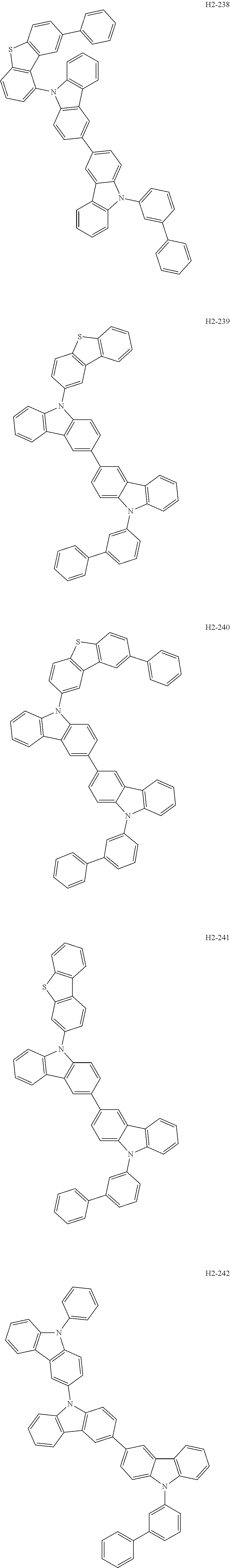 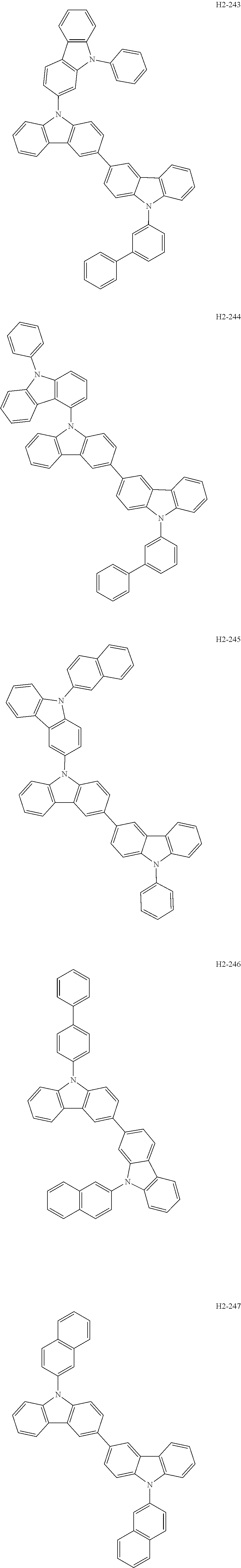 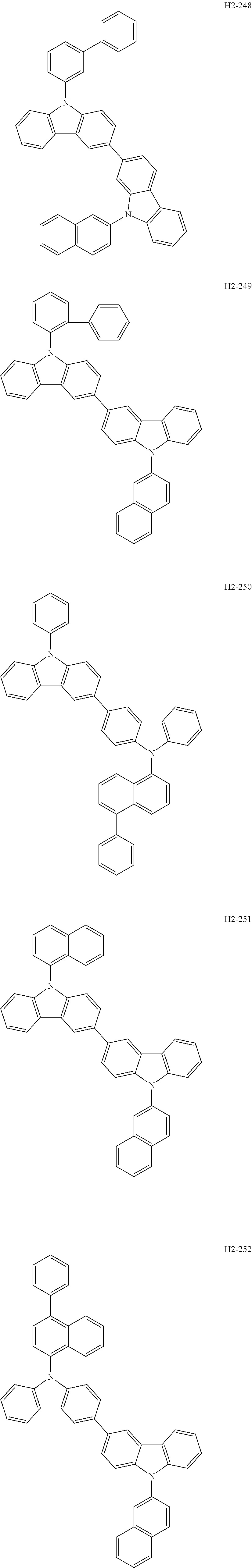 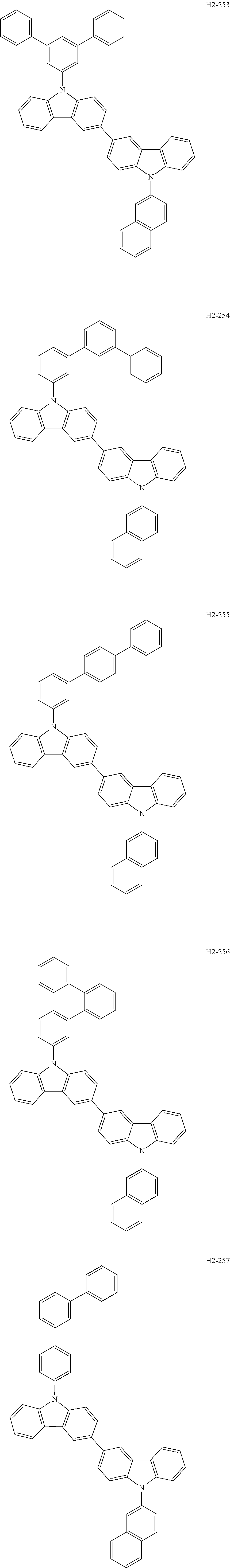 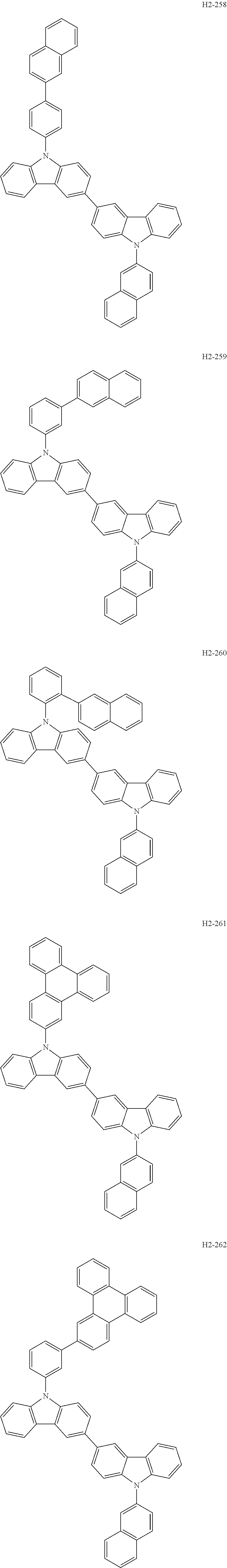 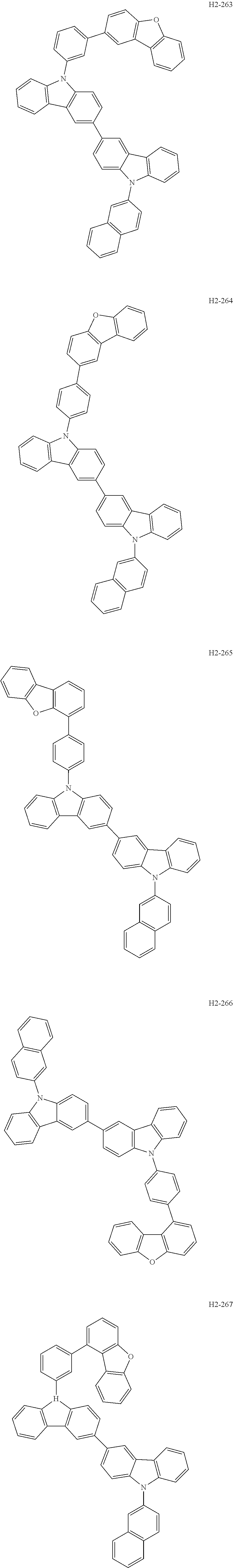 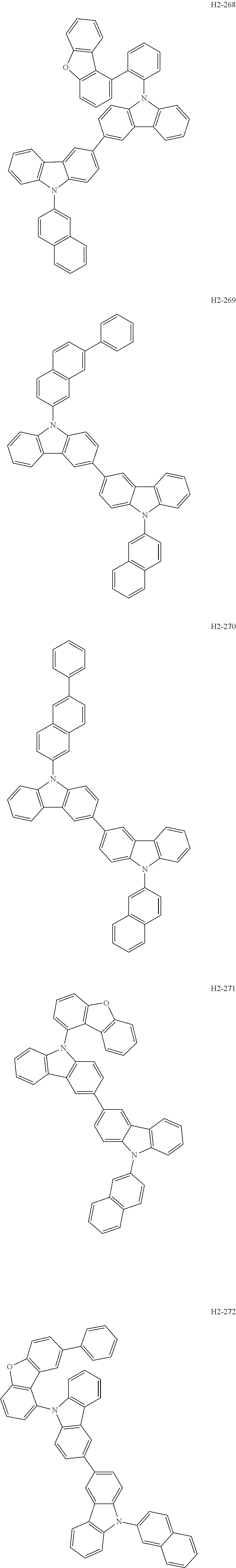 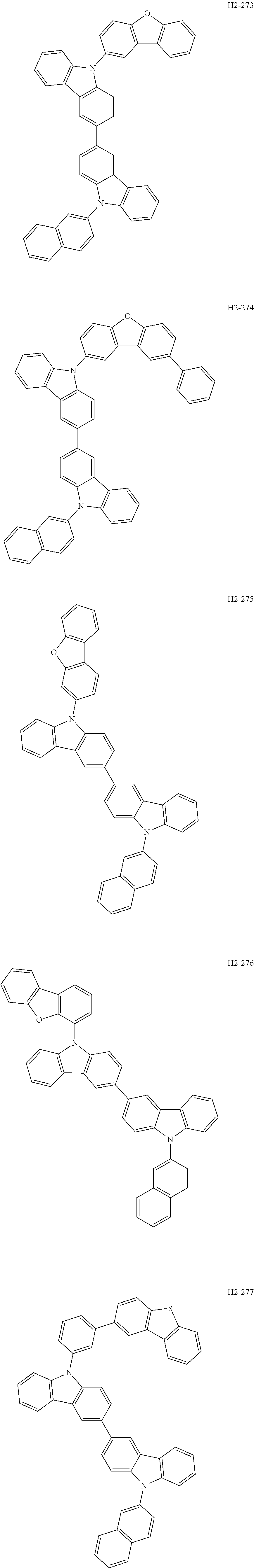 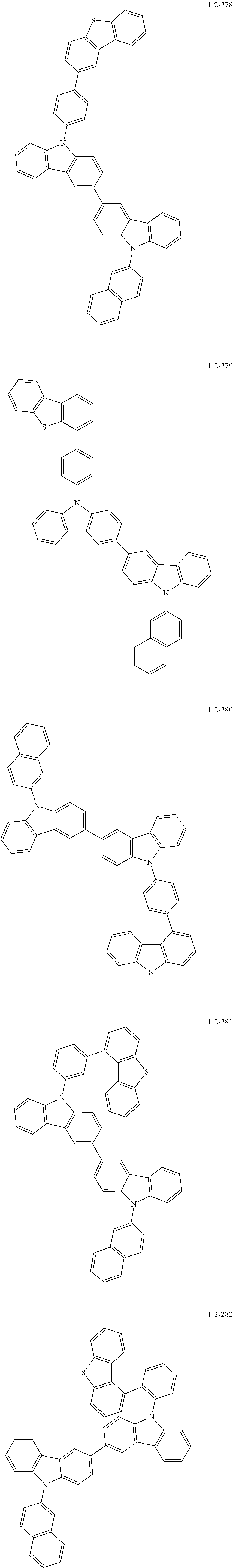 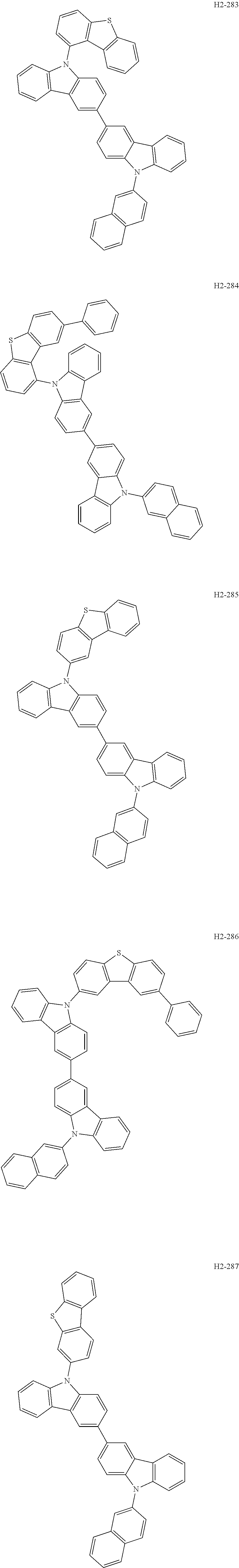 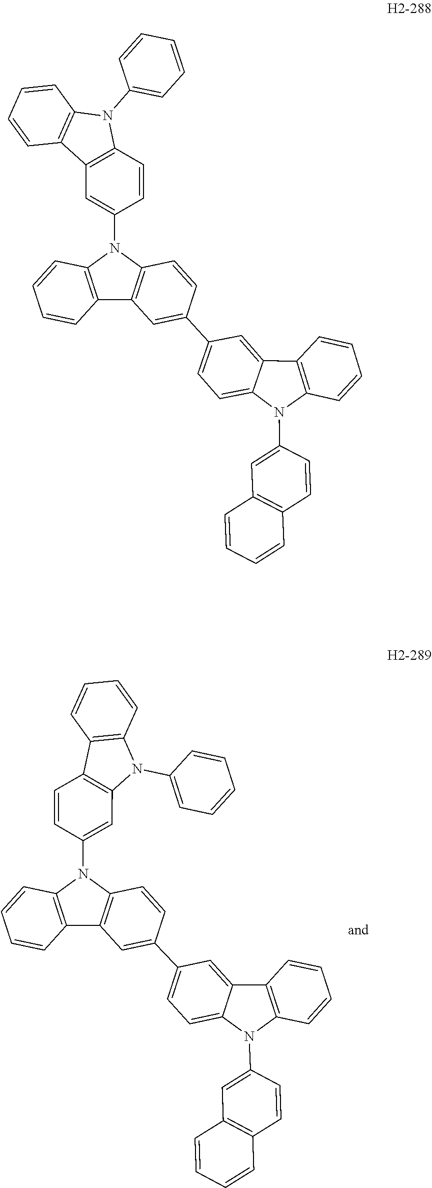 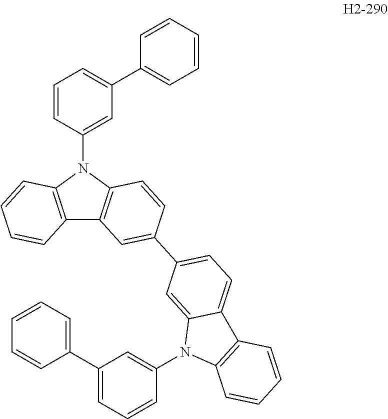
wherein, Din signifies that in hydrogens are replaced with deuterium.

11. An organic electroluminescent compound represented by the following Formula 1: 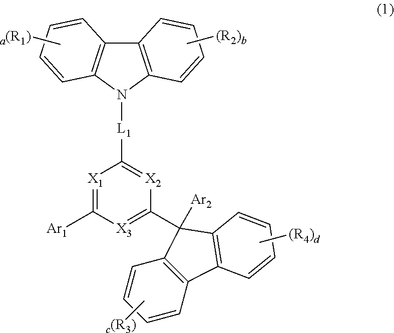
in Formula 1,
X₁ to X₃, each independently, represent N or CRₐ, with a proviso that at least two of X₁ to X₃ represent N;
L₁ represents a single bond, or a substituted or unsubstituted (C6-C30)arylene;
Rₐ, and R₁ to R₄, each independently, represent hydrogen, deuterium, a halogen, a cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30)aromatic ring(s), a substituted or unsubstituted mono- or di- (C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
Ar₁ and Ar₂, each independently, represent hydrogen, deuterium, a substituted or unsubstituted (C6-C30)aryl, or a substituted or unsubstituted (3- to 30-membered)heteroaryl;
a to d, each independently, represent an integer of 1 to 4;
and if a to d represent an integer of 2 or more, each of R₁ to each of R₄ may be the same as or different from each other.

12. The organic electroluminescent compound according to claim 11, wherein the compound represented by Formula 1 is selected from the following compounds:

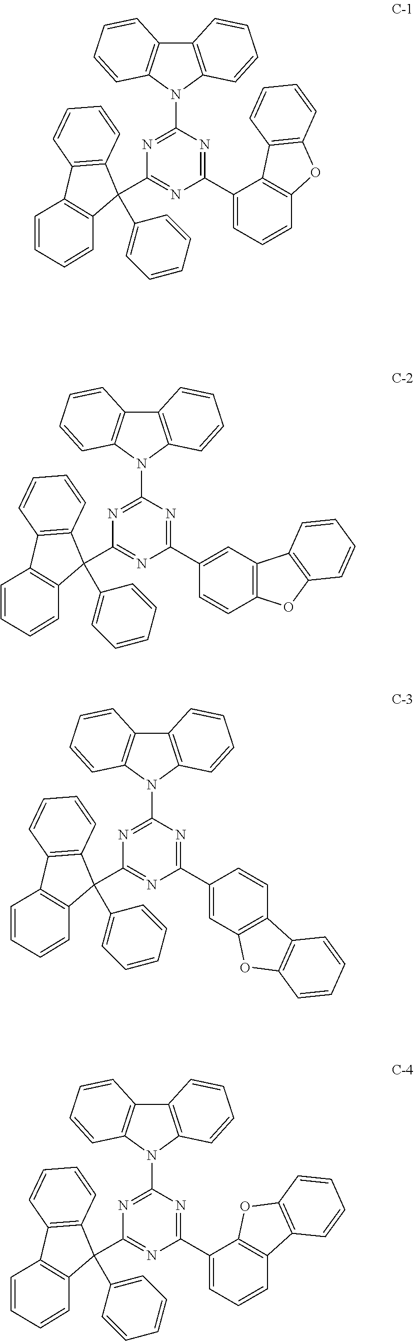 [SMILES: c1ccc(C2(c3nc(-c4cccc5oc6ccccc6c45)nc(-n4c5ccccc5c5ccccc54)n3)c3ccccc3-c3ccccc32)cc1 ; c1ccc(C2(c3nc(-c4ccc5oc6ccccc6c5c4)nc(-n4c5ccccc5c5ccccc54)n3)c3ccccc3-c3ccccc32)cc1 ; c1ccc(C2(c3nc(-c4ccc5c(c4)oc4ccccc45)nc(-n4c5ccccc5c5ccccc54)n3)c3ccccc3-c3ccccc32)cc1 ; c1ccc(C2(c3nc(-c4cccc5c4oc4ccccc45)nc(-n4c5ccccc5c5ccccc54)n3)c3ccccc3-c3ccccc32)cc1]

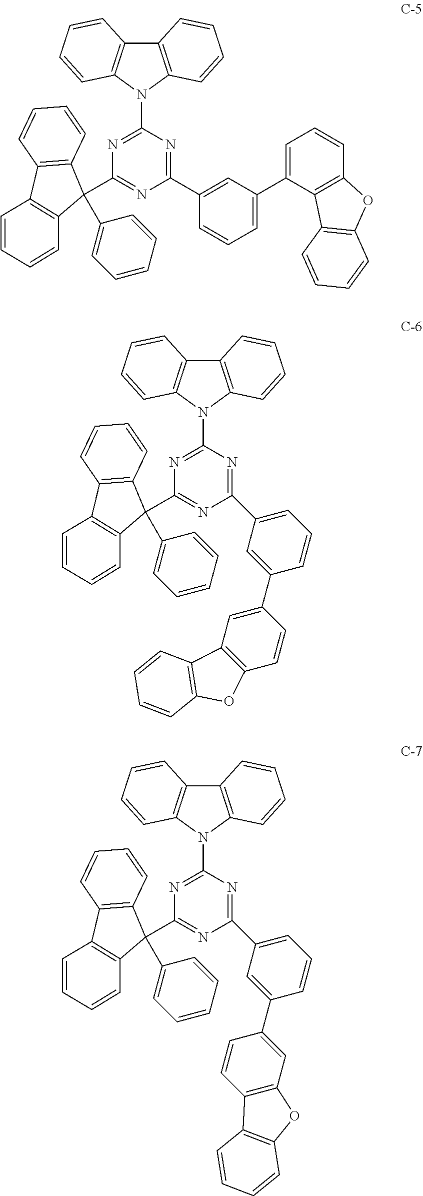

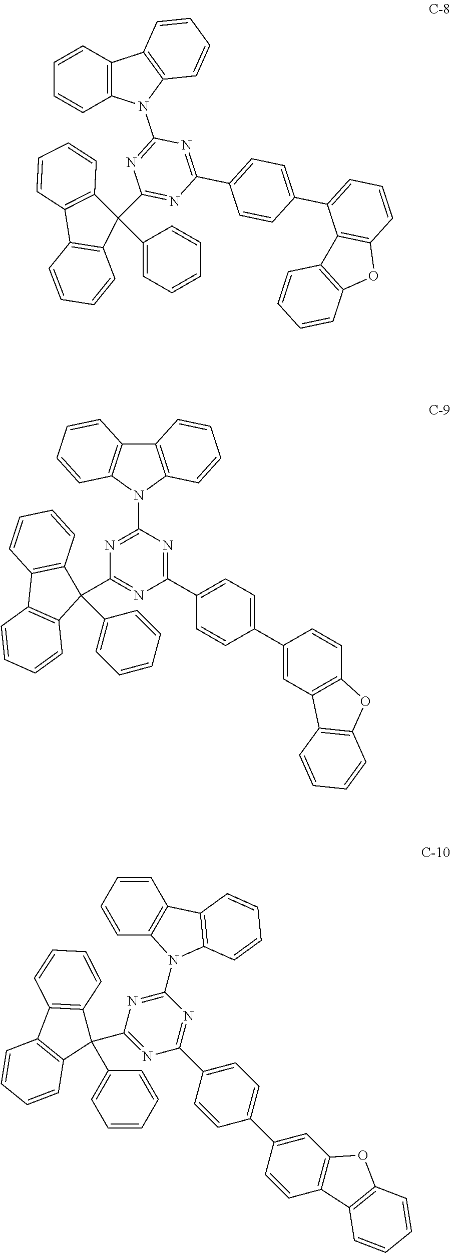 [SMILES: c1ccc(C2(c3nc(-c4ccc(-c5cccc6oc7ccccc7c56)cc4)nc(-n4c5ccccc5c5ccccc54)n3)c3ccccc3-c3ccccc32)cc1 ; c1ccc(C2(c3nc(-c4ccc(-c5ccc6oc7ccccc7c6c5)cc4)nc(-n4c5ccccc5c5ccccc54)n3)c3ccccc3-c3ccccc32)cc1 ; c1ccc(C2(c3nc(-c4ccc(-c5ccc6c(c5)oc5ccccc56)cc4)nc(-n4c5ccccc5c5ccccc54)n3)c3ccccc3-c3ccccc32)cc1]

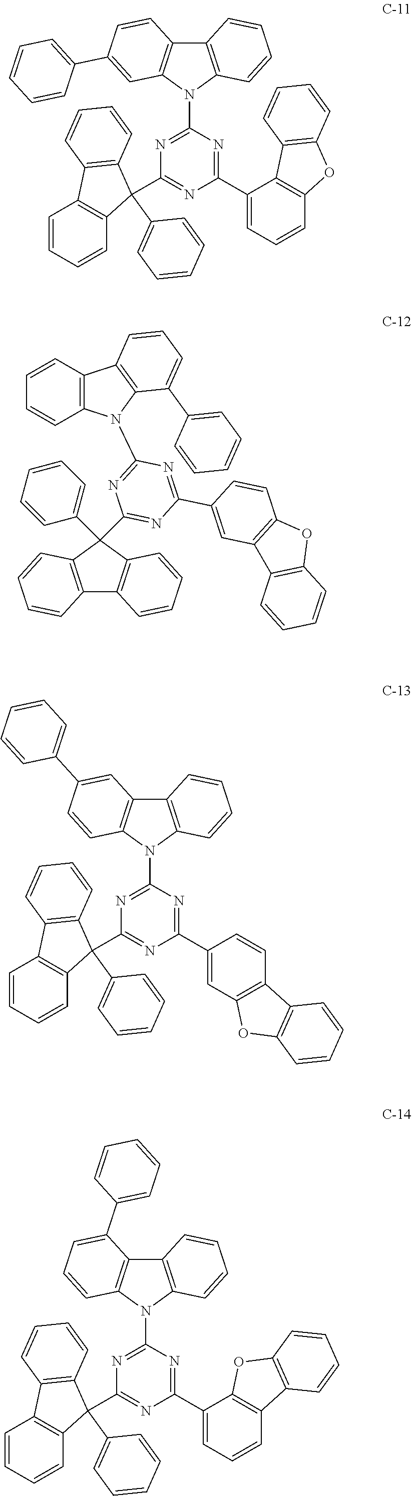 [SMILES: c1ccc(-c2ccc3c4ccccc4n(-c4nc(-c5cccc6oc7ccccc7c56)nc(C5(c6ccccc6)c6ccccc6-c6ccccc65)n4)c3c2)cc1 ; c1ccc(-c2cccc3c4ccccc4n(-c4nc(-c5ccc6oc7ccccc7c6c5)nc(C5(c6ccccc6)c6ccccc6-c6ccccc65)n4)c23)cc1 ; c1ccc(-c2ccc3c(c2)c2ccccc2n3-c2nc(-c3ccc4c(c3)oc3ccccc34)nc(C3(c4ccccc4)c4ccccc4-c4ccccc43)n2)cc1 ; c1ccc(-c2cccc3c2c2ccccc2n3-c2nc(-c3cccc4c3oc3ccccc34)nc(C3(c4ccccc4)c4ccccc4-c4ccccc43)n2)cc1]

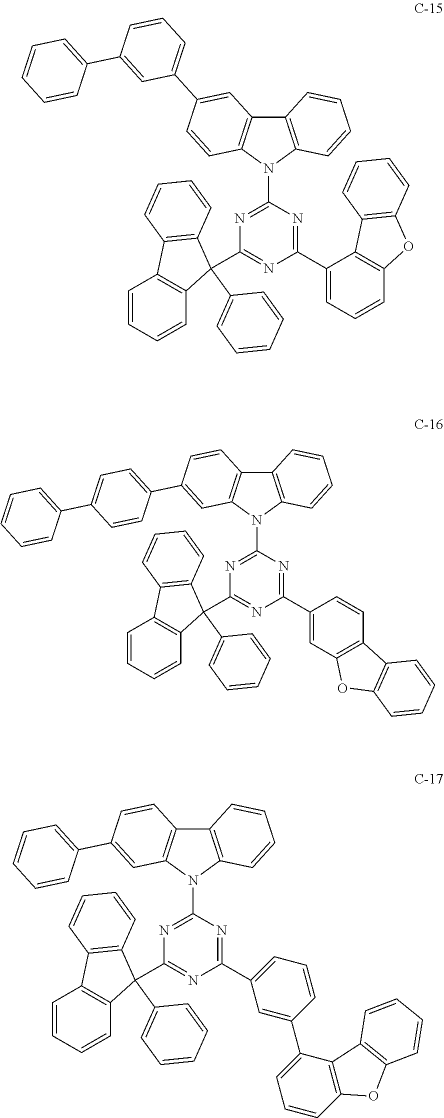 [SMILES: c1ccc(-c2cccc(-c3ccc4c(c3)c3ccccc3n4-c3nc(-c4cccc5oc6ccccc6c45)nc(C4(c5ccccc5)c5ccccc5-c5ccccc54)n3)c2)cc1 ; c1ccc(-c2ccc(-c3ccc4c5ccccc5n(-c5nc(-c6ccc7c(c6)oc6ccccc67)nc(C6(c7ccccc7)c7ccccc7-c7ccccc76)n5)c4c3)cc2)cc1 ; c1ccc(-c2ccc3c4ccccc4n(-c4nc(-c5cccc(-c6cccc7oc8ccccc8c67)c5)nc(C5(c6ccccc6)c6ccccc6-c6ccccc65)n4)c3c2)cc1]

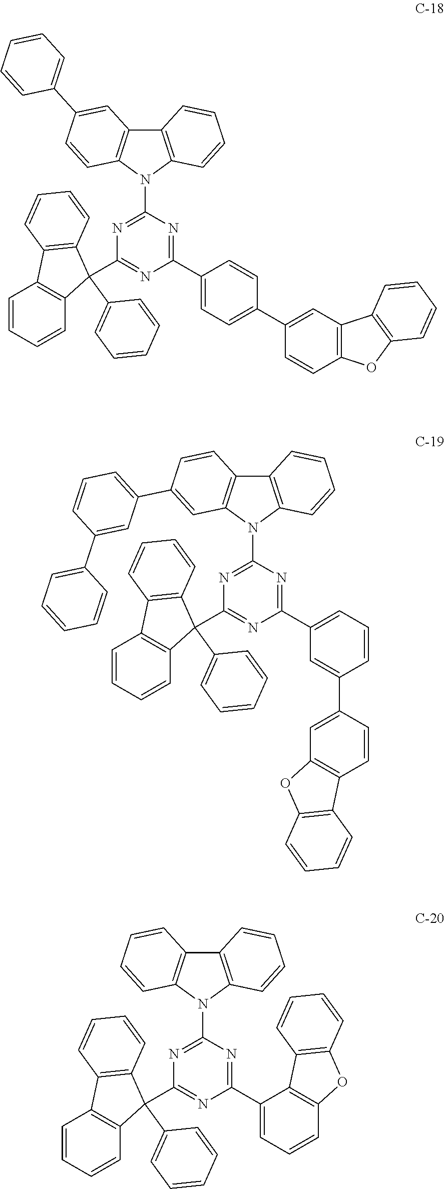 [SMILES: c1ccc(-c2ccc3c(c2)c2ccccc2n3-c2nc(-c3ccc(-c4ccc5oc6ccccc6c5c4)cc3)nc(C3(c4ccccc4)c4ccccc4-c4ccccc43)n2)cc1 ; c1ccc(-c2cccc(-c3ccc4c5ccccc5n(-c5nc(-c6cccc(-c7ccc8c(c7)oc7ccccc78)c6)nc(C6(c7ccccc7)c7ccccc7-c7ccccc76)n5)c4c3)c2)cc1 ; c1ccc(C2(c3nc(-c4cccc5oc6ccccc6c45)nc(-n4c5ccccc5c5ccccc54)n3)c3ccccc3-c3ccccc32)cc1]

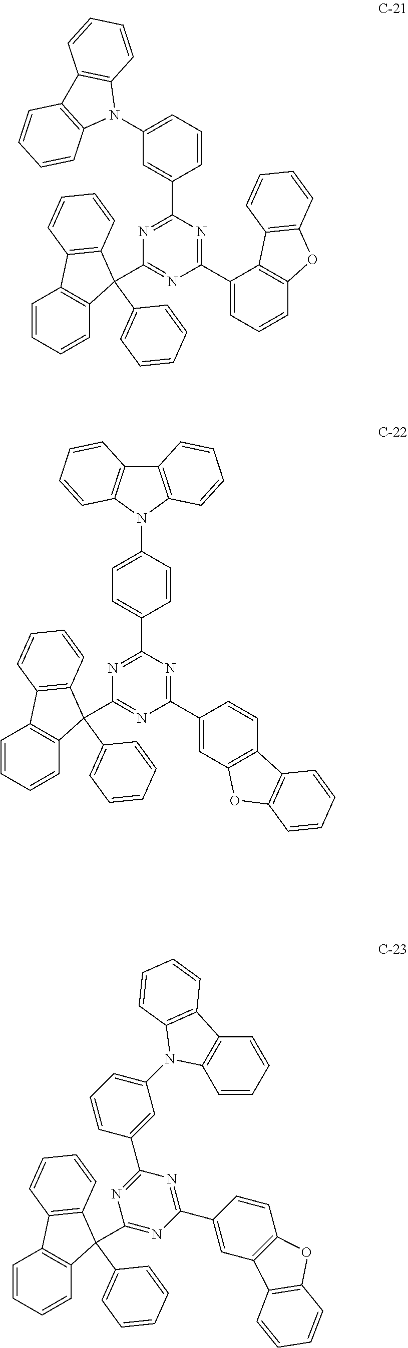 [SMILES: c1ccc(C2(c3nc(-c4cccc(-n5c6ccccc6c6ccccc65)c4)nc(-c4cccc5oc6ccccc6c45)n3)c3ccccc3-c3ccccc32)cc1 ; c1ccc(C2(c3nc(-c4ccc(-n5c6ccccc6c6ccccc65)cc4)nc(-c4ccc5c(c4)oc4ccccc45)n3)c3ccccc3-c3ccccc32)cc1 ; c1ccc(C2(c3nc(-c4cccc(-n5c6ccccc6c6ccccc65)c4)nc(-c4ccc5oc6ccccc6c5c4)n3)c3ccccc3-c3ccccc32)cc1]

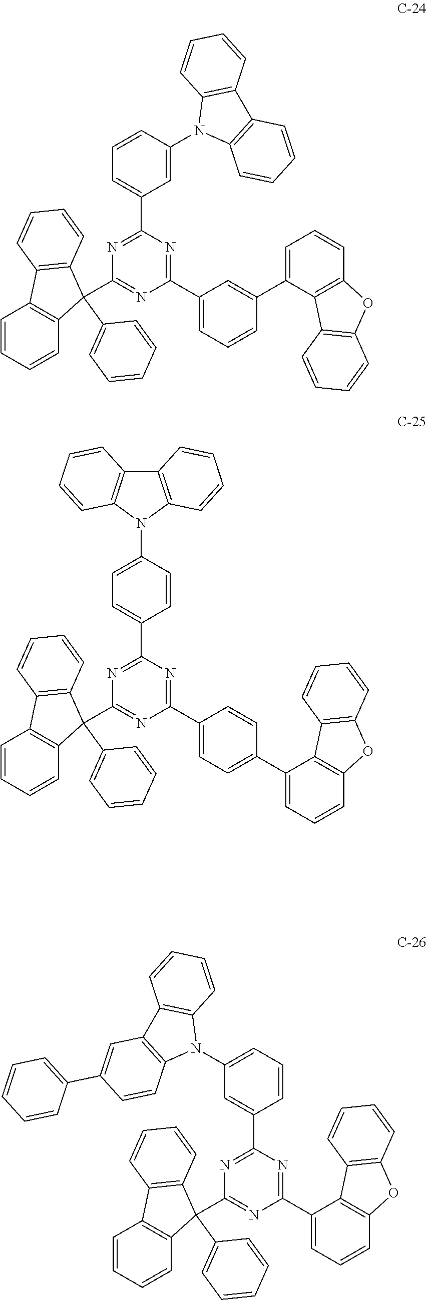 [SMILES: c1ccc(C2(c3nc(-c4cccc(-c5cccc6oc7ccccc7c56)c4)nc(-c4cccc(-n5c6ccccc6c6ccccc65)c4)n3)c3ccccc3-c3ccccc32)cc1 ; c1ccc(C2(c3nc(-c4ccc(-c5cccc6oc7ccccc7c56)cc4)nc(-c4ccc(-n5c6ccccc6c6ccccc65)cc4)n3)c3ccccc3-c3ccccc32)cc1 ; c1ccc(-c2ccc3c(c2)c2ccccc2n3-c2cccc(-c3nc(-c4cccc5oc6ccccc6c45)nc(C4(c5ccccc5)c5ccccc5-c5ccccc54)n3)c2)cc1]

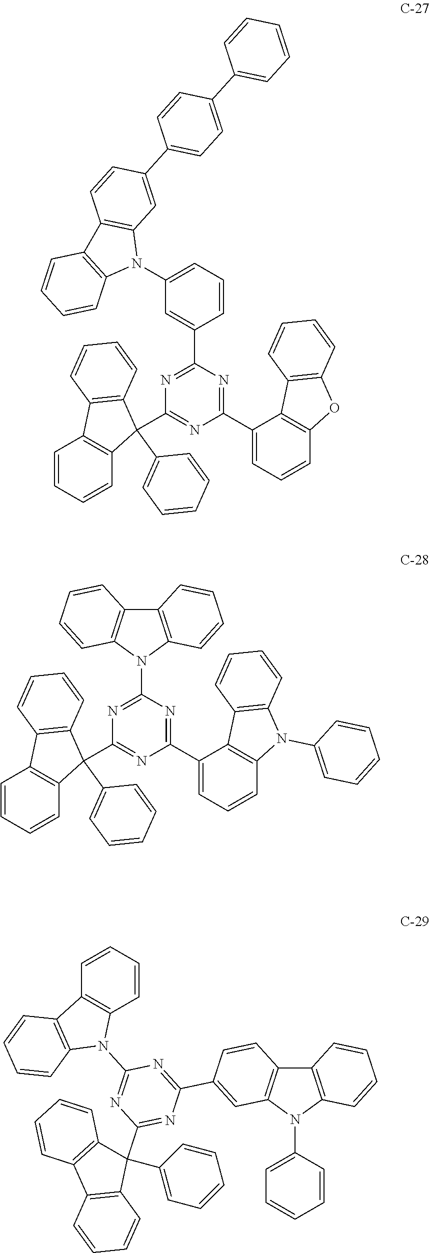

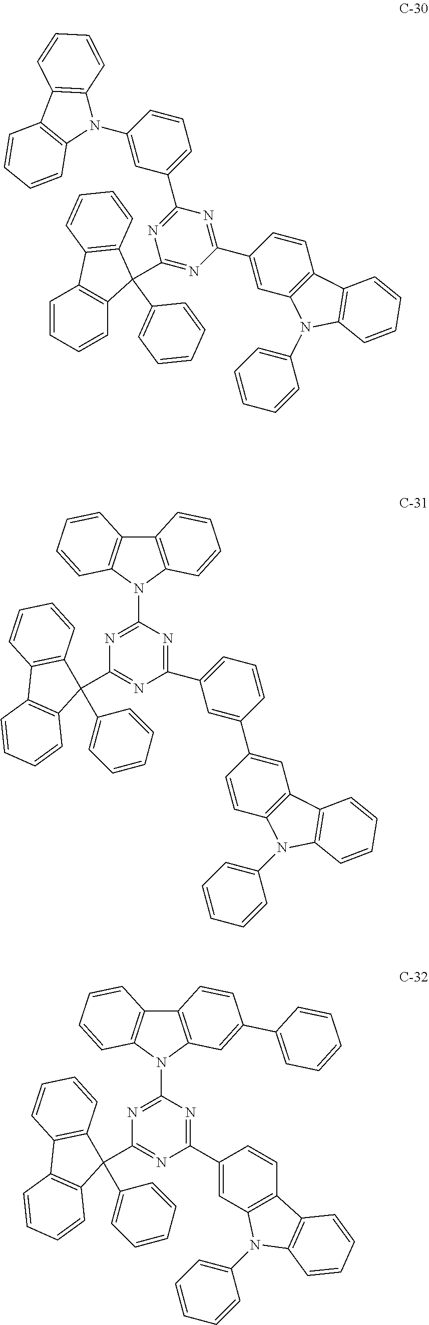 [SMILES: c1ccc(-n2c3ccccc3c3ccc(-c4nc(-c5cccc(-n6c7ccccc7c7ccccc76)c5)nc(C5(c6ccccc6)c6ccccc6-c6ccccc65)n4)cc32)cc1 ; c1ccc(-n2c3ccccc3c3cc(-c4cccc(-c5nc(-n6c7ccccc7c7ccccc76)nc(C6(c7ccccc7)c7ccccc7-c7ccccc76)n5)c4)ccc32)cc1 ; c1ccc(-c2ccc3c4ccccc4n(-c4nc(-c5ccc6c7ccccc7n(-c7ccccc7)c6c5)nc(C5(c6ccccc6)c6ccccc6-c6ccccc65)n4)c3c2)cc1]

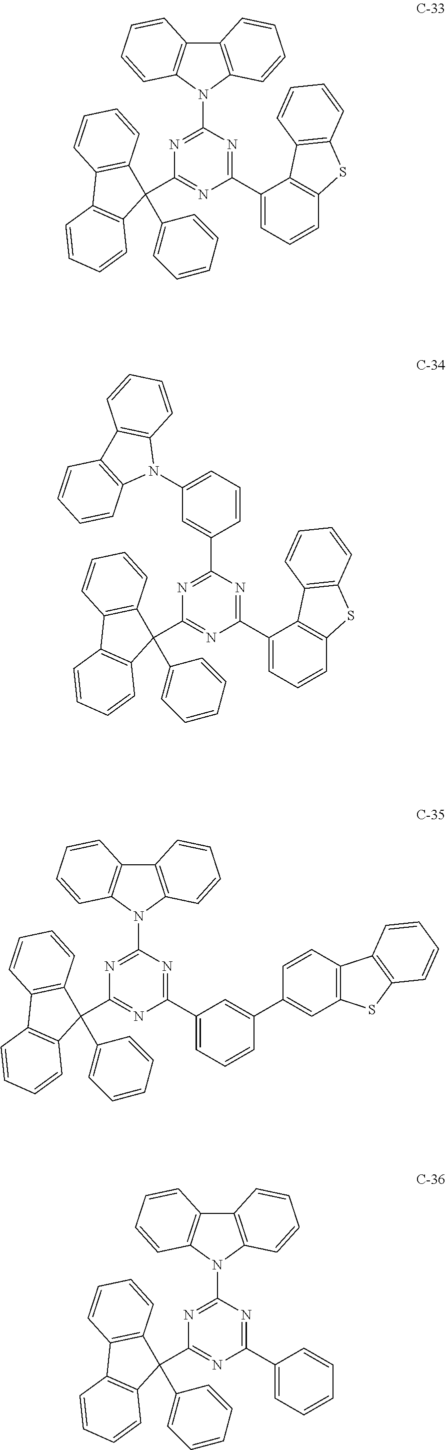 [SMILES: c1ccc(C2(c3nc(-c4cccc5sc6ccccc6c45)nc(-n4c5ccccc5c5ccccc54)n3)c3ccccc3-c3ccccc32)cc1 ; c1ccc(C2(c3nc(-c4cccc(-n5c6ccccc6c6ccccc65)c4)nc(-c4cccc5sc6ccccc6c45)n3)c3ccccc3-c3ccccc32)cc1 ; c1ccc(C2(c3nc(-c4cccc(-c5ccc6c(c5)sc5ccccc56)c4)nc(-n4c5ccccc5c5ccccc54)n3)c3ccccc3-c3ccccc32)cc1 ; c1ccc(-c2nc(-n3c4ccccc4c4ccccc43)nc(C3(c4ccccc4)c4ccccc4-c4ccccc43)n2)cc1]

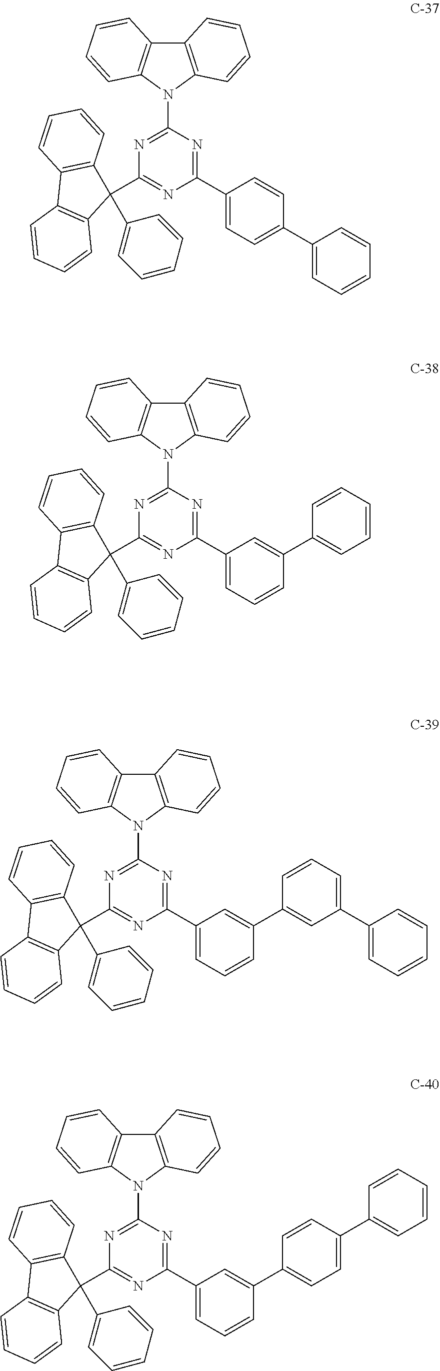 [SMILES: c1ccc(-c2ccc(-c3nc(-n4c5ccccc5c5ccccc54)nc(C4(c5ccccc5)c5ccccc5-c5ccccc54)n3)cc2)cc1 ; c1ccc(-c2cccc(-c3nc(-n4c5ccccc5c5ccccc54)nc(C4(c5ccccc5)c5ccccc5-c5ccccc54)n3)c2)cc1 ; c1ccc(-c2cccc(-c3cccc(-c4nc(-n5c6ccccc6c6ccccc65)nc(C5(c6ccccc6)c6ccccc6-c6ccccc65)n4)c3)c2)cc1 ; c1ccc(-c2ccc(-c3cccc(-c4nc(-n5c6ccccc6c6ccccc65)nc(C5(c6ccccc6)c6ccccc6-c6ccccc65)n4)c3)cc2)cc1]

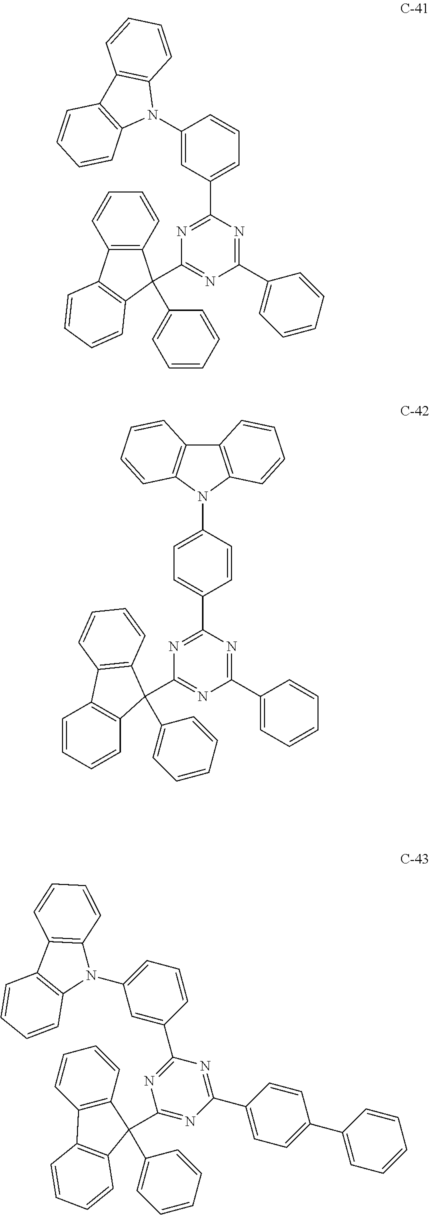 [SMILES: c1ccc(-c2nc(-c3cccc(-n4c5ccccc5c5ccccc54)c3)nc(C3(c4ccccc4)c4ccccc4-c4ccccc43)n2)cc1 ; c1ccc(-c2nc(-c3ccc(-n4c5ccccc5c5ccccc54)cc3)nc(C3(c4ccccc4)c4ccccc4-c4ccccc43)n2)cc1 ; c1ccc(-c2ccc(-c3nc(-c4cccc(-n5c6ccccc6c6ccccc65)c4)nc(C4(c5ccccc5)c5ccccc5-c5ccccc54)n3)cc2)cc1]

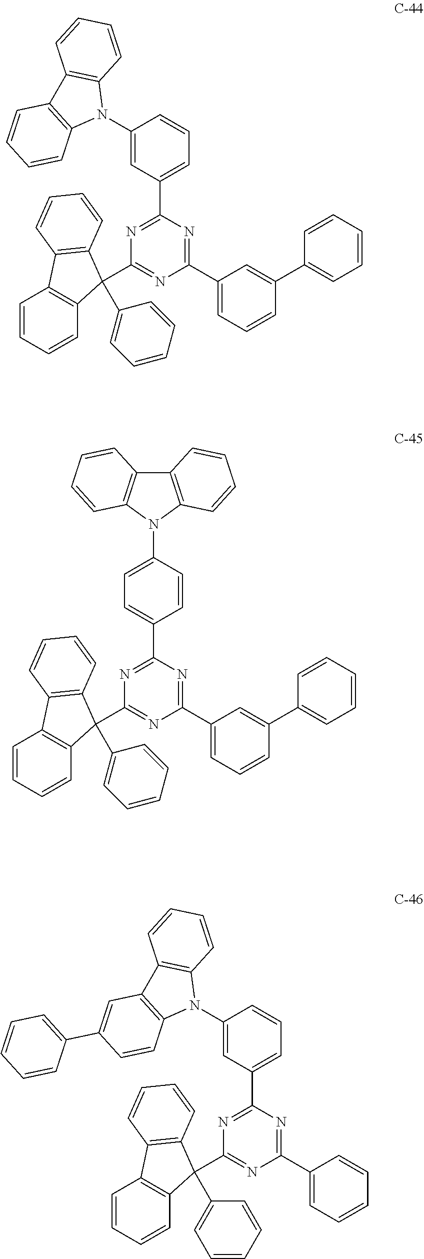 [SMILES: c1ccc(-c2cccc(-c3nc(-c4cccc(-n5c6ccccc6c6ccccc65)c4)nc(C4(c5ccccc5)c5ccccc5-c5ccccc54)n3)c2)cc1 ; c1ccc(-c2cccc(-c3nc(-c4ccc(-n5c6ccccc6c6ccccc65)cc4)nc(C4(c5ccccc5)c5ccccc5-c5ccccc54)n3)c2)cc1 ; c1ccc(-c2ccc3c(c2)c2ccccc2n3-c2cccc(-c3nc(-c4ccccc4)nc(C4(c5ccccc5)c5ccccc5-c5ccccc54)n3)c2)cc1]

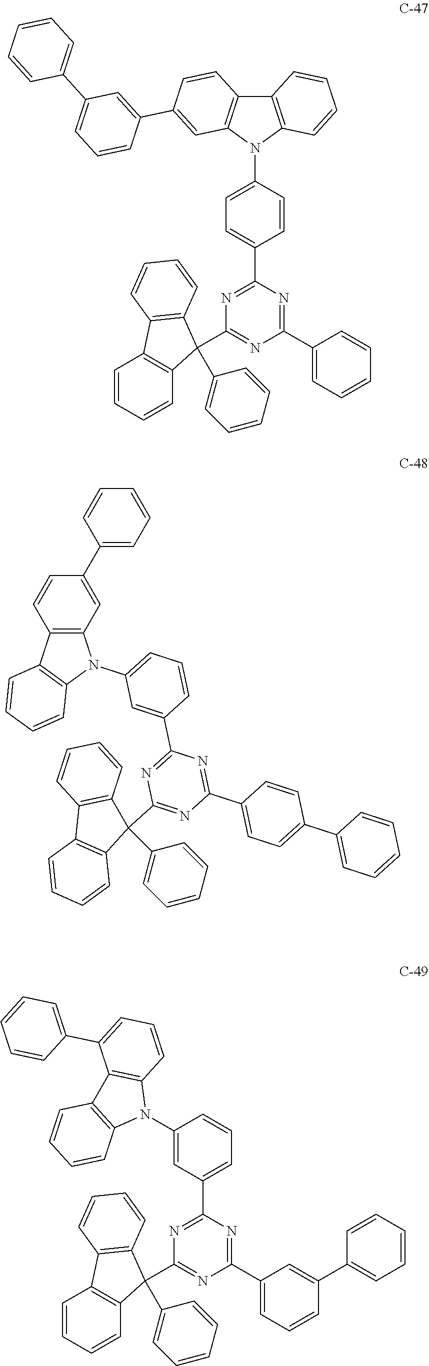 [SMILES: c1ccc(-c2cccc(-c3ccc4c5ccccc5n(-c5ccc(-c6nc(-c7ccccc7)nc(C7(c8ccccc8)c8ccccc8-c8ccccc87)n6)cc5)c4c3)c2)cc1 ; c1ccc(-c2ccc(-c3nc(-c4cccc(-n5c6ccccc6c6ccc(-c7ccccc7)cc65)c4)nc(C4(c5ccccc5)c5ccccc5-c5ccccc54)n3)cc2)cc1 ; c1ccc(-c2cccc(-c3nc(-c4cccc(-n5c6ccccc6c6c(-c7ccccc7)cccc65)c4)nc(C4(c5ccccc5)c5ccccc5-c5ccccc54)n3)c2)cc1]

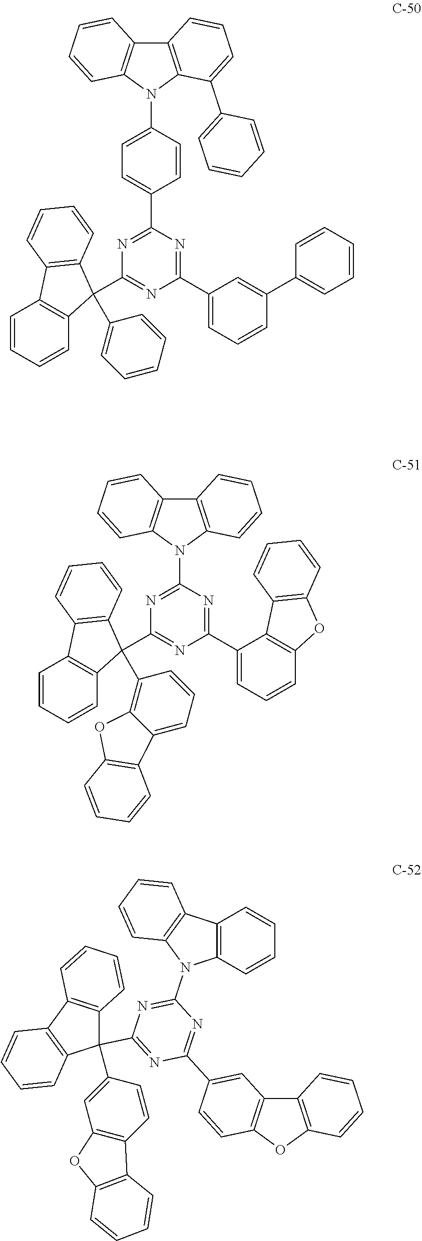 [SMILES: c1ccc(-c2cccc(-c3nc(-c4ccc(-n5c6ccccc6c6cccc(-c7ccccc7)c65)cc4)nc(C4(c5ccccc5)c5ccccc5-c5ccccc54)n3)c2)cc1 ; c1ccc2c(c1)-c1ccccc1C2(c1nc(-c2cccc3oc4ccccc4c23)nc(-n2c3ccccc3c3ccccc32)n1)c1cccc2c1oc1ccccc12 ; c1ccc2c(c1)-c1ccccc1C2(c1ccc2c(c1)oc1ccccc12)c1nc(-c2ccc3oc4ccccc4c3c2)nc(-n2c3ccccc3c3ccccc32)n1]

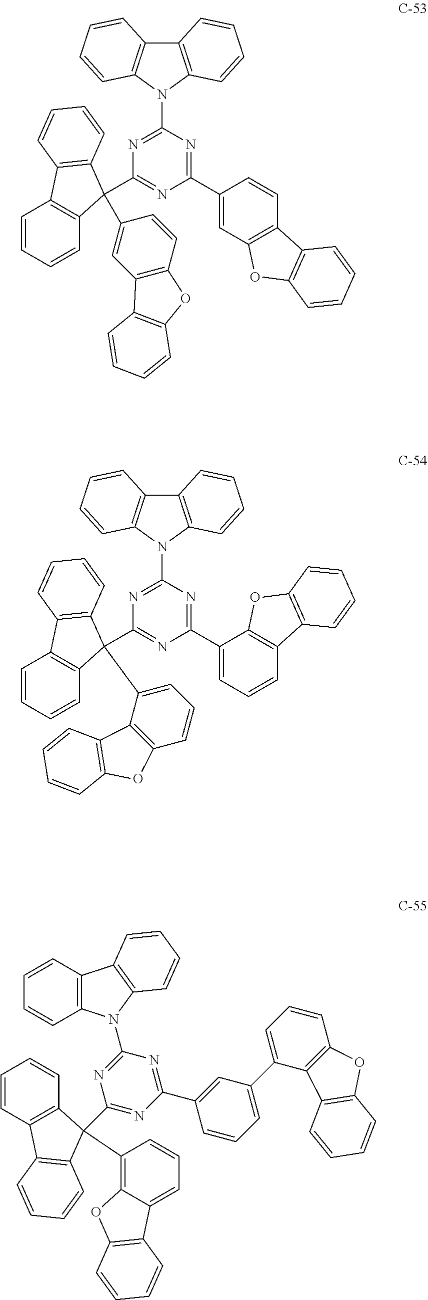 [SMILES: c1ccc2c(c1)-c1ccccc1C2(c1ccc2oc3ccccc3c2c1)c1nc(-c2ccc3c(c2)oc2ccccc23)nc(-n2c3ccccc3c3ccccc32)n1 ; c1ccc2c(c1)-c1ccccc1C2(c1nc(-c2cccc3c2oc2ccccc23)nc(-n2c3ccccc3c3ccccc32)n1)c1cccc2oc3ccccc3c12 ; c1cc(-c2nc(-n3c4ccccc4c4ccccc43)nc(C3(c4cccc5c4oc4ccccc45)c4ccccc4-c4ccccc43)n2)cc(-c2cccc3oc4ccccc4c23)c1]

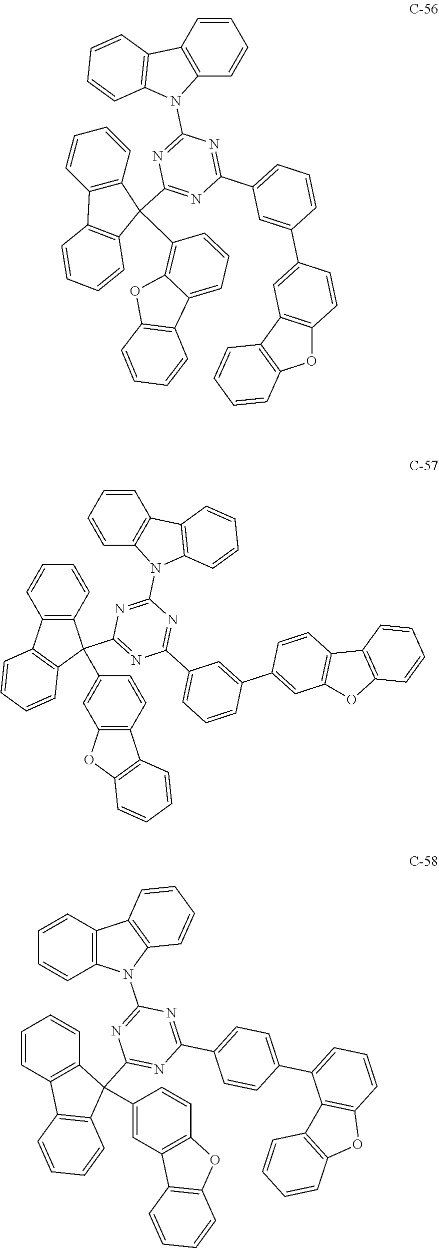 [SMILES: c1cc(-c2ccc3oc4ccccc4c3c2)cc(-c2nc(-n3c4ccccc4c4ccccc43)nc(C3(c4cccc5c4oc4ccccc45)c4ccccc4-c4ccccc43)n2)c1 ; c1cc(-c2ccc3c(c2)oc2ccccc23)cc(-c2nc(-n3c4ccccc4c4ccccc43)nc(C3(c4ccc5c(c4)oc4ccccc45)c4ccccc4-c4ccccc43)n2)c1 ; c1ccc2c(c1)-c1ccccc1C2(c1ccc2oc3ccccc3c2c1)c1nc(-c2ccc(-c3cccc4oc5ccccc5c34)cc2)nc(-n2c3ccccc3c3ccccc32)n1]

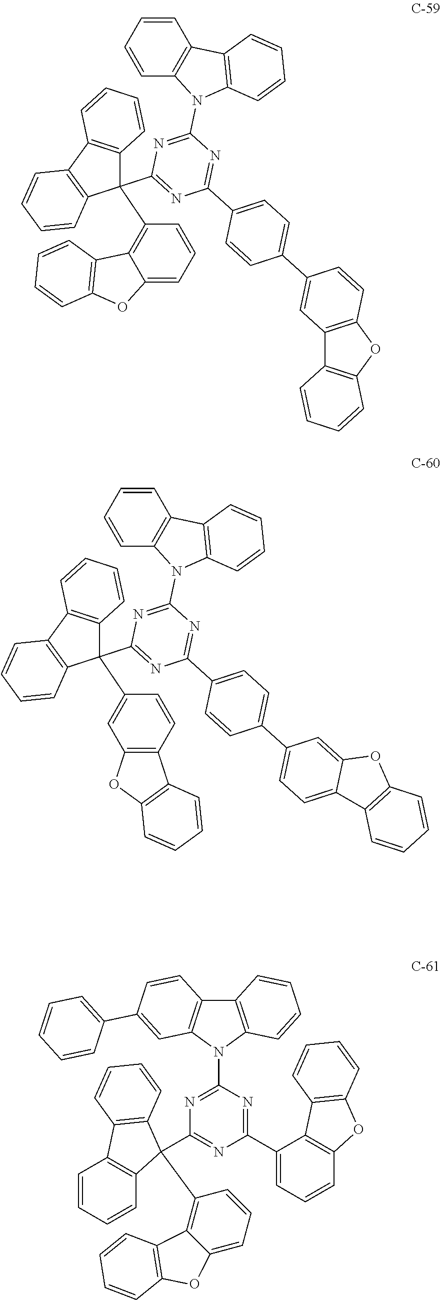 [SMILES: c1ccc2c(c1)-c1ccccc1C2(c1nc(-c2ccc(-c3ccc4oc5ccccc5c4c3)cc2)nc(-n2c3ccccc3c3ccccc32)n1)c1cccc2oc3ccccc3c12 ; c1ccc2c(c1)-c1ccccc1C2(c1ccc2c(c1)oc1ccccc12)c1nc(-c2ccc(-c3ccc4c(c3)oc3ccccc34)cc2)nc(-n2c3ccccc3c3ccccc32)n1 ; c1ccc(-c2ccc3c4ccccc4n(-c4nc(-c5cccc6oc7ccccc7c56)nc(C5(c6cccc7oc8ccccc8c67)c6ccccc6-c6ccccc65)n4)c3c2)cc1]

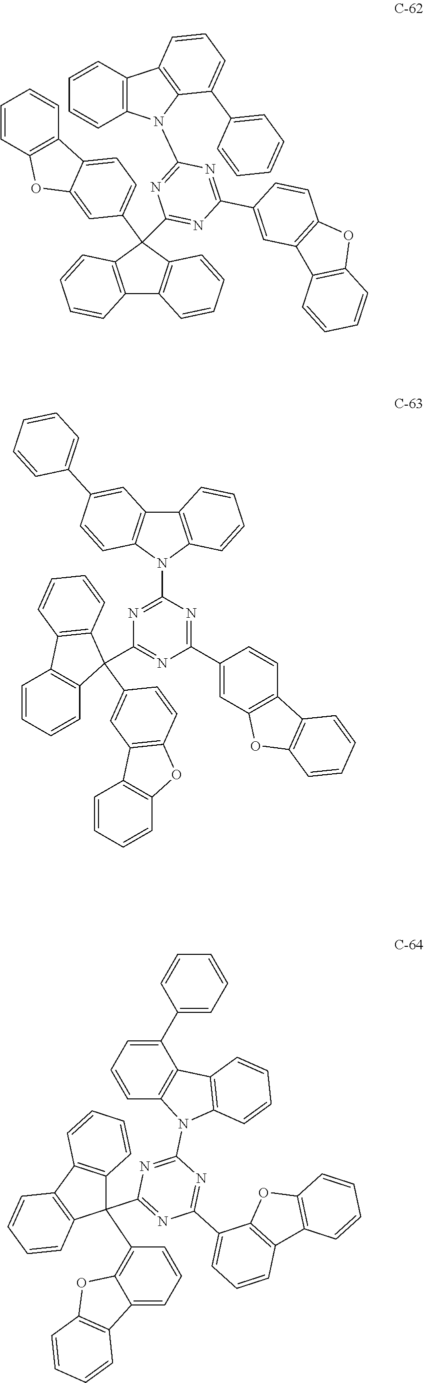 [SMILES: c1ccc(-c2cccc3c4ccccc4n(-c4nc(-c5ccc6oc7ccccc7c6c5)nc(C5(c6ccc7c(c6)oc6ccccc67)c6ccccc6-c6ccccc65)n4)c23)cc1 ; c1ccc(-c2ccc3c(c2)c2ccccc2n3-c2nc(-c3ccc4c(c3)oc3ccccc34)nc(C3(c4ccc5oc6ccccc6c5c4)c4ccccc4-c4ccccc43)n2)cc1 ; c1ccc(-c2cccc3c2c2ccccc2n3-c2nc(-c3cccc4c3oc3ccccc34)nc(C3(c4cccc5c4oc4ccccc45)c4ccccc4-c4ccccc43)n2)cc1]

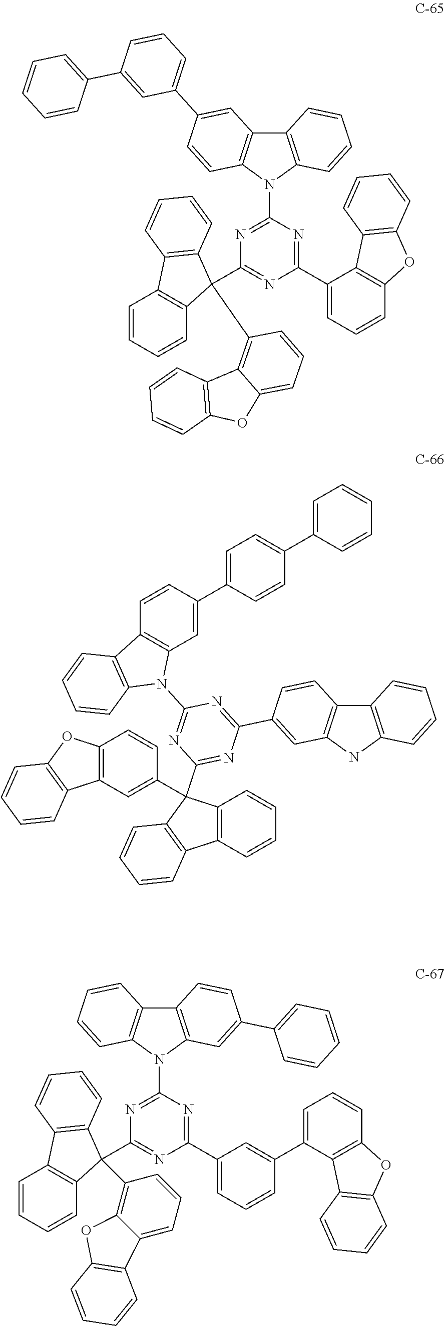 [SMILES: c1ccc(-c2cccc(-c3ccc4c(c3)c3ccccc3n4-c3nc(-c4cccc5oc6ccccc6c45)nc(C4(c5cccc6oc7ccccc7c56)c5ccccc5-c5ccccc54)n3)c2)cc1 ; c1ccc(-c2ccc(-c3ccc4c5ccccc5n(-c5nc(-c6ccc7c(c6)[nH]c6ccccc67)nc(C6(c7ccc8oc9ccccc9c8c7)c7ccccc7-c7ccccc76)n5)c4c3)cc2)cc1 ; c1ccc(-c2ccc3c4ccccc4n(-c4nc(-c5cccc(-c6cccc7oc8ccccc8c67)c5)nc(C5(c6cccc7c6oc6ccccc67)c6ccccc6-c6ccccc65)n4)c3c2)cc1]

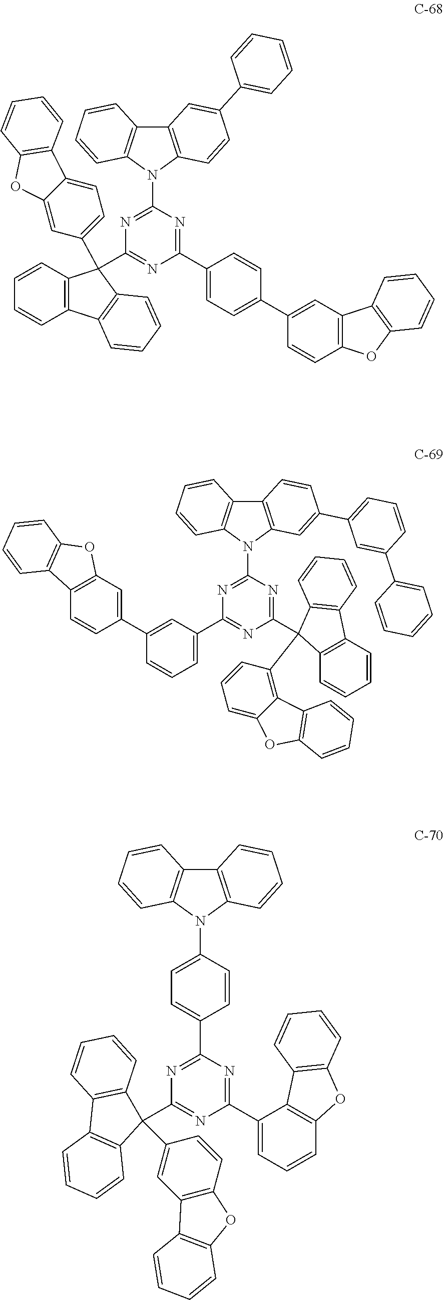 [SMILES: c1ccc(-c2ccc3c(c2)c2ccccc2n3-c2nc(-c3ccc(-c4ccc5oc6ccccc6c5c4)cc3)nc(C3(c4ccc5c(c4)oc4ccccc45)c4ccccc4-c4ccccc43)n2)cc1 ; c1ccc(-c2cccc(-c3ccc4c5ccccc5n(-c5nc(-c6cccc(-c7ccc8c(c7)oc7ccccc78)c6)nc(C6(c7cccc8oc9ccccc9c78)c7ccccc7-c7ccccc76)n5)c4c3)c2)cc1 ; c1ccc2c(c1)-c1ccccc1C2(c1ccc2oc3ccccc3c2c1)c1nc(-c2ccc(-n3c4ccccc4c4ccccc43)cc2)nc(-c2cccc3oc4ccccc4c23)n1]

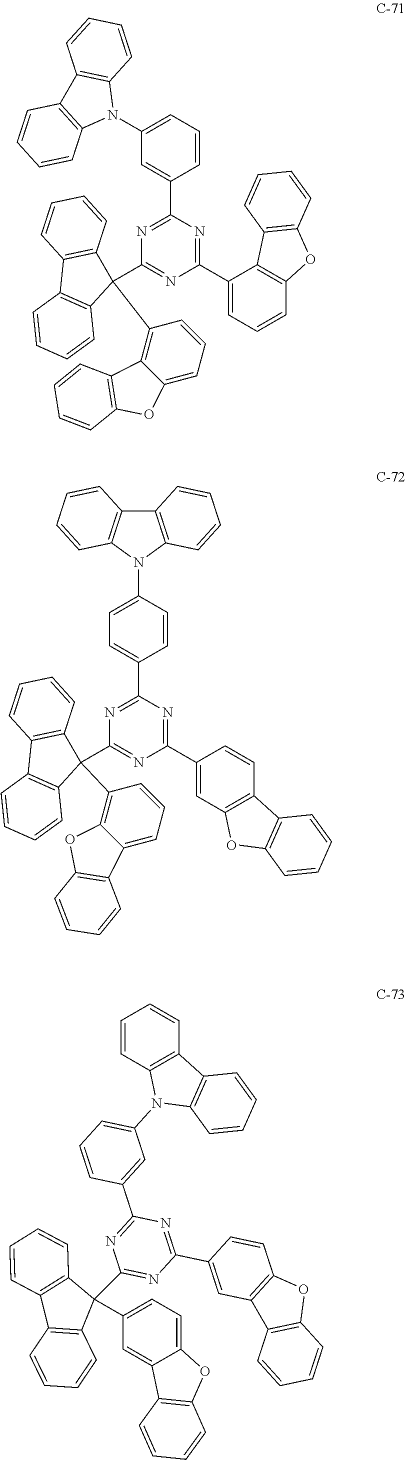 [SMILES: c1cc(-c2nc(-c3cccc4oc5ccccc5c34)nc(C3(c4cccc5oc6ccccc6c45)c4ccccc4-c4ccccc43)n2)cc(-n2c3ccccc3c3ccccc32)c1 ; c1ccc2c(c1)-c1ccccc1C2(c1nc(-c2ccc(-n3c4ccccc4c4ccccc43)cc2)nc(-c2ccc3c(c2)oc2ccccc23)n1)c1cccc2c1oc1ccccc12 ; c1cc(-c2nc(-c3ccc4oc5ccccc5c4c3)nc(C3(c4ccc5oc6ccccc6c5c4)c4ccccc4-c4ccccc43)n2)cc(-n2c3ccccc3c3ccccc32)c1]

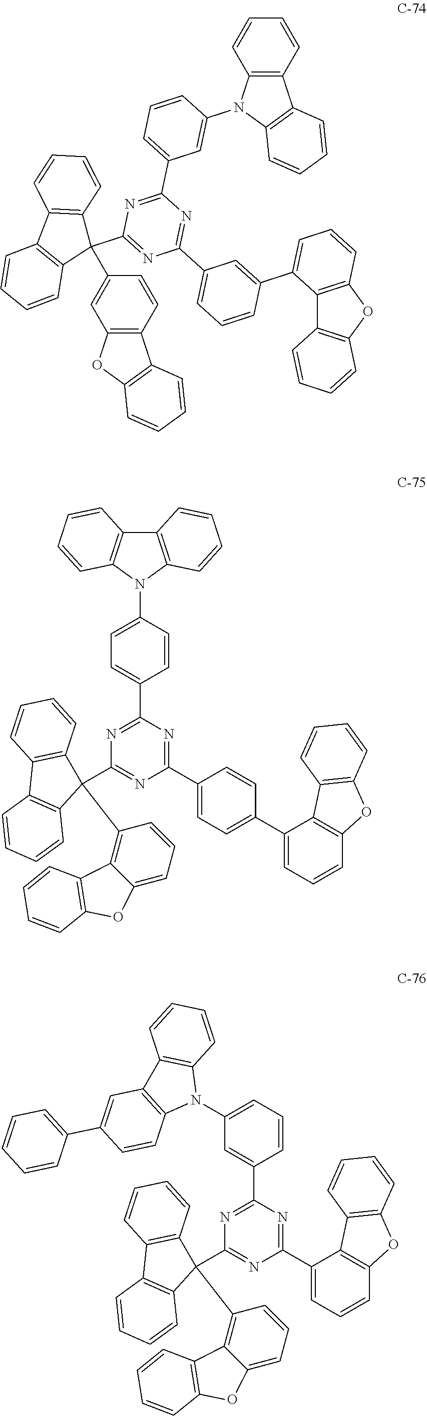 [SMILES: c1cc(-c2nc(-c3cccc(-n4c5ccccc5c5ccccc54)c3)nc(C3(c4ccc5c(c4)oc4ccccc45)c4ccccc4-c4ccccc43)n2)cc(-c2cccc3oc4ccccc4c23)c1 ; c1ccc2c(c1)-c1ccccc1C2(c1nc(-c2ccc(-c3cccc4oc5ccccc5c34)cc2)nc(-c2ccc(-n3c4ccccc4c4ccccc43)cc2)n1)c1cccc2oc3ccccc3c12 ; c1ccc(-c2ccc3c(c2)c2ccccc2n3-c2cccc(-c3nc(-c4cccc5oc6ccccc6c45)nc(C4(c5cccc6oc7ccccc7c56)c5ccccc5-c5ccccc54)n3)c2)cc1]

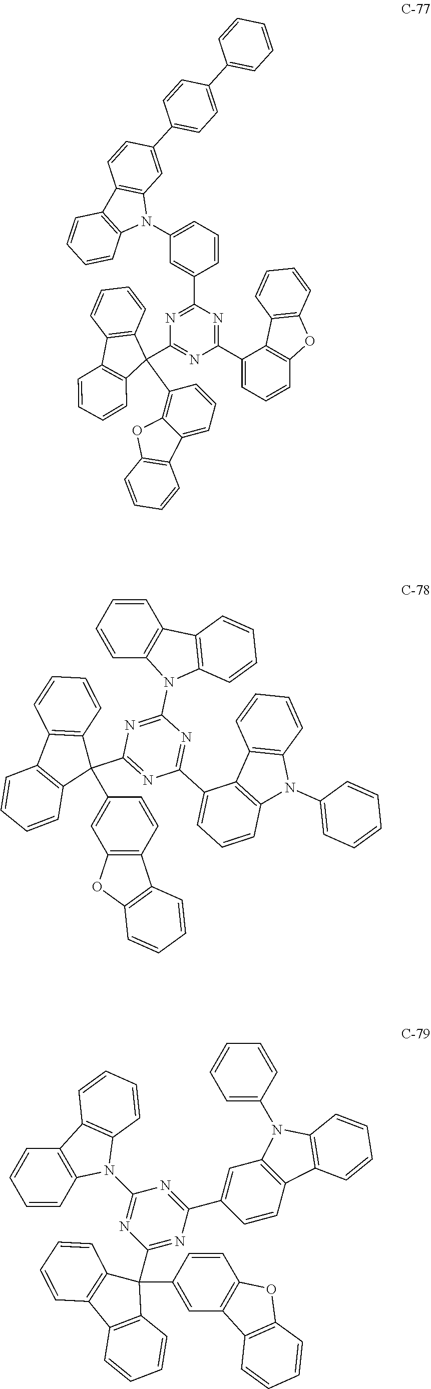 [SMILES: c1ccc(-c2ccc(-c3ccc4c5ccccc5n(-c5cccc(-c6nc(-c7cccc8oc9ccccc9c78)nc(C7(c8cccc9c8oc8ccccc89)c8ccccc8-c8ccccc87)n6)c5)c4c3)cc2)cc1 ; c1ccc(-n2c3ccccc3c3c(-c4nc(-n5c6ccccc6c6ccccc65)nc(C5(c6ccc7c(c6)oc6ccccc67)c6ccccc6-c6ccccc65)n4)cccc32)cc1 ; c1ccc(-n2c3ccccc3c3ccc(-c4nc(-n5c6ccccc6c6ccccc65)nc(C5(c6ccc7oc8ccccc8c7c6)c6ccccc6-c6ccccc65)n4)cc32)cc1]

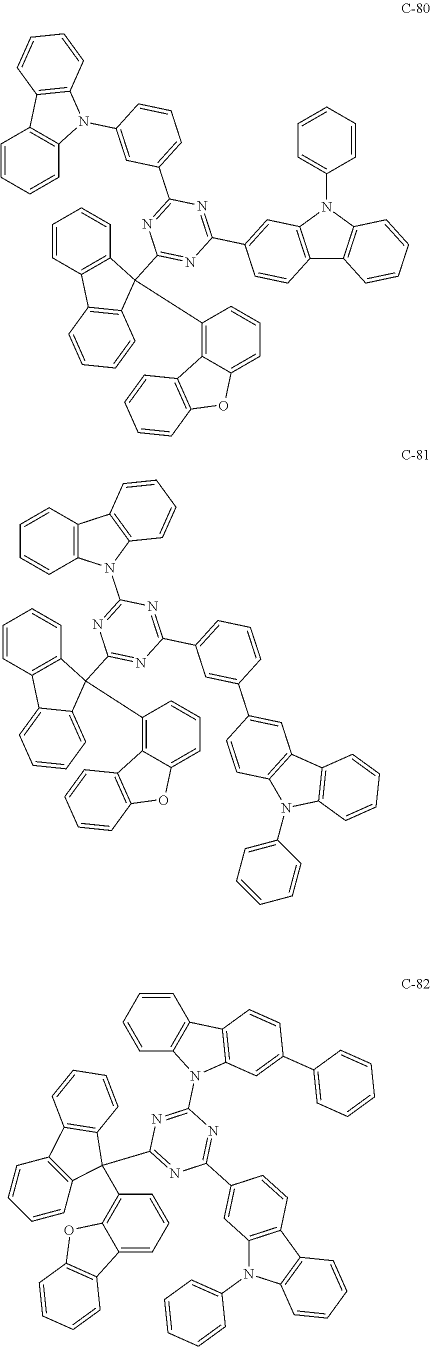

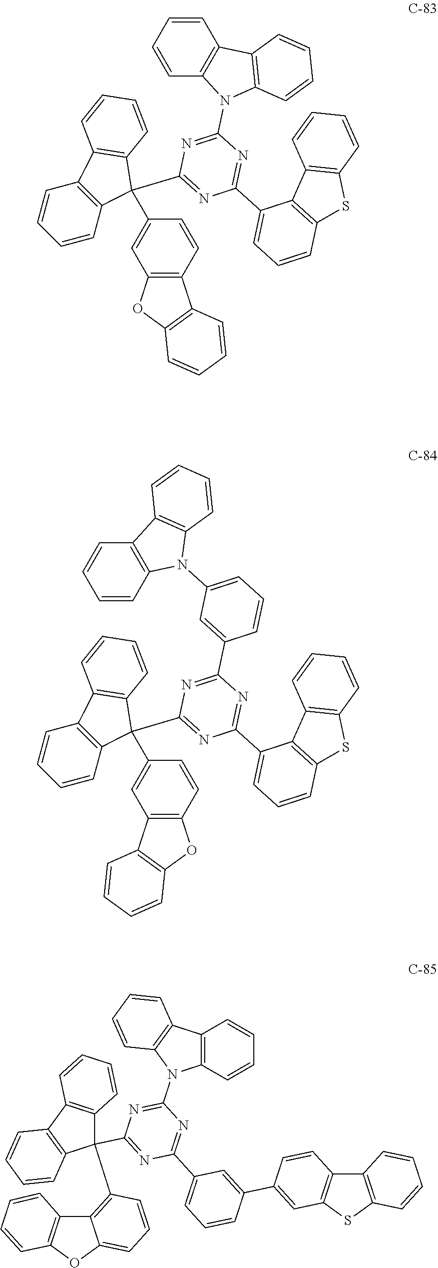 [SMILES: c1ccc2c(c1)-c1ccccc1C2(c1ccc2c(c1)oc1ccccc12)c1nc(-c2cccc3sc4ccccc4c23)nc(-n2c3ccccc3c3ccccc32)n1 ; c1cc(-c2nc(-c3cccc4sc5ccccc5c34)nc(C3(c4ccc5oc6ccccc6c5c4)c4ccccc4-c4ccccc43)n2)cc(-n2c3ccccc3c3ccccc32)c1 ; c1cc(-c2ccc3c(c2)sc2ccccc23)cc(-c2nc(-n3c4ccccc4c4ccccc43)nc(C3(c4cccc5oc6ccccc6c45)c4ccccc4-c4ccccc43)n2)c1]

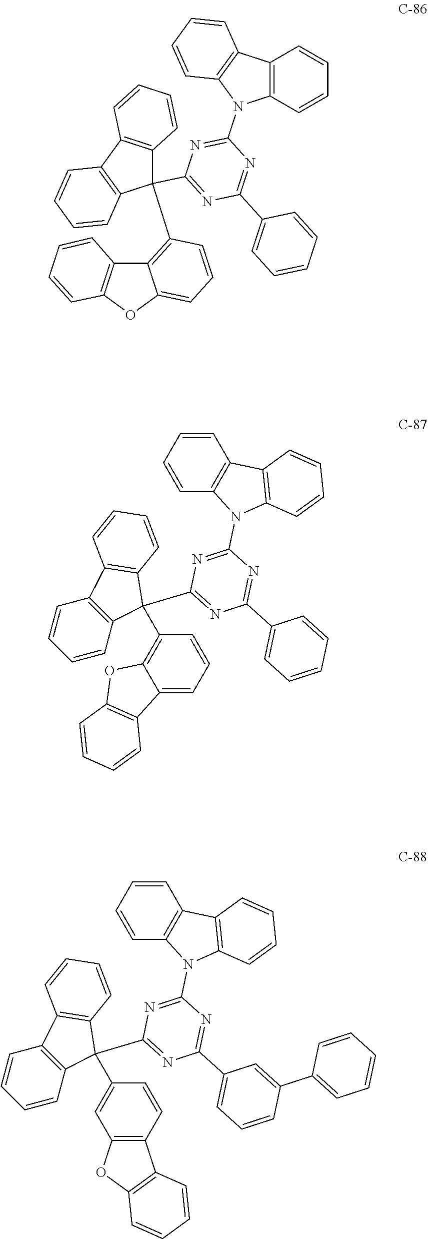 [SMILES: c1ccc(-c2nc(-n3c4ccccc4c4ccccc43)nc(C3(c4cccc5oc6ccccc6c45)c4ccccc4-c4ccccc43)n2)cc1 ; c1ccc(-c2nc(-n3c4ccccc4c4ccccc43)nc(C3(c4cccc5c4oc4ccccc45)c4ccccc4-c4ccccc43)n2)cc1 ; c1ccc(-c2cccc(-c3nc(-n4c5ccccc5c5ccccc54)nc(C4(c5ccc6c(c5)oc5ccccc56)c5ccccc5-c5ccccc54)n3)c2)cc1]

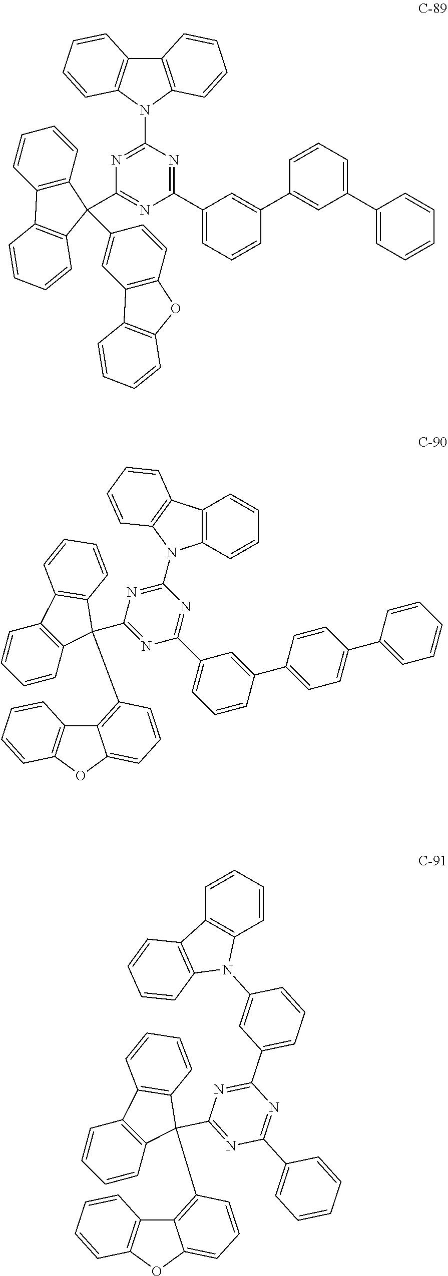 [SMILES: c1ccc(-c2cccc(-c3cccc(-c4nc(-n5c6ccccc6c6ccccc65)nc(C5(c6ccc7oc8ccccc8c7c6)c6ccccc6-c6ccccc65)n4)c3)c2)cc1 ; c1ccc(-c2ccc(-c3cccc(-c4nc(-n5c6ccccc6c6ccccc65)nc(C5(c6cccc7oc8ccccc8c67)c6ccccc6-c6ccccc65)n4)c3)cc2)cc1 ; c1ccc(-c2nc(-c3cccc(-n4c5ccccc5c5ccccc54)c3)nc(C3(c4cccc5oc6ccccc6c45)c4ccccc4-c4ccccc43)n2)cc1]

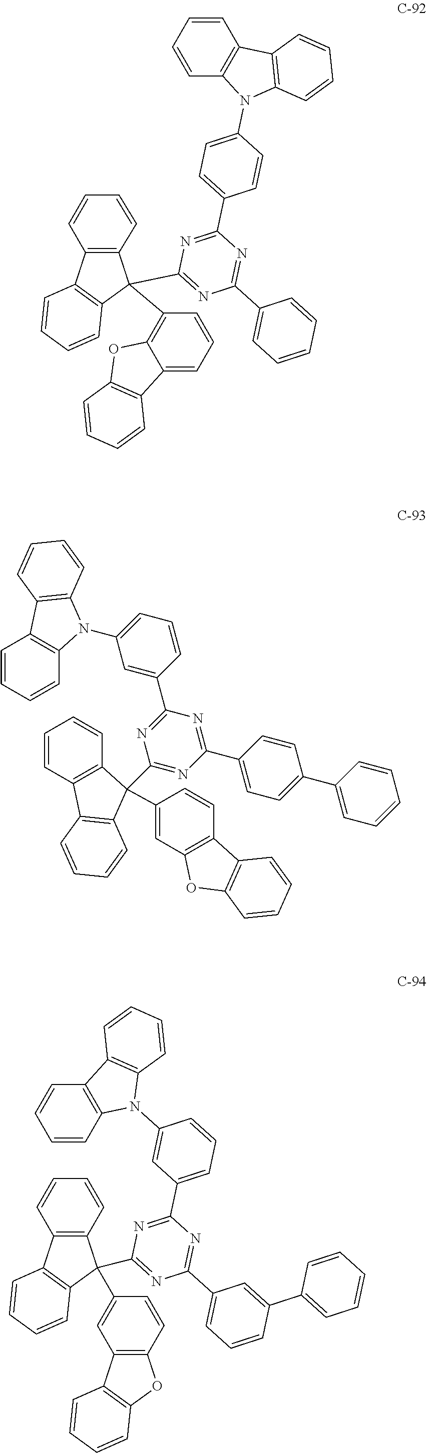 [SMILES: c1ccc(-c2nc(-c3ccc(-n4c5ccccc5c5ccccc54)cc3)nc(C3(c4cccc5c4oc4ccccc45)c4ccccc4-c4ccccc43)n2)cc1 ; c1ccc(-c2ccc(-c3nc(-c4cccc(-n5c6ccccc6c6ccccc65)c4)nc(C4(c5ccc6c(c5)oc5ccccc56)c5ccccc5-c5ccccc54)n3)cc2)cc1 ; c1ccc(-c2cccc(-c3nc(-c4cccc(-n5c6ccccc6c6ccccc65)c4)nc(C4(c5ccc6oc7ccccc7c6c5)c5ccccc5-c5ccccc54)n3)c2)cc1]

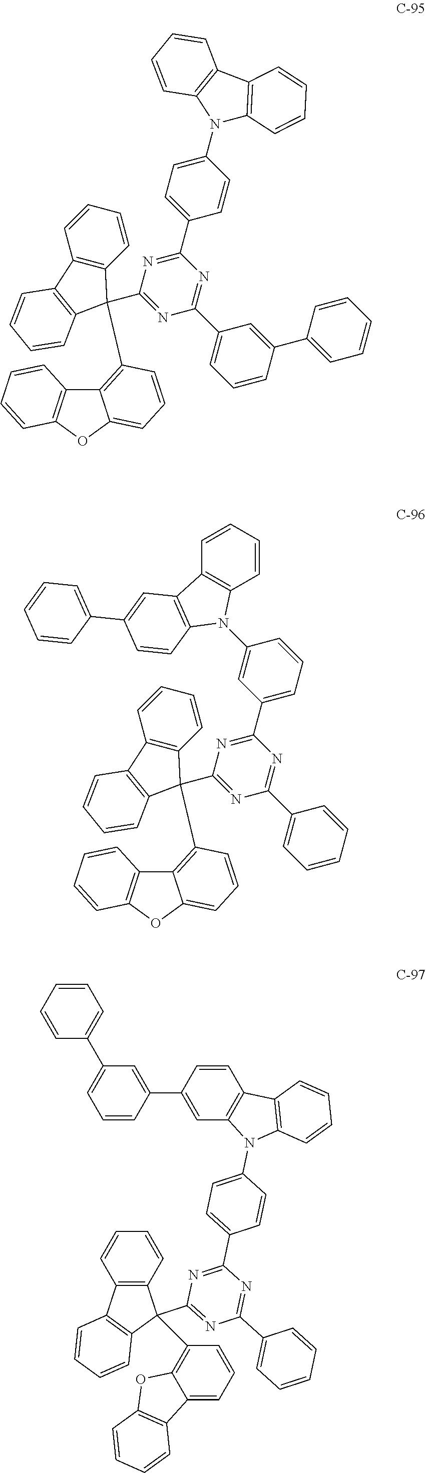 [SMILES: c1ccc(-c2cccc(-c3nc(-c4ccc(-n5c6ccccc6c6ccccc65)cc4)nc(C4(c5cccc6oc7ccccc7c56)c5ccccc5-c5ccccc54)n3)c2)cc1 ; c1ccc(-c2ccc3c(c2)c2ccccc2n3-c2cccc(-c3nc(-c4ccccc4)nc(C4(c5cccc6oc7ccccc7c56)c5ccccc5-c5ccccc54)n3)c2)cc1 ; c1ccc(-c2cccc(-c3ccc4c5ccccc5n(-c5ccc(-c6nc(-c7ccccc7)nc(C7(c8cccc9c8oc8ccccc89)c8ccccc8-c8ccccc87)n6)cc5)c4c3)c2)cc1]

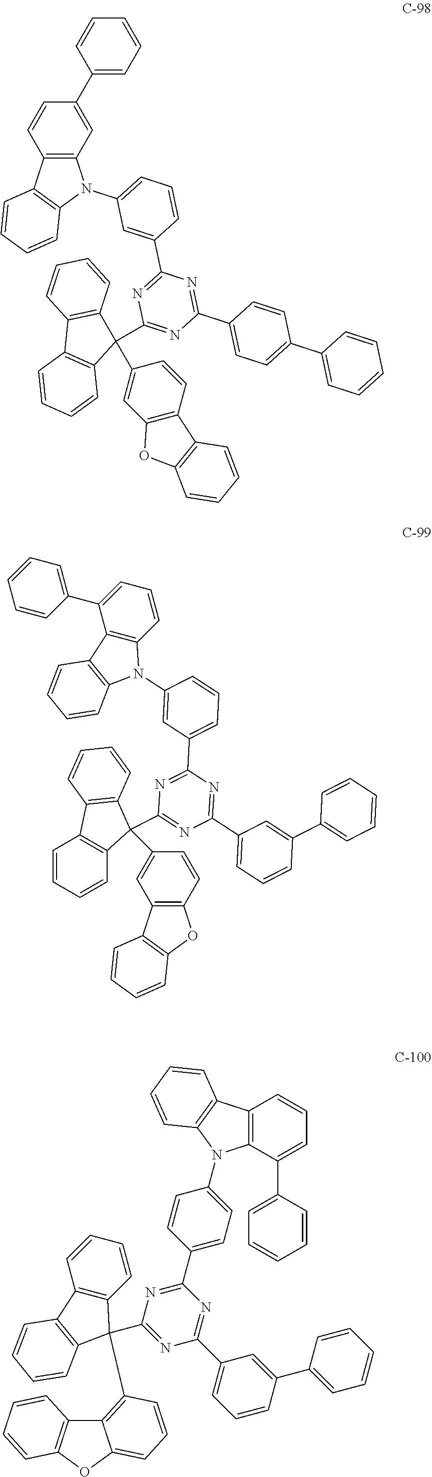 [SMILES: c1ccc(-c2ccc(-c3nc(-c4cccc(-n5c6ccccc6c6ccc(-c7ccccc7)cc65)c4)nc(C4(c5ccc6c(c5)oc5ccccc56)c5ccccc5-c5ccccc54)n3)cc2)cc1 ; c1ccc(-c2cccc(-c3nc(-c4cccc(-n5c6ccccc6c6c(-c7ccccc7)cccc65)c4)nc(C4(c5ccc6oc7ccccc7c6c5)c5ccccc5-c5ccccc54)n3)c2)cc1 ; c1ccc(-c2cccc(-c3nc(-c4ccc(-n5c6ccccc6c6cccc(-c7ccccc7)c65)cc4)nc(C4(c5cccc6oc7ccccc7c56)c5ccccc5-c5ccccc54)n3)c2)cc1]

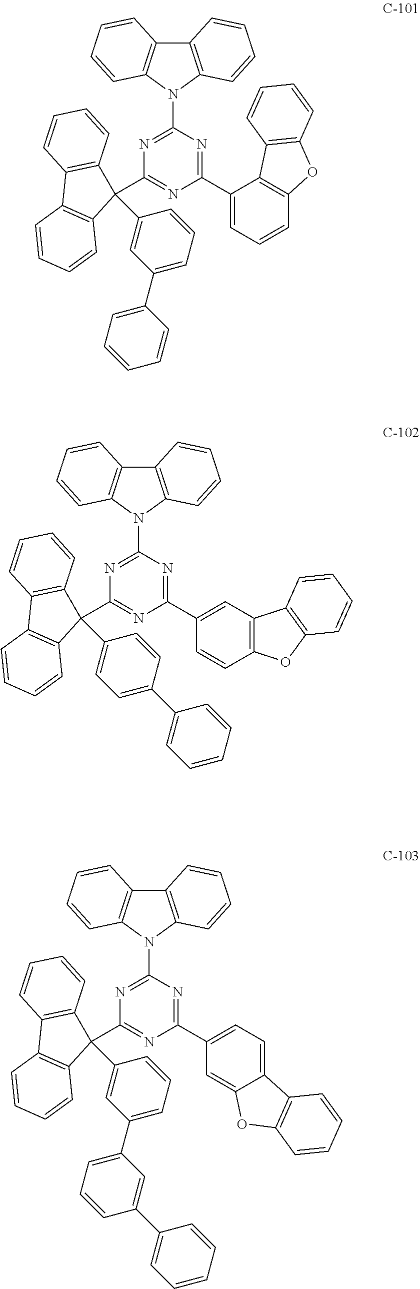 [SMILES: c1ccc(-c2cccc(C3(c4nc(-c5cccc6oc7ccccc7c56)nc(-n5c6ccccc6c6ccccc65)n4)c4ccccc4-c4ccccc43)c2)cc1 ; c1ccc(-c2ccc(C3(c4nc(-c5ccc6oc7ccccc7c6c5)nc(-n5c6ccccc6c6ccccc65)n4)c4ccccc4-c4ccccc43)cc2)cc1 ; c1ccc(-c2cccc(-c3cccc(C4(c5nc(-c6ccc7c(c6)oc6ccccc67)nc(-n6c7ccccc7c7ccccc76)n5)c5ccccc5-c5ccccc54)c3)c2)cc1]

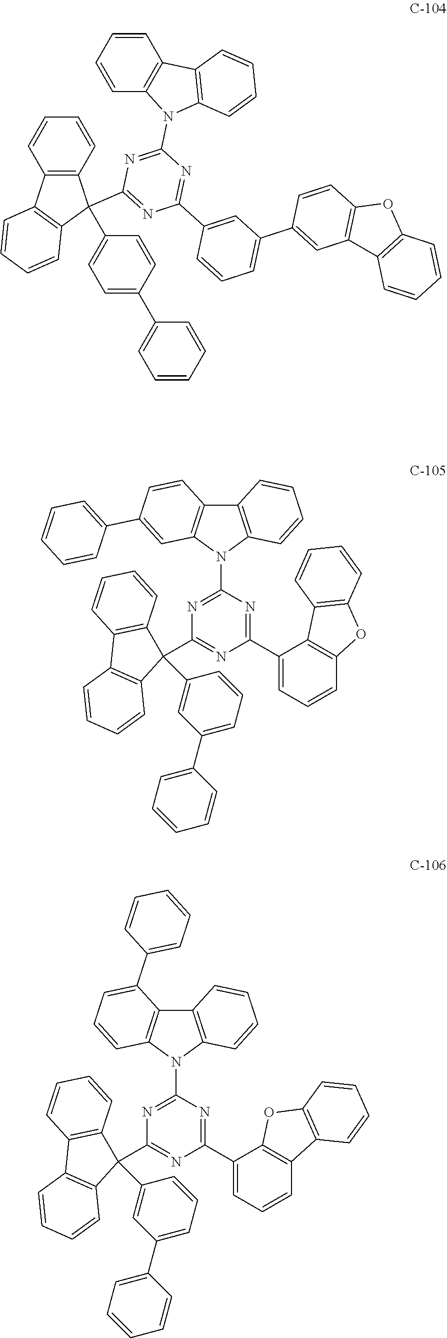 [SMILES: c1ccc(-c2ccc(C3(c4nc(-c5cccc(-c6ccc7oc8ccccc8c7c6)c5)nc(-n5c6ccccc6c6ccccc65)n4)c4ccccc4-c4ccccc43)cc2)cc1 ; c1ccc(-c2cccc(C3(c4nc(-c5cccc6oc7ccccc7c56)nc(-n5c6ccccc6c6ccc(-c7ccccc7)cc65)n4)c4ccccc4-c4ccccc43)c2)cc1 ; c1ccc(-c2cccc(C3(c4nc(-c5cccc6c5oc5ccccc56)nc(-n5c6ccccc6c6c(-c7ccccc7)cccc65)n4)c4ccccc4-c4ccccc43)c2)cc1]

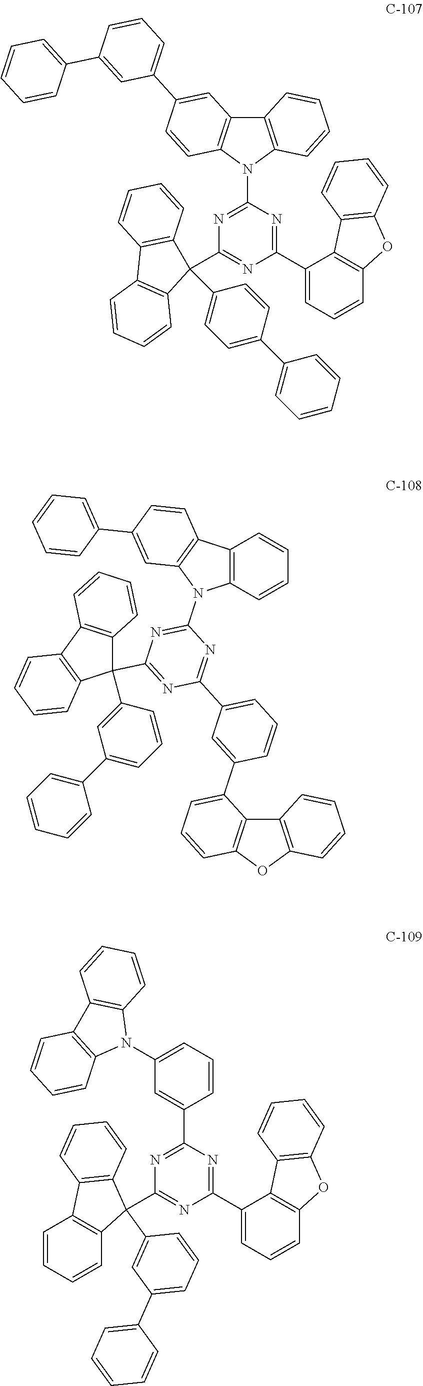 [SMILES: c1ccc(-c2ccc(C3(c4nc(-c5cccc6oc7ccccc7c56)nc(-n5c6ccccc6c6cc(-c7cccc(-c8ccccc8)c7)ccc65)n4)c4ccccc4-c4ccccc43)cc2)cc1 ; c1ccc(-c2cccc(C3(c4nc(-c5cccc(-c6cccc7oc8ccccc8c67)c5)nc(-n5c6ccccc6c6ccc(-c7ccccc7)cc65)n4)c4ccccc4-c4ccccc43)c2)cc1 ; c1ccc(-c2cccc(C3(c4nc(-c5cccc(-n6c7ccccc7c7ccccc76)c5)nc(-c5cccc6oc7ccccc7c56)n4)c4ccccc4-c4ccccc43)c2)cc1]

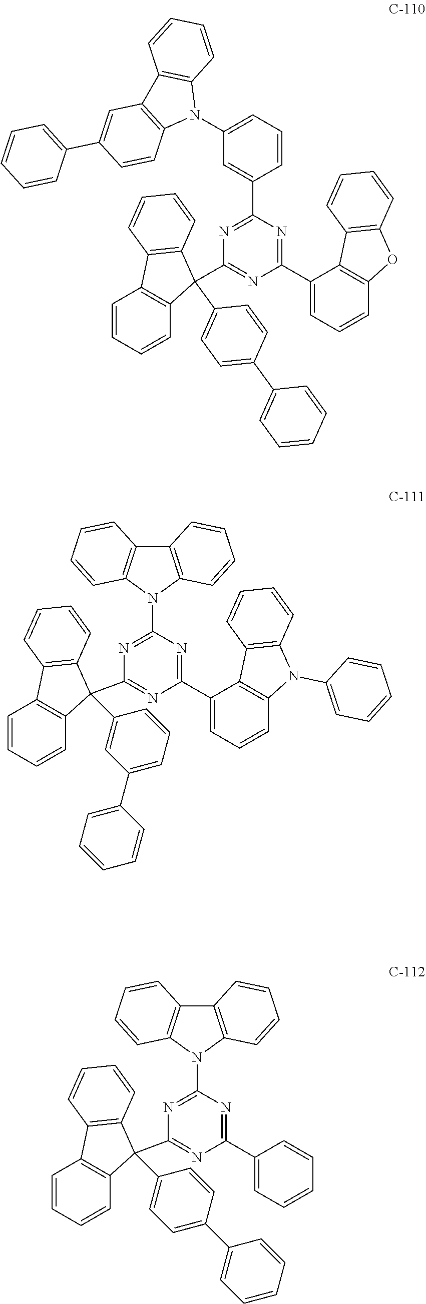 [SMILES: c1ccc(-c2ccc(C3(c4nc(-c5cccc(-n6c7ccccc7c7cc(-c8ccccc8)ccc76)c5)nc(-c5cccc6oc7ccccc7c56)n4)c4ccccc4-c4ccccc43)cc2)cc1 ; c1ccc(-c2cccc(C3(c4nc(-c5cccc6c5c5ccccc5n6-c5ccccc5)nc(-n5c6ccccc6c6ccccc65)n4)c4ccccc4-c4ccccc43)c2)cc1 ; c1ccc(-c2ccc(C3(c4nc(-c5ccccc5)nc(-n5c6ccccc6c6ccccc65)n4)c4ccccc4-c4ccccc43)cc2)cc1]

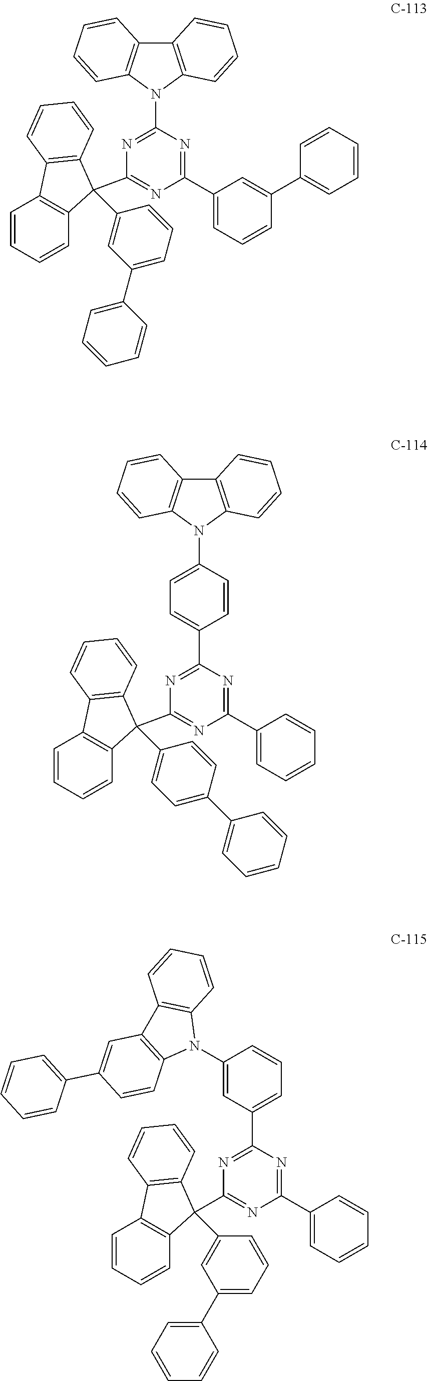 [SMILES: c1ccc(-c2cccc(-c3nc(-n4c5ccccc5c5ccccc54)nc(C4(c5cccc(-c6ccccc6)c5)c5ccccc5-c5ccccc54)n3)c2)cc1 ; c1ccc(-c2ccc(C3(c4nc(-c5ccccc5)nc(-c5ccc(-n6c7ccccc7c7ccccc76)cc5)n4)c4ccccc4-c4ccccc43)cc2)cc1 ; c1ccc(-c2cccc(C3(c4nc(-c5ccccc5)nc(-c5cccc(-n6c7ccccc7c7cc(-c8ccccc8)ccc76)c5)n4)c4ccccc4-c4ccccc43)c2)cc1]

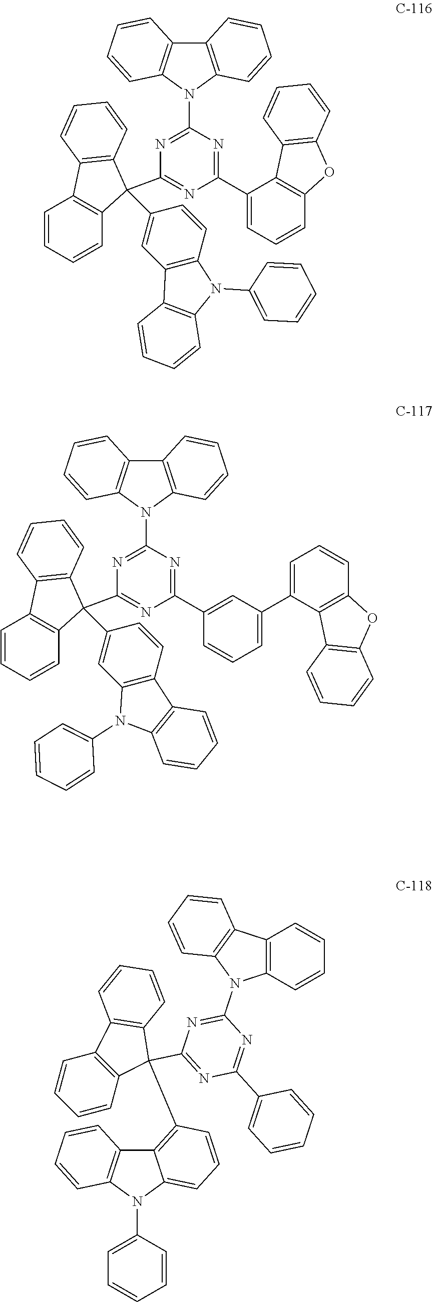

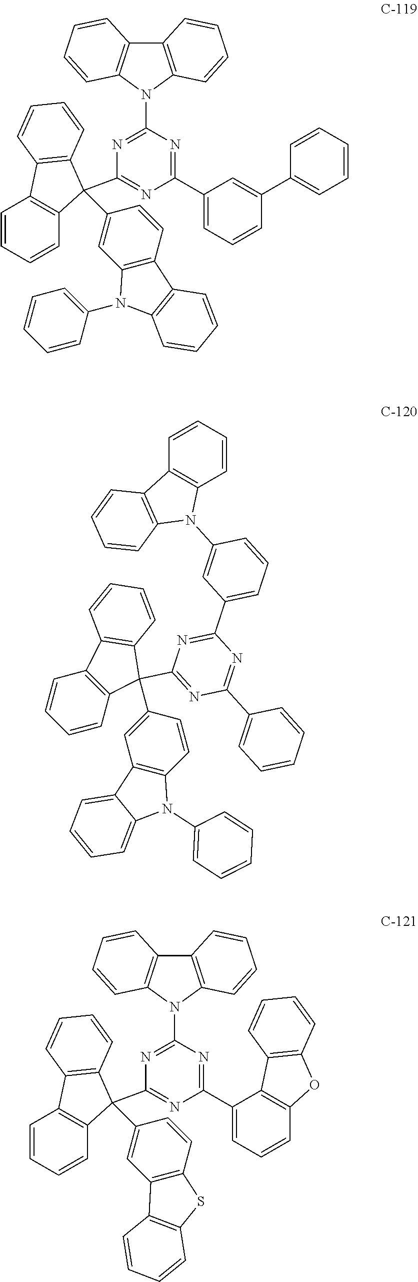 [SMILES: c1ccc(-c2cccc(-c3nc(-n4c5ccccc5c5ccccc54)nc(C4(c5ccc6c7ccccc7n(-c7ccccc7)c6c5)c5ccccc5-c5ccccc54)n3)c2)cc1 ; c1ccc(-c2nc(-c3cccc(-n4c5ccccc5c5ccccc54)c3)nc(C3(c4ccc5c(c4)c4ccccc4n5-c4ccccc4)c4ccccc4-c4ccccc43)n2)cc1 ; c1ccc2c(c1)-c1ccccc1C2(c1ccc2sc3ccccc3c2c1)c1nc(-c2cccc3oc4ccccc4c23)nc(-n2c3ccccc3c3ccccc32)n1]

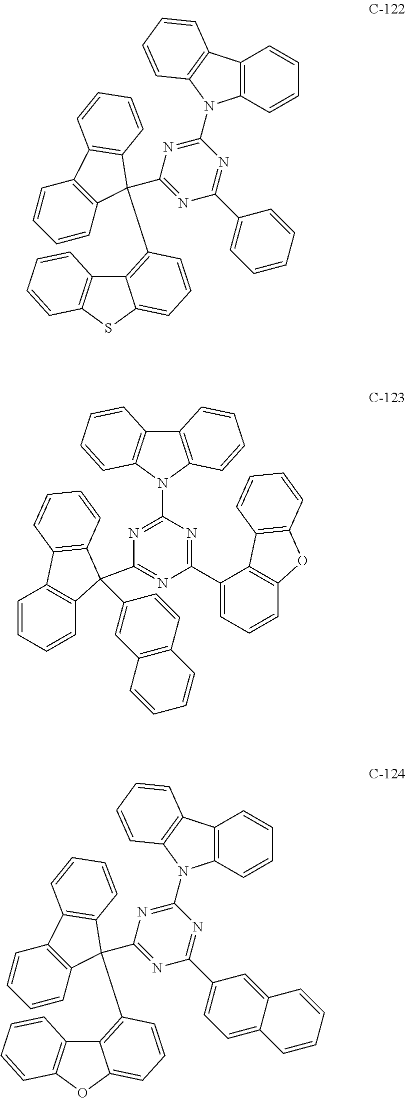 [SMILES: c1ccc(-c2nc(-n3c4ccccc4c4ccccc43)nc(C3(c4cccc5sc6ccccc6c45)c4ccccc4-c4ccccc43)n2)cc1 ; c1ccc2c(c1)-c1ccccc1C2(c1ccc2ccccc2c1)c1nc(-c2cccc3oc4ccccc4c23)nc(-n2c3ccccc3c3ccccc32)n1 ; c1ccc2c(c1)-c1ccccc1C2(c1nc(-c2ccc3ccccc3c2)nc(-n2c3ccccc3c3ccccc32)n1)c1cccc2oc3ccccc3c12]

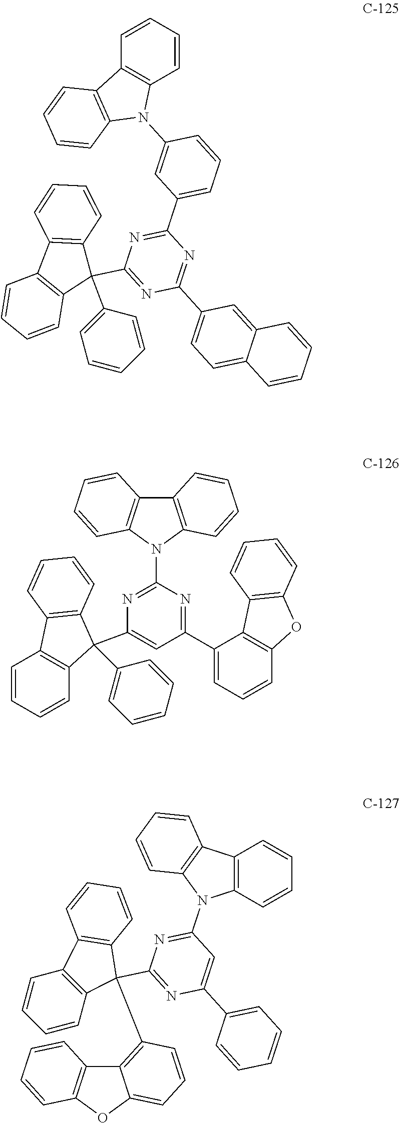 [SMILES: c1ccc(C2(c3nc(-c4cccc(-n5c6ccccc6c6ccccc65)c4)nc(-c4ccc5ccccc5c4)n3)c3ccccc3-c3ccccc32)cc1 ; c1ccc(C2(c3cc(-c4cccc5oc6ccccc6c45)nc(-n4c5ccccc5c5ccccc54)n3)c3ccccc3-c3ccccc32)cc1 ; c1ccc(-c2cc(-n3c4ccccc4c4ccccc43)nc(C3(c4cccc5oc6ccccc6c45)c4ccccc4-c4ccccc43)n2)cc1]

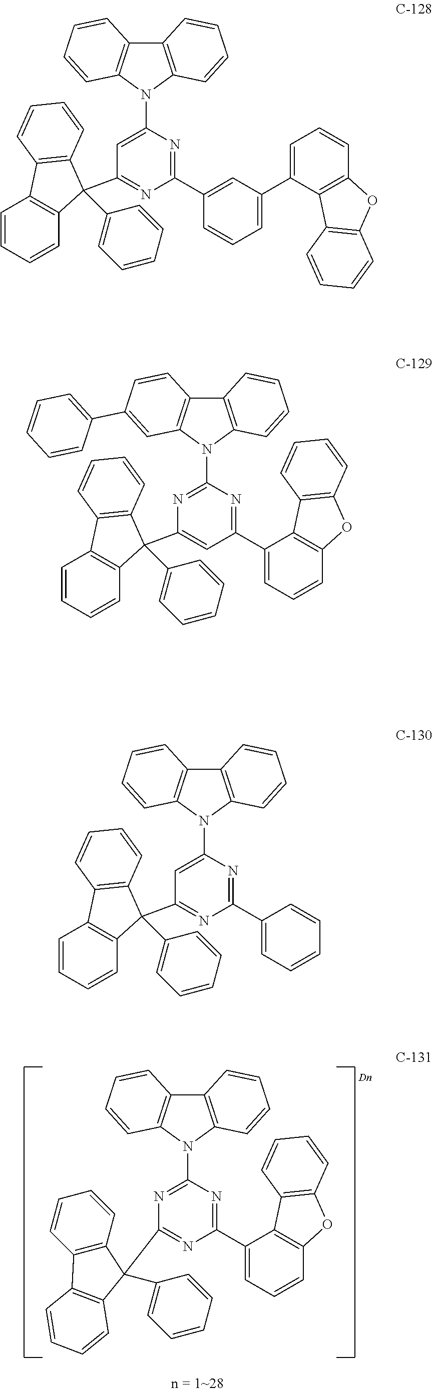

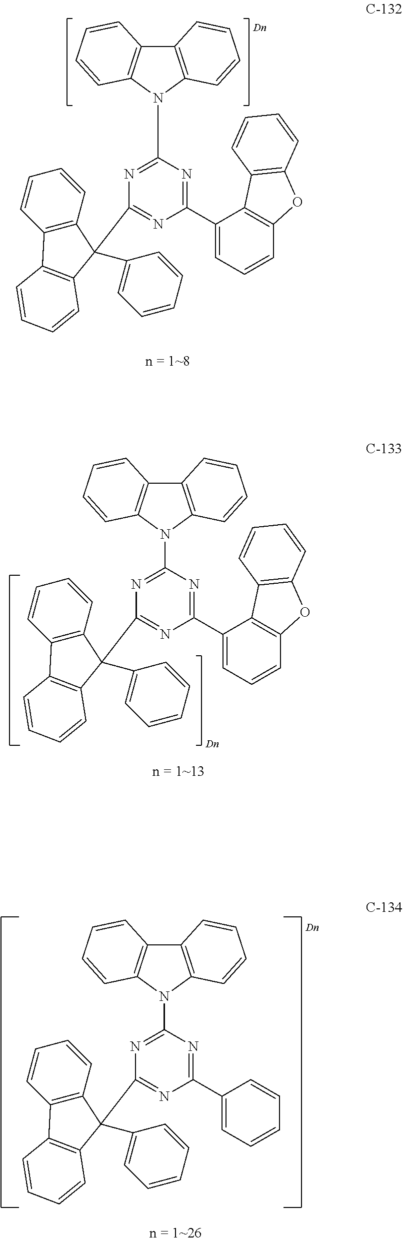

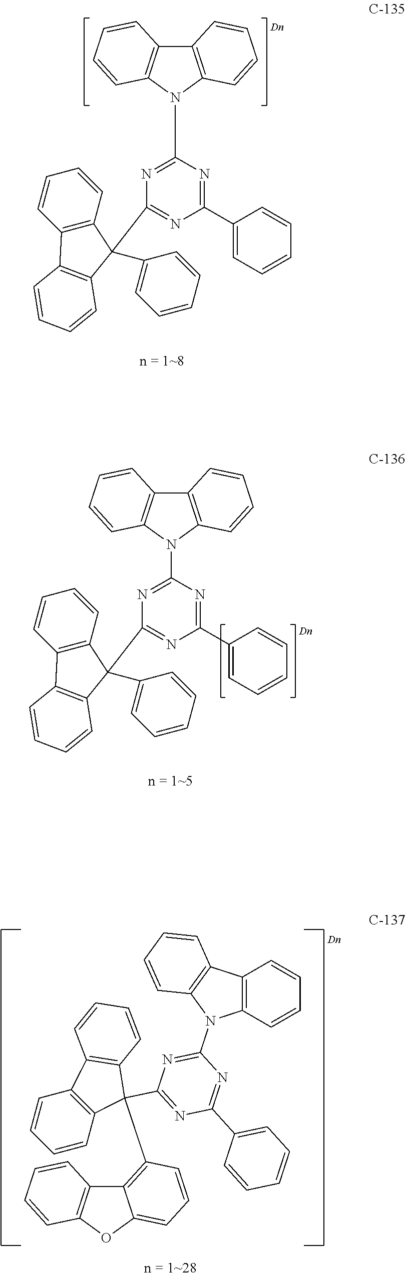

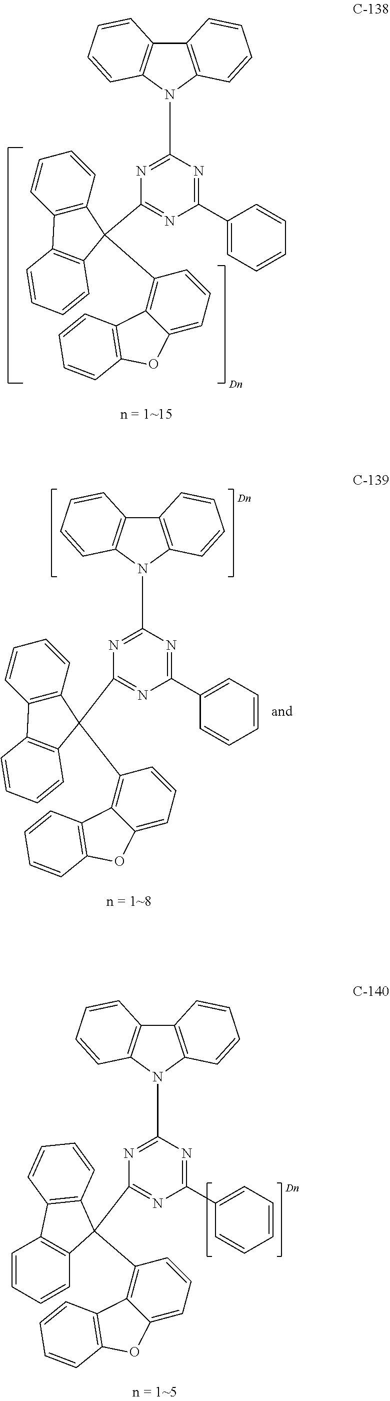

13. An organic electroluminescent device comprising an anode; a cathode; and at least one light-emitting layer between the anode and the cathode, wherein at least one layer of the light-emitting layers comprises the plurality of host materials according to claim 1.

14. An organic electroluminescent device comprising an anode, a cathode, and at least one light-emitting layer between the anode and the cathode, wherein at least one layer of the light-emitting layers comprises the organic electroluminescent compound according to claim 11.
